SEQUENCE LISTING (65 sequences)

SEQ ID NO: 1 - DNA, 24 bp, Artificial Sequence
  MHcL1 primer
atggacttgctggatacttgagct

SEQ ID NO: 2 - DNA, 27 bp, Artificial Sequence
  MHcL2 primer
atggaatggagcctgggatctttctct

SEQ ID NO: 3 - DNA, 27 bp, Artificial Sequence
  MHcL3 primer
atgaaagtgttgagtctgttgtacctg

SEQ ID NO: 4 - DNA, 33 bp, Artificial Sequence
  MHcL4 primer
atggaagcttgctgggtctacaagcttgggatt

SEQ ID NO: 5 - DNA, 21 bp, Artificial Sequence
  MG1-3Seq primer
agatgggggtgtcgttttggc

SEQ ID NO: 6 - DNA, 24 bp, Artificial Sequence
  MG2ab-3Seq primer
gacygatggggstgttgttttggc

SEQ ID NO: 7 - DNA, 22 bp, Artificial Sequence
  MKcL-1 primer
atgaagttgcctgttaggctgt

SEQ ID NO: 8 - DNA, 22 bp, Artificial Sequence
  MKcL-2 primer
atggactttcaggtgcagatct

SEQ ID NO: 9 - DNA, 22 bp, Artificial Sequence
  MKcL-3 primer
ttgctgttctgggtatctggta

SEQ ID NO: 10 - DNA, 25 bp, Artificial Sequence
  MKcL-4 primer
atggagacagacacactcctgctat

SEQ ID NO: 11 - DNA, 18 bp, Artificial Sequence
  MKC1 primer
ggatacagttggtgcagc

SEQ ID NO: 12 - protein, 117 aa, Mus musculus
EVQLQQSGPELVKAGASMKISCKASGYSFTGYTMNWVKQSHGENLEWIGLINPFNGGTVYNQKLKGRATLTLDMSSSTAYMELLSLTSEDSAVYYCARNYRYAMDYWGQGTSVTVSS

SEQ ID NO: 13 - DNA, 350 bp, Mus musculus
gaggtccagctgcaacagtctggacctgagctggtgaaggctggagcttcaatgaagatatcctgcaaggcttctggttactcattcactggctacaccatgaactgggtgaaacagagccatggagagaaccttgagtggattggacttattaatcctttcaatggtggtactgtctacaaccagaagctcaagggcagggccacattaactttagacatgtcatccagcacagcctacatggaactcctcagtctgacatctgaggactctgcagtctattactgtgcaagaaactataggtatgctatggactactggggtcaaggaacctcagtcaccgtctcctc

SEQ ID NO: 14 - DNA, 294 bp, Mus musculus
gaggtccagctgcaacagtctggacctgagctggtgaagcctggagcttcaatgaagatatcctgcaaggcttctggttactcattcactggctacaccatgaactgggtgaagcagagccatggaaagaaccttgagtggattggacttattaatccttacaatggtggtaatagctacaaccagaagttcaagggcaaggccacattaactgtagacaagtcatccagcacagcctacatggagctcctcagtctgacatctgaggactctgcagtctattactgtgcaaga

SEQ ID NO: 15 - protein, 98 aa, Mus musculus
EVQLQQSGPELVKPGASMKISCKASGYSFTGYTMNWVKQSHGKNLEWIGLINPYNGGNSYNQKFKGKATLTVDKSSSTAYMELLSLTSEDSAVYYCAR

SEQ ID NO: 16 - DNA, 291 bp, Mus musculus
aggtccaagtgcaacagtctggacctgagctggtgaagcctggagcttcaatgaagatatcgtgcaaggcttctggttactcattcactggctacaccatgaactgggtgaagcagagccatggaaagaaccttgactggattggacttattaatccttaccatggtggtactagctacaaccagaagttcaagggcaaggccacattaactgtagacaagtcatccagcacagcctacacggagctcctcagtctgacatctgaggactctgcagtctattactgtgcaa

SEQ ID NO: 17 - DNA, 294 bp, Mus musculus
gaggtccagctgcaacagtctggacctgagctggtgaagcctggggcttcagtgaagataccctgcaaggcttctggatacacattcactggctacaacatggactgggtgaagcagagccatggaaagagccttgagtggattggagatattaatcctaacaatggtggtactatctacaaccagaagttcaagggcaaggccacattgactgtagacaagtcctccagcacagcctacatggagctccgcagcctgacatctgaggacactgcagtctattactgtgcaaga

SEQ ID NO: 18 - DNA, 10 bp, Mus musculus
actataggta

SEQ ID NO: 19 - DNA, 7 bp, Mus musculus
actatag

SEQ ID NO: 20 - DNA, 47 bp, Mus musculus
tatgctatggactactggggtcaaggaacctcagtcaccgtctcctc

SEQ ID NO: 21 - DNA, 17 bp, Mus musculus
gactactggggccaagg

SEQ ID NO: 22 - DNA, 14 bp, Mus musculus
tactggggccaagg

SEQ ID NO: 23 - protein, 117 aa, Mus musculus
QVQLQQSGAELARPGASVKLSCKASGYTFTDYYINWVKQRTGQGLEWIGEIYPGSGITYYNEKFKGKATLTADKSSSTAYMQLSSLASEDSAVYFCARRRDYYRYEDYWGQGTTLTV

SEQ ID NO: 24 - DNA, 350 bp, Mus musculus
caggttcagctgcagcagtctggagctgagctggcgaggcccggggcttcagtgaagctgtcctgcaaggcttctggctacaccttcactgactactatataaactgggtgaagcagaggactggacagggccttgagtggattggagagatttatcctggaagtggtattacttactacaatgagaagttcaagggcaaggccacactgactgcagacaaatcctccagcacagcctacatgcagctcagcagcctggcatctgaggactctgcagtctatttctgtgcaagaagaagggactactataggtacgaggactactggggccaaggcaccactctcacagt

SEQ ID NO: 25 - DNA, 294 bp, Mus musculus
caggttcagctgcagcagtctggagctgagctggcgaggcccggggcttcagtgaagctgtcctgcaaggcttctggctacaccttcactgactactatataaactgggtgaagcagaggactggacagggccttgagtggattggagagatttatcctggaagtggtaatacttactacaatgagaagttcaagggcaaggccacactgactgcagacaaatcctccagcacagcctacatgcagctcagcagcctgacatctgaggactctgcagtctatttctgttcaaga

SEQ ID NO: 26 - protein, 98 aa, Mus musculus
QVQLQQSGAELARPGASVKLSCKASGYTFTDYYINWVKQRTGQGLEWIGEIYPGSGNTYYNEKFKGKATLTADKSSSTAYMQLSSLTSEDSAVYFCSR

SEQ ID NO: 27 - DNA, 294 bp, Mus musculus
caggtccagctgaagcagtctggagctgagctggtgaagcctggggcttcagtgaagatatcctgcaaggcttctggctacaccttcactgactactatataaactgggtgaagcagaggcctggacagggccttgagtggattggaaagattggtcctggaagtggtagtacttactacaatgagaagttcaagggcaaggccacactgactgcagacaaatcctccagcacagcctacatgcagctcagcagcctgacatctgaggactctgcagtctatttctgtgcaaga

SEQ ID NO: 28 - DNA, 294 bp, Mus musculus; identical to SEQ ID NO: 27

SEQ ID NO: 29 - DNA, 15 bp, Mus musculus
ctactataggtacga

SEQ ID NO: 30 - DNA, 9 bp, Mus musculus
ctactatag

SEQ ID NO: 31 - DNA, 32 bp, Mus musculus
gactactggggccaaggcaccactctcacagt

SEQ ID NO: 32 - DNA, 14 bp, Mus musculus
tactggggccaagg

SEQ ID NO: 33 - DNA, 18 bp, Mus musculus
ggactactggggtcaagg

SEQ ID NO: 34 - protein, 117 aa, Mus musculus
EVQLQQSGPELVKPGASVKISCKASGYSFTGYFMNWVMQSHGKSLEWIGRINPYNGGTSYNQKFKGKATLTVDKSSSTAYMELLSLTSEDSAVYYCARILRPPYAMDYWGQGTSVTV

SEQ ID NO: 35 - DNA, 350 bp, Mus musculus
gaggttcagctgcagcagtctggacctgagctggtgaagcctggggcttcagtgaagatatcctgcaaggcttctggttactcatttactggctactttatgaactgggtgatgcagagccatggaaagagccttgagtggattggacgtattaatccttacaatggtggtactagctacaaccagaagttcaagggcaaggccacattaactgtagacaagtcatccagcacagcctacatggagctcctcagtctgacatctgaggactctgcagtctattactgtgcaaggatactacggccaccctatgctatggactactggggtcaaggaacctcagtcaccgt

SEQ ID NO: 36 - DNA, 293 bp, Mus musculus
gaggtccagctgcaacagtctggacctgagctggtgaagcctggagcttcaatgaagatatcctgcaaggcttctggttactcattcactggctacaccatgaactgggtgaagcagagccatggaaagaaccttgagtggattggacttattaatccttacaatggtggtaatagctacaaccagaagttcaagggcaaggccacattaactgtagacaagtcatccagcacagcctacatggagctcctcagtctgacatctgaggactctgcagtctattactgtgcaag

SEQ ID NO: 37 - protein, 97 aa, Mus musculus
EVQLQQSGPELVKPGASMKISCKASGYSFTGYTMNWVKQSHGKNLEWIGLINPYNGGNSYNQKFKGKATLTVDKSSSTAYMELLSLTSEDSAVYYCA

SEQ ID NO: 38 - DNA, 293 bp, Mus musculus
gaggttcagctgcagcagtctggacctgagctggtgaagcctggggattcagtgaagatatcctgcaaggcttctggttactcatttactggctactttatgaactgggtgatgcagagccatggaaagagccttgagtggattggacgtattaatccttacaatggtgatactttctacaaccagaagttcaagggcaaggccacattaactgtagacaaatcctctagcacagcccacatggagctccggagcctgacatctgaggactctgcagtctattattgtgcaag

SEQ ID NO: 39 - DNA, 293 bp, Mus musculus
gaggttcagctgcagcagtctggacctgagctggtgaagcctggggcttcagtgaagatatcctgcaaggcttctggttactcatttactggctactttatgaactgggtgaagcagagccatggaaagagccttgagtggattggacgtattaatccttacaatggtgatactttctacaaccagaagttcaagggcaaggccacattaactgtagacaaatcctctagcacagcccacatggagctcctgagcctgacatctgaggactttgcagtctattattgtgcaag

SEQ ID NO: 40 - DNA, 9 bp, Mus musculus
tactacggc

SEQ ID NO: 41 - DNA, 8 bp, Mus musculus
tactacgg

SEQ ID NO: 42 - DNA, 7 bp, Mus musculus
tactacg

SEQ ID NO: 43 - DNA, 42 bp, Mus musculus
ctatgctatggactactggggtcaaggaacctcagtcaccgt

SEQ ID NO: 44 - DNA, 17 bp, Mus musculus
gactactggggccaagg

SEQ ID NO: 45 - DNA, 14 bp, Mus musculus
tactggggccaagg

SEQ ID NO: 46 - protein, 117 aa, Mus musculus
EIQLQQTGPELVKPGASVKISCKASGYSFTDYFMLWVKQSHGKSLEWIGNINPYYGNTNYNLKFEGKATLTVDKSSNTAYMQLSSLTSEDSAVYYCARCGNYGNYFDYWGQGTTLTV

SEQ ID NO: 47 - DNA, 350 bp, Mus musculus
gagatccagctgcagcagactggacctgagctggtgaagcctggggcttcagtgaagatatcctgcaaggcttctggttattcattcactgactacttcatgctctgggtgaagcagagccatggaaagagccttgagtggattggaaatattaatccttactatggtaatactaactacaatctgaaattcgagggcaaggccacattgactgttgacaaatcttccaacactgcctacatgcagctcagcagtctgacatctgaggactctgcagtctattactgtgcaagatgcggtaactacgggaactactttgactactggggccagggcaccactctcaccgt

SEQ ID NO: 48 - DNA, 294 bp, Mus musculus
gagatccagctgcagcagtctggacctgagctgatgaagcctggggcttcagtgaagatatcctgcaaggcttctggttactcattcactagctactacatgcactgggtgaagcagagccatggaaagagccttgagtggattggatatattgatcctttcaatggtggtactagctacaaccagaaattcaagggcaaggccacattgactgtagacaaatcttccagcacagcctacatgcatctcagcagcctgacatctgaggactctgcagtctattactgtgcaaga

SEQ ID NO: 49 - protein, 98 aa, Mus musculus
EIQLQQSGPELMKPGASVKISCKASGYSFTSYYMHWVKQSHGKSLEWIGYIDPFNGGTSYNQKFKGKATLTVDKSSSTAYMHLSSLTSEDSAVYYCAR

SEQ ID NO: 50 - DNA, 294 bp, Mus musculus
gagttccagctgcagcagtctggacctgagctggtgaagcctggcgcttcagtgaagatatcctgcaaggcttctggttactcattcactgactacaacatgaactgggtgaagcagagcaatggaaagagccttgagtggattggagtaattaatcctaactatggtactactagctacaatcagaagttcaagggcaaggccacattgactgtagaccaatcttccagcacagcctacatgcagctcaacagcctgacatctgaggactctgcagtctattactgtgcaaga

SEQ ID NO: 51 - DNA, 294 bp, Mus musculus
gagatccagctgcagcagtctggacctgagctggtgaagcctggggcttcagtgaaggtatcctgcaaggcttctggttactcattcactgactacaacatgtactgggtgaagcagagccatggaaagagccttgagtggattggatatattgatccttacaatggtggtactagctacaaccagaagttcaagggcaaggccacattgactgttgacaagtcctccagcacagccttcatgcatctcaacagcctgacatctgaggactctgcagtctattactgtgcaaga

SEQ ID NO: 52 - DNA, 9 bp, Mus musculus
ggtaactac

SEQ ID NO: 53 - DNA, 38 bp, Mus musculus
tactttgactactggggccaaggcaccactctcacagt

SEQ ID NO: 54 - DNA, 11 bp, Mus musculus
tactggggcca

SEQ ID NO: 55 - DNA, 10 bp, Mus musculus
actactgggg

SEQ ID NO: 56 - protein, 117 aa, Mus musculus
EVQLQQSGPELVKPGASVKISCKASGSSFTAYTMNWVKQSHGKNLEWIGLINPYNGGSTYNHNFKGKATLTLDRSSSTAYMELLSLTPEDSGVYYCARATFVRPFYFDVWGAGTTVT

SEQ ID NO: 57 - DNA, 350 bp, Mus musculus
gaggtccagctgcagcagtctggacctgagctggtgaagcctggagcttcagtgaagatatcctgcaaggcttccggttcctcattcactgcctacaccatgaactgggtgaagcagagccatggaaagaaccttgagtggattggacttattaatccttacaatggtggttctacttacaaccacaatttcaagggcaaggccacattaactttagacaggtcatccagcacagcctacatggagctcctcagtctgacacctgaggactctggtgtctattactgtgcaagagcgacatttgtacggcccttctacttcgatgtctggggcgcagggaccacggtcac

SEQ ID NO: 58 - DNA, 294 bp, Mus musculus; identical to SEQ ID NO: 14

SEQ ID NO: 59 - protein, 98 aa, Mus musculus; identical to SEQ ID NO: 15

SEQ ID NO: 60 - DNA, 292 bp, Mus musculus
gaggtccaagtgcaacagtctggacctgagctggtgaagcctggagcttcaatgaagatatcgtgcaaggcttctggttactcattcactggctacaccatgaactgggtgaagcagagccatggaaagaaccttgactggattggacttattaatccttaccatggtggtactagctacaaccagaagttcaagggcaaggccacattaactgtagacaagtcatccagcacagcctacacggagctcctcagtctgacatctgaggactctgcagtctattactgtgcaa

SEQ ID NO: 61 - DNA, 294 bp, Mus musculus
gaggtccagctgcaacagtctggacctgagctggtgaagcctggggcttcagtgaagataccctgcaaggcttctggatacacattcactgactacaacatggactgggtgaagcagagccatggaaagagccttgagtggattggagatattaatcctaacaatggtggtactatctacaaccagaagttcaagggcaaggccacattgactgtagacaagtcctccagcacagcctacatggagctccgcagcctgacatctgaggacactgcagtctattactgtgcaaga

SEQ ID NO: 62 - DNA, 6 bp, Mus musculus
tacggc

SEQ ID NO: 63 - DNA, 5 bp, Mus musculus
tacgg

SEQ ID NO: 64 - DNA, 35 bp, Mus musculus
tacttcgatgtctggggcgcagggaccacggtcac

SEQ ID NO: 65 - DNA, 7 bp, Mus musculus
ctggggc

(19) United States
(12) Patent Application Publication
(10) Pub. No.: US 2015/0301045 A1
(43) Pub. Date: Oct. 22, 2015

(54) NON-B-LINEAGE CELLS CAPABLE OF PRODUCING ANTIBODY

(71) Applicant: Becton, Dickinson and Company, Franklin Lakes, NJ (US)

(72) Inventors: Efthalia Chronopoulou, La Jolla, CA (US); Alejandro Uribe, San Diego, CA (US); Jeanne Elia, San Diego, CA (US); Andrea Nguyen, San Diego, CA (US); Jody Berry, Carlsbad, CA (US)

(21) Appl. No.: 14/691,183

(22) Filed: Apr. 20, 2015

Related U.S. Application Data
(60) Provisional application No. 61/982,252, filed on Apr. 21, 2014.
(60) Provisional application No. 61/982,239, filed on Apr. 21, 2014.

(51) Int. Cl. G01N 33/569; G01N 33/68

(52) U.S. Cl. CPC G01N 33/56972; G01N 33/686; G01N 33/6872; G01N 33/56966; G01N 33/6845

(57) ABSTRACT

Disclosed herein are kits for detection and isolation of antibody-producing cells and cells capable of producing antibodies, antibody-producing cells and cells capable of producing antibodies, including V cells, a non-B-cell-lineage class of cells capable of producing antibody. Disclosed herein are methods of identifying and methods of isolating antibody-producing cells and cells capable of producing antibodies. Disclosed herein are methods of making antibodies.

### CROSS-REFERENCE TO RELATED APPLICATIONS

This application claims the benefit of U.S. Provisional Patent Application No. 61/982,252, filed Apr. 21, 2014, and U.S. Provisional Patent Application No. 61/982,239, filed Apr. 21, 2014, each of which is hereby incorporated by reference in its entirety. This application is related to U.S. patent application Ser. No. 14/420,314, which is the national phase of PCT Application No. PCT/US2013/065982 filed Oct. 21, 2013, and is incorporated herein by reference in its entirety.

### REFERENCE TO SEQUENCE LISTING, TABLE, OR COMPUTER PROGRAM LISTING

The present application is being filed along with a Sequence Listing in electronic format. The Sequence Listing is provided as a file entitled SEQUENCE_BDPHA_{—}003PR2.TXT, created Apr. 7, 2014, last saved Apr. 8, 2014, which is 23,583 bytes in size. The information in the electronic format of the Sequence Listing is incorporated herein by reference in its entirety.

### BACKGROUND

Antigen-binding proteins, such as antibodies, can bind to specific antigens, and can be useful, for example for research, diagnostic (in vitro and/or in vivo), and/or therapeutic applications. Antibody-producing cells of the hematopoietic system can produce antibodies. The development of cells of hematopoietic lineages has been summarized previously, for example is U.S. Pat. No. 5,622,853. Briefly, three major hematopoietic lineages have been identified: the erythroid lineage which matures into red blood cells; the myelomonocytic lineage which matures into granulocytes (including neutrophils, eosinophils and basophils) and monocytes; and the lymphoid lineage which matures into B lymphocytes, T lymphocytes and NK cells. (The megakaryocytes may be considered a fourth lineage which gives rise to platelets). B lymphocytes have been identified and characterized as antibody-producing cells. Within each lineage and between each lineage, molecules are expressed differentially on the surface and in the cytoplasm of the cells in a lineage. The expression of one or more molecules (such as cell surface markers) and/or the intensity of expression can be used to distinguish between maturational stages within a lineage and between lineages. Thus, such molecules can be useful as markers of lineage commitment and/or maturational stages for hematopoietic cells.

B lymphocytes are known to function as antibody-producing cells. Typically, an antibody-producing cell precursor undergoes gene arrangement to generate gene sequences that can encode portions of an antibody capable of binding to an antigen. The V and J segments of a gene encoding a light chain are rearranged to encode a variant of an antibody's variable light (V_{L}) chain, and the V, D, and J gene segments of a gene encoding a heavy chain are rearranged to encode a variant of an antibody's variable heavy chain (V_{H}) region. This process typically results in a population of antibody-producing cell precursors, each of which encodes a unique combination of V_{L} and V_{H} regions. When an antibody-producing cell precursor is stimulated by exposure to an antigen that is bound by the combination of V_{L} and V_{H} regions produced by the cell, the antibody-producing cell precursor can proliferate, producing a clonal population of cells capable of producing antibodies that bind to that stimulating antigen. Typically, multiple generations of this population of cells undergo a process of affinity maturation, in which the V_{L} and V_{H} encoding gene regions accumulate point mutations at a mutation rate much greater than for other genes (somatic hypermutation). Cells with improved affinity are selected for in the germinal center by receiving a survival signal from T cells. Each generation of cells can undergo one or more point mutations in each V_{L} or V_{H} encoding region, a process that is followed by antigen stimulation of clones with higher affinity for the antigen. This process results in enrichment for one or more clonal populations that contains a combination of V_{L} and V_{H}-encoding regions that produce antibody with high affinity for the antigen. Thus, a mature antibody-producing cell can produce a single antibody clone, which has undergone selection for having high affinity for a certain antigen. This process, like class switching, is dependent upon expression of the enzyme AID (activation induced cytidine deaminase) which is temporally expressed in differentiating B cells.

Previously, all antibody-producing cells were thought to belong to the B cell lineage, and express markers specific to B cells, while lacking markers specific for other lymphocyte lineages. Disclosed herein are methods for isolating, identifying, and generating antibodies from “V Cells,” a class of antibody-producing cells that express markers and possess other characteristics unique from B cells. Also disclosed are compositions, and kits therefor.

Previously, the identification of antigen-specific antibody-producing cells involved conjugating a fluorochrome to an antigen for the identification and isolation of antigen specific antibody producing cells. Presented herein are methods of identifying antigen-specific antibody producing cells that do not require the conjugation of a fluorophore to the antigen. Recombinant antibody technology and hybridoma/fusion technology can be applied to antibody-producing cells to generate antibodies.

### FIELD

The field relates generally to antibody-producing cells, methods of isolating and identifying antibody-producing cells, and methods of producing antibodies.

### SUMMARY

According to some aspects, a method of producing an antibody is provided. The method can comprise administering an antigen to a host organism. The method can comprise isolating at least one Ig-producing cell of the host organism, in which the cell comprises at least an IgG or IgE immunoglobulin that binds specifically to the antigen, and in which the cell is IgG+ IgE+ CD49b+, negative for B-cell specific markers, and positive for at least one of CD16/CD32, CD24, CD43, CD45, CD48, CD54, CD79b, CD123, CD200R, CD244.2, or FcεR1. In some embodiments the cells are negative for B-cell specific markers and negative for basophil-specific markers. The method can comprise generating an IgG or IgE antibody comprising a heavy chain variable region encoded by rearranged variable gene segments of the cell, and a light chain variable region encoded by rearranged variable gene segments of the cell. In some embodiments, the IgG or IgE immunoglobulin that binds specifically to the antigen is produced at least within about 15 days after administering the antigen to the host organism. In some embodiments, the IgG or IgE immunoglobulin that binds specifically to the antigen is produced at least within about 10 days after first administering the antigen to the host organism. In some embodiments, the IgG or IgE immunoglobulin that binds specifically to the antigen is produced at least within about 8 days after first administering the antigen to the host organism. In some embodiments, the IgG or IgE immunoglobulin is surface-bound. In some embodiments, the cell comprising at least an IgG or IgE immunoglobulin that binds specifically to the antigen is identified without the use of labeled antigen. In some embodiments, the cell comprises a V cell. In some embodiments, the method further comprises generating a first nucleic acid sequence of rearranged variable gene segments of the cell encoding the heavy chain variable region, and a second nucleic acid sequence of rearranged variable gene segments of the cell encoding the light chain variable region. In some embodiments, the method further comprises culturing a plurality of antibody-producing cells comprising genomic variable gene rearrangements encoding a heavy chain variable region of the surface-bound immunoglobulin and a light chain variable region of the surface-bound immunoglobulin. In some embodiments, the method further comprises engineering a humanized antibody comprising at least an HCDR1 of the heavy chain variable region, an HCDR2 of the heavy chain variable region, an HCDR3 of the heavy chain variable region, an LCDR1 of the light chain variable region, an LCDR2 of the light chain variable region, and an LCDR3 of the light chain variable region. In some embodiments, the host organism is immunocompromised, and prior to administering an antigen to the host organism, a naïve IgG+ IgE+ cell negative for B-cell specific markers, and positive for at least one of CD16/CD32, CD24, CD43, CD45, CD48, CD54, CD79b, CD123, CD200R, CD244.2, or FcεR1 is delivered to the host organism. In some embodiments, the antigen is administered to the host organism only once. In some embodiments, the cell comprises a human or murine cell. In some embodiments, the basophil-specific marker comprises 2D7, BB1, or both. In some embodiments, the basophil-specific marker comprises 2D7. In some embodiments, the basophil-specific marker comprises BB1. In some embodiments, the cell comprises a human or murine cell. In some embodiments, the cell comprises a murine cell. In some embodiments, the basophil specific marker comprises 2D7 and the cell comprises a murine cell. In some embodiments, the cell comprises a human cell. In some embodiments, the basophil specific marker comprises 2D7 and the cell comprises a human cell. In some embodiments, the basophil specific marker comprises BB1 and the cell comprises a human cell. In some embodiments, the antibody that binds specifically to basophils binds specifically to 2D7. In some embodiments, the antibody that binds specifically to basophils binds specifically to BB1.

According to some aspects, a complex is provided. The complex can comprise an isolated antibody-producing cell. The complex can comprise at least one of an IgE-specific antibody, CD49b-specific antibody, CD244.2-specific antibody, or CD200R-specific antibody bound to the cell. The complex can comprise an IgG-specific antibody bound to the cell. In some embodiments, the isolated antibody-producing cell is negative for B cell-specific markers. In some embodiments, the isolated antibody-producing cell is negative for basophil-specific markers. In some embodiments, the complex is not specifically bound by any of an antibody targeting B220, CD19, or CD20. In some embodiments, the complex is not specifically bound by an antibody targeting a basophil-specific marker. In some embodiments, an IgE-specific antibody is bound to the cell. In some embodiments, a CD49b-specific antibody is bound to the cell. In some embodiments, the complex is not specifically bound by antibody targeting CD5, CD21/CD35, CD22.2, CD72 GL-7, IgD, IgM, Ly6-k, Ly6-D, Ly-51 CD127, CD138, CD154, AA4.1, Pax-5, NK1.1, CD49a, CD122 or CD226/NKp46. In some embodiments, a CD200R-specific antibody is bound to the cell. In some embodiments, a CD244.2-specific antibody is bound to the cell. In some embodiments, a FcεR1-specific antibody is bound to the cell. In some embodiments, the complex further comprises at least one of a CD24-specific, CD43-specific, CD45-specific, or CD48-specific antibody bound to the cell. In some embodiments, each of the bound antibodies comprises a detectable marker. In some embodiments, at least one of the bound antibodies is attached to a separable phase. In some embodiments, the separable phase comprises a magnetic bead. In some embodiments, the cell produces or has produced at least one cytokine selected from the group consisting of IL-4, TNF, IL-13, and Il-10. In some embodiments, the cell comprises a murine cell. In some embodiments, the cell comprises a human cell. In some embodiments, the cell comprises a polymorphonuclear morphology. In some embodiments, the cell comprises an annular-shaped nucleus. In some embodiments, the cell comprises a V cell. In some embodiments, the isolated antibody-producing cell is negative for at least one basophil-specific marker selecting from the group consisting of: 2D7 and BB1. In some embodiments, the basophil-specific marker comprises 2D7. In some embodiments, the basophil-specific marker comprises BB1. In some embodiments, the cell comprises a human or murine cell. In some embodiments, the basophil-specific marker comprises 2D7 and the cell comprises a human or murine cell. In some embodiments, wherein the basophil-specific marker comprises 2D7 and the cell comprises a murine cell. In some embodiments, the basophil-specific marker comprises BB1 and the cell comprises a human cell. In some embodiments, the IgG+ IgE+ cells comprise a human or murine cell. In some embodiments, the IgG+ IgE+ cells comprise a murine cell. In some embodiments, the IgG+ IgE+ cells comprise a human cell.

According to some aspects, a method of detecting the presence of a cell capable of producing antigen-specific antibody is provided. The method can comprise providing a population of mammalian cells. The method can comprise detecting from the population the presence or absence of one or more IgG+ IgE+ cells, in which the IgG+ IgE+ cells are negative for B-cell specific markers and negative for basophil-specific markers and positive for at least one of CD49b, CD16/CD32, CD24, CD43, CD45, CD48, CD54, CD79b, CD123, CD200R, CD244.2, or FcεR1, and in which the IgG+ IgE+ cells are capable of producing an antibody. In some embodiments, the IgG+ IgE+ cells are positive for CD49b. In some embodiments, the IgG+ IgE+ cells are positive for CD200R. In some embodiments, the IgG+ IgE+ cells specifically bind to antigen (Ag). The IgG+ IgE+ cells can comprise a surface immunoglobulin that binds specifically to Ag. In some embodiments, the detecting comprises contacting the population of mammalian cells with: an antibody that specifically binds to CD49b; an antibody that specifically binds IgE; an antibody that specifically binds to IgG; an antibody that specifically binds to a B cell; and an antibody that binds specifically to a basophil, and also comprises determining the presence or absence of one or more IgG+ IgE+ CD49b+ cells that are negative for B-cell specific markers and negative for basophil specific markers. In some embodiments, the detecting comprises contacting the population of mammalian cells with: an antibody that specifically binds to CD49b, an antibody that specifically binds to IgG, an antibody that specifically binds IgE, and an antibody that specifically binds to B220, and also comprises determining the presence or absence of one or more B220− IgG+ IgE+ CD49b+ cells. In some embodiments, the antibody that binds specifically to basophils binds specifically to BB1. In some embodiments, the antibody that binds specifically to basophils binds specifically to 2D7. In some embodiments, the detecting comprises contacting the population of mammalian cells with: an antibody that specifically binds to CD49b, an antibody that specifically binds to IgG, an antibody that specifically binds IgE, and an antibody that specifically binds to CD19 or CD20, and determining the presence or absence of one or more CD19− IgG+ IgE+ CD49b+ cells or CD20− IgG+ IgE+ CD49b+ cells. In some embodiments, the detecting comprises contacting the population of mammalian cells with: an antibody that specifically binds to IgE, an antibody that specifically binds to IgG, and an antibody that specifically binds to B220, and also comprises determining the presence or absence of one or more IgE+ IgG+B220− cell. In some embodiments, the detecting comprises contacting the population of mammalian cells with: an antibody that specifically binds to IgE, an antibody that specifically binds to IgG, and an antibody that specifically binds to CD19 or CD20, and also comprises determining the presence or absence of an IgE+ IgG+ CD19− cell or IgE+ IgG+ CD20− cell. In some embodiments, the detecting comprises contacting the population of mammalian cells with: an antibody that specifically binds to IgG, an antibody that specifically binds to CD200R, and an antibody that specifically binds to B220, and also comprises determining the presence or absence of a IgG+ CD200R+B220-cell. In some embodiments, the detecting comprises contacting the population of mammalian cells with an antibody that specifically binds to IgG, an antibody that specifically binds to at least one of CD16/CD32, CD24, CD43, CD45, CD48, CD54, CD79b, CD123, CD200R, CD244.2, or FcεR1, and an antibody that specifically binds to a CD19 or CD20, and also comprises determining the presence or absence of a IgG+ CD19− or IgG+ CD20− cell that is positive for at least one of at least one of CD16/CD32, CD24, CD43, CD45, CD48, CD54, CD79b, CD123, CD200R, CD244.2, or FcεR1. In some embodiments, the method further comprises contacting the population of mammalian cells with an antibody that specifically binds to CD16/CD32, CD24, CD43, CD45, CD48, CD54, CD79b, CD123, CD200R, CD244.2, or FcεR1. In some embodiments, the method further comprises contacting the population of mammalian cells with antigen (Ag) and detecting binding or an absence of binding of the IgG+ IgE+ cell to Ag. In some embodiments, the method further comprises contacting the population of mammalian cells with: an antibody that specifically binds to CD49b+, and detecting the presence or absence of CD49b+ IgG+ IgE+ cells that are negative for B-cell specific markers. In some embodiments, the method further comprises contacting the population of mammalian cells with: an antibody that specifically binds to CD49b+, and determining the presence or absence of CD49b+ IgG+ IgE+ cells that are negative for basophil-specific markers. In some embodiments, the antibody that specifically binds to a B cell specifically binds to an antigen selected from the group consisting of B220, CD5, CD19, CD20, CD21/CD35, CD22.2, CD72, GL-7, IgD, IgM, Ly6-k, Ly6-D, Ly-51 CD127, CD138, CD154, AA4.1 and Pax-5. In some embodiments, the method further comprises the antibody that specifically binds to a B cell specifically binds to B220. In some embodiments, the method further comprises the antibody that specifically binds to a B cell specifically binds to one of CD19 or CD20. In some embodiments, the method further comprises detecting the presence of at least one additional marker on the IgG+ IgE+ cells, in which the at least one additional marker is selected from the group consisting of: CD24, CD43, CD45, or CD48. In some embodiments, the presence or absence of IgE is detected at the same time as the presence or absence of IgG. In some embodiments, the method further comprises determining the presence or absence of secretion of at least one of IL-4, TNF, IL-13, or Il-10 by the population of cells. In some embodiments, the method further comprises determining the absence of at least one additional marker on the IgG+ IgE+ cells, in which the at least one additional marker is selected from the group consisting of NK1.1, CD1d, CD3, CD4, CD8, CD25, CD38, CD134, CD11c, CD273, CD49a, CD122, CD226/NKp46, CD34, Sca-1, c-Kit, CD150, CD11b, and Ly-6G. In some embodiments the population of mammalian cells comprises human cells. In some embodiments, the population of mammalian cells comprises murine cells. In some embodiments, the population of mammalian cells comprises non-human cells. In some embodiments, the cell is part of a host immune system, and the method further comprises administering an antigen to the host. In some embodiments, the basophil-specific marker comprises 2D7, BB1, or both. In some embodiments, the basophil-specific marker comprises 2D7. In some embodiments, the basophil-specific marker comprises BB1. In some embodiments, the antibody-producing cell comprises a human or murine cell. In some embodiments, the antibody-producing cell comprises a murine cell. In some embodiments, the antibody-producing cell comprises a human cell. In some embodiments, the basophil maker comprises 2D7 and the antibody-producing cell comprises a human or murine cell. In some embodiments the basophil maker comprises 2D7 and the antibody-producing cell comprises a human or murine cell. In some embodiments, the basophil maker comprises 2D7 and the antibody-producing cell comprises a human cell. In some embodiments the basophil maker comprises 2D7 and the antibody-producing cell comprises a murine cell. In some embodiments the basophil maker comprises BB1 and the antibody-producing cell comprises a human cell.

According to some aspects, a method of enriching a cell-containing sample for IgG+ IgE+ cells capable of producing antibody in which the IgG+ IgE+ cells are negative for B-cell specific markers, negative for basophil-specific markers, and positive for at least one of CD49b, CD16/CD32, CD24, CD43, CD45, CD48, CD54, CD79b, CD123, CD200R, CD244.2, or FcεR1 is provided. The method can comprise contacting the sample with an enrichment antibody that specifically binds to B cells. The method can comprise contacting the sample with at least one of: an enrichment antibody that specifically binds to T cells, an enrichment antibody that specifically binds to monocytes, an enrichment antibody that specifically binds to dendritic cells, an enrichment antibody that specifically binds to NK cells, an enrichment antibody that specifically binds to erythrocytes, an enrichment antibody that specifically binds to hematopoietic stem cells, and an enrichment antibody that specifically binds to basophils, in which none of the enrichment antibodies binds specifically to B220− IgG+ IgE+ CD49b+ CD200R+ cells, CD19− IgG+ IgE+ CD49b+ CD200R+ cells, or CD20− IgG+ IgE+ CD49b+ CD200R+ cells. The method can comprise separating at least one of the IgG+ IgE+ cells capable of producing antibody with at least one enrichment antibody bound to said at least one IgG+ IgE+ cell from other cells of the sample. In some embodiments, the IgG+ IgE+ cells are further positive for CD49b. In some embodiments, the IgG+ IgE+ cells are further positive for CD200R. In some embodiments, the method further comprises detecting the presence of at least one of the IgG+ IgE+ cells. In some embodiments, the enrichment antibody that specifically binds to T cells binds specifically to one of CD1d, CD3, CD4, CD8, CD25, CD38 or CD134. In some embodiments, the enrichment antibody that specifically binds to dendritic cells binds specifically to one of CD11c or CD273. In some embodiments, the enrichment antibody that specifically binds to NK cells binds specifically to one of NK1.1, NK1.2, CD49a, CD122 or CD226/NKp46. In some embodiments, the enrichment antibody that specifically binds to hematopoietic stem cells binds specifically to one of CD34, Sca-1, c-Kit or CD150. In some embodiments, the enrichment antibody that specifically binds to basophils, specifically binds to secretory granules (e.g. marker 2D7) and/or basogranulin (e.g. marker BB1). In some embodiments, separating the cell or cells comprises fluorescence activated cell sorting. In some embodiments, separating the cell or cells comprises applying a composition comprising the cell or cells to an affinity column. In some embodiments, separating the cell or cells comprises contacting the cell or cells with affinity beads with or without a magnetic or physical separation. In some embodiments, separating the at least one cell comprises applying a magnetic field to a magnetic particle associated with the at least one cell. In some embodiments, the method further comprises contacting the sample with at least two of: an enrichment antibody that specifically binds to T cells, an enrichment antibody that specifically binds to monocytes; an enrichment antibody that specifically binds to dendritic cells, an enrichment antibody that specifically binds to NK cells, an enrichment antibody that specifically binds to erythrocytes, an enrichment antibody that specifically binds to hematopoietic stem cells, or an enrichment antibody that specifically binds to basophils. In some embodiments, the method further comprises contacting the sample with at least three of: an enrichment antibody that specifically binds to T cells, an enrichment antibody that specifically binds to monocytes; an enrichment antibody that specifically binds to dendritic cells, an enrichment antibody that specifically binds to NK cells, an enrichment antibody that specifically binds to erythrocytes, an enrichment antibody that specifically binds to hematopoietic stem cells, or an enrichment antibody that specifically binds to basophils. In some embodiments, the method further comprises contacting the sample with at least four of: an enrichment antibody that specifically binds to T cells, an enrichment antibody that specifically binds to monocytes; an enrichment antibody that specifically binds to dendritic cells; an enrichment antibody that specifically binds to NK cells, an enrichment antibody that specifically binds to erythrocytes, an enrichment antibody that specifically binds to hematopoietic stem cells, or an enrichment antibody that specifically binds to basophils. In some embodiments, the method further comprises contacting the sample with at least five of: an enrichment antibody that specifically binds to T cells, an enrichment antibody that specifically binds to monocytes; an enrichment antibody that specifically binds to dendritic cells; an enrichment antibody that specifically binds to NK cells, an enrichment antibody that specifically binds to erythrocytes, an enrichment antibody that specifically binds to hematopoietic stem cells, or an enrichment antibody that specifically binds to basophils. In some embodiments, the method further comprises contacting the sample with each of: an enrichment antibody that specifically binds to T cells, an enrichment antibody that specifically binds to monocytes, an enrichment antibody that specifically binds to dendritic cells; an enrichment antibody that specifically binds to NK cells, an enrichment antibody that specifically binds to erythrocytes, an enrichment antibody that specifically binds to hematopoietic stem cells; and an enrichment antibody that specifically binds to basophils. In some embodiments, the enrichment antibody that specifically binds to B cells binds specifically to one of B220, CD5, CD21/CD35, CD22.2, CD72, GL-7, IgD, IgM, Ly6-k, Ly6-D, Ly-51 CD127, CD138, CD154, AA4.1 or Pax-5. In some embodiments, the enrichment antibody that specifically binds to T cells binds specifically to one of CD1d, CD3, CD4, CD8, CD25, CD38 or CD134. In some embodiments, the enrichment antibody that specifically binds to dendritic cells binds specifically to one of CD11c or CD273. In some embodiments, the enrichment antibody that specifically binds to NK cells binds specifically to one of NK1.1, CD49a, CD122 or CD226/NKp46. In some embodiments, the enrichment antibody that specifically binds to hematopoietic stem cells binds specifically to one of CD34, Sca-1, c-Kit or CD150. In some embodiments, the enrichment antibody that specifically binds to basophils specifically binds to BB1 or 2D7.

According to some aspects, a kit for the detection of IgG+ IgE+ CD49b+ cells negative for B-cell-specific markers, negative for basophil-specific markers, and capable of producing antibody is provided. The kit can comprise a first antibody that specifically binds to IgG, in which the first antibody comprises a first detectable marker. The kit can comprise a second antibody that specifically binds to IgE, in which the second antibody comprises a second detectable marker. The kit can comprise a third antibody that specifically binds to CD49b, in which the third antibody comprises a third detectable marker. The kit can comprise a fourth antibody that specifically binds to a B-cell-specific marker. The kit can comprise a fifth antibody that binds specifically to a basophil-specific marker, in which the fifth antibody comprises a fifth detectable marker. The fourth antibody can comprise a fourth detectable marker, in which the first detectable marker, the second detectable marker, the third detectable marker, and the fourth detectable marker are each different from one another. The fourth detectable marker and the fifth detectable marker can be either the same as each other or different from each other. In some embodiments, the first detectable marker, the second detectable marker, the third detectable marker, the fourth detectable marker, and the fifth detectable marker are each different from one another. In some embodiments, two or more antibodies directed to a “negative” cellular marker (see, e.g. Table 1.2) comprise the same detectable marker, and the absence of multiple “negative” markers can be determined by the absence of the appropriate detectable marker. In some embodiments, the kit further comprises a sixth antibody that binds specifically to CD16/CD32, CD24, CD43, CD45, CD48, CD54, CD79b, CD123, CD200R, CD244.2, or FcεR1, in which the sixth antibody comprises a sixth detectable marker that is different from the first, second, third, fourth, and fifth detectable markers. In some embodiments, the fourth antibody specifically binds to a B-cell-specific marker selected from the group consisting of B220, CD19, and CD20. In some embodiments, the sixth antibody that binds specifically to one of CD24, CD43, CD45, or CD48. In some embodiments, the kit further comprises at least one of an antibody that binds specifically to CD1d, CD3, CD4, CD8, CD25, CD38 CD134, CD11c, CD273, CD49a, CD122, 2D7, BB1, CD200R, CD226/NKp46, CD34, Sca-1, c-Kit, CD150, CD11b, Ly-6G, or NKP46. In some embodiments, the kit further comprises at least one of an antibody that binds specifically to CD5, CD21/CD35, CD22.2, CD72, GL-7, IgD, IgM, Ly6-k, Ly6-D, Ly-51, CD127, CD138, CD154, AA4.1 and Pax-5. In some embodiments, the kit further comprises an antibody that binds specifically to NK cells, in which the antibody that binds specifically to NK cells does not bind specifically to CD49b, and wherein the antibody comprises a fourth detectable marker. In some embodiments, the kit further comprises a mammalian CD49b+ IgG+ IgE+ cell that is negative for B cell-specific markers and positive for at least one of CD16/CD32, CD24, CD43, CD45, CD48, CD54, CD79b, CD123, CD200R, CD244.2, or FcεR1. In some embodiments, the fourth antibody specifically binds to B220. In some embodiments, the fourth antibody specifically binds to CD19. In some embodiments, the fourth antibody specifically binds to CD20. In some embodiments, the sixth antibody binds specifically to one of CD200R, CD244.2, or FcεR1. In some embodiments, the kit further comprises an antibody that specifically binds to a cytokine selected from the group consisting of IL-4, TNF, IL-13, and Il-10, and that comprises a seventh detectable marker different from the first, second, third, fourth, fifth, and sixth detectable markers. In some embodiments, the basophil-specific marker comprises 2D7, BB1, or both. In some embodiments, the basophil-specific marker comprises 2D7. In some embodiments, the basophil-specific marker comprises BB1. In some embodiments, the fourth detectable marker is the same as the fifth detectable marker. In some embodiments, the fourth detectable marker is different from the fifth detectable marker. In some embodiments, the IgG+ IgE+ CD49b+ cells comprise human or murine cells. In some embodiments, the IgG+ IgE+ CD49b+ cells comprise human cells. In some embodiments, the IgG+ IgE+ CD49b+ cells comprise murine cells. In some embodiments the basophil specific marker comprises 2D7 and the cells comprise human or murine cells. In some embodiments, the basophil specific marker comprises 2D7 and the cells comprise human cells. In some embodiments, wherein the basophil specific marker comprises 2D7 and the cells comprise murine cells. In some embodiments, wherein the basophil specific marker comprises BB1 and the cells comprise human cells.

According to some aspects, a kit for enriching a sample for a population of IgG+ IgE+ cells capable of producing antibody is provided, in which the IgG+ IgE+ cells are negative for B-cell specific markers, negative for basophil specific markers, and positive for at least one of CD16/CD32, CD24, CD43, CD45, CD48, CD54, CD79b, CD123, CD200R, CD244.2, or FcεR. The kit can comprise an enrichment antibody that specifically binds to an antigen selected from the group consisting of B220, CD19, CD20, CD5, CD21/CD35, CD22.2, CD72, GL-7, IgD, IgM, Ly6-k, Ly6-D, Ly-51, CD127, CD138, CD154, AA4.1 and Pax-5. The kit can comprise at least one of an enrichment antibody that specifically binds to T Cells, an enrichment antibody that specifically binds to Monocytes, an enrichment antibody that specifically binds to dendritic cells, an enrichment antibody that specifically binds to NK cells, an enrichment antibody that specifically binds to hematopoietic stem cells, or an enrichment antibody that specifically binds to basophils. The kit can comprise a collection of separable phases bound to or capable of specifically complexing with the antibodies of the kit, in which the enrichment antibodies of the kit do not bind to the IgG+ IgE+ cells. In some embodiments, the IgG+ IgE+ cells are further CD49b+, and the enrichment antibodies of the kit do not bind to the IgG+ IgE+ CD49b+ cells. In some embodiments, the collection of separable phases comprises magnetic beads. In some embodiments, the enrichment antibodies are biotinylated and the separable phase comprises streptavidin. In some embodiments, the enrichment antibodies comprise a detectable marker, and the separable phase comprises a collection of separable phase particles that bind specifically to the detectable marker. In some embodiments, the enrichment antibodies comprise a detectable marker, and the separable phase comprises a collection of magnetic particles that bind specifically to the detectable marker. In some embodiments, the kit further comprises a detection antibody that binds specifically to CD49b and comprises a first detectable marker, a detection antibody that binds specifically to IgG and comprises a second detectable marker, and a detection antibody that binds specifically to IgE and comprises a third detectable marker. The first, second, and third detectable markers can be different from each other. In some embodiments, the enrichment antibody that specifically binds to T Cells binds to a marker from the group consisting of: CD1d, CD3, CD4, CD8, CD25, CD38 and CD134. In some embodiments, the enrichment antibody that specifically binds to monocytes specifically binds to a marker from the group consisting of: CD11b and Ly-6G. In some embodiments, the enrichment antibody that specifically binds to dendritic cells specifically binds to a marker from the group consisting of CD11c and CD273. In some embodiments, the enrichment antibody that specifically binds to NK Cells specifically binds to a marker from the group consisting of CD49a, CD122 and CD226/NKp46. In some embodiments, the enrichment antibody that specifically binds to hematopoietic stem cells specifically binds to a marker from the group consisting of CD34, Sca-1, c-Kit and CD150. In some embodiments, the enrichment antibody that specifically binds to basophils specifically binds to a marker consisting of: 2D7 or BB1. In some embodiments, the kit comprises at least two of: an enrichment antibody that specifically binds to T cells, an enrichment antibody that specifically binds to Monocytes, an enrichment antibody that specifically binds to dendritic cells, an enrichment antibody that specifically binds to NK cells, an enrichment antibody that specifically binds to hematopoietic stem cells, or an enrichment antibody that specifically binds to basophils. In some embodiments, the kit comprises at least three of: an enrichment antibody that specifically binds to T cells, an enrichment antibody that specifically binds to monocytes, an enrichment antibody that specifically binds to dendritic cells, an enrichment antibody that specifically binds to NK cells, an enrichment antibody that specifically binds to hematopoietic stem cells, or an enrichment antibody that specifically binds to basophils. In some embodiments, the kit comprises at least four of: an enrichment antibody that specifically binds to T Cells, an enrichment antibody that specifically binds to monocytes, an enrichment antibody that specifically binds to dendritic cells, an enrichment antibody that specifically binds to NK cells; an enrichment antibody that specifically binds to hematopoietic stem cells, or an enrichment antibody that specifically binds to basophils. In some embodiments, the kit comprises at least five of: an enrichment antibody that specifically binds to T Cells, an enrichment antibody that specifically binds to monocytes, an enrichment antibody that specifically binds to dendritic cells, an enrichment antibody that specifically binds to NK cells, an enrichment antibody that specifically binds to hematopoietic stem cells, or an enrichment antibody that specifically binds to basophils. In some embodiments, the kit comprises each of: an enrichment antibody that specifically binds to T Cells, an enrichment antibody that specifically binds to Monocytes, an enrichment antibody that specifically binds to dendritic cells, an enrichment antibody that specifically binds to NK Cells, an enrichment antibody that specifically binds to hematopoietic stem cells, and an enrichment antibody that specifically binds to basophils. In some embodiments, the kit further comprises a CD49b+ IgG+ IgE+ cell that is negative for B-cell specific markers and basophil specific markers (2D7 and/or BB1) and positive for at least one of CD16/CD32, CD24, CD43, CD45, CD48, CD54, CD79b, CD200R, CD244.2, or FcεR1.

According to some aspects, a hybridoma is provided. The hybridoma can comprise the fusion product of a CD49b+ IgG+ IgE+ cell that is negative for B-cell specific markers, negative for basophil specific markers and positive for at least one of: CD16/CD32, CD24, CD43, CD45, CD48, CD54, CD79b, CD123, CD200R, CD244.2, or FcεR1; and an immortalized cell, in which the hybridoma is an isolated, immortalized antibody-producing cell population. In some embodiments, the CD49b+ IgG+ IgE+ cell is negative for B220. In some embodiments, the CD49b+ IgG+ IgE+ cell is negative for CD19 and CD20. In some embodiments, the CD49b+ IgG+ IgE+ cell is CD200R+. In some embodiments, the CD49b+ IgG+ IgE+ cell is CD244.2+. In some embodiments, the CD49b+ IgG+ IgE+ cell is FcεR1+. In some embodiments, the basophil-specific marker comprises 2D7, BB1, or both. In some embodiments, the basophil-specific marker comprises 2D7. In some embodiments, the basophil-specific marker comprises BB1. In some embodiments, the CD49b+ IgG+ IgE+ cell comprises a human cell or murine cell. In some embodiments, the CD49b+ IgG+ IgE+ cell comprises a human cell. In some embodiments, the CD49b+ IgG+ IgE+ cell comprises a murine cell. In some embodiments, wherein the basophil-specific marker comprises 2D7, the cells comprise human or murine cells. In some embodiments, wherein the basophil-specific marker comprises 2D7, the cells comprise human cells. In some embodiments, wherein the basophil-specific marker comprises 2D7, the cells comprise murine cells. In some embodiments, wherein the basophil-specific marker comprises BB1, the cells comprise human cells.

According to some aspects, a method of making a hybridoma is provided. The method can comprise providing a cell immunized with an antigen (Ag), in which the cell is a CD49b+ IgG+ IgE+ cell that is negative for B cell-specific markers and negative for basophil specific markers. The method can comprise fusing the immunized cell with an immortalized cell. The method can comprise generating an isolated culture derived from a single clone of the fusion. In some embodiments, the cell is a V cell. In some embodiments, the basophil-specific marker comprises at least one of 2D7 or BB1. In some embodiments, the basophil-specific marker comprises 2D7. In some embodiments, the basophil-specific marker comprises BB1. In some embodiments, the CD49b+ IgG+ IgE+ cell comprises a human cell or murine cell. In some embodiments, the CD49b+ IgG+ IgE+ cell comprises a human cell. In some embodiments, the CD49b+ IgG+ IgE+ cell comprises a murine cell. In some embodiments the basophil-specific marker comprises at least one of 2D7 or BB1, and the cell comprises a human cell or murine cell. In some embodiments the basophil-specific marker comprises 2D7 and the cell comprises a human cell. In some embodiments the basophil-specific marker comprises 2D7 and the cell comprises a murine cell. In some embodiments the basophil-specific marker comprises BB1 and the cell comprises a human cell.

According to some aspects, a method of generating an IgG-encoding cDNA from an antibody-producing cell is provided. The method can comprise isolating an IgG-encoding mRNA from a CD49b+ IgG+ IgE+ cell that is negative for B cell-specific markers, and positive for at least one of: CD16/CD32, CD24, CD43, CD45, CD48, CD54, CD79b, CD123, CD200R, CD244.2, or FcεR1. The method can comprise generating a cDNA complementary to the IgG-encoding mRNA. In some embodiments, generating a cDNA comprises contacting an mRNA of the cell or an amplicon of an mRNA of the cell with at least one forward primer comprising SEQ ID NO: 1, SEQ ID NO: 2, SEQ ID NO: 3, or SEQ ID NO: 4, and at least one reverse primer comprising SEQ ID NO: 5 or SEQ ID NO: 6. In some embodiments, generating a cDNA comprises contacting an mRNA of the cell or an amplicon of an mRNA of the cell with at least one forward primer comprising SEQ ID NO: 7, SEQ ID NO: 8, SEQ ID NO: 9, or SEQ ID NO: 10, and at least one reverse primer comprising SEQ ID NO: 11. In some embodiments, the antibody-producing cell is CD200R+. In some embodiments, the antibody-producing cell is CD244.2+. In some embodiments, the antibody-producing cell is FcεR1+. In some embodiments, the antibody-producing cell is a V cell.

In some embodiments, for any of the above methods, complexes, hybridomas, or kits, the basophil-specific marker comprises 2D7, BB1, or both. In some embodiments, for any of the above methods, complexes, hybridomas, or kits, the basophil-specific marker comprises 2D7. In some embodiments, the basophil-specific marker comprises BB1. In some embodiments, for any of the above methods, complexes, hybridomas, or kits, the cell comprises a human or murine cell. In some embodiments, the cell comprises a murine cell. In some embodiments, for any of the above methods, complexes, hybridomas, or kits, the cell comprises a human cell. In some embodiments, for any of the above methods, complexes, hybridomas, or kits, the basophil-specific marker comprises 2D7, and the cell comprises a human or murine cell. In some embodiments the basophil-specific marker comprises 2D7, and the cell comprises a human cell. In some embodiments, for any of the above methods, complexes, hybridomas, or kits, the basophil-specific marker comprises 2D7, and the cell comprises a murine cell. In some embodiments, for any of the above methods, complexes, hybridomas, or kits, the basophil-specific marker comprises BB1, and the cell comprises a human cell.

### BRIEF DESCRIPTION OF THE DRAWINGS

FIG. 1A is a series of graphs illustrating the profile of the spleen of a non-immunized BALB/C mouse, which serves as a control for 9 different surface markers (Spleen Control). These markers identify B cells (B220+ and CD19+) (panels i and ii); cyclic ADP ribose hydroxylase (CD38) (panel iii) which is found on many immune cells including T cells (CD4+ and CD8+), B cells and Natural Killer cells; Syndecan-1 (CD138) (panel v) expressed on plasma cells; Natural Killer cells (Panel iv) (NKp46 and CD49b); Macrophages (CD11b) (panel iii) and Immunoglobulins G (IgG) and D (IgD) (panel vi) which are antibody isotypes expressed on the surface of B cells at different stages of differentiation.

FIG. 1B is a series of graphs illustrating the profile of the spleen of a non-immunized BALB/C mouse serves as a control for 3 additional markers (Spleen Control). These markers identify CD45+ cells (present on all differentiated hematopoietic cells with the exception of erythrocytes and plasma cells); Major-histocompatibility Complex class II+ cells (I-A^{d}/I-E^{d}) and Immunoglobulin M which is an antibody isotype expressed on the surface of B cells at different stages of differentiation (panels i, ii and iii).

FIG. 1C is a series of graphs illustrating the profile of the bone marrow of a non-immunized BALB/C mouse serves as a control for 7 different markers (Bone Marrow Control). These markers identify B cells (B220+) (panel i); Immunoglobulins G (IgG) and D (IgD) (panels iii and vi) which are antibody isotypes expressed on the surface of B cells at different stages of differentiation; CD45+ cells (present on all differentiated hematopoietic cells with the exception of erythrocytes and plasma cells) (panel iv); Major-histocompatibility Complex class II cells (I-A^{d}/I-E^{d}) (panel v) and Natural Killer cells (CD49b+) (panel iv).

FIG. 2A is a series of graphs illustrating the profile of the spleen of a Phycoerythrin (PE) immunized BALB/C mouse (Spleen from a mouse immunized with PE (4×)). Five different markers identify B cells (B220+); T cells (CD4+ and CD8+); Macrophages (CD11b/Mac-1+) and Granulocytes (Ly-6G/Gr-1+). B cells producing PE specific antibodies can be observed in quadrant Q2-1 of panel iv stain making 0.4% of the total lymphocyte population. It is also observed that macrophages (panel i) and granulocytes (panel ii) can stain positive (0.2%) for the antigen. However; an antigen specific population is also observed on the lower right hand side quadrant of panels i through v (Q4 and Q4-1), in which the cells are negative for the five aforementioned markers. The population varies from 0.6% to 1% depending on the stain.

FIG. 2B is a series of graphs illustrating the profile of spleen of an Allophycocyanin (APC)-immunized BALB/C mouse (Spleen from a mouse immunized with APC (4×)). Five different markers identify B cells (B220+) (panel iv); T cells (CD4+ and CD8+) (panels iii and v); Macrophages (CD11b/Mac-1+) (panel i) and Granulocytes (Ly-6G/Gr-1+) (panel ii). B cells producing APC specific antibodies can be observed in quadrant Q2 of panel iv making 0.3% of the total lymphocyte population. It is also observed that macrophages (panel i) and granulocytes (panel ii) can stain positive (0.1-0.2%) for the antigen. However; an antigen specific population is observed on the lower right hand side quadrant (Q4 and Q4-1) in each of the panels i through v, in which the cells are negative for the five aforementioned markers. The population varies from 0.5% to 0.8% depending on the stain.

FIG. 2C is a series of graphs illustrating the profile of the spleen of an Allophycocyanin (APC)-immunized BALB/C mouse (Spleen from a mouse immunized with APC (4×)). Six different markers identify B cells (B220+) (panel i); B-1 cells (CD5+) (panel iii); Syndecan-1 (CD138) expressed by plasma cells (panel ii); T-cell and B-cell activation antigen (GL-7) (panel iv); CD11c+ cells (dendritic cells, CD4− CD8+ intestinal intraepithelial lymphocytes and some NK cells) (panel v) and CD49b (found on NK-T, NK cells and fibroblasts cells) (panel vi). APC staining occurs on the X axis for all six markers. B cells producing APC specific antibodies can be observed in quadrant Q2 of panel i making 3.5% of the total lymphocyte population analyzed. The same antigen specific population described on FIG. 2B is observed on the lower right hand side quadrant (Q4 and Q4-1) in each of panels i through vi, in which the cells are negative for five of the aforementioned markers but is positive for CD49b with 0.6% of the total lymphocyte population analyzed.

FIG. 2D is a series of graphs illustrating further analysis of the profile of a spleen of an Allophycocyanin (APC)-immunized BALB/C mouse focusing on antigen specific antibody producing cells (Spleen from a mouse immunized with APC (4×)). A gate was placed on the B220+ Antigen specific+ cells (quadrant Q2 of panel i). A second gate was placed on B220− Antigen specific+ cells (quadrant Q4 of panel i). Both of these subpopulations were then analyzed against CD19 (panel ii), CD38 (panels iii and v), CD11b (panel v) and IgD (panel iv). Antigen specific B cells (top panels ii and iii) were positive for CD19 and partially positive for IgD (33%), CD38 (52.7%) and negative for CD11b. The B220−Ag+ cells (bottom panels iv and v) were negative to CD19, CD38, IgD and CD11b. B220−CD19−CD38−IgD−CD11b−Ag+ are labeled as “V cells” in subsequent FIGS. 2E-5B.

FIG. 2E is a series of graphs illustrating that immunization with various protein antigens induces antigen-specific V cells. Splenocytes derived from BALB/C mice immunized 4× with either APC (dot plots i, iv), PE (dot plots ii, v) or OVA (dot plots iii, vi) were stained with anti-B220-FITC (plots iv, v) or anti-B220−V500 (plot vi), antigen (APC, PE, OVA-PE depending on the immunogen used to induce the antigen specific cells) and 7AA−D. Gates were drawn to include events with forward and side scatter characteristics of viable cells (7-AAD−). The total number of events collected was 100,000 per sample. All three protein antigens indicated above could successfully induce V cells, identified as B220− Ag+7AAD−, in the spleen of immunized mice.

FIG. 2F is a series of graphs illustrating V cell distribution in various mouse tissues. Cells derived from the spleen (plots i and ii), bone marrow (plots iii and iv), and peripheral blood (plots v and vi) of 4× immunized mice with APC were stained with B220 V500 (clone RA3-6B2), APC, and 7AA−D. Gates were drawn to include events with forward and side scatter characteristics of viable cells (7-AAD−). The total number of events collected was 100,000 per sample. V cells (B220−Ag+7AAD−) were observed in the spleen (plot ii), bone marrow (plot iv), and peripheral blood (plot vi) of APC-inoculated mice but not in the PECs, lymph nodes and thymus.

FIG. 2G is a series of graphs illustrating V cell distribution in various mouse tissues. Cells derived from the peritoneal exudate cells (PEC) (plots vii and viii), lymph nodes (plots ix and x), and thymus (plots xi and xii) of 4× immunized mice with APC were stained with B220 V500 (clone RA3-6B2), APC, and 7AA−D. Gates were drawn to include events with forward and side scatter characteristics of viable cells (7-AAD−). The total number of events collected was 100,000 per sample. V cells (B220− Ag+ 7AAD−) were not observed in the PECs,

FIGS. 2H-2K are a series of graphs illustrating that antigen-specific V cells can be detected 8 days after a single immunization. Splenocytes derived from either naive (FIG. 2H, plots i, ii) or immunized (4×APC) (FIG. 2I, plots iii, iv) 12 week old BALB/C mice were stained with anti-B220−V500 (clone RA3-6B2), APC and 7-AAD. Gates were drawn to include events with forward and side scatter characteristics of viable cells (7-AAD−). The total number of events collected was 100,000 per sample. Following immunization, an APC-specific cell population was observed that is B220− (plots iv, vi and viii). A single injection with APC in C57BL/6 mice was sufficient to induce V cells (B220− Ag+7AAD−) in both the spleen (FIG. 2J, plots v and vi) and the bone marrow (FIG. 2K, plots vii and viii) of inoculated mice, as early as day 8 following immunization.

FIGS. 3A and 3B are a series of graphs illustrating phenotypic characterization of cell surface markers expressed on antigen-specific V cells. Cells derived from the spleen and bone marrow of C57BL/6 mice immunized 4× with APC were stained with anti-mouse B220, IgG, IgE, CD49b, APC, 7-AAD, and antibodies to cell surface markers. V cells were negative for a variety of HSC (CD34, c-Kit, Sca-1, and CD150), T- and NKT-cell (CD1d, CD3, CD4, CD8, CD25, and CD134), NK-cell (CD49a, CD122, and CD226/NKp46), dendritic-cell (CD11c and CD273), monocyte (Ly-6G), and a variety of B-cell lineage (CD5, CD19, B220, CD22.2, CD23, CD62P, CD72, GL-7, IgD, IgM, Ly-6K, Ly-6D, Ly-51, CD127, CD138, CD154, AA4.1) markers (Table 1.2). As shown in FIG. 3A, V cells were positive for surface IgG and CD49b (shown in Panel i). As shown in FIG. 3B, V cells were positive for CD24 (Column ii), CD43 (Column iii).

FIGS. 3C and 3D are a series of graphs illustrating phenotypic characterization of cell surface markers expressed on antigen-specific V cells. Cells derived from the spleen and bone marrow of C57BL/6 mice immunized 4× with APC were stained with anti-mouse B220, IgG, IgE, CD49b, APC, 7-AAD, and antibodies to cell surface markers. As shown in FIG. 3C, V cells were positive for CD45 (Column i), CD48 (Column ii), CD79b (Column iii). As shown in FIG. 3D, V cells were positive for CD200R (Column iv), FceR1 and IgE (Column v)

FIG. 3E is a series of graphs illustrating phenotypic characterization of cell surface markers expressed on antigen-specific V cells. V cells were positive for surface IgG and CD49b (shown in Panel i).

FIG. 3F is a series of graphs illustrating phenotypic characterization of cell surface markers expressed on antigen-specific V cells. Cells derived from the spleen and bone marrow of C57BL/6 mice immunized 4× with APC were stained with anti-mouse B220, IgG, IgE, CD49b, APC, 7-AAD, and antibodies to cell surface markers. V cells were positive for CD54 (Column i), CD16/CD32 (Column ii), CD244.2 (Column iii), IgE (present in Columns i through iii).

FIG. 3G is series of graphs illustrating a phenotypic characterization of cell surface markers expressed on antigen-specific V cells. As shown, cells derived from the spleen and bone marrow of BALB/c mice immunized 4× with APC were stained with anti-mouse B220, IgE, CD49b, APC, 7-AAD, and antibodies to cell surface markers. V cells were positive for CD123 (Column L) in both spleen and bone marrow and negative for basophil-specific antibody 2D7.

FIG. 3H is a series of graphs illustrating that V cells cycle in the bone marrow of an immunized mouse (24 hr BrdU pulsing). The profile of the bone marrow of an immunized BALB/C mouse with B lymphoma Mo-MLV insertion region 1 homolog (BMI-1) recombinant protein pulsed for 24 hours with BrdU to detect V cell cycling. BALB/C mice were injected with 1 mg BrdU in vivo (IP) for 24 hrs. Mice were sacrificed and single cell suspensions made from both spleen and bone marrow. Cells were surface stained for IgG and CD49b and then fixed/stained for BrdU using the BrdU flow kit staining procedure. V cells (CD49b+ IgG+) are identified by gate P2 in plot i. Upon further analysis of the BrdU+ population in plot i, gate P4 in panel iv shows that approximately 53% of the V cell population has incorporated BrdU in 18 hrs. In comparison, CD49b+ IgG− cells (identified in plot i by gate P3), incorporate BrdU in 18 hrs at a lower level of 30% (panel v gate p5).

FIG. 3I is a series of graphs illustrating that V cells cycle in the spleen of an immunized mouse (24 hr BrdU pulsing). The profile of the spleen of an immunized BALB/C mouse with B lymphoma Mo-MLV insertion region 1 homolog (BMI-1) recombinant protein pulsed for 24 hours with BrdU to detect V cell cycling. BALB/C mice were injected with 1 mg BrdU in vivo (IP) for 24 hrs. Mice were sacrificed and single cell suspensions made from both spleen and bone marrow. Cells were surface stained for IgG and CD49b and then fixed/stained for BrdU using the BrdU flow kit staining procedure. V cells (CD49b+ IgG+) are identified by gate P2 in plot i. Upon further analysis of the BrdU+ population in plot i, gate P4 in panel iv shows that approximately 22% of the V cell population has incorporated BrdU in 18 hrs. In comparison, CD49b+ IgG− cells (identified in plot i by gate P3), incorporate BrdU in 18 hrs at a lower level of 18% (panel v gate p5). Splenic V cells incorporate BrdU at a lower level then V cells found in the Bone Marrow.

FIG. 3J is a series of graphs illustrating V cells cycle in the bone marrow of an immunized mouse (48 hr BrdU pulsing). The profile of the bone marrow of an immunized BALB/C mouse with B lymphoma Mo-MLV insertion region 1 homolog (BMI-1) pulsed for 48 hours with BrdU to detect V cell cycling. BALB/C mice were injected with 1 mg BrdU in vivo (IP) for 48 hrs. Mice were sacrificed and single cell suspensions made from both spleen and bone marrow. Cells were surface stained for IgG and CD49b and then fixed/stained for BrdU using the BrdU flow kit staining procedure. V cells (CD49b+ IgG+) are identified by gate P2 in plot i. Upon further analysis of the BrdU+ population in plot i, gate P4 in panel iv shows that approximately 58% of the V cell population has incorporated BrdU in 42 hrs. In comparison, CD49b+ IgG− cells (identified in plot i by gate P3), incorporate BrdU in 18 hrs at a lower level of 37% (panel v gate p5). Each of the cell populations increased BrdU incorporation by approximately 5%.

FIG. 3K is a series of graphs illustrating that V cells cycle in the spleen of an immunized mouse (48 hr BrdU pulsing). The profile of the spleen of an immunized BALB/C mouse with B lymphoma Mo-MLV insertion region 1 homolog (BMI-1) pulsed for 48 hours with BrdU to detect V cell cycling. BALB/C mice were injected with 1 mg BrdU in vivo (IP) for 48 hrs. Mice were sacrificed and single cell suspensions made from both spleen and bone marrow. Cells were surface stained for IgG and CD49b and then fixed/stained for BrdU using the BrdU flow kit staining procedure. V cells (CD49b+ IgG+) are identified by gate P2 in plot i. Upon further analysis of the BrdU+ population in plot i, gate P4 in panel iv shows that approximately 43% of the V cell population has incorporated BrdU in 42 hrs. In comparison, CD49b+ IgG− cells (identified in plot i by gate P3), incorporate BrdU in 18 hrs at a lower level of 17% (panel v gate p5). The additional 24 hour BrdU load shows a dramatic increase in splenic V cell BrdU incorporation. On the other hand, the CD49b+ IgG− subset remained unchanged.

FIG. 3L is a series of graphs illustrating enrichment and sorting of antigen-specific V cells from Spleen and Bone Marrow from immunized mice. Cells derived from both the spleen (row i) and bone marrow (row ii) of immunized C57BL/6 mice (injected 4× with APC) were enriched for V cells using the BD IMag™ buffer, a custom biotinylated cocktail containing CD3e, CD11b, LY-6G and LY-6C, TER-119, and BD Imag™ Streptavidin Particles Plus. Enriched cells were then stained with B220 V500, CD49b V450, IgE FITC, IgG PE, APC(Ag), and 7-AAD. V cells were identified as B220− CD49b+IgG+IgE+Ag+7AAD− in both spleen and bone marrow and then sorted using a BD FACSAria™ III system (100-micron nozzle, drop drive frequency 31.0 kHz, sheath pressure 20.5 psi).

FIG. 3M is a photograph of a gel illustrating rearranged V gene cDNA from V cells from two compartments. Rearranged V gene cDNA from V cells obtained via sorting in a FACS Aria III from an Allophycocyanin (APC)-immunized BALB/C mouse. Ethidium bromide stained 1% agarose gel showing immunoglobulin variable region gene cDNAs after amplification using heavy chain leader region primers and IgG CH1 isotype specific reverse primers and i) RNA from V cells sorted from bone marrow, or ii) RNA from V cells sorted from spleen.

FIG. 3N is a series of photographs of gels illustrating that V cells express re-arranged immunoglobulin V-region mRNA. Cells derived from the bone marrow and spleen of BALB/C or C57BL/6 mice immunized 4× with APC (Ag) were magnetically enriched for V cells, and subsequently stained with B220 V500, anti-mouse IgE FITC, IgG PE, CD49b V450, APC and 7-AAD. V cells (B220−IgG+IgE+CD49b+APC+7-AAD−) were bulk sorted and then used for mRNA isolation, PCR amplification, cloning, and sequencing of their VH and VL genes. Bulk sorted V cells expressed re-arranged VH and VL genes in both the bone marrow and spleen. Recovery of rearranged IgG cDNA and rearranged kappa light chain cDNA from V cells sorted from the bone marrow is shown in Panel i, while nested IgE PCR with a gradient for optimization is shown on Panel ii.

FIG. 3O is a series of confocal microscope images illustrating that V cells are polymorphonuclear and express IgG and IgE simultaneously on their surface. Cells derived from the bone marrow and spleen of BALB/C or C57BL/6 mice immunized 4× with APC were magnetically enriched for V cells, and subsequently stained with B220 V500, anti-mouse IgE FITC, IgG PE, CD49b V450, APC and 7-AAD. V cells (B220− IgG+IgE+CD49b+APC+7-AAD−) were bulk sorted and then used for cytospins followed by methanol fixation and DAPI staining. Confocal microscopy analysis indicated that V cells are polymorphonuclear (Panels i and ii) and confirmed presence of both antigen specific IgG and IgE on the cell surface. Antibody capping was observed on 95% of the cells analyzed (Panel i), while 5% of the cells showed dispersed antigen, IgG and IgE on the cell surface (Panel ii). V cell nuclear morphology is distinct when compared to classical B cell subsets.

FIG. 3P is a series of microscope images illustrating that V cells have two distinct nuclear shapes. Cells derived from the bone marrow and spleen of BALB/C mice immunized 4× with APC (Ag) were magnetically enriched for V cells, and subsequently stained with B220 V500, anti-mouse IgE FITC, IgG PE, CD49b V450, APC and 7-AAD. V cells (B220−IgG+IgE+CD49b+APC+7-AAD−) were bulk sorted and were then used for either cytospins followed by methanol fixation and Giemsa staining or DAPI staining. Two distinct nuclear shapes can be observed: the first is an annular or ring shaped nucleus with a circular void running down through its center (panels i and ii), and the second is a multi-lobed nucleus (panels iii and iv) that shows no distinguishable chromatin filaments between each lobe (a characteristic shown by neutrophils).

FIG. 3Q is a series of electron micrographs illustrating that V cells have a distinct ultrastructure by Electron Microscopy (EM). Cells derived from the bone marrow and spleen of BALB/C or C57BL/6 mice immunized 4× with APC were magnetically enriched for V cells, and subsequently stained with B220 V500, anti-mouse IgE FITC, IgG PE, CD49b V450, APC and 7-AAD. V cells (B220−IgG+IgE+CD49b+APC+7-AAD−) were bulk sorted in a BD FACSAria™ III and then used for EM. EM analysis performed on a Tecnai spirit TEM by FEI at 80 KV equipped with Gatan 4 k×4 k digital camera showed the V cells having a different ultrastructure when compared to normal lymphocytes and appear to be richer in organelles, have more cytoplasm and many granular structures. The granular structures could be peroxisomes, but could also be primary or secondary lysosomes, or secretory granules. Two distinct types of nucleus are discernible: a multi-lobed mono-nuclear version on both spleen and bone marrow cells (Panels i and ii) and a second ring shaped (annular) version (Panel iii) confirmed by Giemsa stain and confocal microscopy (FIG. 3N).

FIG. 3R is a series of tailed EM images of organelles of V cells in spleen and bone marrow. Photomicrograph of organelles and general cellular ultrastructure of V cells from mouse spleen (panels i and ii) and bone marrow (panels iii and iv) taken at 10,000 times amplification. V cells have a characteristically large amount of rough endoplasmic reticulum (panel i), a large amount of cytoplasm which is very rich in organelles (panel ii) and many granular structures (panels iii and iv). Without being limited by any particular theory, it is contemplated herein that the granular structures could be peroxisomes, but could also be primary or secondary lysosomes, or secretory granules.

FIG. 3S is a microscope image illustrating that sorted V cells can be maintained in tissue culture. Cells derived from the bone marrow or spleen of BALB/C or C57BL/6 mice immunized 4× with APC were magnetically enriched for V cells, and subsequently stained with B220 V500, anti-mouse IgE FITC, IgG PE, CD49b V450, APC and 7-AAD. V cells (B220−IgG+IgE+CD49b+APC+7-AAD−) were bulk sorted aseptically in a BD FACSAria™ III and then used for tissue culture. 12,000-30,000 sorted V cells were plated in a 24-well plate on a feeder layer of M2-10B4 cells (ATCC CRL-1972) treated for 3 hrs with 1 μg of Mitomycin C (SIGMA M4287). Treated M2-10B4 cells were washed twice with complete RPMI media prior to adding sorted cells. The V cells were grown in 50% MyeloCult media (Stemcell Technologies M5300) and 50% complete RPMI Media (RPMI-1640+7.5% FBS (low IgG Hyclone)+1% Penn/Strep/Glutamine+5×10-5 M 2ME). Colony formation was observed 3 days post sort.

FIG. 3T is a series of microscope images illustrating a comparison between V cells from bone marrow and spleen against hematopoietic stem cells (HSC) in tissue culture. Images of sorted V cell and HSC colonies grown on M2-10B4 feeder cells. Bulk sorted BALB/C V cells from bone marrow (panel i), spleen (panel ii) and bulk sorted C57BL/6 HSC (KLS) cells (panel iii) were plated on mitomycin C-treated M2-10B4 cells and cultured for 10 to 13 days using Myelocult medium (StemCell Technologies M5300). Equal numbers of bone marrow and spleen V cells were plated on M2-10B4 feeder layer. HSC were plated at ½ the cell concentration. Cell colonies grew in all 3 sorted cell populations. HSC colonies began to appear at day 3, V cell colonies began to appear between days 3 and 5.

FIG. 4A is a series of graphs illustrating that naïve V cells are present in the spleen of nude mice. Due to a genetic mutation, nude mice (CD57BL/6 background) lack or have a severely deteriorated thymus and cannot generate mature T lymphocytes. This characteristic makes the mice unable to mount most types of immune responses, including: antibody formation that requires CD4+ helper T cells, cell-mediated immune responses (require CD4+ and/or CD8+ T cells) and delayed-type hypersensitivity responses (require CD4+ T cells) amongst others. Cells derived from the spleen of nude mice (C57BL/6 background) were stained with markers that characterized antigen specific V cells (anti-mouse B220, IgG, IgE, CD49b) and 7-AAD. An initial gate was drawn on all B200− cells, followed by a secondary gate that focused on CD49b+IgE+ cells. Naïve V cells from the spleen share the same phenotype as their antigen-specific counterpart and they are B220− IgG+IgE+CD49b+.

FIG. 4B and FIG. 4C are a series of graphs illustrating phenotypic characterization of naïve V cells from the spleen of nude mice. Cells derived from the spleen of nude mice (C57BL/6 background) were stained with markers that characterized antigen specific V cells (anti-mouse B220, IgG, IgE, CD244.2, CD200R) and 7-AAD. Gates were drawn on B220−IgE+, CD200R+IgE+hi and CD244.2+IgE+hi cells highlighting the V cell population. Naïve V cells from the spleen share the same phenotype markers as their antigen-specific counterpart and they are B220−IgG+IgE+CD49b+CD244.2+CD200R+.

FIG. 4D is a series of graphs illustrating that naïve V cells are present in the bone marrow of nude mice. Following the same strategy to detect naïve V cells in spleen (see FIGS. 4C-4D), cells derived from the bone marrow of nude mice (C57BL/6 background) were stained with markers that characterized antigen specific V cells (anti-mouse B220, IgG, IgE, CD49b) and 7-AAD. An initial gate was drawn on all B220− cells, followed by a secondary gate that focused on CD49b+IgE+ cells. Naïve V cells from the bone marrow share the same phenotype as their antigen-specific counterpart, and are B220− IgG+IgE+CD49b+.

FIG. 4E and FIG. 4F are a series of graphs illustrating phenotypic characterization of naïve V cells in the bone marrow of nude mice. Cells derived from the bone marrow of nude mice (C57BL/6 background) were stained with markers that characterized antigen specific V cells (anti-mouse B220, IgG, IgE, CD244.2, CD200R) and 7-AAD. Gates were drawn on the subpopulation of IgE+, CD200R+IgE+^{hi} and CD244.2+IgE+^{hi} cells highlighting the V cell population. Naïve V cells from the bone marrow share the same phenotype markers as their antigen-specific counterpart and they are B220− IgG+ IgE+ CD49b+ CD244.2+ CD200R+.

FIGS. 5A, 5B, and 5C are a series of graphs illustrating phenotypic characterization of naïve V cells in human peripheral blood. Human blood was collected from two different donors (FIGS. 5A and 5B, respectively) and PBMCs were isolated using the Ficoll-Paque protocol. PBMCs were then stained with CD19 and a cocktail of positive markers for V cells (CD49b, IgG, IgE and CD200R). Gates were drawn on CD19− cells and then on the V cell population to highlight their presence. V cells can be identified as CD19− CD49b+IgG+IgE+CD200R+. In FIG. 5C, human blood was collected from a donor and PBMCs were isolated using the Ficoll-Paque protocol as well. PBMCs were then stained with CD19 and a cocktail of positive markers for V cells (CD49b, IgG, IgE) and basophil-specific marker 2D7. Without being limited by any theory, it is contemplated that V-cells and basophils can share a large number of markers. As such, 2D7 can serve as a useful negative marker to separate these two populations. V-cells can be identified as CD19− CD49b+IgG+IgE+2D7−.

FIG. 6 is a flow diagram illustrating a method of making an antigen binding protein.

FIG. 7A is a sequence alignment illustrating immunoglobulin V gene utilization of a representative variable region sequence from a V cell isolated from bone marrow (“2-1 Bone Marrow VH”).

FIG. 7B is a sequence alignment illustrating immunoglobulin V gene utilization of a representative variable region sequence from a V cell isolated from bone marrow (“2-22 Bone Marrow VH”).

FIG. 7C is a sequence alignment illustrating immunoglobulin V gene utilization of a representative variable region sequence from a V cell isolated from bone marrow (“2-25 Bone Marrow VH”).

FIG. 7D is a sequence alignment illustrating immunoglobulin V gene utilization of a representative variable region sequence from a V cell isolated from a spleen (“3-1 Spleen VH”).

FIG. 7E is a sequence alignment illustrating immunoglobulin V gene utilization of a representative variable region sequence from a V cell isolated from a spleen (“3-3 Spleen VH”).

FIG. 8 is a light microscope image illustrating human V cells. Human V cells were sorted based on the phenotype IgE+ IgG+CD200R+CD49b+CD19−, and stained with May-Grüenwald Giemsa stains. Shown are cells with both high levels of IgE (panel i) and low levels of IgE (panel ii).

FIG. 9 is a light microscope image illustrating murine V cell colonies that formed in culture in vitro.

FIGS. 10A and 10B are a series of graph illustrating that V cells can be identified without the use of labeled antigen. Shown is flow cytometry data for V cells stained with various markers. FIG. 10A illustrates results for V cells in which antigen was present in the cocktail during the staining procedure. FIG. 10B illustrates results for V cells in which antigen was absent from the cocktail during the staining procedure.

FIGS. 11A-E are a series of graphs illustrating the profile of the spleen of a control HLA-DR2 1501 mouse. Spleens from naïve HLA-DR2 1501 mice were isolated and single cell suspensions were prepared. Cells were then stained with an antibody cocktail that identifies V cells: IgG, IgE, CD49b and CD200R. Cells were also gated based on viability (7-AAD−). V-cells are IgG+IgE−CD49b+CD200R+. Shown are: all events (FIG. 11A), Live/Dead cells (FIG. 11B), CD49b+ cells (FIG. 11C), CD49b+Ige+IgG+ cells (FIG. 11D), and CD49b+IgE+CD200R+ cells (FIG. 11E).

FIGS. 12A-E are a series of graphs illustrating the profile of the spleen of immunized HLA-DR2 1501 (EAE model) male mice, and the increase of the V-cell population. HLA-DR2 1501 male mice between age 8 and 12 wk were immunized subcutaneously at the lower back with MBP-85-99 peptide emulsified in CFA. On day 0 and 2 of immunization mice were injected with 200 ng of pertussis toxin. Tissues were isolated from EAE mice 14 days following immunization with clinical signs of disease severity of 2.5. Spleens from immunized (EAE mice) mice were isolated and single cell suspensions prepared. Cells were then stained with an antibody cocktail that identifies V cells: IgG, IgE, CD49b and CD200R. Cells were also gated based on viability (7-AAD−). V cells are IgG+IgE− CD49b+CD200R+. Samples were acquired on Accuri C6 (BD Biosciences) and analyzed using the Accuri C6 software. The V-cell population in the spleen increased almost 2 fold in the diseased state when compared to the control (3.7% vs 9%). Shown are: all events (FIG. 12A), Live/Dead cells (FIG. 12B), CD49b+ cells (FIG. 12C), CD49b+Ige+IgG+ cells (FIG. 12D), and CD49b+IgE+CD200R+ cells (FIG. 12E).

FIG. 13 is a series of graphs illustrating the V-cell population in the spinal cord of immunized HLA-DR2 1501 (EAE model) mice. HLA-DR2 1501 male mice between age 8 and 12 wk were immunized subcutaneously at the lower back with MBP-85-99 peptide emulsified in CFA. On day 0 and 2 of immunization mice were injected with 200 ng of pertussis toxin. Tissues were isolated from EAE mice 14 days following immunization with clinical signs of disease severity of 2.5. Spinal cords from immunized animals (no cell infiltrate is observed in the spinal cord of a naïve animal) were isolated and single cell suspensions were prepared. Cells were then stained with an antibody cocktail that identifies V cells: IgG, IgE, CD49b and CD200R. Cells were also gated based on viability (7-AAD−). V cells are IgG+IgE−CD49b+CD200R+. Shown are: all events (FIG. 13A), Live/Dead cells (FIG. 13B), CD49b+ cells (FIG. 13C), CD49b+Ige+IgG+ cells (FIG. 13D), and CD49b+IgE+CD200R+ cells (FIG. 13E).

FIGS. 14A-E are a series of graphs illustrating phenotypic characteristics of naïve V cells in human peripheral blood from a donor not suffering from any major illness or disease. Human blood was collected from a donor and PBMCs were isolated using the Ficoll-Paque protocol. PBMCs were then stained with CD19 and a cocktail of positive markers for V cells (CD49b, IgG, IgE) and Basophil-specific marker 2D7. Due to the fact that V-cells and basophils can share a large number of cell surface markers, 2D7 can serve as a useful negative marker to separate these two populations. V-cells can be identified as CD19− CD49b+IgG+IgE+2D7−. Shown are: all events (FIG. 14A), CD19 (FIG. 14B), CD49b (FIG. 14C), IgG (FIG. 14D), and 2D7 (FIG. 14E).

FIGS. 15A-E are a series of graphs illustrating phenotypic characteristics of a patient suffering Diabetes mellitus Type I and chronic inflammation. Human blood was collected from a donor that suffers Type I Diabetes and chronic inflammation from the extremities. PBMCs were isolated using the Ficoll-Paque protocol. PBMCs were then stained with CD19 and a cocktail of positive markers for V cells (CD49b, IgG, IgE) and Basophil-specific marker 2D7. When compared to a control sample (see FIG. 14), it is easily established that this sample lacks the presence of V-cells and only basophils can be detected. Shown are: all events (FIG. 15A), CD19 (FIG. 15B), CD49b (FIG. 15C), IgG (FIG. 15D), and 2D7 (FIG. 15E).

FIG. 16 is a flow diagram illustrating a method of determining a disease state according to some embodiments herein.

FIG. 17 is a flow diagram illustrating a method of increasing an antibody-producing cell count in a subject according to some embodiments herein.

### DETAILED DESCRIPTION

Disclosed herein are V cells, a previously-unidentified type of antibody-producing cell. V cells produce and express on their surface affinity-matured IgG antibody and IgE, following exposure to a specific antigen, thereby making them partly responsible for humoral and adaptive immunity alongside B cells. However, V cells differ from B cells, for example with respect to surface markers, antibody gene utilization, and types of antibody produced. Provided herein are isolated V cells, and complexes useful for isolating V cells. Provided herein are methods of identifying and/or isolating V cells, methods and compositions for enriching a cell population for V cells, and methods and compositions for using V cells to produce antibodies. Provided herein are kits for identifying and isolating V cells. Provided herein are methods of making antibodies and antigen binding proteins.

### V Cells

As used herein, “V cell” refers to an IgG+ IgE+ cell that is negative for B-cell-specific markers, and is positive for at least one of CD16/CD32, CD24, CD43, CD45, CD48, CD54, CD79b, CD123, CD200R, CD244.2, or FcεR1, and that is capable of expressing immunoglobulin. V cells with antigen-specific binding activity have been identified herein, as have naïve V cells. Naïve V cells are found in non-immunized animals and are not antigen specific (though without being limited by any theory, it is at least formally possible that naïve V cells have a specificity for some unknown antigen). Phenotypically, V cells are characterized by the markers as shown in Table 1.1 and Table 1.2, and further can be positive (or negative) for additional markers as described herein. Morphologically, V cells can also be characterized as polymorphonucleated, or having an annular-shaped nucleus. Some V-cells produce surface-bound IgG and IgE antibodies, which can be antigen-specific. Many V cells are CD49b+, but CD49b can be downregulated in some V-cells, for example naïve human V cells, so that these naïve human V cells have a CD49b^{low} or CD49b− phenotype. Without being limited to any particular theory, CD49b can be upregulated in V cells that are mounting an adaptive immune response, for example in response to infection by virus, parasite, or bacteria. As understood by the skilled artisan, the identity of particular “B-cell-specific markers” can depend upon the organism from which the host cell is derived from. For example, B220 is a B-cell-specific marker for mice, and CD19 and CD20 are each B-cell-specific markers in humans. As such, the skilled artisan can readily identify a B-cell-specific marker based on the host cell type. Moreover, when a V cell is negative for at least one B-cell-specific marker of the host organism, the skilled artisan may infer that the cell is (or at least is very likely) negative for other B cell-specific markers (e.g. if a human cell is CD19−, that human cell is very likely CD20− as well, and thus can be inferred to be “negative for B-cell-specific markers”). A V cell can be identified as disclosed herein, at least, for example, as an IgG+ IgE+ CD49b+ cell that is negative for B-cell-specific markers and negative for basophil-specific markers (2D7 and/or BB1), and that is also positive for at least one of CD16/CD32, CD24, CD43, CD45, CD48, CD54, CD79b, CD123, CD200R, CD244.2, or FcεR1. As such, a V cell may be identified by at least as the following properties (noting that B220 is a B-cell specific marker in mice): B220− IgG+ IgE+ CD49b+ CD200R+ 2D7− BB1−, or B220− IgG+ IgE+ CD49b+ CD200R+ 2D7− or B220− IgG+ IgE+ CD49b+ CD200R+ BB1− or B220− IgG+ IgE+ CD49b+ CD200R+ 2D7− BB1−, or B220− IgG+ IgE+ CD49b+ CD200R+ 2D7−, or B220− IgG+ IgE+ CD49b+ CD244.2+ BB1−, or B220− IgG+ IgE+Cd49b+ FcεR1+, 2D7− BB1−, or B220− IgG+ IgE+Cd49b+ FcεR1+2D7−, or B220− IgG+ IgE+Cd49b+ FcεR1+ BB1, or B220− IgG+ IgE+Cd49b+ CD16/CD32+ 2D7− BB1−, or B220− IgG+ IgE+Cd49b+ CD16/CD32+, 2D7−, or B220− IgG+ IgE+Cd49b+ CD16/CD32+ BB1−, or B220− IgG+ IgE+Cd49b+ CD24+2D7− BB1−, or B220− IgG+ IgE+Cd49b+ CD24+2D7−, or B220− IgG+ IgE+Cd49b+ CD24+ BB1−, or B220− IgG+ IgE+Cd49b+ CD43+2D7− BB1−, or B220− IgG+ IgE+ Cd49b+ CD43+2D7−, or B220− IgG+ IgE+Cd49b+ CD43+, BB1−, or B220− IgG+ IgE+ Cd49b+ CD45+2D7− BB1−, or B220− IgG+ IgE+Cd49b+ CD45+2D7−, or B220− IgG+ IgE+ Cd49b+ CD45+ BB1−, or B220− IgG+ IgE+ Cd49b+ CD48+2D7− BB1−, or B220− IgG+ IgE+ Cd49b+ CD48+ 2D7−, or B220− IgG+ IgE+ Cd49b+ CD48+ BB1−, or B220− IgG+ IgE+Cd49b+ CD54+2D7− BB1−, or B220− IgG+ IgE+ Cd49b+ CD54+ 2D7−, or B220− IgG+ IgE+Cd49b+ CD54+, BB1−, or B220− IgG+ IgE+Cd49b+ CD79b+ 2D7−, BB1−, or B220− IgG+ IgE+ Cd49b+ CD79b+2D7−, or B220− IgG+ IgE+ Cd49b+ CD79b+ BB1−, or B220− IgG+ IgE+ Cd49b+ 2D7− BB1−, or B220− IgG+ IgE+ Cd49b+ 2D7−, or B220− IgG+ IgE+Cd49b+ BB1−, or any combination thereof. As such, a V cell may be identified by at least the following properties in humans (noting that CD19 and CD20 are each a B-cell-specific marker in human) CD19− IgG+ IgE+ CD49b+ CD200R+ 2D7− BB1− or CD19− IgG+ IgE+ CD49b+ CD200R+ 2D7−, or CD19− IgG+ IgE+ CD49b+ CD200R+ BB1−, or CD19− IgG+ IgE+ CD49b+CD200R+ 2D7− BB1−, or CD19− IgG+ IgE+ CD49b+ CD200R+ 2D7−, or CD19− IgG+ IgE+ CD49b+ CD244.2+ BB1−, or CD19− IgG+ IgE+Cd49b+ FcεR1+ 2D7− BB1−, or CD19− IgG+ IgE+Cd49b+ FcεR1+ 2D7−, or CD19− IgG+ IgE+Cd49b+ FcεR1+ BB1−, or CD19− IgG+ IgE+Cd49b+ CD16/CD32+ 2D7− BB1−, or CD19− IgG+ IgE+Cd49b+ CD16/CD32+ 2D7−, or CD19− IgG+ IgE+Cd49b+ CD16/CD32+ BB1−, or CD19− IgG+ IgE+Cd49b+ CD24+ 2D7− BB1−, CD19− IgG+ IgE+Cd49b+ CD24+ 2D7−, or CD19− IgG+ IgE+Cd49b+ CD24+ BB1−, or CD19− IgG+ IgE+Cd49b+ CD43+ 2D7− BB1−, or CD19− IgG+ IgE+Cd49b+ CD43+ 2D7−, or CD19− IgG+ IgE+Cd49b+ CD43+ BB1−, or CD19− IgG+ IgE+Cd49b+ CD45+ 2D7− BB1−, or CD19− IgG+ IgE+Cd49b+ CD45+ 2D7−, or CD19− IgG+ IgE+Cd49b+ CD45+ BB1−, or CD19− IgG+ IgE+Cd49b+ CD48+ 2D7− BB1−, or CD19− IgG+ IgE+Cd49b+ CD48+ 2D7−, or CD19− IgG+ IgE+Cd49b+ CD48+ BB1−, or CD19− IgG+ IgE+Cd49b+ CD54+ 2D7− BB1−, or CD19− IgG+ IgE+Cd49b+ CD54+ 2D7−, or CD19− IgG+ IgE+Cd49b+ CD54+ BB1−, or CD19− IgG+ IgE+Cd49b+ CD79b+ 2D7− BB1−, or CD19− IgG+ IgE+Cd49b+ CD79b+ 2D7−, or CD19− IgG+ IgE+Cd49b+ CD79b+ BB1−, or CD19− IgG+ IgE+Cd49b+ 2D7− BB1−, or CD19− IgG+ IgE+Cd49b+ 2D7−, or CD19− IgG+ IgE+Cd49b+ BB1−, or CD20− IgG+ IgE+ CD49b+ CD200R+ 2D7− BB1−, CD20− IgG+ IgE+ CD49b+ CD200R+2D7−, or CD20− IgG+ IgE+ CD49b+ CD200R+ BB1−, or CD20− IgG+ IgE+ CD49b+ 2D7− BB1−, or CD20− IgG+ IgE+ CD49b+ 2D7−, or CD20− IgG+ IgE+ CD49b+ BB1−, or CD20− IgG+ IgE+ FcεR1+ 2D7− BB1−, or CD20− IgG+ IgE+ FcεR1+ 2D7, or CD20− IgG+ IgE+ FcεR1+ BB1−, or CD19− IgG+ IgE+ Cd49b+ CD16/CD32+ 2D7− BB1−, or CD19− IgG+ IgE+ Cd49b+ CD16/CD32+ 2D7−, or CD19− IgG+ IgE+ Cd49b+ CD16/CD32+ BB1−, or CD19− IgG+ IgE+ Cd49b+ CD24+ 2D7− BB1−, or CD19− IgG+ IgE+ Cd49b+ CD24+ 2D7−, or CD19− IgG+ IgE+ Cd49b+ CD24+ BB1−, or CD19− IgG+ IgE+ Cd49b+ CD43+, CD19− IgG+ IgE+ Cd49b+ CD45+, CD19− IgG+ IgE+ Cd49b+ CD48+2D7− BB1−, or CD19− IgG+ IgE+ Cd49b+ CD43+, CD19− IgG+ IgE+ Cd49b+ CD45+, CD19− IgG+ IgE+ Cd49b+ CD48+ 2D7−, or CD19− IgG+ IgE+ Cd49b+ CD43+, CD19− IgG+ IgE+Cd49b+ CD45+, CD19− IgG+ IgE+ Cd49b+ CD48+ BB1−, or CD19− IgG+ IgE+Cd49b+ CD54+ 2D7− BB1−, or CD19− IgG+ IgE+Cd49b+ CD54+ 2D7−, or CD19− IgG+ IgE+Cd49b+ CD54+ BB1−, or CD19− IgG+ IgE+ Cd49b+ CD79b+ 2D7− BB1−, or CD19− IgG+ IgE+Cd49b+ CD79b+ 2D7−, or CD19− IgG+ IgE+Cd49b+ CD79b+ BB1−, or CD19− IgG+ IgE+Cd49b+ 2D7− BB1−, or CD19− IgG+ IgE+Cd49b+ 2D7−, or CD19− IgG+ IgE+Cd49b+ BB1−, or any combination thereof. In some embodiments, for example if the V cell is a naïve human V cell, the V cell can have a phenotype as listed above, except that it is CD49b^{low} or CD49b− instead of CD49+, and does not have antigen specificity (or has specificity for some unknown antigen).

Additional phenotypic characteristics of V cells are illustrated in Tables 1.1-1.3, herein. The skilled artisan will appreciate that there are several phenotypes that are distinct to V cells. As such, identification of a V cell based on a particular phenotype supports the inference that the identified V cells also possess other V cell-specific phenotypes consistent with the identified phenotype. For example, if a human CD19− IgG+ IgE+ CD49b+ CD200R+ cell with a polymorphonuclear morphology is identified, it can be inferred that this cell is a V cell, and thus it can be inferred that that cell is also CD20− IgG+ IgE+ CD49b+ CD200R+ 2D7 BB1−, CD20− IgG+ IgE+ CD49b+ CD200R+ 2D7, CD20− IgG+ IgE+ CD49b+ CD200R+ BB1−, CD19− IgG+ IgE+ CD49b+ CD200R+ 2D7− BB1−, CD19− IgG+ IgE+ CD49b+ CD200R+ 2D7−, CD19− IgG+ IgE+ CD49b+CD244.2+ BB1, CD20− IgG+ IgE+ CD49b+ CD200R+ 2D7− BB1−, CD20− IgG+ IgE+ CD49b+ CD200R+ 2D7−, CD20− IgG+ IgE+ CD49b+ CD244.2+ BB1−, CD19− IgG+ IgE+ CD49b+ FcεR1+ 2D7− BB1−, CD19− IgG+ IgE+ CD49b+ FcεR1+ 2D7−, CD19− IgG+ IgE+ CD49b+ FcεR1+ BB1−, CD20− IgG+ IgE+ CD49b+ FcεR1+ 2D7− BB1−, CD20− IgG+ IgE+ CD49b+ FcεR1+ 2D7−, CD20− IgG+ IgE+ CD49b+ FcεR1+ BB1−, etc., consistent with the disclosure herein (see, e.g., Tables 1.1-1.3).

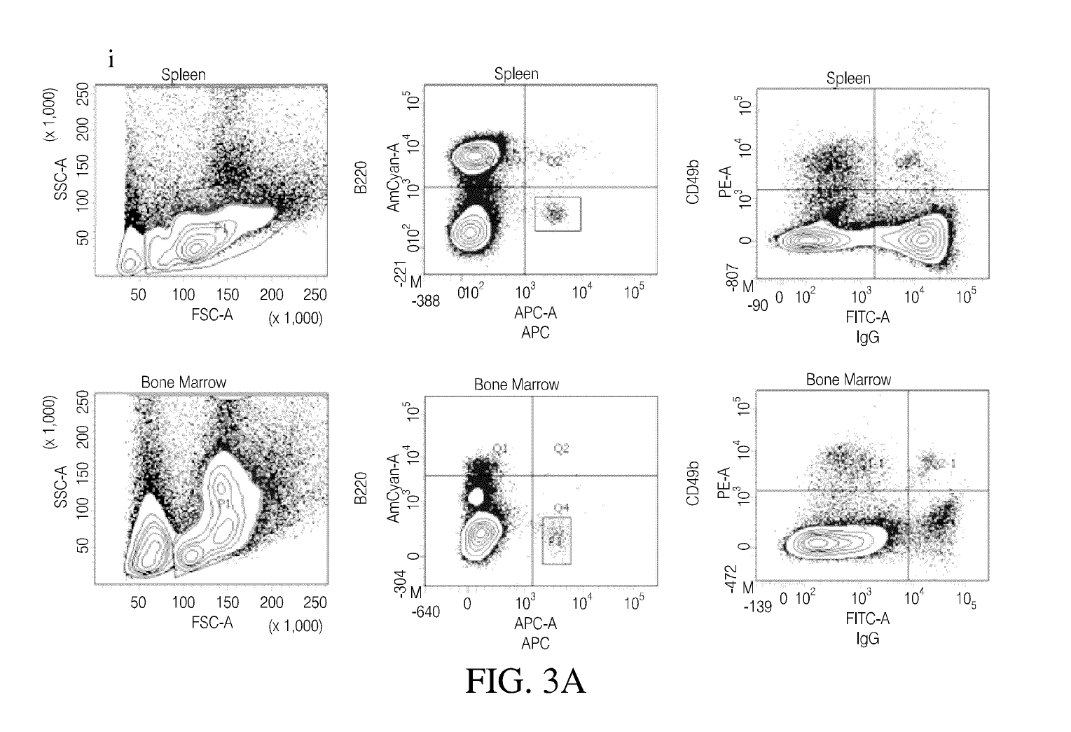

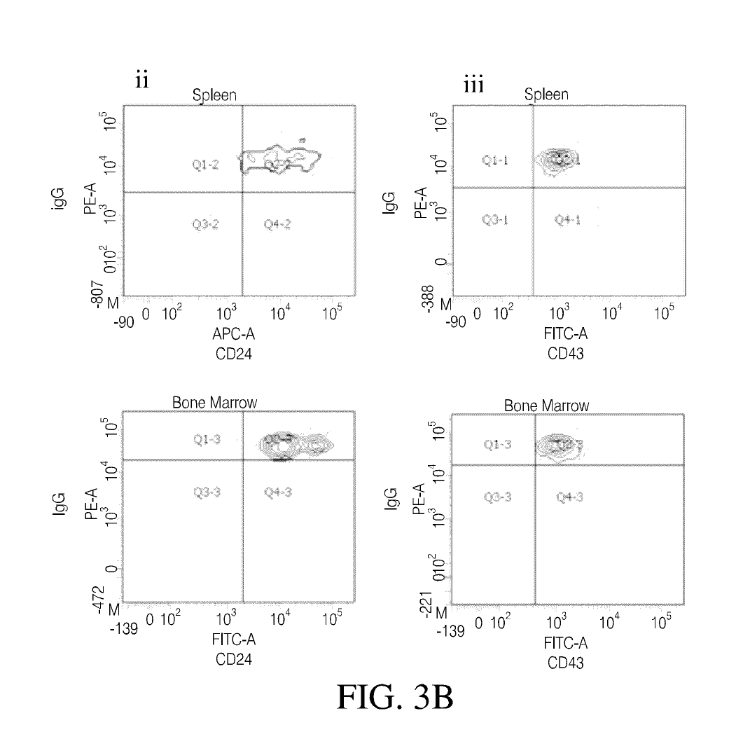

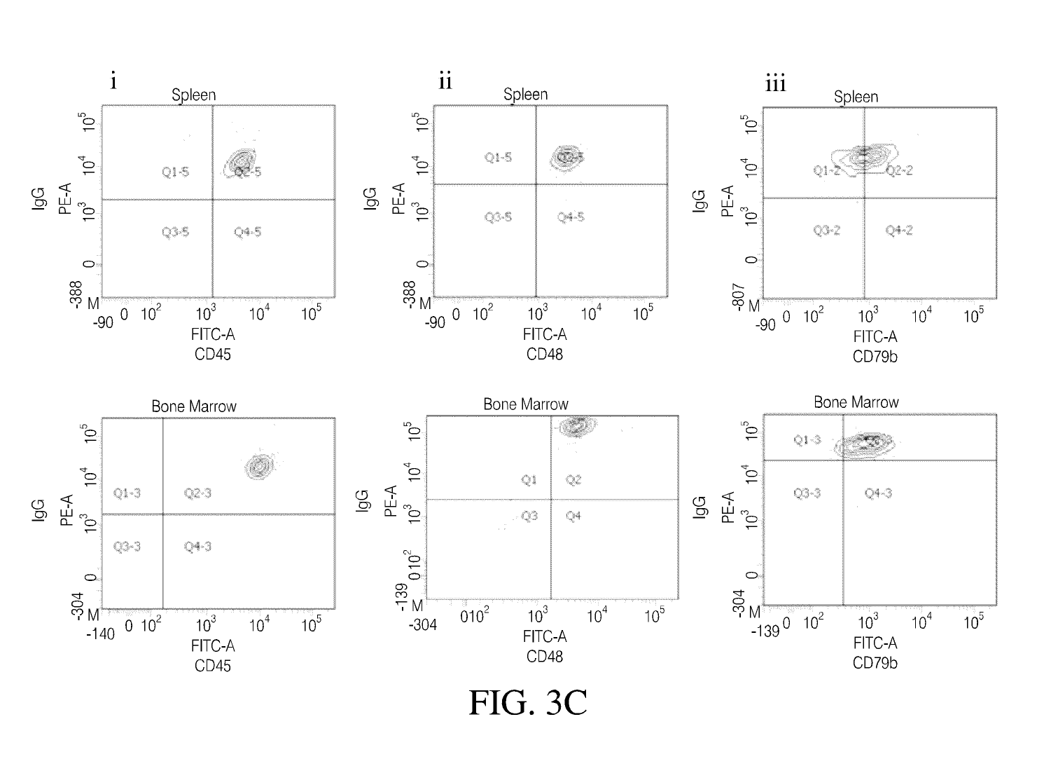

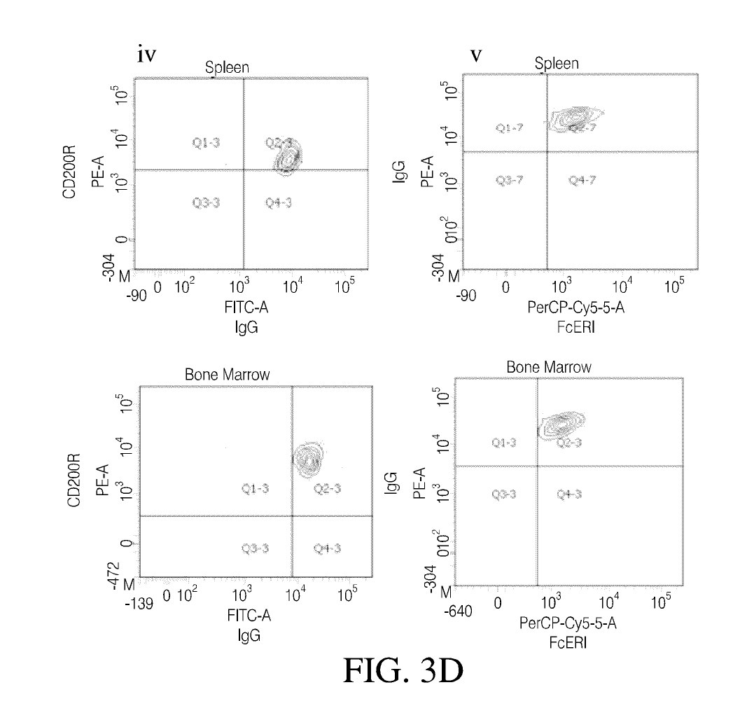

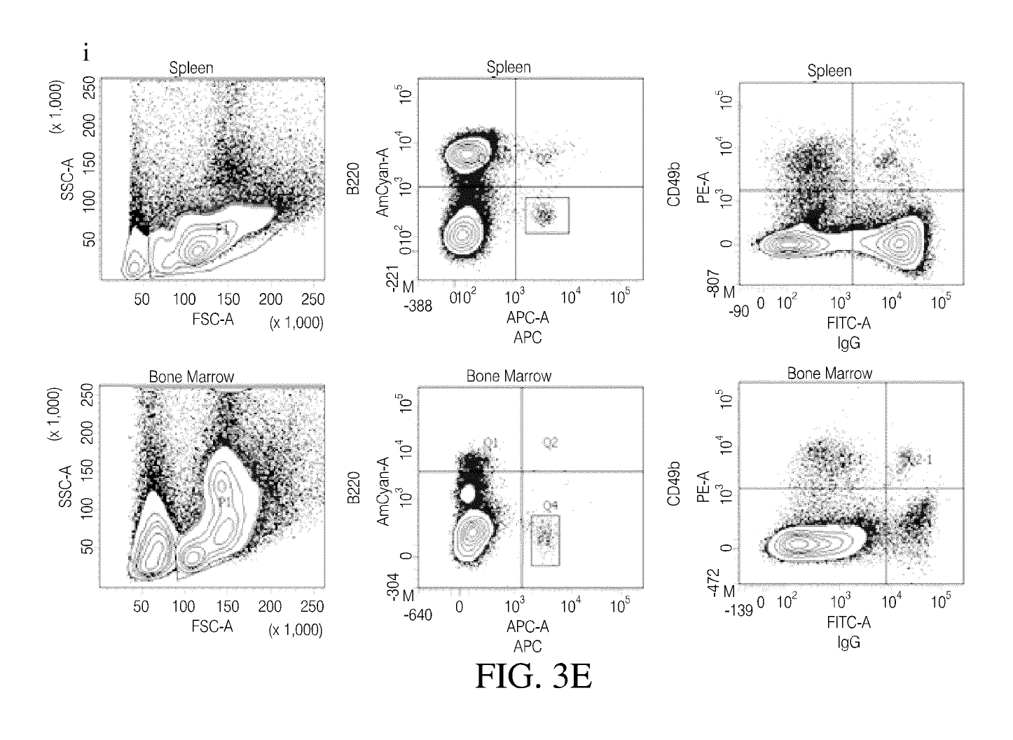

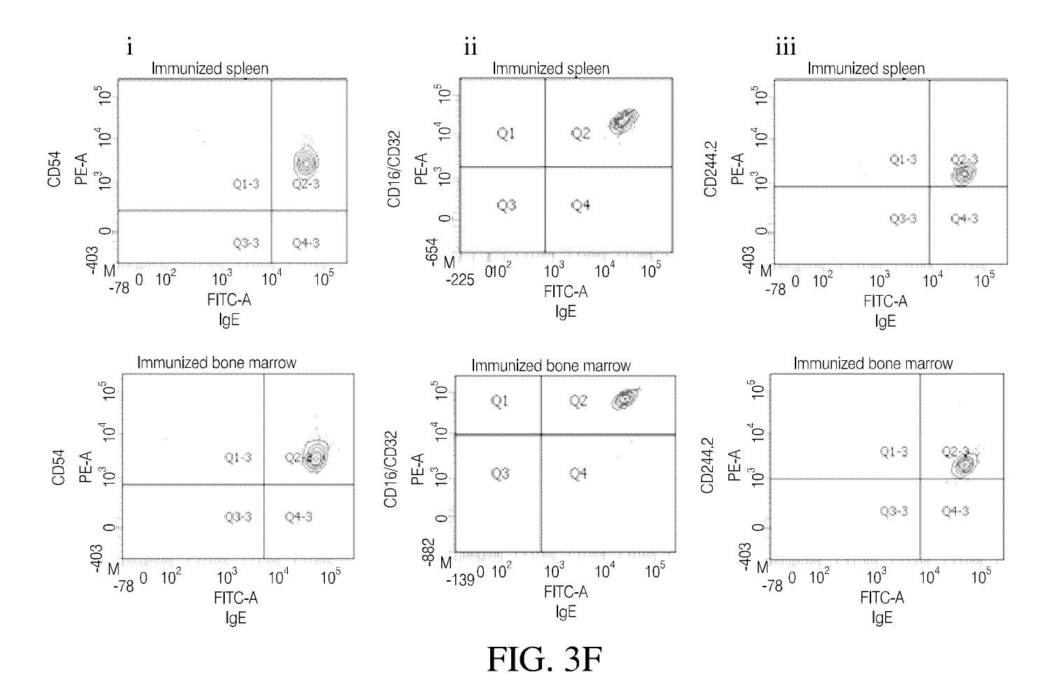

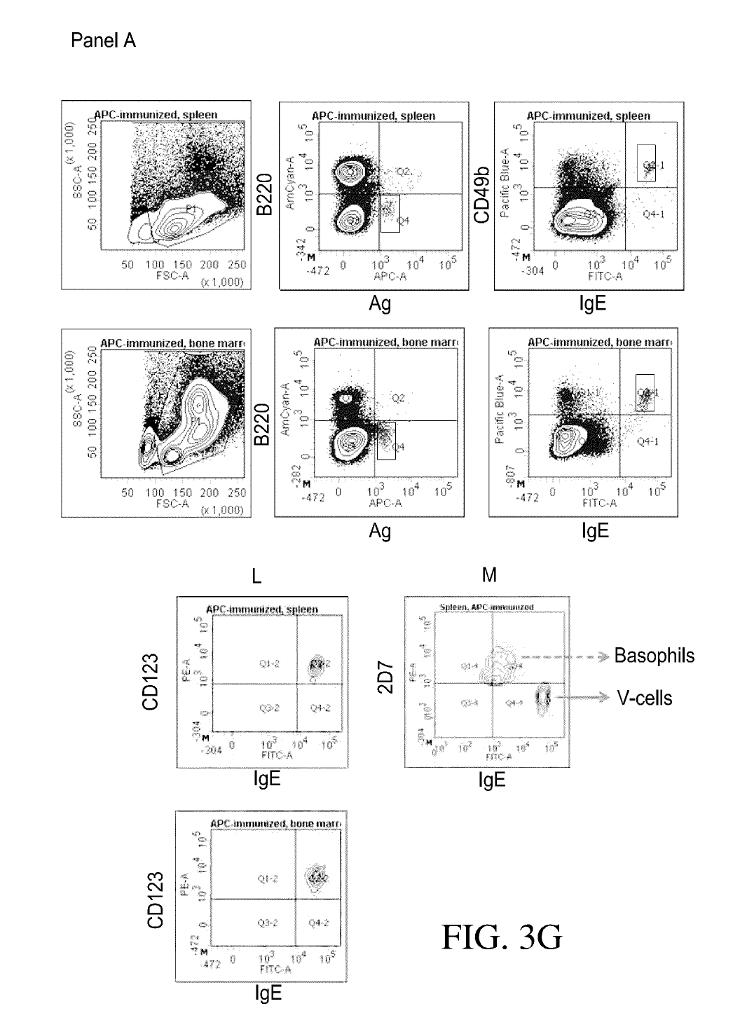

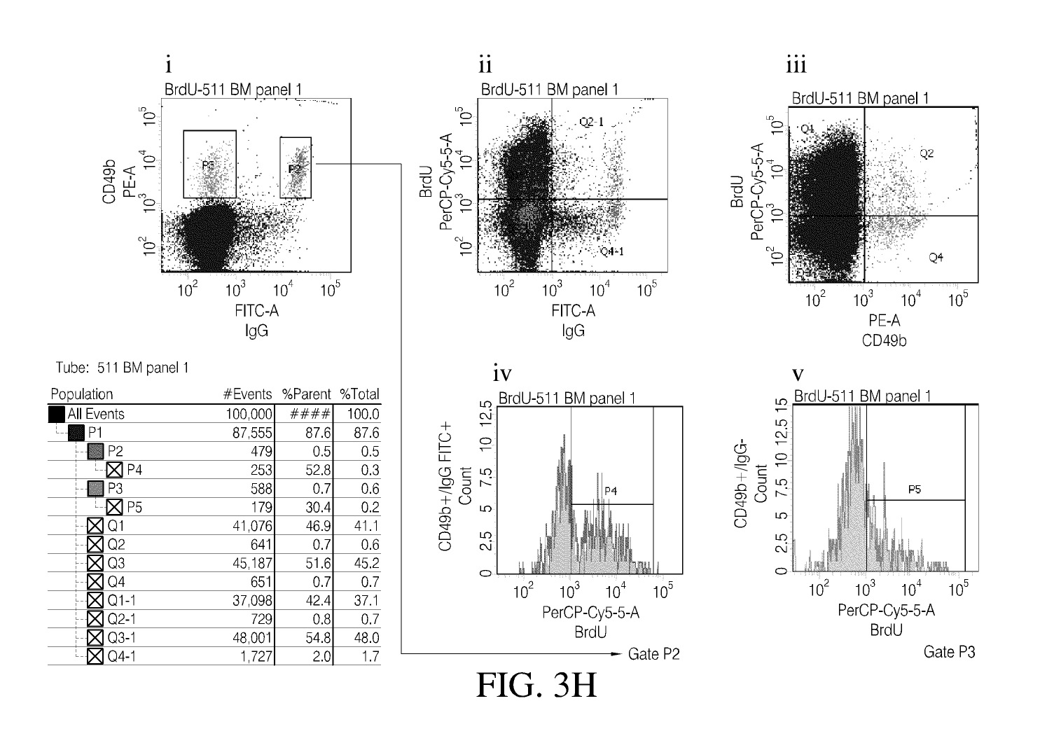

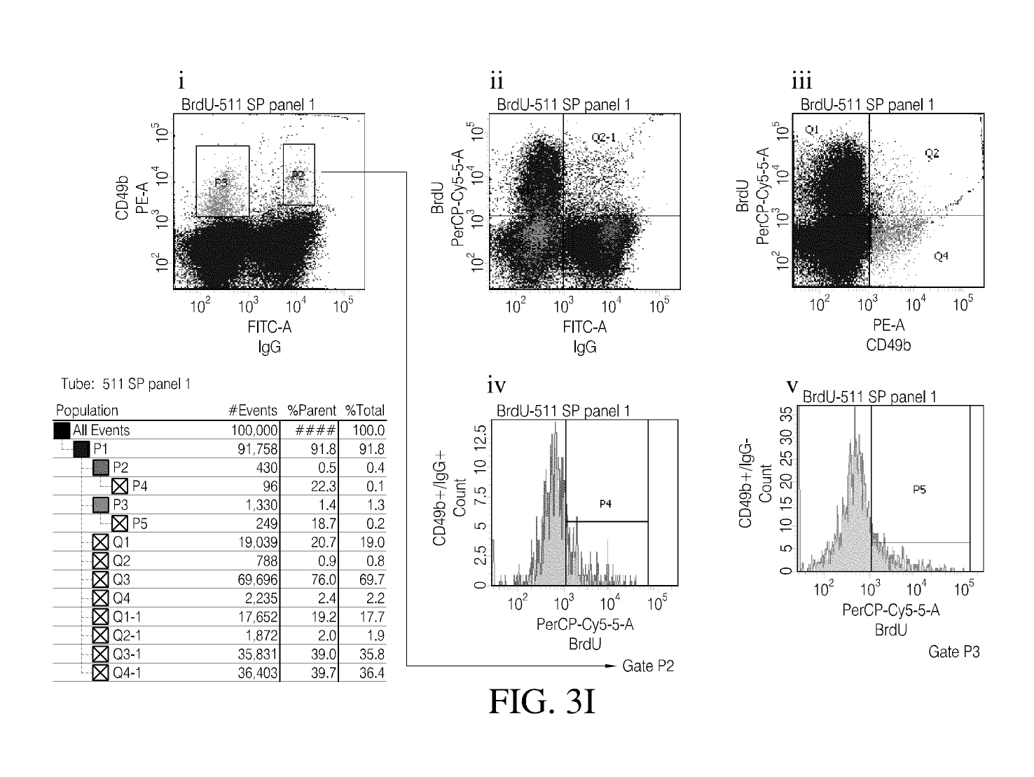

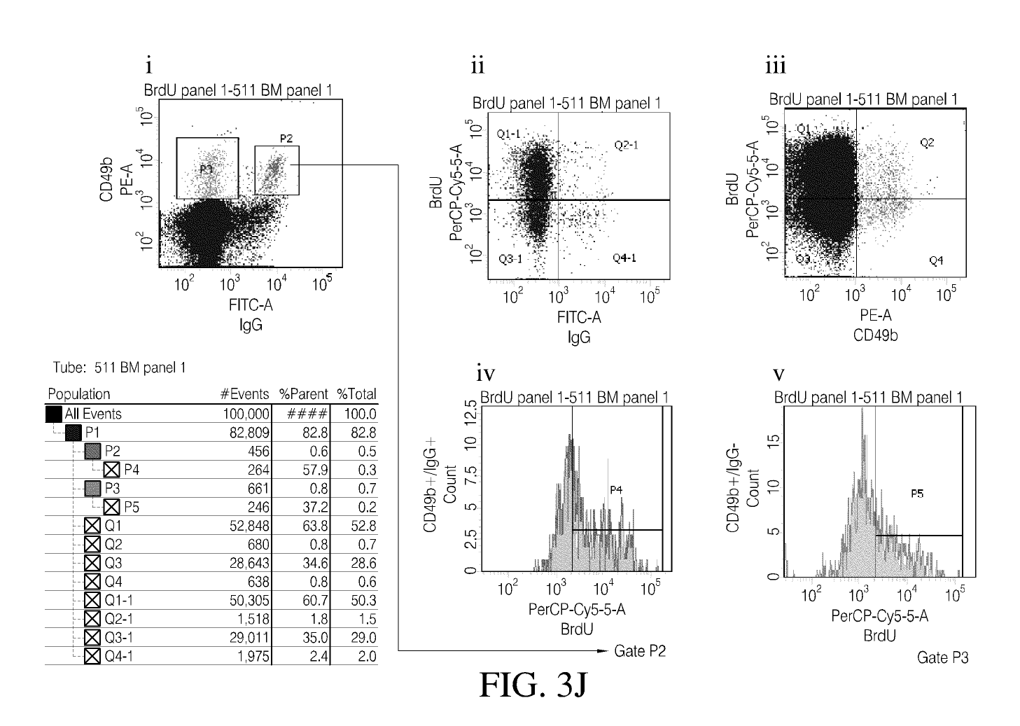

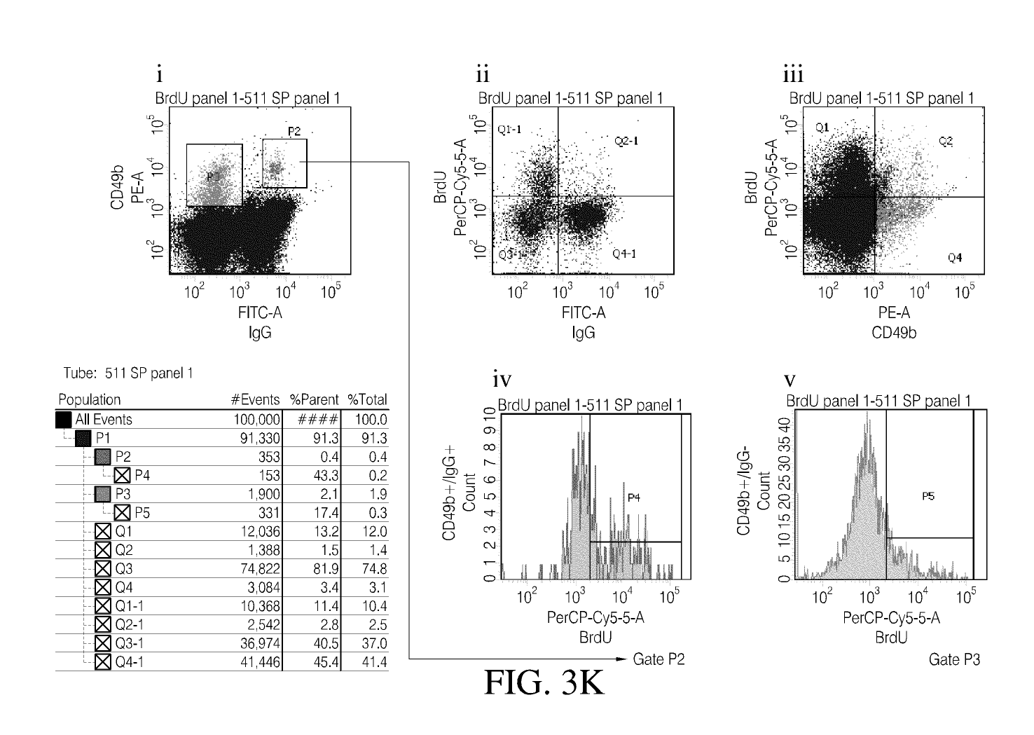

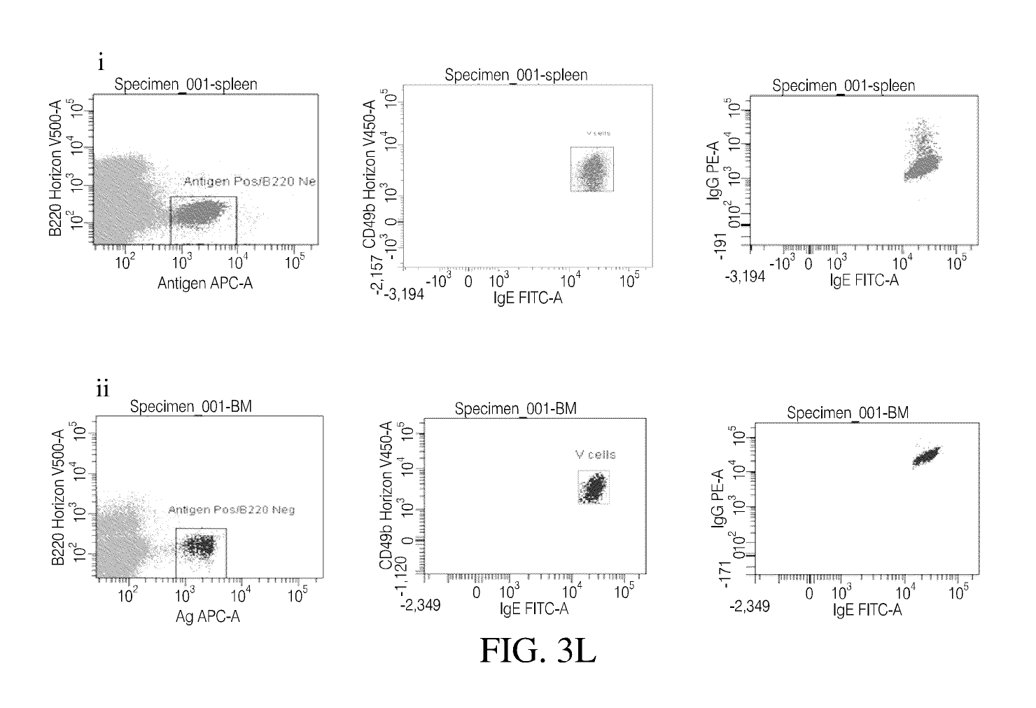

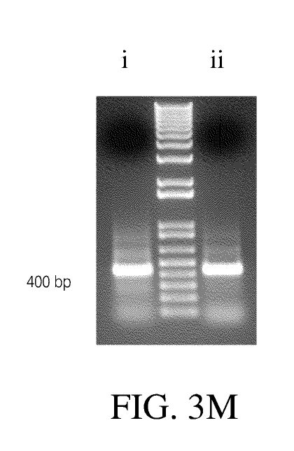

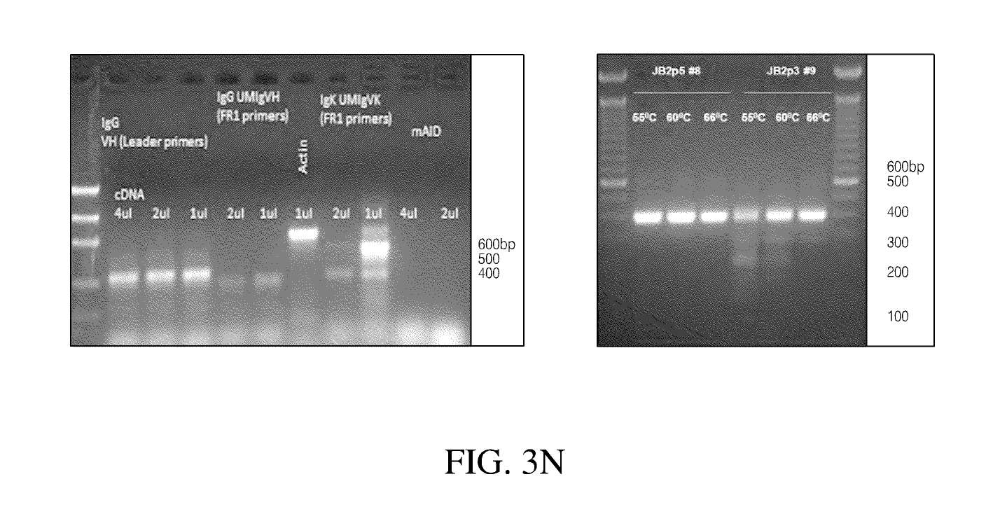

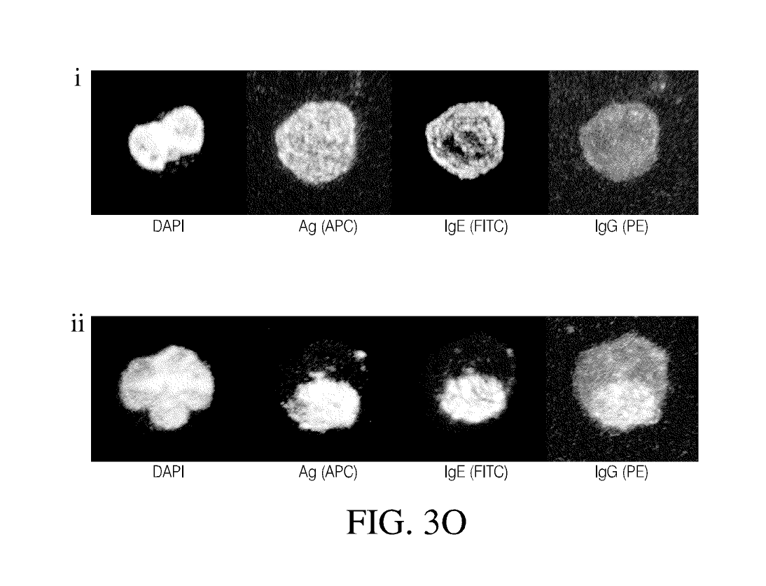

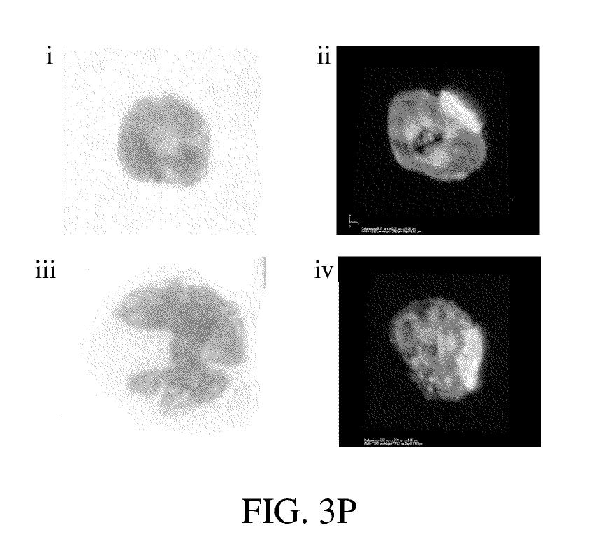

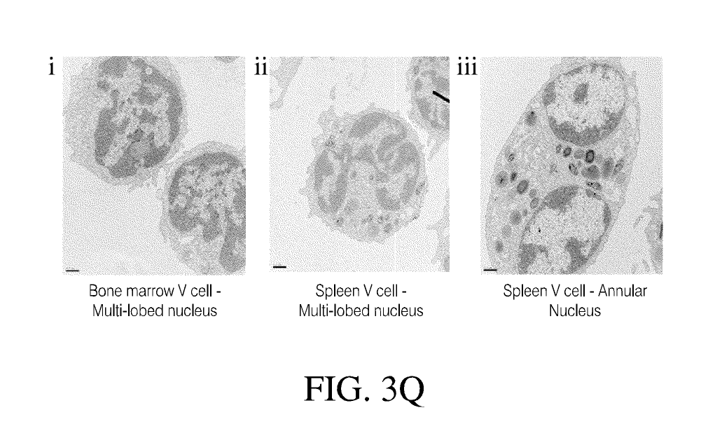

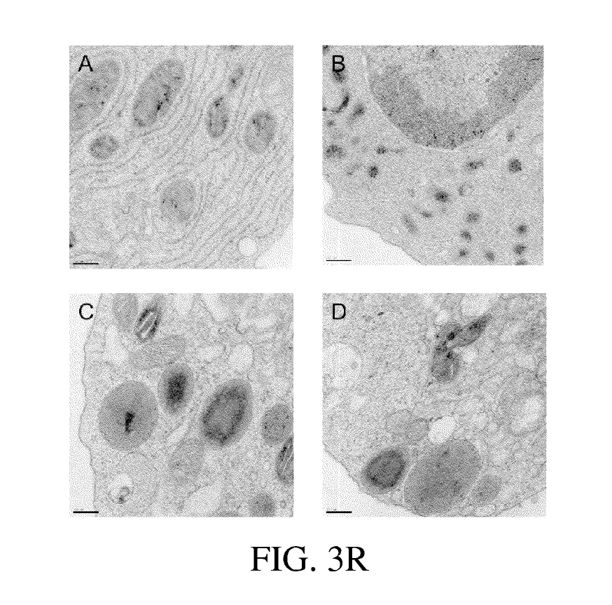

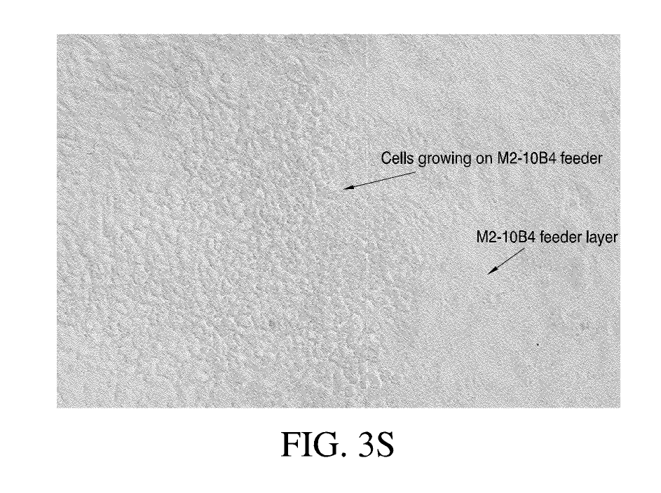

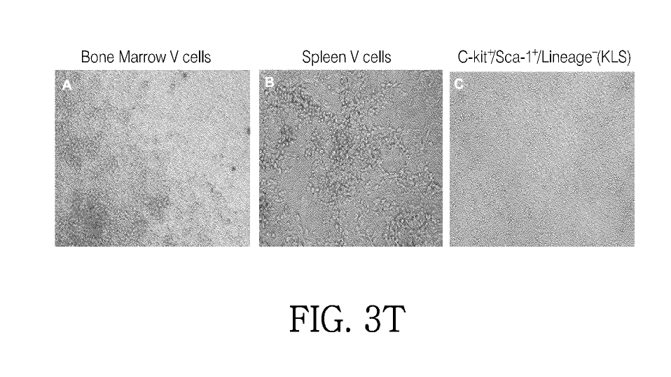

V cells are typically generated in animals 7 to 14 days following a single immunization and have been found in at least the bone marrow, the spleen, and the blood. Applicants believe V cells are likely to be found in other tissues and/or fluids as well. As of the filing of this application, V cells have not been detected in lymph nodes, peritoneal exudate cells (PEC), or thymus, but different immunization protocols and/or different measures of sensitivity and specificity may lead their identification in these tissues. V cells are not terminally differentiated cells and are actively cycling in both the bone marrow (50%-60% range), the spleen (20%-50% range), and blood (15%-16% range) as determined by in-vivo BrdU pulsing (18-42 hrs). V cells can form a population that can vary from 0.3% to 3.5% of total leukocytes depending on the immune response of the animal and their location. The nuclei of V cells are characteristically polymorphonuclear or annular. V cells from both spleen and bone marrow have been shown to exhibit this nuclear morphology (see, e.g. FIGS. 3M-O). V cells can produce cytokines. For example, bone marrow-derived V cells have been shown to produce IL-4, TNF, and occasionally IL-13. For example, spleen-derived V cells have been shown to produce IL-4, TNF, and occasionally IL-13. V cells have been identified in mice and humans, and Applicants believe V cells are likely to be present in other organisms as well.

While V cells produce antibody of an IgG isotype or IgE isotype (and have been shown to simultaneously express IgE and IgG), B cells can produce and express on their surface antibody of an IgM, IgD, IgG, IgA, or IgE isotype. Without being bound by any particular theory, Applicants note that it has previously been believed that a cell cannot simultaneously express both IgG and IgE.

The variable region genes of V cells are typically somatically mutated away from their closest germ line counterparts (i.e. the variable genes of V cells are not in germ line state), unlike the B-1 B cell population in which the V genes are essentially pristine. Without being limited to any theory or theories, V cells appear to have already performed affinity maturation and/or a recruitment of already matured V genes with a fit for the antigen recognized by an individual V cell.

V cells are positive for some markers of non-B cell lineages, and V cells are also negative for markers known to specifically identify B cell lineages. V cells are also known to be negative for basophil specific markers (2D7 and/or BB1). V cells are positive for surface IgG, surface IgE, CD49b (DX5 and HMa2 clones; as used herein, unless explicitly stated otherwise, “CD49b” refers to clones DX5 and HMa2), CD200R, CD244.2, and FcεR1, but are negative to reported B cell lineage markers (including B220, CD5, CD19, CD20, CD21/CD35, CD22.2, CD72, GL-7, IgD, IgM, Ly6-k, Ly6-D, Ly-51 CD127, CD138, CD154, AA4.1 and Pax-5). V cells are also positive for CD16/CD32, CD24, CD43, CD45, CD48, CD49b, CD54, CD79b, and FcεR1. V cells also include a subpopulation that is positive to CD27, CD73, CD45RB and CD80. V cells are also negative for T cell markers (including CD1d, CD3, CD4, CD8, CD25, CD38 and CD134), dendritic cell markers (including CD11c & CD273) and negative to NK markers (including CD49a, CD122 and CD226/NKp46). V cells are also negative to hematopoietic stem cell (HSC) markers (CD34, Sca-1, c-Kit and CD150) and to monocyte markers (Ly-6G). In some embodiments, V cells are positive for CD123. It is noted that B220 can be B cell lineage marker in mice, and CD19 and CD20 can be B cell lineage markers in humans. In some embodiments, V cells are identified as negative for B lineage markers, for example B220, CD5, CD19, CD20, CD21/CD35, CD22.2, CD72, GL-7, IgD, IgM, Ly6-k, Ly6-D, Ly-51 CD127, CD138, CD154, AA4.1 and Pax-5. In some embodiments, V cells are identified as negative to T cell markers (including CD1d, CD3, CD4, CD8, CD25, CD38 and CD134), Dendritic cell markers (including CD11c & CD273) and negative to NK markers (including CD49a, CD122 and CD226/NKp46), HSC markers (CD34, Sca-1, c-Kit and CD150), monocyte markers (CD11b and Ly-6G), and/or basophil markers (2D7 and/or BB1).

Markers for which V cells are positive are summarized in Table 1.1, below. The skilled artisan will appreciate that an identified V cell is positive for each of the markers identified in Table 1.1, but that identification of a V cell does not necessarily require confirmation of the presence of each of these markers.

TABLE 1.1
Phenotypic analysis of V cells: Positive CD Markers and Immunoglobulins

| | | Ligands & | |
|---|---|---|---|
| CD/Ab | | Associated | |
| Name | Alternative Name | molecules | Function |
| CD16/ | FcγRIII/FcγRII, Ly- | IgG Fc | Low affinity IgG Fc receptor III/II |
| CD32 | 17, FCGR3, IGFR3 | | |
| CD24 | Heat Stable Antigen, | CD62P (P-Selectin) | T and B lymphocyte activation |
| | Ly-52, Nectadrin | | and differentiation, adhesion |
| CD43 | Ly-48, Sialophorin, | CD54 | Cell adhesion and T-cell activation |
| | Leukosialin, | | |
| | Galactoglycoprotein | | |
| CD45 | Leukocyte Common | Osteopontin | Regulator of T- and B-cell antigen |
| | Antigen (LCA) | | receptor signaling, regulator of |
| | | | cell growth and differentiation |
| CD48 | Blast-1, Hulym3, | CD2, lck, fyn, | Lymphocyte adhesion and |
| | BCM-1, OX-45, | CD229, CD244 | activation |
| | MEM-102 | | |
| CD49b | VLA-2α, Integrin α2, | Collagen, Laminin, | Cell adhesion |
| | gPIa | MMP-1 | |
| CD54 | ICAM-1, Ly-47, | LFA-1, Mac-1, | Cell adhesion, lymphocyte |
| | MALA-2 | CD43, | activation and migration |
| | | CD11a/CD18, | |
| | | CD11b/CD18, | |
| | | Rhinovirus, CD227 | |
| CD79b | Igb, B29 | Ig, CD5, CD19, | Signal transduction, cell surface |
| | | CD22, CD79a | expression, |
| | | | differentiation/development |
| CD123 | IL-3 Receptor α chain | IL-3 | IL-3 receptor/coreceptor |
| CD200R | OX2, MRC, MOX1, | CD200R1 | Co-stimulates T-cell proliferation. |
| | MOX2 | | May regulate myeloid cell activity |
| CD244.2 | 2B4, C9.1, Ly90, | CD48 | Signal transduction |
| | NAIL, Nmrk, | | |
| | NKR2B4, SLAMF4 | | |
| FcεR1 | High affinity Fc E | IgE | Controls allergic responses and |
| | receptor | | production of immune mediators |
| | | | that promote inflammation |
| IgG | Immunoglobulin G | | Main antibody isotype |
| IgE | Immunoglobulin E | | Immunity to parasites and type I |
| | | | hypersensitivity |
| MHC | H2-D, H2-K | | |
| Class I | | | |
| MHC | I-A/I-E | | Antigen Presentation |
| Class II | | | |

Markers for which V cells are negative are summarized in Table 1.2, below. The skilled artisan will appreciate that an identified V cell is negative for all of the markers listed in Table 1.2, but that identification of a V cell does not necessarily require confirming the absence of each of the listed markers.

TABLE 1.2
Phenotypic Characterization of V cells - Negative CD Markers and
Immunoglobulins

| | | Ligands & | |
|---|---|---|---|
| CD/Ab | | Associated | |
| Name | Alternative Name | molecules | Function |
| CD1d | CD1.1, CD1.2, Ly-38 | Lipid, Glycolipid | Antigen presentation |
| | | Ag | |
| CD3e | CD3ε, CD3ε chain, | TCR complex | Signal transduction |
| | CD3, T3ε | | |
| CD4 | L3T4, Ly-4 | MHC class II, HIV | Signal transduction, |
| | | gp120, IL-16 | receptor/coreceptor |
| CD5 | Ly-1, Lyt-1, Ly-12, | CD72 | Adhesion, regulates T-B |
| | Ly-A | | lymphocyte interaction |
| CD8a | Ly-2, Lyt-2, Ly-B, Ly- | MHC class I | Signal transduction, |
| | 35 | | receptor/coreceptor for MHC |
| | | | class I molecules |
| CD11c | ITGAx [Integrin αx], | iC3b, Fibronectin, | Adhesion, cell migration, survival, |
| | CR4 [complement | ICAM-1 | and proliferation |
| | receptor-4], iC3b | | |
| | receptor, Leu M5, | | |
| | p150,95, | | |
| | CD18/CD11c | | |
| CD19 | B4 | CD21, CD81 | Signal transduction, |
| | | | receptor/coreceptor |
| CD21/CD35 | CR2/CR1 | C3d, EBV, CD23, | Signal transduction |
| | | CD19, CD81 | |
| CD22.2 | Lyb-8.2, Siglec-2 | N-Glycolyl | B cell adhesion, |
| | | neuraminic acid | immunoregulation, |
| | | | receptor/coreceptor, signal |
| | | | transduction |
| CD23 | FceRII, Ly-42 | IgE, CD21, | Regulates B cell activation |
| | | CD11b, CD11c | |
| CD25 | Ly-43, IL-2 Receptor | IL-2 Receptor α | Activation/costimulation, |
| | α chain, p55 | | receptor/coreceptor |
| CD27 | T14, s152, tnfrs7, | CD70, TRAF2, | Activation/costimulation, |
| | Tp55 | TRAF5 | receptor/coreceptor |
| CD34 | Mucosialin | CD62L (L- | Cell adhesion |
| | | Selectin) | |
| CD38 | ADP-ribosyl cyclase, | CD31, Hyaluronic | Cell adhesion and signal |
| | T10, Cyclic ADP- | acid, CD3/TcR | transduction |
| | ribose hydrolase 1 | complex, CD16, | |
| | | HLA Class II | |
| CD45R | B220, Ly-5, Lyt-4, | | Regulator of T- and B-cell antigen |
| | T200, Protein tyrosine | | receptor signaling, regulator of |
| | phosphatase receptor | | cell growth and differentiation |
| | type C (PTPRC) | | |
| CD45RB | | | |
| CD49a | VLA-1α, Integrin α1 | Collagen, Laminin | Cell adhesion |
| CD62P | P-Selectin, GMP-140, | CD162, CD24 | Cell adhesion |
| | PADGEM | | |
| CD64 | FcγR1, Fc-γ receptor 1 | IgG | Ig Fc receptor |
| CD69 | Very Early Activation | | Activation/costimulation, |
| | Antigen | | differentiation/development |
| CD72 | Lyb-2, Ly-m19 | CD5, CD100 | Activation/costimulation, |
| | | | differentiation/development |
| CD73 | NT, Ecto-5′- | NMP | Enzymatic activity |
| | nucleotidase | | |
| CD80 | B7/BB1, B7-1, Ly-53 | CD28, CD152 | Activation/costimulation, |
| | | | immunoregulation |
| CD93 | AA4.1, C1qRp | CCL21 | Potentially involved in |
| | | | angiogenesis, endothelial cell |
| | | | migration, and clearance of dying |
| | | | cells |
| CD117 | c-kit, Steel factor | c-Kit Ligand (Steel, | Signal transduction, |
| | receptor, Dominant | stem-cell, or mast- | differentiation/development, |
| | white spotting | cell growth factor) | receptor/coreceptor |
| CD122 | IL-2 and IL-15 | IL-2, IL-15 | Signal transduction, |
| | Receptor b chain | | immunoregulation, |
| | | | receptor/coreceptor |
| CD127 | IL-7 Receptor α chain | IL-7 | Signal transduction, |
| | | | differentiation/development, |
| | | | receptor/coreceptor |
| CD134 | Ly-70, OX-40 antigen, | OX-40 Ligand | Activation/costimulation |
| | ACT35 antigen | | |
| CD138 | Syndecan-1, Sdc1 | Interstitial matrix | Adhesion |
| | | proteins | |
| CD150 | IPO-3, ESTM51, | Measles virus, | Signal transduction |
| | Slam | CD45 | |
| CD154 | gp39, CD40 Ligand, | CD40 | Activation/costimulation |
| | Ly-62, HIGM1, | | |
| | IMD3, T-BAM, | | |
| | Tnfsf5 | | |
| CD226 | DNAX accessory | CD112, CD155, | Involved in platelet adhesion and |
| | molecule 1 (DNAM- | LFA-1 | activation, megakaryocyte |
| | 1), Platelet and T cell | | adhesion and |
| | activation antigen 1 | | maturation, and adhesion of |
| | (PTA-1), T lineage- | | cytotoxic T and NK cells to target |
| | specific activation | | cells. Important |
| | antigen 1 antigen | | for tumor immunosurveillance. |
| | (TLiSA1) | | |
| CD273 | B7DC, PD-L2, Btdc, | PD1 (CD279) | Costimulation, inhibition |
| | PD-L2, MGC124039, | | |
| | MGC124040, | | |
| | F730015O22Rik, | | |
| | Pdcd1lg2 | | |
| CD284 | TLR4, Ly87, Ran/M1, | CD14, MD-2 | Lymphocyte maturation |
| | Rasl2-8 | | |
| GL7 | T & B cell Activation | | Activation/costimulation |
| | antigen, Ly-77 | | |
| Ly-51 | 6C3/BP-1 Antigen | | Cell surface differentiation |
| Ly-6D | ThB | Rag-1 | B cell lineage differentiation |
| Ly6-G & | Gr-1, Myeloid | | Granulocyte marker |
| Ly6-C | differentiation antigen | | |
| Ly6-K | CO-16 | | Cell growth and plasma cell |
| | | | marker |
| Pax5 | BSAP | TLE4, DAXX | Early stages of B cell |
| | | | differentiation |
| IgA | Immunoglobulin A | | |
| IgD | IGHD; Igh-5; | | |
| | Immunoglobulin heavy | | |
| | chain 5; Ig delta chain | | |
| | C region | | |
| IgM | Immunoglobulin M | | |
| 2D7 | Basophil-specific | detects-a basophil- | Not currently determined |
| | marker | specific protein of | |
| | | 7.2-7.5 kDa | |
| | | localized in | |
| | | secretory granules | |
| BB1 | Basophil-specific | Basogranulin | |
| | marker | | |

Markers that appear to be weakly expressed on V cells are summarized in Table 1.3, below:

TABLE 1.3
Phenotypic Characterization of V cells - Low CD Markers. (In some
instances, these two markers appear to be expressed at low
levels in the V cells.)

| | | Ligands & | |
|---|---|---|---|
| CD/Ab | | Associated | |
| Name | Alternative Name | molecules | Function |
| CD11b | Integrin αM, Ly-40, | CD54, iC3b, | Adhesion, chemotaxis, |
| | CR3, CR3A, MAC1 | Fibronectin | apoptosis |
| CD90.2 | Thy-1.2, q-C3H | | Signal transduction, |
| | | | activation/costimulation, |
| | | | adhesion, differentiation/ |
| | | | development |

In some embodiments, V cells are characterized as having at least one of the following combinations of markers, and the skilled artisan will readily appreciate that V cells can be identified with reagents targeting one or more of the following combinations of markers: CD49b+IgE+ IgG+ CD200R+ B220− 2D7− BB1−, CD49b+ IgE+ IgG+ CD200R+ B220− 2D7−, CD49b+ IgE+ IgG+ CD200R+ B220− BB1−, CD49b+ IgE+ IgG+ CD244.2+ B220− 2D7− BB1−, CD49b+ IgE+ IgG+ CD244.2+ B220− 2D7−, CD49b+IgE+ IgG+ CD244.2+ B220− BB1−, CD49b+ IgE+ IgG+ FcεR1+ B220− 2D7− BB1−, CD49b+ IgE+ IgG+ FcεR1+ B220− 2D7−, CD49b+ IgE+ IgG+ FcεR1+ B220− BB1−, CD49b+ IgE+ IgG+ CD200R+ B220− NK1.1−2D7− BB1−, CD49b+ IgE+ IgG+ CD200R+ B220− NK1.1−2D7, CD49b+ IgE+ IgG+ CD200R+ B220− NK1.1−BB1−, CD49b+ IgE+ IgG+ CD244.2+ B220− NK1.1−2D7− BB1−, CD49b+ IgE+ IgG+ CD244.2+ B220− NK1.1−2D7−, CD49b+ IgE+ IgG+ CD244.2+ B220− NK1.1−BB1−, CD49b+ IgE+ IgG+ FcεR1+ B220− NK1.1−2D7− BB1−, CD49b+ IgE+ IgG+ FcεR1+ B220− NK1.1−2D7−, CD49b+ IgE+ IgG+ FcεR1+ B220− NK1.1−BB1−, CD49b+ IgE+ IgG+ CD200R+ B220− NKp46−2D7− BB1−, CD49b+ IgE+ IgG+ CD200R+ B220− NKp46−2D7−, CD49b+ IgE+ IgG+ CD200R+ B220− NKp46−BB1, CD49b+ IgE+ IgG+ CD244.2+ B220− NKp46−2D7− BB1−, CD49b+ IgE+ IgG+ CD244.2+ B220− NKp46−2D7−, CD49b+ IgE+ IgG+ CD244.2+ B220− NKp46−BB1−, CD49b+ IgE+ IgG+ FcεR1+ B220− NKp46−2D7− BB1−, CD49b+ IgE+ IgG+ FcεR1+ B220− NKp46−2D7−, CD49b+ IgE+ IgG+ FcεR1+ B220− NKp46−BB1−, CD49b+ IgE+ IgG+ CD200R+ B220− CD122−2D7− BB1−, CD49b+ IgE+ IgG+ CD200R+ B220− CD122−2D7−, CD49b+ IgE+IgG+ CD200R+ B220− CD122−BB1−, CD49b+ IgE+ IgG+ CD244.2+ B220− CD122−, CD49b+ IgE+ IgG+ FcεR1+ B220− CD122−2D7− BB1−, CD49b+ IgE+ IgG+ CD244.2+ B220− CD122−, CD49b+ IgE+ IgG+ FcεR1+ B220− CD122− 2D7−, CD49b+ IgE+ IgG+ CD244.2+ B220− CD122−, CD49b+ IgE+ IgG+ FcεR1+ B220− CD122−BB1−, CD49b+ IgE+ IgG+ CD200R+ B220− Ag+ 2D7− BB1−, CD49b+ IgE+ IgG+ CD200R+ B220− Ag+ 2D7−, CD49b+ IgE+ IgG+ CD200R+ B220− Ag+ BB1−, CD49b+ IgE+ IgG+ CD244.2+ B220− Ag+ 2D7− BB1−, CD49b+ IgE+ IgG+ CD244.2+ B220− Ag+ 2D7−, CD49b+ IgE+ IgG+ CD244.2+ B220− Ag+ BB1−, CD49b+ IgE+ IgG+ FcεR1+ B220− Ag+ 2D7− BB1−, CD49b+ IgE+ IgG+ FcεR1+ B220− Ag+ 2D7−, CD49b+ IgE+ IgG+ FcεR1+ B220− Ag+ BB1−, CD49b+ IgE+ IgG+ CD200R+ B220− NK1.1−Ag+ 2D7− BB1−, CD49b+ IgE+ IgG+ CD200R+ B220− NK1.1−Ag+ 2D7−, CD49b+ IgE+ IgG+ CD200R+ B220− NK1.1−Ag+ 2D7−, CD49b+ IgE+ IgG+ CD244.2+ B220− NK1.1−Ag+ 2D7− BB1−, CD49b+ IgE+ IgG+ CD244.2+ B220− NK1.1−Ag+ 2D7−, CD49b+ IgE+ IgG+ CD244.2+ B220− NK1.1−Ag+ BB1−, CD49b+ IgE+ IgG+ FcεR1+ B220− NK1.1−Ag+ 2D7− BB1−, CD49b+ IgE+ IgG+ FcεR1+ B220− NK1.1−Ag+ 2D7−, CD49b+ IgE+ IgG+ FcεR1+ B220− NK1.1−Ag+ BB1−, CD49b+ IgE+ IgG+ CD200R+ B220− NKp46− Ag+ 2D7− BB1−, CD49b+ IgE+ IgG+ CD200R+ B220− NKp46− Ag+ 2D7−, CD49b+ IgE+ IgG+ CD200R+ B220− NKp46− Ag+ BB1−, CD49b+ IgE+ IgG+ CD244.2+ B220− NKp46− Ag+ 2D7− BB1−, CD49b+ IgE+ IgG+ CD244.2+ B220− NKp46− Ag+ 2D7−, CD49b+ IgE+ IgG+ CD244.2+ B220− NKp46− Ag+ BB1−, CD49b+ IgE+ IgG+ FcεR1+ B220− NKp46− Ag+ 2D7− BB1−, CD49b+ IgE+ IgG+ FcεR1+ B220− NKp46− Ag+ 2D7−, CD49b+ IgE+ IgG+ FcεR1+ B220− NKp46− Ag+ BB1−, CD49b+ IgE+ IgG+ CD200R+ B220− CD122− Ag+ 2D7− BB1−, CD49b+ IgE+ IgG+ CD200R+ B220− CD122− Ag+ 2D7−, CD49b+ IgE+ IgG+ CD200R+ B220− CD122− Ag+ BB1−, CD49b+ IgE+ IgG+ CD244.2+ B220− CD122− Ag+ 2D7− BB1−, CD49b+ IgE+ IgG+ CD244.2+ B220− CD122− Ag+ 2D7−, CD49b+ IgE+ IgG+ CD244.2+ B220− CD122− Ag+ BB1−, CD49b+ IgE+ IgG+ FcεR1+ B220− CD122− Ag+ 2D7− BB1−, CD49b+ IgE+ IgG+ FcεR1+ B220− CD122− Ag+ 2D7−, CD49b+ IgE+ IgG+ FcεR1+ B220− CD122− Ag+ BB1−, CD49b+ IgE+ IgG+ CD200R+ CD19−, CD49b+ IgE+ IgG+ CD244.2+ CD19− 2D7− BB1−, CD49b+ IgE+ IgG+ CD200R+ CD19−, CD49b+ IgE+ IgG+ CD244.2+ CD19− 2D7−, CD49b+ IgE+ IgG+ CD200R+ CD19−, CD49b+ IgE+ IgG+ CD244.2+ CD19− BB1−, CD49b+ IgE+ IgG+ FcεR1+ CD19− 2D7− BB1−, CD49b+ IgE+ IgG+ FcεR1+ CD19− 2D7−, CD49b+ IgE+ IgG+ FcεR1+ CD19− BB1−, CD49b+ IgE+ IgG+ CD200R+ CD19− NK1.1− 2D7− BB1−, CD49b+ IgE+ IgG+ CD200R+ CD19− NK1.1− 2D7−, CD49b+ IgE+ IgG+ CD200R+ CD19− NK1.1− BB1−, CD49b+ IgE+ IgG+ CD244.2+ CD19− NK1.1− 2D7− BB1−, CD49b+ IgE+ IgG+ CD244.2+ CD19− NK1.1− 2D7−, CD49b+ IgE+ IgG+ CD244.2+ CD19− NK1.1− BB1−, CD49b+ IgE+ IgG+ FcεR1+ CD19− NK1.1− 2D7− BB1−, CD49b+ IgE+ IgG+ FcεR1+ CD19− NK1.1− 2D7−, CD49b+ IgE+ IgG+ FcεR1+ CD19− NK1.1− BB1−, CD49b+ IgE+ IgG+ CD200R+ CD19− NKp46− 2D7− BB1−, CD49b+ IgE+ IgG+ CD200R+ CD19− NKp46− 2D7−, CD49b+ IgE+ IgG+ CD200R+ CD19− NKp46− BB1−, CD49b+ IgE+ IgG+ CD244.2+ CD19− NKp46− 2D7− BB1−, CD49b+ IgE+ IgG+ CD244.2+ CD19− NKp46− 2D7−, CD49b+ IgE+ IgG+ CD244.2+ CD19− NKp46− BB1−, CD49b+ IgE+ IgG+ FcεR1+ CD19− NKp46− 2D7− BB1−, CD49b+ IgE+ IgG+ FcεR1+ CD19− NKp46− 2D7−, CD49b+ IgE+ IgG+ FcεR1+ CD19− NKp46− BB1−, CD49b+ IgE+ IgG+ CD200R+ CD19− CD122− 2D7− BB1−, CD49b+ IgE+ IgG+ CD200R+ CD19− CD122− 2D7−, CD49b+ IgE+ IgG+ CD200R+ CD19− CD122− BB1−, CD49b+ IgE+ IgG+ CD244.2+ CD19− CD122− 2D7− BB1−, CD49b+ IgE+ IgG+ CD244.2+ CD19− CD122− 2D7−, CD49b+ IgE+ IgG+ CD244.2+ CD19− CD122− BB1−, CD49b+ IgE+ IgG+ FcεR1+ CD19− CD122− 2D7− BB1−, CD49b+ IgE+ IgG+ FcεR1+ CD19− CD122− 2D7−, CD49b+ IgE+ IgG+ FcεR1+ CD19− CD122− BB1−, CD49b+ IgE+ IgG+ CD200R+ CD19− Ag+ 2D7− BB1−, CD49b+ IgE+ IgG+ CD200R+ CD19− Ag+ 2D7−, CD49b+ IgE+ IgG+ CD200R+ CD19− Ag+ BB1−, CD49b+ IgE+ IgG+ CD244.2+ CD19− Ag+ 2D7− BB1−, CD49b+ IgE+ IgG+ CD244.2+ CD19− Ag+ 2D7−, CD49b+ IgE+ IgG+ CD244.2+ CD19− Ag+ BB1−, CD49b+ IgE+ IgG+ FcεR1+ CD19− Ag+, CD49b+ IgE+ IgG+ CD200R+ CD19− NK1.1− Ag+ 2D7− BB1−, CD49b+ IgE+ IgG+ FcεR1+ CD19− Ag+, CD49b+ IgE+ IgG+ CD200R+ CD19− NK1.1− Ag+ 2D7−, CD49b+ IgE+ IgG+ FcεR1+ CD19− Ag+, CD49b+ IgE+ IgG+ CD200R+ CD19− NK1.1− Ag+ BB1−, CD49b+ IgE+ IgG+ CD244.2+ CD19− NK1.1− Ag+ 2D7− BB1−, CD49b+ IgE+ IgG+ CD244.2+ CD19− NK1.1− Ag+ 2D7−, CD49b+ IgE+ IgG+ CD244.2+ CD19− NK1.1− Ag+ BB1−, CD49b+ IgE+ IgG+ FcεR1+ CD19− NK1.1− Ag+ 2D7− BB1−, CD49b+ IgE+ IgG+ FcεR1+ CD19− NK1.1− Ag+ 2D7−, CD49b+ IgE+ IgG+ FcεR1+ CD19− NK1.1− Ag+ BB1−, CD49b+ IgE+ IgG+ CD200R+ CD19− NKp46− Ag+ 2D7− BB1−, CD49b+ IgE+ IgG+ CD200R+ CD19− NKp46− Ag+ 2D7−, CD49b+ IgE+ IgG+ CD200R+ CD19− NKp46− Ag+ BB1−, CD49b+ IgE+ IgG+ CD244.2+ CD19− NKp46− Ag+ 2D7− BB1−, CD49b+ IgE+ IgG+ CD244.2+ CD19− NKp46− Ag+ 2D7−, CD49b+ IgE+ IgG+ CD244.2+ CD19− NKp46− Ag+ BB1−, CD49b+ IgE+ IgG+ FcεR1+ CD19− NKp46− Ag+ 2D7− BB1−, CD49b+ IgE+ IgG+ FcεR1+ CD19− NKp46− Ag+ BB1−, CD49b+ IgE+ IgG+ FcεR1+ CD19− NKp46− Ag+ BB1−, CD49b+ IgE+ IgG+ CD200R+ CD19− CD122− Ag+ 2D7− BB1−, CD49b+ IgE+ IgG+ CD200R+ CD19− CD122− Ag+ 2D7−, CD49b+ IgE+ IgG+ CD200R+ CD19− CD122− Ag+ BB1−, CD49b+ IgE+ IgG+ CD244.2+ CD19− CD122− Ag+ 2D7− BB1−, CD49b+ IgE+ IgG+ CD244.2+ CD19− CD122− Ag+ 2D7−, CD49b+ IgE+ IgG+ CD244.2+ CD19− CD122− Ag+ BB1−, CD49b+ IgE+ IgG+ FcεR1+ CD19− CD122− Ag+ 2D7− BB1−, CD49b+ IgE+ IgG+ FcεR1+ CD19− CD122− Ag+ 2D7−, CD49b+ IgE+ IgG+ FcεR1+ CD19− CD122− Ag+ BB1−, CD49b+ IgE+ IgG+ CD200R+ CD20− 2D7− BB1−, CD49b+ IgE+ IgG+ CD200R+ CD20− 2D7−, CD49b+ IgE+ IgG+ CD200R+ CD20− BB1−, CD49b+ IgE+ IgG+ CD244.2+ CD20− 2D7− BB1−, CD49b+ IgE+ IgG+ CD244.2+ CD20− 2D7−, CD49b+ IgE+ IgG+ CD244.2+ CD20− BB1−, CD49b+ IgE+ IgG+ FcεR1+ CD20− 2D7− BB1−, CD49b+ IgE+ IgG+ FcεR1+ CD20− 2D7−, CD49b+ IgE+ IgG+ FcεR1+ CD20− BB1−, CD49b+ IgE+ IgG+ CD200R+ CD20− NK1.1− 2D7− BB1−, CD49b+ IgE+ IgG+ CD200R+ CD20− NK1.1− 2D7−, CD49b+ IgE+ IgG+ CD200R+ CD20− NK1.1− BB1−, CD49b+ IgE+ IgG+ CD244.2+ CD20− NK1.1− 2D7− BB1−, CD49b+ IgE+ IgG+ CD244.2+ CD20− NK1.1− 2D7−, CD49b+ IgE+ IgG+ CD244.2+ CD20− NK1.1− 2D7− BB1−, CD49b+ IgE+ IgG+ FcεR1+ CD20− NK1.1− BB1−, CD49b+ IgE+ IgG+ CD200R+ CD20− NKp46− 2D7− BB1−, CD49b+ IgE+ IgG+ CD200R+ CD20− NKp46− 2D7−, CD49b+ IgE+ IgG+ CD200R+ CD20− NKp46− 2D7− BB1− CD49b+ IgE+ IgG+ CD244.2+ CD20− NKp46− BB1−, CD49b+ IgE+ IgG+ FcεR1+ CD20− NKp46− 2D7− BB1−, CD49b+ IgE+ IgG+ FcεR1+ CD20− NKp46− 2D7−, CD49b+ IgE+ IgG+ FcεR1+ CD20− NKp46− BB1−, CD49b+ IgE+ IgG+ CD200R+ CD20− CD122− 2D7− BB1−, CD49b+ IgE+ IgG+ CD200R+ CD20− CD122− 2D7−, CD49b+ IgE+ IgG+ CD200R+ CD20− CD122− BB1−, CD49b+ IgE+ IgG+ CD244.2+ CD20− CD122− 2D7− BB1−, CD49b+ IgE+ IgG+ CD244.2+ CD20− CD122− 2D7−, CD49b+ IgE+ IgG+ CD244.2+ CD20− CD122− BB1−, CD49b+ IgE+ IgG+ FcεR1+ CD20− CD122− 2D7− BB1−, CD49b+ IgE+ IgG+ FcεR1+ CD20− CD122− 2D7−, CD49b+ IgE+ IgG+ FcεR1+ CD20− CD122− BB1−, CD49b+ IgE+ IgG+ CD200R+ CD20− Ag+ 2D7− BB1−, CD49b+ IgE+ IgG+ CD200R+ CD20− Ag+ 2D7−, CD49b+ IgE+ IgG+ CD200R+ CD20− Ag+ BB1−, CD49b+ IgE+ IgG+ CD244.2+ CD20− Ag+ 2D7− BB1−, CD49b+ IgE+ IgG+ CD244.2+ CD20− Ag+ 2D7−, CD49b+ IgE+ IgG+ CD244.2+ CD20− Ag+ BB1−, CD49b+ IgE+ IgG+ FcεR1+ CD20− Ag+ 2D7− BB1−, CD49b+ IgE+ IgG+ FcεR1+ CD20− Ag+ 2D7−, CD49b+ IgE+ IgG+ FcεR1+ CD20− Ag+ BB1−, CD49b+ IgE+ IgG+ CD200R+ CD20− NK1.1− Ag+ 2D7− BB1−, CD49b+ IgE+ IgG+ CD200R+ CD20− NK1.1− Ag+ 2D7−, CD49b+ IgE+ IgG+ CD200R+ CD20− NK1.1− Ag+ BB1−, CD49b+ IgE+ IgG+ CD244.2+ CD20− NK1.1− Ag+ 2D7− BB1−, CD49b+ IgE+ IgG+ CD244.2+ CD20− NK1.1− Ag+ 2D7−, CD49b+ IgE+ IgG+ CD244.2+ CD20− NK1.1− Ag+ BB1−, CD49b+ IgE+ IgG+ FcεR1+ CD20− NK1.1− Ag+ 2D7− BB1−, CD49b+ IgE+ IgG+ FcεR1+ CD20− NK1.1− Ag+ 2D7−, CD49b+ IgE+ IgG+ FcεR1+ CD20− NK1.1− Ag+ BB1−, CD49b+ IgE+ IgG+ CD200R+ CD20− NKp46− Ag+ 2D7− BB1−, CD49b+ IgE+ IgG+ CD200R+ CD20− NKp46− Ag+ 2D7−, CD49b+ IgE+ IgG+ CD200R+ CD20− NKp46− Ag+ BB1−, CD49b+ IgE+ IgG+ CD244.2+ CD20− NKp46− Ag+ 2D7− BB1−, CD49b+ IgE+ IgG+ CD244.2+ CD20− NKp46− Ag+ 2D7−, CD49b+ IgE+ IgG+ CD244.2+ CD20− NKp46− Ag+ BB1−, CD49b+ IgE+ IgG+ FcεR1+ CD20− NKp46− Ag+ 2D7− BB1−, CD49b+ IgE+ IgG+ FcεR1+ CD20− NKp46− Ag+ 2D7−, CD49b+ IgE+ IgG+ FcεR1+ CD20− NKp46− Ag+ BB1−, CD49b+ IgE+ IgG+ CD200R+ CD20− CD122− Ag+ 2D7− BB1−, CD49b+ IgE+ IgG+ CD200R+ CD20− CD122− Ag+ 2D7−, CD49b+ IgE+ IgG+ CD200R+ CD20− CD122− Ag+ BB1−, CD49b+ IgE+ IgG+ CD244.2+ CD20− CD122− Ag+ 2D7− BB1−, CD49b+ IgE+ IgG+ CD244.2+ CD20− CD122− Ag+ 2D7−, CD49b+ IgE+ IgG+ CD244.2+ CD20− CD122− Ag+ BB1−, CD49b+ IgE+ IgG+ FcεR1+ CD20− CD122− Ag+ 2D7− BB1−, CD49b+ IgE+ IgG+ FcεR1+ CD20− CD122− Ag+ 2D7−, CD49b+ IgE+ IgG+ FcεR1+ CD20− CD122− Ag+ BB1−, CD49b+ IgE+ IgG+ CD200R+ CD19− CD20− 2D7− BB1−, CD49b+ IgE+ IgG+ CD200R+ CD19− CD20− 2D7−, CD49b+ IgE+ IgG+ CD200R+ CD19− CD20− BB1−, CD49b+ IgE+ IgG+ CD244.2+ CD19− CD20− 2D7− BB1−, CD49b+ IgE+ IgG+ CD244.2+ CD19− CD20− 2D7−, CD49b+ IgE+ IgG+ CD244.2+ CD19− CD20− BB1−, CD49b+ IgE+ IgG+ FcεR1+ CD19− CD20− 2D7− BB1−, CD49b+ IgE+ IgG+ FcεR1+ CD19− CD20− 2D7−, CD49b+ IgE+ IgG+ FcεR1+ CD19− CD20− BB1−, CD49b+ IgE+ IgG+ CD200R+ CD19− CD20− NK1.1− 2D7− BB1−, CD49b+ IgE+ IgG+ CD200R+ CD19− CD20− NK1.1− 2D7−, CD49b+ IgE+ IgG+ CD200R+ CD19− CD20−NK1.1− 2D7− BB1−, CD49b+ IgE+ IgG+ CD244.2+ CD19− CD20− NK1.1− BB1−, CD49b+ IgE+ IgG+ FcεR1+ CD19− CD20− NK1.1− 2D7− BB1−, CD49b+ IgE+ IgG+ FcεR1+ CD19− CD20− NK1.1− 2D7−, CD49b+ IgE+ IgG+ FcεR1+ CD19− CD20− NK1.1− 2BB1−, CD49b+ IgE+ IgG+ CD200R+ CD19− CD20− NKp46− 2D7− BB1−, CD49b+ IgE+ IgG+ CD200R+ CD19− CD20− NKp46− 2D7−, CD49b+ IgE+ IgG+ CD200R+ CD19− CD20− NKp46− BB1−, CD49b+ IgE+ IgG+ CD244.2+ CD19− CD20− NKp46− 2D7− BB1−, CD49b+ IgE+ IgG+ CD244.2+ CD19− CD20− NKp46− 2D7−, CD49b+ IgE+ IgG+ CD244.2+ CD19− CD20− NKp46− BB1−, CD49b+ IgE+ IgG+ FcεR1+ CD19− CD20− NKp46− 2D7− BB1−, CD49b+ IgE+ IgG+ FcεR1+ CD19− CD20− NKp46− BB1−, CD49b+ IgE+ IgG+ FcεR1+ CD19− CD20− NKp46− BB1−, CD49b+ IgE+ IgG+ CD200R+ CD19− CD20− CD122− 2D7− BB1−, CD49b+ IgE+ IgG+ CD200R+ CD19− CD20− CD122− 2D7−, CD49b+ IgE+ IgG+ CD200R+ CD19− CD20− CD122− BB1−, CD49b+ IgE+ IgG+ CD244.2+ CD19− CD20− CD122− 2D7− BB1−, CD49b+ IgE+ IgG+ CD244.2+ CD19− CD20− CD122− 2D7−, CD49b+ IgE+ IgG+ CD244.2+ CD19− CD20− CD122− BB1−, CD49b+ IgE+ IgG+ FcεR1+ CD19− CD20− CD122− 2D7− BB1−, CD49b+ IgE+ IgG+ FcεR1+ CD19− CD20− CD122− 2D7−, CD49b+ IgE+ IgG+ FcεR1+ CD19− CD20− CD122− BB1−, CD49b+ IgE+ IgG+ CD200R+ CD19− CD20− Ag+ 2D7− BB1−, CD49b+ IgE+ IgG+ CD200R+ CD19− CD20− Ag+ 2D7−, CD49b+ IgE+ IgG+ CD200R+ CD19− CD20− Ag+ BB1−, CD49b+ IgE+ IgG+ CD244.2+ CD19− CD20− Ag+ 2D7− BB1−, CD49b+ IgE+ IgG+ CD244.2+ CD19− CD20− Ag+ 2D7−, CD49b+ IgE+ IgG+ CD244.2+ CD19− CD20− Ag+ BB1−, CD49b+ IgE+ IgG+ FcεR1+ CD19− CD20− Ag+ 2D7− BB1−, CD49b+ IgE+ IgG+ FcεR1+ CD19− CD20− Ag+ 2D7−, CD49b+ IgE+ IgG+ FcεR1+ CD19− CD20− Ag+ BB1−, CD49b+ IgE+ IgG+ CD200R+ CD19− CD20− NK1.1− Ag+ 2D7− BB1−, CD49b+ IgE+ IgG+ CD200R+ CD19− CD20− NK1.1− Ag+ 2D7−, CD49b+ IgE+ IgG+ CD200R+ CD19− CD20− NK1.1− Ag+ BB1−, CD49b+ IgE+ IgG+ CD244.2+ CD19− CD20− NK1.1− Ag+ 2D7− BB1−, CD49b+ IgE+ IgG+ CD244.2+ CD19− CD20− NK1.1− Ag+ 2D7−, CD49b+ IgE+ IgG+ CD244.2+ CD19− CD20− NK1.1− Ag+ BB1−, CD49b+ IgE+ IgG+ FcεR1+ CD19− CD20− NK1.1− Ag+ 2D7− BB1−, CD49b+ IgE+ IgG+ FcεR1+ CD19− CD20− NK1.1− Ag+ 2D7−, CD49b+ IgE+ IgG+ FcεR1+ CD19− CD20− NK1.1− Ag+ BB1−, CD49b+ IgE+ IgG+ CD200R+ CD19− CD20− NKp46− Ag+ 2D7− BB1−, CD49b+ IgE+ IgG+ CD200R+ CD19− CD20− NKp46− Ag+ 2D7−, CD49b+IgE+ IgG+ CD200R+ CD19− CD20− NKp46− Ag+ BB1−, CD49b+ IgE+ IgG+ CD244.2+ CD19− CD20− NKp46− Ag+ 2D7− BB1−, CD49b+ IgE+ IgG+ CD244.2+ CD19− CD20− NKp46− Ag+ 2D7−, CD49b+ IgE+ IgG+ CD244.2+ CD19− CD20− NKp46− Ag+ BB1−, CD49b+ IgE+ IgG+ FcεR1+ CD19− CD20− NKp46− Ag+ 2D7− BB1−, CD49b+ IgE+ IgG+ FcεR1+ CD19− CD20− NKp46− Ag+ 2D7−, CD49b+ IgE+ IgG+ FcεR1+ CD19− CD20− NKp46− Ag+ BB1−, CD49b+ IgE+ IgG+ CD200R+ CD19− CD20− CD122− Ag+ 2D7− BB1−, CD49b+ IgE+ IgG+ CD200R+ CD19− CD20− CD122− Ag+ 2D7−, CD49b+ IgE+ IgG+ CD200R+ CD19− CD20− CD122− Ag+ BB1−, CD49b+ IgE+ IgG+ CD244.2+ CD19− CD20− CD122− Ag+ 2D7− BB1−, CD49b+ IgE+ IgG+ CD244.2+ CD19− CD20− CD122− Ag+ 2D7−, CD49b+ IgE+ IgG+ CD244.2+ CD19− CD20− CD122− Ag+ BB1−, CD49b+ IgE+ IgG+ FcεR1+ CD19− CD20− CD122− Ag+ 2D7− BB1−, CD49b+ IgE+ IgG+ FcεR1+ CD19− CD20− CD122− Ag+ 2D7−, CD49b+ IgE+ IgG+ FcεR1+ CD19− CD20− CD122− Ag+ BB1−, or naïve V cells, for example IgE+ IgG+ CD200R+ CD19− 2D7− BB1−, IgE+ IgG+ CD200R+ CD19− 2D7−, IgE+ IgG+ CD200R+ CD19− BB1−, IgE+ IgG+ CD244.2+ CD19− 2D7− BB1−, IgE+ IgG+ CD244.2+ CD19− 2D7−, IgE+ IgG+ CD244.2+ CD19− BB1−, IgE+ IgG+ FcεR1+ CD19− 2D7− BB1−, IgE+ IgG+ FcεR1+ CD19− 2D7−, IgE+ IgG+ FcεR1+ CD19− BB1−, IgE+ IgG+ CD16/CD32+ CD19− 2D7− BB1−, IgE+ IgG+ CD16/CD32+ CD19− 2D7−, IgE+ IgG+ CD16/CD32+ CD19− BB1−, IgE+ IgG+ CD24+ CD19− 2D7− BB1−, IgE+ IgG+ CD24+ CD19− 2D7−, IgE+ IgG+ CD24+ CD19− BB1−, IgE+ IgG+ CD43+ CD19− 2D7− BB1−, IgE+ IgG+ CD43+ CD19− 2D7−, IgE+ IgG+ CD43+ CD19− BB1−, IgE+ IgG+ CD45+ CD19− 2D7− BB1−, IgE+ IgG+ CD45+ CD19− 2D7−, IgE+ IgG+ CD45+ CD19− BB1−, IgE+ IgG+ CD48+ CD19− 2D7− BB1−, IgE+ IgG+ CD48+ CD19− 2D7−, IgE+ IgG+ CD48+ CD19− BB1−, IgE+ IgG+ CD54+ CD19− 2D7− BB1−, IgE+ IgG+ CD54+ CD19− 2D7−, IgE+ IgG+ CD54+ CD19− BB1−, IgE+ IgG+ CD79b+ CD19− 2D7− BB1−, IgE+ IgG+ CD79b+ CD19− 2D7−, IgE+ IgG+ CD79b+ CD19− BB1−, IgE+ IgG+ CD200R+ CD20− 2D7− BB1−, IgE+ IgG+ CD200R+ CD20− 2D7−, IgE+ IgG+ CD200R+ CD20− BB1−, IgE+ IgG+ CD244.2+ CD20− 2D7− BB1−, IgE+ IgG+ CD244.2+ CD20− 2D7−, IgE+ IgG+ CD244.2+ CD20− BB1−, IgE+ IgG+ FcεR1+ CD20− 2D7− BB1−, IgE+ IgG+ FcεR1+ CD20− 2D7−, IgE+ IgG+ FcεR1+ CD20− BB1−, IgE+ IgG+ CD16/CD32+ CD20− 2D7− BB1−, IgE+ IgG+ CD16/CD32+ CD20− 2D7−, IgE+ IgG+ CD16/CD32+ CD20− BB1−, IgE+ IgG+ CD24+ CD20− 2D7− BB1−, IgE+ IgG+ CD24+ CD20− 2D7−, IgE+ IgG+ CD24+ CD20− BB1−, IgE+ IgG+ CD43+ CD20− 2D7− BB1−, IgE+ IgG+ CD43+ CD20− 2D7−, IgE+ IgG+ CD43+ CD20− BB1−, IgE+ IgG+ CD45+ CD20− 2D7− BB1−, IgE+ IgG+ CD45+ CD20− 2D7−, IgE+ IgG+ CD45+ CD20− BB1−, IgE+ IgG+ CD48+ CD20− 2D7− BB1−, IgE+ IgG+ CD48+ CD20− 2D7−, IgE+ IgG+ CD48+ CD20− BB1−, IgE+ IgG+ CD54+ CD20− 2D7− BB1−, IgE+ IgG+ CD54+ CD20− 2D7−, IgE+ IgG+ CD54+ CD20− BB1−, IgE+ IgG+ CD79b+ CD20− 2D7− BB1−, IgE+ IgG+ CD79b+ CD20− 2D7−, or IgE+ IgG+ CD79b+ CD20− BB1−, cells. As such, in some embodiments, V cells are identified using reagents targeting at least one of the listed combinations of markers.

As described herein, various subpopulations of V cells have also been identified. These subpopulations can be positive or negative for additional markers, as described herein.

Without being limited by any particular theory, results reported herein indicate that at least in human V-cells, there are a number of markers that can be upregulated or downregulated depending on the activation stage (or maturity stage) of the V-cell. For example, CD49b can appear to be negative (downregulated) in human V-cells when found in a naïve state. However, if the human immune system is mounting an adaptive immune response, for example, due to an infection, virus, parasite or bacteria, CD49b will be upregulated to allow the V-cells to move to the area affected, causing the cells to be positive for that marker. As such, in some embodiments, V cells have low CD49b, for example naïve V cells. In some embodiments, V cells are CD49b+, for example, V cells mobilizing to an area occupied by an infection, virus, parasite, or bacteria.

Without being limited by any particular theory, results reported herein also indicate that V cells have different stages of maturation or differentiation. As such, in different stages of maturation, different markers can be upregulated while other markers can be downregulated. As such, it is contemplated herein that the phenotypic characteristics of V cells reported herein can be used to positively identify V cells at various stages of maturation and differentiation, but that at some stages of maturation or differentiation, at least some V cells may exhibit downregulation of one or more particular “positive” markers, or upregulation of one or more particular “negative” markers.

Immunization of Hosts and Production of V Cells that Produce Antigen-Specific Antibody

Some embodiments include methods of making V cells that produce antigen-specific antibody. A host animal can be immunized with antigen (Ag) to stimulate cells to generate specific antibody. Optionally, the host can be boosted with one or more administrations of antigen. In some embodiments, only a single administration of antigen is given, with no boosts. In some embodiments, a V cell can produce antigen-specific antibody in as few as 20 days after the first administration of antigen, for example 8, 9, 10, 11, 12, 13, 14, 15, 16, 17, 18, 19, or 20 days, including ranges between any of the listed values. In some embodiments, a V cell can produce antigen-specific antibody in as few as 10 days after the first administration of antigen. In some embodiments, a V cell can produce antigen-specific antibody in as few as 8 days after the first administration of antigen. Tissues and/or cell populations containing V cells can be collected from the host. V cells can be isolated from the tissues and/or cell populations as described herein. Such isolated V cells can be further characterized or studied, can be cultured, or can be used to produce antibodies as described herein. Exemplary methods of immunizing a host animal are described in Examples 1-8 herein.

In some embodiments, the host animal is a mammal. In some embodiments, the host animal is a mouse. In some embodiments, the host animal is one of a guinea pig, rat, hamster, rabbit, pig, goat, sheep, donkey, cow, camel, non-human primate, or horse. In some embodiments, the host animal is a non-human mammal. In some embodiments, the host is genetically modified. In some embodiments, the host is immunized with an antigen. In some embodiments, the antigen includes a recombinant polypeptide. In some embodiments, the antigen includes an isolated protein. In some embodiments, the antigen includes a cell or fragment thereof. In some embodiments, the antigen includes a virus (including for example an inactivated virus), bacterium, toxin or a fragment thereof.

In some embodiments, immunizing the host includes injecting the host with antigen. In some embodiments, the injection is intravenous. In some embodiments, the injection is subcutaneous (for example at the base of the tail of a rodent). In some embodiments, the injection is intraperitoneal. In some embodiments, at least about 1 μg of antigen (Ag) is provided, for example at least about 1 μg, 2, 3, 5, 7, 10, 15, 20, 25, 30, 35, 40, 45, 50, 55, 60, 65, 70, 75, 80, 85, 90, 95, 100, 110, 120, 130, 140, 150, 200, 250, 300, 350, 400, 450, 500, 600, 700, 800, 900, 1000, 1500, or 2000 μg of antigen per immunization. The antigen can be provided in solution, for example Complete Freunds' Adjuvant (CFA) and the like. It has been founds that V cells can be generated without Mycobacteria in CFA, and that similar results have been obtained when inoculating animals with antigen in Incomplete Freund's Adjuvant (IFA) as compared to CFA (see Examples 1-8). Accordingly, in some embodiments, the antigen is provided in incomplete Freund's Adjuvant (IFA).

V cells have been detected in animals following a single immunization with the antigen (50 microgram in CFA) by a combination of i.p and s.c. immunization 7-14 days following inoculation (see Examples 1-8). Accordingly, in some embodiments, immunization includes a single immunization step. Optionally, in some embodiments, the host is boosted at least once. In some embodiments, at least two boosts are performed, for example about 2, 3, 4, 5, 6, 7, 8, 9, or 10 boosts. In some embodiments, tissues are harvested within about seven days of a boost, for example about one, two, three, four, five, six, or seven days.

In some embodiments, V-cell containing tissues or cell populations are harvested using techniques known to one of skill in the art. V cells have been identified in hosts 7-14 days after an initial immunization. Thus, in some embodiments, V cells are harvested at least about 5 days after an initial immunization, for example about 5, 6, 7, 8, 9, 10, 11, 12, 13, 14, 15, 16, 17, 18, 19, 20, 21, or 22 days after inoculation. In some embodiments, the tissues or cell populations are harvested following a boost of at least about 12 hours, for example at least about 12, 24, 36, 48, 60, 72, 84, 96, 108, 120, 132, 144, 168, 182, or 196 hours. In some embodiments, blood is harvested. In some embodiments, bone marrow is harvested. In some embodiments, splenocytes are harvested. In some embodiments, thymus is harvested. In some embodiments, two or more of the listed tissues (or cell populations) are harvested. In some embodiments, for example when V cells are to be cultured, for example to make a hybridoma, the harvest is performed aseptically. In some embodiments, V cells are isolated from the harvested tissues or cell populations using methods described herein.

### Antibodies

Some embodiments include antibodies, and/or methods of making antibodies. As used herein, “antibody” refers to full-size antibodies, and unless stated otherwise, antigen-binding fragments thereof. Antigen-binding fragments of antibodies can be formatted into a variety of protein formats according to embodiments herein. Antigen-binding fragments of antibodies, or “antibody fragments” as used herein include a portion of an intact antibody comprising the antigen binding site or variable region of the intact antibody. Some antibody fragments are free of the constant heavy chain domains (i.e. CH2, CH3, and CH4, depending on antibody isotype) of the Fc region of the intact antibody, or a portion thereof. Examples of antibody fragments include, but are not limited to Fab, Fab′, Fab′-SH, F(ab′).sub.2, and Fv fragments; minibodies; diabodies; any antibody fragment that is a polypeptide having a primary structure consisting of one uninterrupted sequence of contiguous amino acid residues (referred to herein as a “single-chain antibody fragment” or “single chain polypeptide”), including without limitation (1) single-chain Fv (scFv) molecules (2) single chain polypeptides containing only one light chain variable domain, or a fragment thereof that contains the three CDRs of the light chain variable domain, without an associated heavy chain moiety and (3) single chain polypeptides containing only one heavy chain variable region, or a fragment thereof containing the three CDRs of the heavy chain variable region, without an associated light chain moiety; and multispecific or multivalent structures formed from antibody fragments, for example bispecific antibodies. In some embodiments, the antibody is monoclonal. In some embodiments, the antibody is chimeric. In some embodiments, the antibody is murine. In some embodiments, the antibody is humanized. In some embodiments, the antibody is human.

Antibodies can be produced under in vivo, ex vivo, and/or in vitro conditions. The general structure of antibodies has been described, for example, in U.S. Pat. No. 6,156,878, which is hereby incorporated by reference for its disclosure of antibody structure and for all purposes. Naturally-occurring antibodies or immunoglobulins are typically tetramers of four covalently bound peptide chains. For example, an IgG antibody has two light chains and two heavy chains. Each light chain is covalently bound to a heavy chain. In turn each heavy chain is covalently linked to the other to form a “Y” configuration, also known as an immunoglobulin conformation. Fragments of these molecules, or even heavy or light chains alone, can bind antigen. Antibodies, fragments of antibodies, and individual chains are also referred to herein as immunoglobulins.

A normal naturally-occurring antibody heavy or light chain has an N-terminal (NH₂) variable (V) region and a C-terminal (—COOH) constant (C) region. The heavy chain variable region is referred to as V_{H} (including, for example, V_{gamma}), and the light chain variable region is referred to as V_{L} (including Vₖₐₚₚₐ or V_{lambda}). The variable region is the part of the molecule that binds to the antibody's cognate antigen, while the Fc region (the second and third domains of the C region) determines the antibody's effector function (e.g., complement fixation). Full-length immunoglobulin or antibody “light chains” (generally about 25 kDa, about 214 amino acids) are encoded by a variable region gene at the N-terminus (generally about 110 amino acids) and a kappa or lambda constant region gene at the COOH-terminus. Full-length immunoglobulin or antibody “heavy chains” (generally about 50 Kd, about 446 amino acids), are similarly encoded by a variable region gene (generally encoding about 116 amino acids) and one of the constant region genes, e.g., gamma (encoding about 330 amino acids). Typically, the “V_{L}” will include the portion of the light chain encoded by the V_{L} and/or J_{L} (J or joining region) gene segments, and the “V_{H}” will include the portion of the heavy chain encoded by the V_{H} and/or D_{H} (D or diversity region) and J_{H} gene segments. See, generally, Roitt et al., Immunology (2d ed. 1989), Chapter 6 and Paul, Fundamental Immunology (Raven Press, 2d ed., 1989) (each of which is incorporated by reference for all purposes).

An immunoglobulin light or heavy chain variable region consists of a “framework” region (“FR,” which also may be referred to herein as “FWR”) interrupted by three hypervariable regions, also called complementarity-determining regions or CDRs. The CDRs are typically referred to as CDR1, CDR2, and CDR3, numbered sequentially starting from the N-terminus. From N-terminal to C-terminal, both light and heavy chains include domains FR1, CDR1, FR2, CDR2, FR3, CDR3 and FR4. Domains of the heavy chain may be referred to herein as HFR1, HCDR1, HFR2, HCDR2, HFR3, HCDR3, and HFR4. Domains of the light chain may be referred to herein as LFR1, LCDR1, LFR2, LCDR2, LHFR3, LCDR3, and LFR4. The extent of the framework region and CDRs have been defined (see Kabat et al. (1987), “Sequences of Proteins of Immunological Interest,” U.S. Department of Health and Human Services; Chothia et al., J. Mol. Biol. 196:901-917 (1987) (each of which is incorporated by reference herein for all purposes). The sequences of the framework regions of different light or heavy chains are relatively conserved within a species. The framework region of an antibody, that is the combined framework regions of the constituent light and heavy chains, serves to position and align the CDRs in three dimensional space. The CDRs are primarily responsible for binding to an epitope of an antigen.

The constant region of the heavy chain molecule, also known as CH, determines the isotype of the antibody. Antibodies are referred to as IgM, IgD, IgG, IgA, and IgE depending on the heavy chain isotype. The isotypes are encoded in the mu, delta, gamma, alpha, and epsilon segments of the heavy chain constant region, respectively. In addition, there are a number of gamma subtypes. There are two types of light chains, kappa and lambda. The determinants of these subtypes typically reside in the constant region of the light chain, also referred to as the C_{L} in general, and Cₖₐₚₚₐ or C_{lambda} in particular.

The heavy chain isotypes can determine different effector functions of the antibody, such as opsonization or complement fixation. In addition, the heavy chain isotype determines the secreted form of the antibody. Secreted IgG, IgD, and IgE isotypes are typically found in single unit or monomeric form. Secreted IgM isotype is found in pentameric form; secreted IgA can be found in both monomeric and dimeric form.

### Detectable Markers

In some embodiments, an antibody or antigen binding molecule is conjugated to a detectable marker. In some embodiments, a binding compound is attached, directly or indirectly to one or more fluorescent moieties, calorimetric moieties, chemiluminescent moieties, and the like. Detectable markers are described in U.S. Pat. No. 7,816,135, which is hereby incorporated by reference in its entirety. Reviews of labeling methodology that provide guidance for selection and attachment of labels to binding compounds include Haugland, Handbook of Fluorescent Probes and Research Chemicals, Ninth Edition (Molecular Probes, Inc., Eugene, 2002); Keller and Manak, DNA Probes, 2nd Edition (Stockton Press, New York, 1993); Hermanson, Bioconjugate Techniques (Academic Press, New York, 1996); and the like.

In some embodiments, the detectable marker includes an optical label. Particular optical labels, such as dyes are disclosed in the following sample of references: Menchen et al, U.S. Pat. No. 5,188,934 (4,7-dichlorofluorscein dyes); Begot et al, U.S. Pat. No. 5,366,860 (spectrally resolvable rhodamine dyes); Lee et al, U.S. Pat. No. 5,847,162 (4,7-dichlororhodamine dyes); Khanna et al, U.S. Pat. No. 4,318,846 (ether-substituted fluorescein dyes); Lee et al, U.S. Pat. No. 5,800,996 (energy transfer dyes); Lee et al, U.S. Pat. No. 5,066,580 (xanthene dyes): Mathies et al, U.S. Pat. No. 5,688,648 (energy transfer dyes); and the like. Exemplary fluorescent dyes include, but are not limited to, fluorescein isothiocyanate (FITC), 2-[ethylamino)-3-(ethylimino)-2-7-dimethyl-3H-xanthen-9-yl]benzoic acid ethyl ester monohydrochloride (R6G)(emits a response radiation in the wavelength that ranges from about 500 to 560 nm), 1,1,3,3,3′,3′-Hexamethylindodicarbocyanine iodide (HIDC) (emits a response radiation in the wavelength that ranged from about 600 to 660 nm), 6-carboxyfluorescein (commonly known by the abbreviations FAM and F), 6-carboxy-2′,4′,7′,4,7-hexachlorofluorescein (HEX), 6-carboxy-4′,5′-dichloro-2′,7′-dimethoxyfluorescein (JOE or J), N,N,N′,N′-tetramethyl-6-carboxyrhodamine (TAMRA or T), 6-carboxy-X-rhodamine (ROX or R), 5-carboxyrhodamine-6G (R6G5 or G5), 6-carboxyrhodamine-6G (R6G6 or G6), Alexa Fluor™ 350, Alexa Fluor™. 532, Alexa Fluor™. 546, Alexa Fluor™ 568, Alexa Fluor™. 594, Alexa Fluor™. 647, BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethylrhodanine, Texas Red (available from Molecular Probes, Inc., Eugene, Oreg. USA), and Cy2, Cy3.5, Cy5.5, and Cy7 (Amersham Biosciences, Piscataway, N.J. USA, HSF™ technology polymers (Sirigen, Inc, San Diego, Calif. see U.S. Pat. Nos. 8,158,444, and 8,362,193 each if which is hereby incorporated by reference in its entirety), and others. Labeling can also be carried out with quantum dots, as disclosed in the following patents and patent publications, incorporated herein by reference: U.S. Pat. Nos. 6,322,901; 6,576,291; 6,423,551; 6,251,303; 6,319,426; 6,426,513; 6,444,143; 5,990,479; 6,207,392; 2002/0045045; 2003/0017264; and the like. As used herein, the term “fluorescent signal generating moiety” means a signaling means which conveys information through the fluorescent absorption and/or emission properties of one or more molecules. Such fluorescent properties include fluorescence intensity, fluorescence life time, emission spectrum characteristics, energy transfer, and the like. In one aspect, optical labels of the invention are fluorescent signal generating moieties.

Fluorescence resonant energy transfer (FRET) tandem fluorophores may also be used, such as PerCP-Cy5.5, PE-Cy5, PE-Cy5.5, PE-Cy7, PE-Texas Red, and APC-Cy7; also, PE-Alexa dyes (610, 647, 680) and APC-Alexa dyes. PerCP is described in U.S. Pat. No. 4,876,190, which is incorporated by reference. Cyanine resonance energy transfer tandem fluorophores (“tandem fluorophores”, “tandem dyes”, “tricolor stains”) have recently expanded the choices of fluorophore available for single-laser, multi-color flow cytometric analysis. PE-CY5 tandem staining proves particularly well-suited for three-color analysis: the R-PE moiety, excited by the 488 nm light of an argon ion laser, serves as an energy donor, and CY5, acting as an energy acceptor, fluoresces at 670 nm, readily distinguishable from the emission of FITC and PE. Cyanine fluorophores are described in U.S. Pat. Nos. 5,268,486; 4,337,063; 4,404,289; 4,405,711; and in Mujumdar et al., Bioconj. Chem. 4:105-111(1993); Southwick et al., Cytometry 11:418-430 (1990); Ernst et al., Cytometry 10:3-10 (1989); and Mujumdar et al., Cytometry 10:11-19 (1989), and cyanine energy resonance transfer tandem fluorophores are described, inter alia, in U.S. Pat. No. 5,714,386 and in Waggoner et al., Ann. NY Acad. Sci. 677:185-193 (1993) and Lansdorp et al., Cytometry 12:723-30 (1991), the disclosures of which are incorporated herein by reference.

One of skill in the art will appreciate that two or more different detectable markers can be used simultaneously, for example to identify two or more characteristics of a sample, or to perform FRET. Accordingly, in some embodiments, two or more different detectable markers may be selected, for example for inclusion in a kit. In some embodiments, two or more detectable makers are distinctly detectable, for example as spectrally resolvable. In some embodiments, at least two spectrally resolvable optically detectable markers are employed. In some embodiment at least three spectrally resolvable optically detectable markers fluorescent markers are employed.

### Cell Separation Systems

Some embodiments include cell separation systems. Cell separation systems can be used to move a cell from a first position to second position, for example to separate a cell from another cell in solution, or to attach a cell to a separable phase, thereby removing the cell from solution. Cell separation systems are described, for example in U.S. Pat. No. 7,790,458, which is hereby incorporated by reference in its entirety.

In some embodiments, the cell separation system includes a separable phase. Exemplary separable phases can include substrates, such as surfaces of reaction vessels, or microfluidic chambers, solid beads, such as latex beads, agarose beads, metal beads, magnetic beads, nanoparticles, and the like. In some embodiments, the separable phase is attached to one or more antibody that binds specifically to a cell-specific marker, for example a lineage marker. In some embodiments, the separable phase is configured to attach to one or more antibodies. In some embodiments, the separable phase is configured to attach to one or more antibodies via a biotin-streptavidin system (e.g., biotinylated antibody and streptavidin-coated bead), or the like. In some embodiments, the separable phase is configured to attach to one or more antibodies via a GST pulldown system (e.g., GST-tagged antibody and glutathione-coated beads), or the like. In some embodiments, the separable phase is configured to attach to one or more antibodies via a fluorochrome attached to the antibody and an anti-fluorochrome separable phase, or the like. In some embodiments, the separable phase is attached or configured to attach to antibodies via two or more binding systems (e.g., a separable phase can coated in streptavidin and anti-fluorochrome molecules). In some embodiments, the separable phase is attached or configured to attach to antibodies for a single type of marker, for example a monoclonal antibody or polyclonal antibodies against the same antigen. In some embodiments, the separable phase is attached or configured to attach to two or more kinds of antibodies, each of which specifically binds a different type of marker, for example a first and a second monoclonal antibody. In some embodiments, the separable phase is attached or configured to attach to a bispecific antibody.

In some embodiments, the cell separation system includes magnetic bead technology. By way of example, the BD IMag™ system (Becton, Dickinson and Company, NJ) includes a type of magnetic bead technology. In some embodiments, magnetic bead technology includes a separable phase of magnetic nanoparticles. In some embodiments, the magnetic nanoparticle has a diameter of about 10 nm, to 500 nm, for example about 10 nm, 20, 30, 40, 50, 60, 70, 80, 90, 100, 150, 200, 250, 300, 350, 400, 450, or 500 nm. Thus, in some embodiments, the magnetic bead can be suspended in solution, but can be separated from solution for example via the application of a magnetic field. In some embodiments, the magnetic bead can precipitate out of solution without the application of a magnetic field. In some embodiments, each magnetic bead is attached to one or more antibody. In some embodiments, each magnetic bead is configured to attach to antibodies (or the like) via a binding system such as biotinylated markers and streptavidin-coated beads, or GST and glutathione-coated beads, or the like. In some embodiments, each magnetic bead is configured to attach to antibodies via a specific fluorochrome attached to the antibody. In some embodiments, each magnetic bead can attach to antibodies via two or more binding systems

Accordingly, in some embodiments, antibodies attached to the magnetic bead bind to markers on a cell in solution. A magnetic field can be applied to remove the magnetic bead and bound cell from solution. Thus, in some embodiments, large numbers of cells comprising at last one marker, for example one or more lineage marker, are removed by selective magnetic bead separations. In some embodiments, at least about 60%, for example at least about 60%, 65%, 70%, 75%, 80%, 85%, 90%, 95%, 96%, 97%, 98%, or 99% of the cells are removed.

In some embodiments, the cell separation system is a flow cytometry system. Flow cytometry is a well-known technique and suitable instruments are commercially available. In general, flow cytometry utilizes one or more energy sources, typically lasers, to illuminate a stream of liquid carrying detectable particles, such as cells. Flow cytometry is typically used in conjunction with detectable labels, such as labeled antibodies. This technique can be used to both detect cellular markers and to physically separate some cells from other cells based on those markers. Suitable flow cytometers include those available from Becton, Dickinson and Company under the trademark FACS™.

In some embodiments, positive selection is performed to isolate cells that possess one or more markers targeted by an antibody or antibodies. A cell separation system can be used to separate cells bound by the antibodies.

In some embodiments, negative selection is performed to isolate cells that possess one or more markers targeted by an antibody or antibodies, for example undesired cells. A cell separation system can be used to separate cells bound by the antibodies.

### Isolated V Cells

Some embodiments include isolated cells capable of producing antibody, for example V cells. Previously, it was generally believed that antibody-producing cells all belong to the B lymphocyte lineage. However, herein are disclosed antibody-producing cells described, for example V cells that do not belong to the B cell lineage. These isolated antibody-producing cells can be used, for example, for manufacturing antibodies, for performing ex vivo or in vitro diagnostics, or for research. Accordingly, some embodiments include isolated V cells. In some embodiments, the isolated V cell includes V-cell-specific markers as described herein, for example CD49b+, IgG+, IgE+, negative for a B-cell-specific marker, negative for a basophil-specific marker (e.g. 2D7 or BB1), and positive for at least one of CD16/CD32, CD24, CD43, CD45, CD48, CD54, CD79b, CD123, CD200R, CD244.2, or FcεR1. In some embodiments, the isolated V cells are CD49b+, IgG+, IgE+, CD200R+, and B220− 2D7− (for example, mouse V cells), or CD19− CD20− 2D7− and BB1− (for example, human V cells). In some embodiments, the isolated V cells have phenotypic characteristics as described herein. The term “isolated” is used to distinguish from cells that are in their natural environment in the body of a host. It also contemplates a degree of separation from other cells with which they are found in nature. Thus, an “isolated” V cell or V cell population can be completely or substantially free of other cell types, or simply enriched in V cells to a greater degree than in nature. In some embodiments, the host is a mammal. In some embodiments, the host is one of a mouse, a guinea pig, a hamster, a rabbit, a pig, a horse, a donkey, a cow, a sheep, a non-human primate, or a human, including genetically modified versions of these organisms.

In some embodiments the isolated cells capable of producing antibody are outside of a host organism, and substantially free of other cell types, for example lineage committed-cells and stem cells. In some embodiments, the isolated antibody-producing cells are provided as a population of cells, in which at least about 20% of the cells, for example about 20%, 25%, 30%, 35%, 40%, 45%, 50%, 55%, 60%, 65%, 70%, 75%, 80%, 85%, 90%, 95%, 97%, 98%, 99%, 99.5%, and 99.9% are such isolated antibody-producing cells. With respect to other cell types with which V cells are naturally found, enrichment factors versus one or more other non-V cell types of 3×, 5×, 8×, 10×, 20×, or 50× or more, as well as intermediate values within that range, are specifically contemplated. In some embodiments, isolated V cells are provided in a solution that contains cells, of which at least about 50% are V cells, for example at least about 50%, 60%, 70%, 80%, 90%, 95%, 97%, 99%, or 99.9%.

In some embodiments, the isolated V cells are antibody-producing cells, and are CD49b+, IgG+, IgE+, negative for markers specific for B cells, including B220, CD5, CD19, CD20, CD21/CD35, CD22.2, CD72, GL-7, IgD, IgM, Ly6-k, Ly6-D, Ly-51 CD127, CD138, CD154, AA4.1 and Pax-5, and negative for basophil-specific markers, for example 2D7 (for human or mouse V cells) or BB1 (for human V cells). The skilled artisan will appreciate that the particular B-cell-specific marker and/or basophil-specific marker can depend on the host organism, and that an appropriate B-cell-specific and/or basophil-specific marker can readily be selected. In some embodiments, the V cells are positive for at least one of CD16/CD32, CD24, CD43, CD45, CD48, CD54, CD79b, CD123, CD200R, CD244.2, or FcεR1. In some embodiments, the isolated V cells are further positive for at least one of CD24, CD43, CD45, and CD48. In some embodiments, the isolated antibody-producing cells are also IgE+. In some embodiments, the isolated antibody-producing cells are IgE−. In some embodiments, the absence of IgE on human IgG+ V cells can be identified by the presence of CD200R. Accordingly, in some embodiments, the isolated antibody-producing cells are CD49b+ IgG+ IgE− CD200R+. In some embodiments, the naïve V cells exhibit morphological characteristics of V cells as described herein.

In some embodiments, the isolated V cells are naïve V cells. The naïve V cells can be IgG+, IgE+, negative for a B-cell-specific marker, negative for a basophil-specific marker, and positive for at least one of CD16/CD32, CD24, CD43, CD45, CD48, CD54, CD79b, CD123, CD200R, CD244.2, or FcεR1. In some embodiments, for example if the naïve V cells are human, the naïve V cells exhibit low CD49b expression. In some embodiments, the naïve V cells exhibit morphological characteristics of V cells as described herein.

In some embodiments, the isolated V cells are negative for T cell-specific markers, including CD1d, CD3, CD4, CD8, CD25, CD38 and CD134, and negative for a basophil-specific marker. In some embodiments, the isolated antibody-producing cells are negative for NK-specific markers including NK1.1, CD49a, CD122 and CD226/NKp46. In some embodiments, the isolated antibody-producing cells are negative for hematopoietic stem cell (HSC) markers, including CD34, Sca-1, c-Kit and CD150. In some embodiments, the isolated antibody-producing cells are negative for monocyte markers, including CD11b and Ly-6G.

In some embodiments, the isolated V cells can be positive for CD16/CD32, CD24, CD43, CD45, CD48, CD49b (DX5 and HMa2 clones), CD54, CD79b, CD123, CD200R, CD244.2, FcεR1, and surface IgG and IgE The isolated V cells can be negative to B lineage cell markers, including B220, CD5, CD19, CD21/CD35, CD22.2, CD72, GL-7, IgD, IgM, Ly6-k, Ly6-D, Ly-51 CD127, CD138, CD154, AA4.1 and Pax-5 and negative for a basophil-specific markers. The isolated antibody producing cells can be negative for T cell markers, including CD1d, CD3, CD4, CD8, CD25, CD38 and CD134. The isolated antibody producing cells can be negative for dendritic cell markers, including CD11c & CD273, and negative for NK markers, including NK1.1, NK1.2, CD49a, CD122 and CD226/NKp46. The cells can be negative for basophil-specific markers, including CD123, 2D7, and/or BB1. The cells can be negative for HSC markers, including CD34, Sca-1, c-Kit and CD150, and for monocyte markers, including Ly-6G. In some embodiments, the isolated cells are V cells as described herein, for example CD49b+IgE+ IgG+CD200R+B220− 2D7− BB1−, CD49b+ IgE+ IgG+CD200R+ B220− 2D7−, CD49b+ IgE+ IgG+ CD200R+ B220− BB1−, CD49b+ IgE+ IgG+ CD244.2+ B220− 2D7− BB1−, CD49b+ IgE+ IgG+ CD244.2+ B220− 2D7−, CD49b+ IgE+ IgG+ CD244.2+ B220− BB1−, CD49b+ IgE+ IgG+ FcεR1+ B220− 2D7− BB1−, CD49b+ IgE+ IgG+ FcεR1+ B220− 2D7−, CD49b+ IgE+ IgG+ FcεR1+ B220− BB1−, CD49b+ IgE+ IgG+ CD200R+ B220− NK1.1− 2D7− BB1−, CD49b+ IgE+ IgG+ CD200R+ B220− NK1.1− 2D7−, CD49b+ IgE+ IgG+ CD200R+ B220− NK1.1− BB1−, CD49b+ IgE+ IgG+ CD244.2+ B220− NK1.1− 2D7− BB1−, CD49b+ IgE+ IgG+ CD244.2+ B220− NK1.1− 2D7−, CD49b+ IgE+ IgG+ CD244.2+ B220− NK1.1− BB1−, CD49b+ IgE+ IgG+ FcεR1+ B220− NK1.1− 2D7− BB1−, CD49b+ IgE+ IgG+ FcεR1+ B220− NK1.1− 2D7−, CD49b+ IgE+ IgG+ FcεR1+ B220− NK1.1− BB1−, CD49b+ IgE+ IgG+ CD200R+ B220− NKp46− 2D7− BB1−, CD49b+ IgE+ IgG+ CD200R+ B220− NKp46− 2D7−, CD49b+ IgE+ IgG+ CD200R+ B220− NKp46− BB1−, CD49b+ IgE+ IgG+ CD244.2+ B220− NKp46− 2D7− BB1−, CD49b+ IgE+ IgG+ CD244.2+ B220− NKp46− 2D7−, CD49b+ IgE+ IgG+ CD244.2+ B220− NKp46− BB1−, CD49b+ IgE+ IgG+ FcεR1+ B220− NKp46− 2D7− BB1−, CD49b+ IgE+ IgG+ FcεR1+ B220− NKp46− 2D7−, CD49b+ IgE+ IgG+ FcεR1+ B220− NKp46− BB1−, CD49b+ IgE+ IgG+ CD200R+ B220− CD122− 2D7− BB1−, CD49b+ IgE+ IgG+ CD200R+ B220− CD122− 2D7−, CD49b+ IgE+ IgG+ CD200R+ B220− CD122− BB1−, CD49b+ IgE+ IgG+ CD244.2+ B220− CD122− 2D7− BB1−, CD49b+ IgE+ IgG+ CD244.2+ B220− CD122− 2D7−, CD49b+ IgE+ IgG+ CD244.2+ B220− CD122− BB1−, CD49b+ IgE+ IgG+ FcεR1+ B220− CD122− 2D7− BB1−, CD49b+ IgE+ IgG+ FcεR1+ B220− CD122− 2D7−, CD49b+ IgE+ IgG+ FcεR1+ B220− CD122− BB1−, CD49b+ IgE+ IgG+ CD200R+ B220− Ag+ 2D7− BB1−, CD49b+ IgE+ IgG+ CD200R+ B220− Ag+ 2D7−, CD49b+ IgE+ IgG+ CD200R+ B220− Ag+ BB1−, CD49b+ IgE+ IgG+ CD244.2+ B220− Ag+ 2D7− BB1−, CD49b+ IgE+ IgG+ CD244.2+ B220− Ag+ 2D7−, CD49b+ IgE+ IgG+ CD244.2+ B220− Ag+ BB1−, CD49b+ IgE+ IgG+ FcεR1+ B220− Ag+ 2D7− BB1−, CD49b+ IgE+ IgG+ FcεR1+ B220− Ag+ 2D7−, CD49b+ IgE+ IgG+ FcεR1+ B220− Ag+ BB1−, CD49b+ IgE+ IgG+ CD200R+ B220− NK1.1− Ag+ 2D7− BB1−, CD49b+ IgE+ IgG+ CD200R+ B220− NK1.1− Ag+ 2D7−, CD49b+ IgE+ IgG+ CD200R+ B220− NK1.1− Ag+ BB1−, CD49b+ IgE+ IgG+ CD244.2+ B220− NK1.1− Ag+ 2D7− BB1−, CD49b+ IgE+ IgG+ CD244.2+ B220− NK1.1− Ag+ 2D7−, CD49b+ IgE+ IgG+ CD244.2+ B220− NK1.1− Ag+ BB1−, CD49b+ IgE+ IgG+ FcεR1+ B220− NK1.1− Ag+ 2D7− BB1−, CD49b+ IgE+ IgG+ FcεR1+ B220− NK1.1− Ag+ 2D7−, CD49b+ IgE+ IgG+ FcεR1+ B220− NK1.1− Ag+ BB1−, CD49b+ IgE+ IgG+ CD200R+ B220− NKp46− Ag+ 2D7− BB1−, CD49b+ IgE+ IgG+ CD200R+ B220− NKp46− Ag+ 2D7−, CD49b+ IgE+ IgG+ CD200R+ B220− NKp46− Ag+ BB1−, CD49b+ IgE+ IgG+ CD244.2+ B220− NKp46− Ag+ 2D7− BB1−, CD49b+ IgE+ IgG+ CD244.2+ B220− NKp46− Ag+ 2D7−, CD49b+ IgE+ IgG+ CD244.2+B220− NKp46− Ag+ BB1−, CD49b+ IgE+ IgG+ FcεR1+ B220− NKp46− Ag+ 2D7− BB1−, CD49b+ IgE+ IgG+ FcεR1+ B220− NKp46− Ag+ 2D7−, CD49b+ IgE+ IgG+ FcεR1+ B220− NKp46− Ag+ BB1−, CD49b+ IgE+ IgG+ CD200R+ B220− CD122− Ag+ 2D7− BB1−, CD49b+ IgE+ IgG+ CD200R+ B220− CD122− Ag+ 2D7−, CD49b+ IgE+ IgG+ CD200R+ B220− CD122− Ag+ BB1−, CD49b+ IgE+ IgG+ CD244.2+ B220− CD122− Ag+ 2D7− BB1−, CD49b+ IgE+ IgG+ CD244.2+ B220− CD122− Ag+ 2D7−, CD49b+ IgE+ IgG+ CD244.2+ B220− CD122− Ag+ BB1−, CD49b+ IgE+ IgG+ FcεR1+ B220− CD122− Ag+ 2D7− BB1−, CD49b+ IgE+ IgG+ FcεR1+ B220− CD122− Ag+ 2D7−, CD49b+ IgE+ IgG+ FcεR1+ B220− CD122− Ag+ BB1−, CD49b+ IgE+ IgG+ CD200R+ CD19− 2D7− BB1−, CD49b+ IgE+ IgG+ CD200R+ CD19− 2D7−, CD49b+ IgE+ IgG+ CD200R+ CD19− BB1−, CD49b+ IgE+ IgG+ CD244.2+ CD19− 2D7− BB1−, CD49b+ IgE+ IgG+ CD244.2+ CD19− 2D7−, CD49b+ IgE+ IgG+ CD244.2+ CD19− BB1−, CD49b+ IgE+ IgG+ FcεR1+ CD19− 2D7− BB1−, CD49b+ IgE+ IgG+ FcεR1+ CD19− 2D7−, CD49b+ IgE+ IgG+ FcεR1+ CD19− BB1−, CD49b+ IgE+ IgG+ CD200R+ CD19− NK1.1− 2D7− BB1−, CD49b+ IgE+ IgG+ CD200R+ CD19− NK1.1− 2D7−, CD49b+ IgE+ IgG+ CD200R+ CD19− NK1.1− BB1−, CD49b+ IgE+ IgG+ CD244.2+ CD19− NK1.1− 2D7− BB1−, CD49b+ IgE+ IgG+ CD244.2+ CD19− NK1.1− 2D7−, CD49b+ IgE+ IgG+ CD244.2+ CD19− NK1.1− BB1−, CD49b+ IgE+ IgG+ FcεR1+ CD19− NK1.1− 2D7− BB1−, CD49b+ IgE+ IgG+ FcεR1+ CD19− NK1.1− 2D7−, CD49b+ IgE+ IgG+ FcεR1+ CD19− NK1.1− BB1−, CD49b+ IgE+ IgG+ CD200R+ CD19− NKp46− 2D7− BB1−, CD49b+ IgE+ IgG+ CD200R+ CD19− NKp46− 2D7−, CD49b+ IgE+ IgG+ CD200R+ CD19− NKp46− BB1−, CD49b+ IgE+ IgG+ CD244.2+ CD19− NKp46− 2D7− BB1−, CD49b+ IgE+ IgG+ CD244.2+ CD19− NKp46− 2D7−, CD49b+ IgE+ IgG+ CD244.2+ CD19− NKp46− BB1−, CD49b+ IgE+ IgG+ FcεR1+ CD19− NKp46− 2D7− BB1−, CD49b+ IgE+ IgG+ FcεR1+ CD19− NKp46− 2D7−, CD49b+ IgE+ IgG+ FcεR1+ CD19− NKp46− BB1−, CD49b+ IgE+ IgG+ CD200R+ CD19− CD122− 2D7− BB1−, CD49b+ IgE+ IgG+ CD200R+ CD19− CD122− 2D7−, CD49b+ IgE+ IgG+ CD200R+ CD19− CD122− BB1−, CD49b+ IgE+ IgG+ CD244.2+ CD19− CD122− 2D7− BB1−, CD49b+ IgE+ IgG+ CD244.2+ CD19− CD122− 2D7−, CD49b+ IgE+ IgG+ CD244.2+ CD19− CD122− BB1−, CD49b+ IgE+ IgG+ FcεR1+ CD19− CD122− 2D7− BB1−, CD49b+ IgE+ IgG+ FcεR1+ CD19− CD122− 2D7−, CD49b+ IgE+ IgG+ FcεR1+ CD19− CD122− BB1−, CD49b+ IgE+ IgG+ CD200R+ CD19− Ag+ 2D7− BB1−, CD49b+IgE+ IgG+ CD200R+ CD19− Ag+ 2D7−, CD49b+ IgE+ IgG+ CD200R+ CD19− Ag+ BB1−, CD49b+ IgE+ IgG+ CD244.2+ CD19− Ag+ 2D7− BB1−, CD49b+ IgE+ IgG+ CD244.2+ CD19− Ag+ 2D7−, CD49b+ IgE+ IgG+ CD244.2+ CD19− Ag+ BB1−, CD49b+ IgE+ IgG+ FcεR1+ CD19− Ag+ 2D7− BB1−, CD49b+ IgE+ IgG+ FcεR1+ CD19− Ag+ 2D7−, CD49b+ IgE+ IgG+ FcεR1+ CD19− Ag+ BB1−, CD49b+ IgE+ IgG+ CD200R+ CD19− NK1.1− Ag+ 2D7− BB1−, CD49b+ IgE+ IgG+ CD200R+ CD19− NK1.1− Ag+ 2D7−, CD49b+ IgE+ IgG+ CD200R+ CD19− NK1.1− Ag+ BB1−, CD49b+ IgE+ IgG+ CD244.2+ CD19− NK1.1− Ag+ 2D7− BB1−, CD49b+ IgE+ IgG+ CD244.2+ CD19− NK1.1− Ag+ 2D7−, CD49b+ IgE+ IgG+ CD244.2+ CD19− NK1.1− Ag+ BB1−, CD49b+ IgE+ IgG+ FcεR1+ CD19− NK1.1− Ag+ 2D7− BB1−, CD49b+ IgE+ IgG+ FcεR1+ CD19− NK1.1− Ag+ 2D7−, CD49b+ IgE+ IgG+ FcεR1+ CD19− NK1.1− Ag+ BB1−, CD49b+ IgE+ IgG+ CD200R+ CD19− NKp46− Ag+ 2D7− BB1−, CD49b+ IgE+ IgG+ CD200R+ CD19− NKp46− Ag+ 2D7−, CD49b+ IgE+ IgG+ CD200R+ CD19− NKp46− Ag+ BB1−, CD49b+ IgE+ IgG+ CD244.2+ CD19− NKp46− Ag+ 2D7− BB1−, CD49b+ IgE+ IgG+ CD244.2+ CD19− NKp46− Ag+ 2D7−, CD49b+ IgE+ IgG+ CD244.2+ CD19− NKp46− Ag+ BB1−, CD49b+ IgE+ IgG+ FcεR1+ CD19− NKp46− Ag+ 2D7− BB1−, CD49b+ IgE+ IgG+ FcεR1+ CD19− NKp46− Ag+ 2D7−, CD49b+ IgE+ IgG+ FcεR1+ CD19− NKp46− Ag+ BB1−, CD49b+ IgE+ IgG+ CD200R+ CD19− CD122− Ag+ 2D7− BB1−, CD49b+ IgE+ IgG+ CD200R+ CD19− CD122− Ag+ 2D7−, CD49b+ IgE+ IgG+ CD200R+ CD19− CD122− Ag+ BB1−, CD49b+ IgE+ IgG+ CD244.2+ CD19− CD122− Ag+ 2D7− BB1−, CD49b+ IgE+ IgG+ CD244.2+ CD19− CD122− Ag+ 2D7−, CD49b+ IgE+ IgG+ CD244.2+ CD19− CD122− Ag+ BB1−, CD49b+ IgE+ IgG+ FcεR1+ CD19− CD122− Ag+ 2D7− BB1−, CD49b+ IgE+ IgG+ FcεR1+ CD19− CD122− Ag+ 2D7−, CD49b+ IgE+ IgG+ FcεR1+ CD19− CD122− Ag+ BB1−, CD49b+ IgE+ IgG+ CD200R+ CD20− 2D7− BB1−, CD49b+ IgE+ IgG+ CD200R+ CD20− 2D7−, CD49b+ IgE+ IgG+ CD200R+ CD20− BB1−, CD49b+ IgE+ IgG+ CD244.2+ CD20− 2D7− BB1−, CD49b+ IgE+ IgG+ CD244.2+ CD20− 2D7−, CD49b+ IgE+ IgG+ CD244.2+ CD20− BB1−, CD49b+ IgE+ IgG+ FcεR1+ CD20− 2D7− BB1−, CD49b+ IgE+ IgG+ FcεR1+ CD20− 2D7−, CD49b+ IgE+ IgG+ FcεR1+ CD20− BB1−, CD49b+ IgE+ IgG+ CD200R+ CD20− NK1.1− 2D7− BB1−, CD49b+ IgE+ IgG+ CD200R+ CD20− NK1.1− 2D7−, CD49b+ IgE+ IgG+ CD200R+ CD20− NK1.1− BB1−, CD49b+ IgE+ IgG+ CD244.2+ CD20− NK1.1− 2D7− BB1−, CD49b+ IgE+ IgG+ CD244.2+ CD20− NK1.1− 2D7−, CD49b+IgE+ IgG+ CD244.2+ CD20− NK1.1− BB1−, CD49b+ IgE+ IgG+ FcεR1+ CD20− NK1.1− 2D7− BB1−, CD49b+ IgE+ IgG+ FcεR1+ CD20− NK1.1− 2D7−, CD49b+ IgE+ IgG+ FcεR1+ CD20− NK1.1− BB1−, CD49b+ IgE+ IgG+ CD200R+ CD20− NKp46− 2D7− BB1−, CD49b+ IgE+ IgG+ CD200R+ CD20− NKp46− 2D7−, CD49b+ IgE+ IgG+ CD200R+ CD20− NKp46− BB1−, CD49b+ IgE+ IgG+ CD244.2+ CD20− NKp46− 2D7− BB1−, CD49b+ IgE+ IgG+ CD244.2+ CD20− NKp46− 2D7−, CD49b+ IgE+ IgG+ CD244.2+ CD20− NKp46− BB1−, CD49b+ IgE+ IgG+ FcεR1+ CD20− NKp46− 2D7− BB1−, CD49b+ IgE+ IgG+ FcεR1+ CD20− NKp46− 2D7−, CD49b+ IgE+ IgG+ FcεR1+ CD20− NKp46− BB1−, CD49b+ IgE+ IgG+ CD200R+ CD20− CD122− 2D7− BB1−, CD49b+ IgE+ IgG+ CD200R+ CD20− CD122− 2D7−, CD49b+ IgE+ IgG+ CD200R+ CD20− CD122− BB1−, CD49b+ IgE+ IgG+ CD244.2+ CD20− CD122− 2D7− BB1−, CD49b+ IgE+ IgG+ CD244.2+ CD20− CD122− 2D7−, CD49b+ IgE+ IgG+ CD244.2+ CD20− CD122− BB1−, CD49b+ IgE+ IgG+ FcεR1+ CD20− CD122− 2D7− BB1−, CD49b+ IgE+ IgG+ FcεR1+ CD20− CD122− 2D7−, CD49b+ IgE+ IgG+ FcεR1+ CD20− CD122− BB1−, CD49b+ IgE+ IgG+ CD200R+ CD20− Ag+ 2D7− BB1−, CD49b+ IgE+ IgG+ CD200R+ CD20− Ag+ 2D7−, CD49b+ IgE+ IgG+ CD200R+ CD20− Ag+ BB1−, CD49b+ IgE+ IgG+ CD244.2+ CD20− Ag+ 2D7− BB1−, CD49b+ IgE+ IgG+ CD244.2+ CD20− Ag+ 2D7−, CD49b+ IgE+ IgG+ CD244.2+ CD20− Ag+ BB1−, CD49b+ IgE+ IgG+ FcεR1+ CD20− Ag+ 2D7− BB1−, CD49b+ IgE+ IgG+ FcεR1+ CD20− Ag+ 2D7−, CD49b+ IgE+ IgG+ FcεR1+ CD20− Ag+ BB1−, CD49b+ IgE+ IgG+ CD200R+ CD20− NK1.1− Ag+ 2D7− BB1−, CD49b+ IgE+ IgG+ CD200R+ CD20− NK1.1− Ag+ 2D7−, CD49b+ IgE+ IgG+ CD200R+ CD20− NK1.1− Ag+ BB1−, CD49b+ IgE+ IgG+ CD244.2+ CD20− NK1.1− Ag+ 2D7− BB1−, CD49b+ IgE+ IgG+ CD244.2+ CD20− NK1.1− Ag+ 2D7−, CD49b+ IgE+ IgG+ CD244.2+ CD20− NK1.1− Ag+ BB1−, CD49b+ IgE+ IgG+ FcεR1+ CD20− NK1.1− Ag+ 2D7− BB1−, CD49b+ IgE+ IgG+ FcεR1+ CD20− NK1.1− Ag+ 2D7−, CD49b+ IgE+ IgG+ FcεR1+ CD20− NK1.1− Ag+ BB1−, CD49b+ IgE+ IgG+ CD200R+ CD20− NKp46− Ag+ 2D7− BB1−, CD49b+ IgE+ IgG+ CD200R+ CD20− NKp46− Ag+ 2D7−, CD49b+ IgE+ IgG+ CD200R+ CD20− NKp46− Ag+ BB1−, CD49b+ IgE+ IgG+ CD244.2+ CD20− NKp46− Ag+ 2D7− BB1−, CD49b+ IgE+ IgG+ CD244.2+ CD20− NKp46− Ag+ 2D7−, CD49b+ IgE+ IgG+ CD244.2+ CD20− NKp46− Ag+ BB1−, CD49b+ IgE+ IgG+ FcεR1+ CD20− NKp46− Ag+ 2D7− BB1−, CD49b+ IgE+ IgG+ FcεR1+ CD20− NKp46− Ag+ 2D7−, CD49b+ IgE+ IgG+ FcεR1+ CD20− NKp46− Ag+ BB1−, CD49b+ IgE+ IgG+ CD200R+ CD20− CD122− Ag+ 2D7− BB1−, CD49b+ IgE+ IgG+ CD200R+ CD20− CD122− Ag+ 2D7−, CD49b+ IgE+ IgG+ CD200R+ CD20− CD122− Ag+ BB1−, CD49b+ IgE+ IgG+ CD244.2+ CD20− CD122− Ag+ 2D7− BB1−, CD49b+ IgE+ IgG+ CD244.2+ CD20− CD122− Ag+ 2D7−, CD49b+ IgE+ IgG+ CD244.2+ CD20− CD122− Ag+ BB1−, CD49b+ IgE+ IgG+ FcεR1+ CD20− CD122− Ag+ 2D7− BB1−, CD49b+ IgE+ IgG+ FcεR1+ CD20− CD122− Ag+ 2D7−, CD49b+ IgE+ IgG+ FcεR1+ CD20− CD122− Ag+ BB1−, CD49b+ IgE+ IgG+ CD200R+ CD19− CD20− 2D7− BB1−, CD49b+ IgE+ IgG+ CD200R+ CD19− CD20− 2D7−, CD49b+ IgE+ IgG+ CD200R+ CD19− CD20− BB1−, CD49b+ IgE+ IgG+ CD244.2+ CD19− CD20−, CD49b+ IgE+ IgG+ FcεR1+ CD19− CD20− 2D7− BB1−, CD49b+ IgE+ IgG+ CD244.2+ CD19− CD20−, CD49b+ IgE+ IgG+ FcεR1+ CD19− CD20− 2D7−, CD49b+ IgE+ IgG+ CD244.2+ CD19− CD20−, CD49b+ IgE+ IgG+ FcεR1+ CD19− CD20− BB1−, CD49b+ IgE+ IgG+ CD200R+ CD19− CD20− NK1.1− 2D7− BB1−, CD49b+ IgE+ IgG+ CD200R+ CD19− CD20− NK1.1− 2D7−, CD49b+ IgE+ IgG+ CD200R+ CD19− CD20− NK1.1− BB1−, CD49b+ IgE+ IgG+ CD244.2+ CD19− CD20− NK1.1− 2D7− BB1−, CD49b+ IgE+ IgG+ CD244.2+ CD19− CD20− NK1.1− 2D7−, CD49b+ IgE+ IgG+ CD244.2+ CD19− CD20− NK1.1− BB1−, CD49b+ IgE+ IgG+ FcεR1+ CD19− CD20− NK1.1− 2D7− BB1−, CD49b+ IgE+ IgG+ FcεR1+ CD19− CD20− NK1.1− 2D7−, CD49b+ IgE+ IgG+ FcεR1+ CD19− CD20− NK1.1− BB1−, CD49b+ IgE+ IgG+ CD200R+ CD19− CD20− NKp46− 2D7− BB1−, CD49b+ IgE+ IgG+ CD200R+ CD19− CD20− NKp46− 2D7−, CD49b+ IgE+ IgG+ CD200R+ CD19− CD20− NKp46− BB1−, CD49b+ IgE+ IgG+ CD244.2+ CD19− CD20− NKp46− 2D7− BB1−, CD49b+ IgE+ IgG+ CD244.2+ CD19− CD20− NKp46− 2D7−, CD49b+ IgE+ IgG+ CD244.2+ CD19− CD20− NKp46− BB1−, CD49b+ IgE+ IgG+ FcεR1+ CD19− CD20− NKp46− 2D7− BB1−, CD49b+ IgE+ IgG+ FcεR1+ CD19− CD20− NKp46− 2D7−, CD49b+ IgE+ IgG+ FcεR1+ CD19− CD20− NKp46− BB1−, CD49b+ IgE+ IgG+ CD200R+ CD19− CD20− CD122− 2D7− BB1−, CD49b+ IgE+ IgG+ CD200R+ CD19− CD20− CD122− 2D7−, CD49b+ IgE+ IgG+ CD200R+ CD19− CD20− CD122− BB1−, CD49b+ IgE+ IgG+ CD244.2+ CD19− CD20− CD122− 2D7− BB1−, CD49b+ IgE+ IgG+ CD244.2+ CD19− CD20− CD122− 2D7−, CD49b+ IgE+ IgG+ CD244.2+ CD19− CD20− CD122− BB1−, CD49b+ IgE+ IgG+ FcεR1+ CD19− CD20− CD122− 2D7− BB1−, CD49b+ IgE+ IgG+ FcεR1+ CD19− CD20− CD122− 2D7−, CD49b+ IgE+ IgG+ FcεR1+ CD19− CD20− CD122− BB1−, CD49b+ IgE+ IgG+ CD200R+ CD19− CD20− Ag+ 2D7− BB1−, CD49b+ IgE+ IgG+ CD200R+ CD19− CD20− Ag+ 2D7−, CD49b+ IgE+ IgG+ CD200R+ CD19− CD20− Ag+ BB1−, CD49b+ IgE+ IgG+ CD244.2+ CD19− CD20− Ag+ 2D7− BB1−, CD49b+ IgE+ IgG+ CD244.2+ CD19− CD20− Ag+ 2D7−, CD49b+ IgE+ IgG+ CD244.2+ CD19− CD20− Ag+ BB1−, CD49b+ IgE+ IgG+ FcεR1+ CD19− CD20− Ag+ 2D7− BB1−, CD49b+ IgE+ IgG+ FcεR1+ CD19− CD20− Ag+ 2D7−, CD49b+ IgE+ IgG+ FcεR1+ CD19− CD20− Ag+ BB1−, CD49b+ IgE+ IgG+ CD200R+ CD19− CD20− NK1.1− Ag+ 2D7− BB1−, CD49b+ IgE+ IgG+ CD200R+ CD19− CD20− NK1.1− Ag+ 2D7−, CD49b+ IgE+ IgG+ CD200R+ CD19− CD20− NK1.1− Ag+ BB1−, CD49b+ IgE+ IgG+ CD244.2+ CD19− CD20− NK1.1− Ag+ 2D7− BB1−, CD49b+ IgE+ IgG+ CD244.2+ CD19− CD20− NK1.1− Ag+ 2D7−, CD49b+ IgE+ IgG+ CD244.2+ CD19− CD20− NK1.1− Ag+ BB1−, CD49b+ IgE+ IgG+ FcεR1+ CD19− CD20− NK1.1− Ag+ 2D7− BB1−, CD49b+ IgE+ IgG+ FcεR1+ CD19− CD20− NK1.1− Ag+ 2D7−, CD49b+ IgE+ IgG+ FcεR1+ CD19− CD20− NK1.1− Ag+ BB1−, CD49b+ IgE+ IgG+ CD200R+ CD19− CD20− NKp46− Ag+ 2D7− BB1−, CD49b+ IgE+ IgG+ CD200R+ CD19− CD20− NKp46− Ag+ 2D7−, CD49b+ IgE+ IgG+ CD200R+ CD19− CD20− NKp46− Ag+ BB1−, CD49b+ IgE+ IgG+ CD244.2+ CD19− CD20− NKp46− Ag+ 2D7− BB1−, CD49b+ IgE+ IgG+ CD244.2+ CD19− CD20− NKp46− Ag+ 2D7−, CD49b+ IgE+ IgG+ CD244.2+ CD19− CD20− NKp46− Ag+ BB1−, CD49b+ IgE+ IgG+ FcεR1+ CD19− CD20− NKp46− Ag+ 2D7− BB1−, CD49b+ IgE+ IgG+ FcεR1+ CD19− CD20− NKp46− Ag+ 2D7−, CD49b+ IgE+ IgG+ FcεR1+ CD19− CD20− NKp46− Ag+ BB1−, CD49b+ IgE+ IgG+ CD200R+ CD19− CD20− CD122− Ag+ 2D7− BB1−, CD49b+ IgE+ IgG+ CD200R+ CD19− CD20− CD122− Ag+ 2D7−, CD49b+ IgE+ IgG+ CD200R+ CD19− CD20− CD122− Ag+ BB1−, CD49b+ IgE+ IgG+ CD244.2+ CD19− CD20− CD122− Ag+ 2D7− BB1−, CD49b+ IgE+ IgG+ CD244.2+ CD19− CD20− CD122− Ag+ 2D7−, CD49b+ IgE+ IgG+ CD244.2+ CD19− CD20− CD122− Ag+ BB1−, CD49b+ IgE+ IgG+ FcεR1+ CD19− CD20− CD122− Ag+ 2D7− BB1−, CD49b+ IgE+ IgG+ FcεR1+ CD19− CD20− CD122− Ag+ 2D7−, or CD49b+ IgE+ IgG+ FcεR1+ CD19− CD20− CD122− Ag+ BB1−. In some embodiments, the isolated antibody-producing cells are further determined to be positive for one or more marker as identified in Table 1.1 or Table 1.3, or are further determined to be negative for one or more marker as identified in Table 1.2.

In some embodiments a subpopulation of isolated antibody-producing cells is provided. The subpopulation further can be positive for at least one of the following markers: CD27, CD73, CD45RB and CD80.

### Complexes

Some embodiments include complexes. Complexes can include an antibody producing cell, and one or more molecules bound to the antibody producing cell. In some embodiments, complexes are useful for identifying an-antibody producing cell. Accordingly, in some embodiments, complexes include an antibody-producing cell bound to a molecule that includes at least one detectable marker as described herein. In some embodiments, complexes are useful for isolating an antibody-producing cell. Accordingly, in some embodiments, complexes include a binding molecule, for example a magnetic bead for pull-down.

Some embodiments include a complex that comprises an isolated antibody-producing cell, a CD49b-specific antibody bound to the cell, and an IgG-specific antibody bound to the cell. In some embodiments, the complex also contains an IgE− antibody bound to the cell. In some embodiments, the complex also contains at least one of a CD200R-specific antibody, CD244.2-specific antibody, or FcεR1-specific antibody. In some embodiments, the complex further includes an antigen specifically bound by an antibody produced by the antibody-producing cell. Such a complex can be useful for identifying and/or isolating an antibody-producing cell, for example by isolating or purifying cells that express surface antibodies with high affinity for the antigen. In some embodiments, for example if the complex comprises a naïve V cell, the complex does not comprise a cell-produced antibody with affinity for an antigen. In some embodiments, the complex does not contain (or does not contain more than trivial amounts of B220, 2D7, BB1, CD5, CD19, CD20, CD21/CD35, CD22.2, CD72, GL-7, IgD, IgM, Ly6-k, Ly6-D, Ly-51 CD127, CD138, CD154, AA4.1, Pax-5, NK1.1, CD49a, CD122 and CD226/NKp46, or any other marker listed in Table 1.2. Thus, the complex is not specifically bound by antibody targeting B220, CD5, CD19, CD20, CD21/CD35, CD22.2, CD72, GL-7, IgD, IgM, Ly6-k, Ly6-D, Ly-51 CD127, CD138, CD154, AA4.1, Pax-5, NK1.1, NK1.2, CD49a, CD122 or CD226/NKp46, or any other marker listed in Table 1.2. In some embodiments, the complex cannot be specifically bound by antibody targeting B220, 2D7, BB1, CD5, CD19, CD20, CD21/CD35, CD22.2, CD72 GL-7, IgD, IgM, Ly6-k, Ly6-D, Ly-51 CD127, CD138, CD154, AA4.1, Pax-5, NK1.1, NK1.2, CD49a, CD122 or CD226/NKp46, or any other marker listed in Table 1.2.

In some embodiments, an antibody of the complex comprises a detectable marker as described herein. Thus, in some embodiments, for example when an antibody comprising a detectable marker is bound to the complex, the complex comprises a detectable marker. In some embodiments, for example when the complex is not bound to a certain antibody comprising a detectable marker, the complex does not comprise a detectable marker associated with the non-binding antibody.

In some embodiments, the antibody is attached or configured to attach to a cell system as described herein.

In some embodiments, the antibody comprises a therapeutic agent as described herein.

In some embodiments, the complex includes at least one additional antibody. For example, various subpopulations of V cells can further be CD24+, CD43, CD45+, CD48+, CD79b, CD123, or CD16/CD32 (and as such, some subpopulations can also be negative for one or more of these markers). In some embodiments, the additional antibody binds specifically to CD24, CD43, CD45, CD48, CD79b, CD123, or CD16/CD32. In some embodiments, the complex includes two or more additional antibodies, each of which specifically binds to a different marker, and each of which binds to one of CD24, CD43, CD45, CD48, CD79b, CD123, or CD16/CD32. Thus, in some embodiments, the complex includes a first additional antibody and second additional antibody that respectively bind to CD24 and CD43; CD24 and CD45; CD24 and CD48; CD43 and CD45; CD43 and CD48; or CD45 and CD48; CD24 and CD16/CD32, CD24 and CD79b, CD123, CD43 and CD16/CD32, CD43 and CD79b, CD45 and CD16/CD32, CD45 and CD79b.

### Methods of Determining the Presence or Absence of Particular Cells

When characterizing or screening for antibody-producing cells or cells capable of producing antibody, for example V cells or subpopulations thereof, methods of identifying such cells can be useful. Methods of identifying antigen-specific antibody producing cells or cells capable of producing antibody such as V cells can also be useful for many other applications, for example, researching antibody producing cells, drug screening, diagnosis of disease state, and/or determining a prognosis. Accordingly, some aspects of the invention include methods of determining the presence or absence of cell types disclosed herein, for example V cells. The methods can include providing a population of mammalian cells. The methods can include determining the presence or absence of one or more V cells from the population, for example IgG+ IgE+ B220− 2D7− cells that are positive for at least one additional marker indicated in Table 1.1, for example CD49b, CD16/CD32, CD24, CD43, CD45, CD48, CD54, CD79b, CD123, CD200R, CD244.2, or FcεR1. Exemplary V cell phenotypes include, but are not limited to cells are CD49b+ IgG+ IgE+ CD200R+ and that are negative for B-cell specific markers and basophil-specific markers. In some embodiments, the V cells are CD49b+ IgG+ IgE+ CD244.2+ cells that are negative for B-cell specific markers and basophil-specific markers. In some embodiments, the V cells are CD49b+ IgG+ IgE+ FcεR1+ cells that are negative for B-cell specific markers and basophil-specific markers. In some embodiments, the V cell is IgG+ IgE+ CD16/32+ and CD19−. In some embodiments, the V cell is IgG+ IgE+ CD16/32+ 2D7− BB1− and CD20−. In some embodiments, the V cell is IgG+ IgE+ CD16/32+ 2D7− and CD20−. In some embodiments, the V cell is IgG+ IgE+ CD16/32+ BB1− and CD20−. In some embodiments, the V cell is IgG+ IgE+ CD16/32+2D7− B220−. In some embodiments, the V cell is IgG+ IgE+ CD16/32+ 2D7− B220−. In some embodiments, the V cell is IgG+ IgE+ CD24+ 2D7− BB1− and CD19−. In some embodiments, the V cell is IgG+ IgE+ CD24+ 2D7− and CD19−. In some embodiments, the V cell is IgG+ IgE+ CD24+ BB1− and CD19−. In some embodiments, the V cell is IgG+ IgE+ CD24+2D7− BB1− and CD20−. In some embodiments, the V cell is IgG+ IgE+ CD24+ 2D7− and CD20−. In some embodiments, the V cell is IgG+ IgE+ CD24+ BB1− and CD20−. In some embodiments, the V cell is IgG+ IgE+ CD24+ 2D7− BB1− and B220−. In some embodiments, the V cell is IgG+ IgE+ CD24+ 2D7− and B220−. In some embodiments, the V cell is IgG+ IgE+ CD24+ BB1− and B220−. In some embodiments, the V cell is IgG+ IgE+ CD43+ 2D7− BB1− and CD19−. In some embodiments, the V cell is IgG+ IgE+ CD43+ 2D7− and CD19−. In some embodiments, the V cell is IgG+ IgE+ CD43+ BB1− and CD19−. In some embodiments, the V cell is IgG+ IgE+ CD43+ 2D7− BB1− and CD20−. In some embodiments, the V cell is IgG+ IgE+ CD43+ 2D7− and CD20−. In some embodiments, the V cell is IgG+ IgE+ CD43+ BB1− and CD20−. In some embodiments, the V cell is IgG+ IgE+ CD43+ 2D7− BB1− and B220−. In some embodiments, the V cell is IgG+ IgE+ CD43+ 2D7− and B220−. In some embodiments, the V cell is IgG+ IgE+ CD43+ BB1− and B220−. In some embodiments, the V cell is IgG+ IgE+ CD43+ 2D7− BB1− and CD19−. In some embodiments, the V cell is IgG+ IgE+ CD43+ 2D7− and CD19−. In some embodiments, the V cell is IgG+ IgE+ CD43+ BB1− and CD19−. In some embodiments, the V cell is IgG+ IgE+ CD43+ 2D7− BB1− and CD20−. In some embodiments, the V cell is IgG+ IgE+ CD43+ 2D7− and CD20−. In some embodiments, the V cell is IgG+ IgE+ CD43+ BB1− and CD20−. In some embodiments, the V cell is IgG+ IgE+ CD43+ 2D7− BB1− and B220−. In some embodiments, the V cell is IgG+ IgE+ CD43+ 2D7− and B220−. In some embodiments, the V cell is IgG+ IgE+ CD43+ BB1− and B220−. In some embodiments, the V cell is IgG+ IgE+ CD45+ 2D7− BB1− and CD19−. In some embodiments, the V cell is IgG+ IgE+ CD45+ 2D7− and CD19−. In some embodiments, the V cell is IgG+ IgE+ CD45+ BB1− and CD19−. In some embodiments, the V cell is IgG+ IgE+ CD45+ 2D7− BB1− and CD20−. In some embodiments, the V cell is IgG+ IgE+ CD45+ 2D7− and CD20−. In some embodiments, the V cell is IgG+ IgE+ CD45+ BB1− and CD20−. In some embodiments, the V cell is IgG+ IgE+ CD45+ 2D7− BB1− and B220−. In some embodiments, the V cell is IgG+ IgE+ CD45+ 2D7− and B220−. In some embodiments, the V cell is IgG+ IgE+ CD45+ BB1− and B220−. In some embodiments, the V cell is IgG+ IgE+ CD48+2D7− BB1− and CD19−. In some embodiments, the V cell is IgG+ IgE+ CD48+ 2D7− and CD19−. In some embodiments, the V cell is IgG+ IgE+ CD48+ BB1− and CD19−. In some embodiments, the V cell is IgG+ IgE+ CD48+ 2D7− BB1− and CD20−. In some embodiments, the V cell is IgG+ IgE+ CD48+ 2D7− and CD20−. In some embodiments, the V cell is IgG+ IgE+ CD48+ BB1− and CD20−. In some embodiments, the V cell is IgG+ IgE+ CD48+ 2D7− BB1− and B220−. In some embodiments, the V cell is IgG+ IgE+ CD48+ 2D7− and B220−. In some embodiments, the V cell is IgG+ IgE+ CD48+ BB1− and B220−. In some embodiments, the V cell is IgG+ IgE+ CD54+2D7− BB1− and CD19−. In some embodiments, the V cell is IgG+ IgE+ CD54+2D7− and CD19−. In some embodiments, the V cell is IgG+ IgE+ CD54+ BB1− and CD19−. In some embodiments, the V cell is IgG+ IgE+ CD54+2D7− BB1− and CD20−. In some embodiments, the V cell is IgG+ IgE+ CD54+2D7− and CD20−. In some embodiments, the V cell is IgG+ IgE+ CD54+ BB1− and CD20−. In some embodiments, the V cell is IgG+ IgE+ CD54+2D7− BB1− and B220−. In some embodiments, the V cell is IgG+ IgE+ CD54+2D7− and B220−. In some embodiments, the V cell is IgG+ IgE+ CD54+ BB1− and B220−. In some embodiments, the V cell is IgG+ IgE+ CD79b+ 2D7− BB1− and CD19−. In some embodiments, the V cell is IgG+ IgE+ CD79b+ 2D7− and CD19−. In some embodiments, the V cell is IgG+ IgE+ CD79b+ BB1− and CD19−. In some embodiments, the V cell is IgG+ IgE+ CD79b+ 2D7− BB1− and CD20−. In some embodiments, the V cell is IgG+ IgE+ CD79b+ 2D7− and CD20−. In some embodiments, the V cell is IgG+ IgE+ CD79b+ BB1− and CD20−. In some embodiments, the V cell is IgG+ IgE+ CD79b+ 2D7− BB1− and B220−. In some embodiments, the V cell is IgG+ IgE+ CD79b+ 2D7− and B220−. In some embodiments, the V cell is IgG+ IgE+ CD79b+ BB1− and B220−. In some embodiments, the V cell is IgG+ IgE+ CD200R 2D7− BB1− and CD19−. In some embodiments, the V cell is IgG+ IgE+ CD200R 2D7− and CD19−. In some embodiments, the V cell is IgG+ IgE+ CD200R BB1− and CD19−. In some embodiments, the V cell is IgG+ IgE+ CD200R+2D7− BB1− and CD20−. In some embodiments, the V cell is IgG+ IgE+ CD200R+2D7− and CD20−. In some embodiments, the V cell is IgG+ IgE+ CD200R+ BB1− and CD20−. In some embodiments, the V cell is IgG+ IgE+C200R+ 2D7− BB1− and B220−. In some embodiments, the V cell is IgG+ IgE+C200R+ 2D7− and B220−. In some embodiments, the V cell is IgG+ IgE+ CD200R+ 2D7− BB1− and CD19−. In some embodiments, the V cell is IgG+ IgE+ CD200R+ 2D7− and CD19−. In some embodiments, the V cell is IgG+ IgE+ CD244.2+ BB1− and CD19−. In some embodiments, the V cell is IgG+ IgE+ CD200R+ 2D7− BB1− and CD20−. In some embodiments, the V cell is IgG+ IgE+ CD200R+ 2D7− and CD20−. In some embodiments, the V cell is IgG+ IgE+ CD244.2+ BB1− and CD20−. In some embodiments, the V cell is IgG+ IgE+ CD200R+ 2D7− BB1− and B220−. In some embodiments, the V cell is IgG+ IgE+ CD200R+ 2D7− and B220−. In some embodiments, the V cell is IgG+ IgE+ CD244.2+ BB1− and B220−. In some embodiments, the V cell is IgG+ IgE+ FcεR1+ 2D7− BB1− and CD19−. In some embodiments, the V cell is IgG+ IgE+ FcεR1+ 2D7− and CD19−. In some embodiments, the V cell is IgG+ IgE+ FcεR1+ BB1− and CD19−. In some embodiments, the V cell is IgG+ IgE+ FcεR1+ 2D7− BB1− and CD20−. In some embodiments, the V cell is IgG+ IgE+ FcεR1+ 2D7− and CD20−. In some embodiments, the V cell is IgG+ IgE+ FcεR1+ BB1− and CD20−. In some embodiments, the V cell is IgG+ IgE+ FcεR1+ 2D7− BB1− and B220−. In some embodiments, the V cell is IgG+ IgE+ FcεR1+ 2D7− and B220−. In some embodiments, the V cell is IgG+ IgE+ FcεR1+ BB1− and B220−. The V cells listed above can further be CD49b+, though it is noted that some V cells, for example naïve human V cells, can be CD49b^{low} or CD49b−.

In some embodiments, the population of cells is a population of cells of the hematopoietic lineage of a mammal. Exemplary mammals include, but are not limited to humans, non-human primates, mice, rats, guinea pigs, rabbits, cats, dogs, goats, donkeys, sheep, cows, and camels, including genetically modified versions of these organisms. In some embodiments, the population of cells is derived from one of a spleen, bone marrow, tonsils, blood, or peripheral blood mononuclear cells (PBMCs). In some embodiments, the population of cells is isolated from a host. In some embodiments, the population of cells is freshly harvested. In some embodiments, the population of cells is fresh-frozen or otherwise preserved.

Various assays can be used to assay a population of cells for the presence or absence of cells containing certain markers. Exemplary assays include, but are not limited to immunoassays, such as flow cytometry, FACs sorting, western blot, ELISA, spot blot, fluorescent microscopy, immunoassays, immunoseparation, affinity column, affinity beads with or without a magnetic, or physical separation, and the like. Such assays can be used in accordance with some embodiments herein, for example to detect, isolate, or enrich a population for V cells. Kits in accordance with some embodiments herein can appropriate reagents, quantities, and formats for the listed types of assays. Typically, immunoassays involve detecting the binding of a marker-specific antibody to a cell and/or molecule in a biological sample.

In some embodiments, the method includes contacting a cell-containing sample with an antibody that specifically binds to CD49b, an antibody that specifically binds to IgG, an antibody that specifically binds to IgE, an antibody that specifically binds to basophil markers 2D7 and/or BB1, and an antibody that specifically binds to a B cell, in which each antibody comprises a different detectable marker. The method can further include contacting the sample with at least one of: an antibody that specifically binds to CD200R, CD244.2, or FcεR1. As such, the presence of a CD49b+ IgG+ IgE+ CD200R+ cell that is absent for at least one B-cell specific marker and absent of basophil specific markers can indicate the presence of a V cell, as can a CD49b+ IgG+ IgE+ CD244.2+ cell that is absent for at least one B-cell specific marker and absent for basophil specific markers, or a CD49b+ IgG+ IgE+ FcεR1+ cell that is absent for at least one B-cell specific marker and absent for basophil specific markers. In some embodiments, the antibody that binds specifically to a B cell binds specifically to one of B220, CD5, CD19, CD20, CD21/CD35, CD22.2, CD72 GL-7, IgD, IgM, Ly6-k, Ly6-D, Ly-51 CD127, CD138, CD154, AA4.1, Pax-5, NK1.1, NK1.2, CD49a, CD122 or CD226/NKp46, for example B220. In some embodiments, the method includes determining the presence or absence of a V cell that is not bound by a B cell-specific antibody, for example an antibody that binds specifically to one of B220, CD5, CD19, CD20, CD21/CD35, CD22.2, CD72 GL-7, IgD, IgM, Ly6-k, Ly6-D, Ly-51 CD127, CD138, CD154, AA4.1, Pax-5, NK1.1, NK1.2, CD49a, CD122 or CD226/NKp46, and is not bound by a basophil specific marker, for example an antibody that binds specifically to one of 2D7 and BB1. Exemplary V cells that are not bound by a B cell-specific antibody can be identified as CD49b+ IgG+ IgE+ CD200R+ B220− 2D7−, CD49b+ IgG+ IgE+ CD200R+ B220− BB1−, CD49b+ IgG+ IgE+ CD200R+ CD5− 2D7− BB1−, CD49b+ IgG+ IgE+ CD200R+ CD5− 2D7−, CD49b+ IgG+ IgE+ CD200R+ CD5− BB1−, CD49b+ IgG+ IgE+ CD200R+ CD19− 2D7− BB1−, CD49b+ IgG+ IgE+ CD200R+ CD19− 2D7−, CD49b+ IgG+ IgE+ CD200R+ CD19− BB1−, CD49b+ IgG+ IgE+ CD200R+ CD20− 2D7− BB1−, CD49b+ IgG+ IgE+ CD200R+ CD20− 2D7−, CD49b+ IgG+ IgE+ CD200R+ CD20− BB1−, CD49b+ IgG+ IgE+ CD200R+ CD21/CD35− 2D7− BB1−, CD49b+ IgG+ IgE+ CD200R+ CD21/CD35− 2D7−, CD49b+ IgG+ IgE+ CD200R+ CD21/CD35− BB1−, CD49b+ IgG+ IgE+ CD200R+ CD22.2− 2D7− BB1−, CD49b+ IgG+ IgE+ CD200R+ CD22.2− 2D7−, CD49b+ IgG+ IgE+ CD200R+ CD22.2− BB1−, CD49b+ IgG+ IgE+ CD200R+ CD72− 2D7− BB1−, CD49b+ IgG+ IgE+ CD200R+ CD72− 2D7−, CD49b+ IgG+ IgE+ CD200R+ CD72− BB1−, CD49b+ IgG+ IgE+ CD200R+ IgD− 2D7− BB1, CD49b+ IgG+ IgE+ CD200R+IgD− 2D7−, CD49b+ IgG+ IgE+ CD200R+ IgD− BB1−, CD49b+ IgG+ IgE+ CD200R+IgM− 2D7− BB1−, CD49b+ IgG+ IgE+ CD200R+ IgM− 2D7−, CD49b+ IgG+ IgE+ CD200R+IgM− BB1−, CD49b+ IgG+ IgE+ CD200R+Ly6− K− 2D7− BB1−, CD49b+ IgG+ IgE+ CD200R+Ly6−K− 2D7−, CD49b+ IgG+ IgE+ CD200R+ Ly6-K− BB1−, CD49b+ IgG+ IgE+ CD200R+Ly6-D− 2D7− BB1−, CD49b+ IgG+ IgE+ CD200R+Ly6-D− 2D7−, CD49b+ IgG+ IgE+ CD200R+Ly6-D− BB1−, CD49b+ IgG+ IgE+ CD200R+Ly-51− 2D7− BB1−, CD49b+ IgG+ IgE+ CD200R+Ly-51− 2D7−, CD49b+ IgG+ IgE+ CD200R+Ly-51− BB1−, CD49b+ IgG+ IgE+ CD200R+ CD127− 2D7− BB1−, CD49b+ IgG+ IgE+ CD200R+ CD127− 2D7−, CD49b+ IgG+ IgE+ CD200R+ CD127− BB1−, CD49b+ IgG+ IgE+ CD200R+ CD138− 2D7− BB1−, CD49b+ IgG+ IgE+ CD200R+ CD138− 2D7−, CD49b+ IgG+ IgE+ CD200R+ CD138− BB1−, CD49b+ IgG+ IgE+ CD200R+ CD154− 2D7− BB1−, CD49b+ IgG+ IgE+ CD200R+ CD154− 2D7−, CD49b+ IgG+ IgE+ CD200R+ CD154− BB1−, CD49b+ IgG+ IgE+ CD200R+AA4.1− 2D7− BB1−, CD49b+ IgG+ IgE+ CD200R+ AA4.1− 2D7−, CD49b+ IgG+ IgE+ CD200R+AA4.1− BB1−, CD49b+ IgG+ IgE+ CD200R+ Pax-5− 2D7− BB1−, CD49b+ IgG+ IgE+ CD200R+ Pax-5− 2D7−, CD49b+ IgG+ IgE+ CD200R+ Pax-5− 2D7−, CD49b+ IgG+ IgE+ CD200R+ Pax-5− BB1−, CD49b+ IgG+ IgE+ CD200R+ NK1.1− 2D7− BB1−, CD49b+ IgG+ IgE+ CD200R+ NK1.1− 2D7−, CD49b+ IgG+ IgE+ CD200R+ NK1.1− BB1−, CD49b+ IgG+ IgE+ CD200R+ CD49a− 2D7− BB1−, CD49b+ IgG+ IgE+ CD200R+ CD49a− 2D7−, CD49b+ IgG+ IgE+ CD200R+ CD49a− BB1−, CD49b+ IgG+ IgE+ CD200R+ CD122− 2D7− BB1−, CD49b+ IgG+ IgE+ CD200R+ CD122− 2D7−, CD49b+ IgG+ IgE+ CD200R+ CD122− BB1−, CD49b+ IgG+ IgE+ CD200R+ CD226− 2D7− BB1−, CD49b+ IgG+ IgE+ CD200R+ CD226− 2D7−, or CD49b+ IgG+ IgE+ CD200R+ CD226− BB1− cells.

In some embodiments, the method includes contacting the cell-containing sample with an antibody that specifically binds to NK cells, in which the antibody binds specifically to a marker other than CD49b. In some embodiments, the antibody that binds specifically to NK cells binds specifically to one of NK1.1, KN1.2, NKp46, or CD122. In some embodiments, the method includes determining the presence or absence of a CD49b+ IgG+ cell that is not bound by a such an NK cell-specific antibody, for example a CD49b+ IgG+NK1.1−, CD49b+IgG+NK1.2−, CD49b+ IgG+ NKp46−, or CD49b+ IgG+CD122− cell.

In some embodiments, the method includes contacting the cell-containing sample with an antibody that specifically binds to one of NK1.1, NK1.2, CD1d, CD3, CD4, CD8, CD25, CD38 and CD134; CD11c & CD273; CD49a, CD122 and CD226/NKp46; CD34, Sca-1, c-Kit and CD150; CD11b and Ly-6G; and CD123. The method can include identifying the cell as negative for one or more of NK1.1, NK1.2, CD1d, CD3, CD4, CD8, CD25, CD38 and CD134; CD11c & CD273; CD49a, CD122 and CD226/NKp46; CD34, Sca-1, c-Kit and CD150; Ly-6G; and CD123.

In some embodiments, the cells capable of producing antibody (for example V cells) are detected at least partially based on morphology, for example a polymorphonucleated nucleus or an annular-shaped nucleus. Morphological features of V cells can be detected using microscopy methods well-known to one of skill in the art, for example confocal microscopy, electron microscopy, and the like.

In some embodiments, the cells capable of producing antibody (for example V cells) are detected at least partly based on their specific affinity for antigen (Ag). Affinity for antigen can indicate the presence of antigen-specific surface-bound antibody on the V cell. The presence of antigen can be determined in a variety of ways. For example, a V cell can be contacted with antigen comprising a detectable marker and the amount of detectable marker associated with the V cell can be detected. For example, a V cell can be contacted with antigen immobilized on a solid phase, and the amount binding of V cells the solid phase can be detected. In some optional embodiments, the method includes determining the presence or absence of a CD49b+ IgG+ IgE+ Ag+ cell that is absent for at least one B-cell-specific marker and basophil-specific markers (2D7 and/or BB1), CD49b+ IgG+ IgE+ CD200+ Ag+ cell that is absent for at least one B-cell-specific marker and basophil-specific markers (2D7 and/or BB1), CD49b+ IgG+ IgE+ CD244.2+ Ag+ cell that is absent for at least one B-cell-specific marker and basophil-specific markers (2D7 and/or BB1), CD49b+ IgG+ IgE+ FcεR1+Ag+ cell that is absent for at least one B-cell-specific marker, and absent for basophil-specific markers (2D7 and/or BB1).

Without being limited by any theory, it is reported herein that V cells can upregulate or downregulate various markers depending on their activation stage or maturity stage. As such, in some embodiments, relative amounts of one or more marker are detected. In some embodiments, amounts of a “positive” marker, for example one or more markers disclosed in Table 1.1 are detected. In some embodiments, amounts of a “negative” marker are detected, for example one or more markers disclosed in Table 1.2 are detected. In some embodiments, amounts of a marker expressed at low levels, for example one or more makers disclosed in Table 1.3 are detected. Amounts of markers can be determined using various methods, for example via flow cytometry, immunohistochemistry, or immunoblotting. In some embodiments, relative amounts of a marker are compared between among two or more V cells. In some embodiments, relative amounts of two or more markers are compared on a single V cell, or among two or more V cells. In some embodiments, relative amounts of at least one marker are monitored in a population of V cells over time. In some embodiments, relative amounts of at least one marker are monitored in a single V cell over time. It is noted that because V cells that can produce affinity matured antibody for an antigen can also be identified based on other markers, in some embodiments, an antibody-producing V cell that produces antibody that binds specifically to an antigen can be identified without contacting the cell with the antigen itself. Furthermore, because some V cells are naïve V cells, in some embodiments, a V cell capable of producing Ag specific antibody, but that does not yet produce Ag specific antibody is identified (see, e.g., Example 4). In some embodiments, the naïve V cell has a B220−IgG+IgE+CD49b+.

### Methods of Enriching a Cell Population

It can be useful to enrich a cell population for antibody-producing cells, for example to identify and isolate antibodies that bind specifically to an antigen, to produce quantities of antibody, or for therapeutic applications, such as autologous or allogeneic cell transplant. Accordingly, some aspects of the invention include methods of enriching a population of cells for antibody-producing cells. The antibody-producing cells can be V cells as described herein, for example CD49b+ IgG+ IgE+ cells that are negative for B-cell specific markers, and positive for at least one “positive” marker indicated in Table 1.1, for example, at least one of CD16/CD32, CD24, CD43, CD45, CD48, CD54, CD79b, CD200R, CD244.2, or FcεR1. In some embodiments, the V cells are CD49b+ IgG+ IgE+ CD200R+ cells that are negative for B-cell specific markers and basophil-specific markers (2D7 and/or BB1). In some embodiments, the V cells are CD49b+ IgG+ IgE+ CD244.2+ cells that are negative for B-cell specific markers and basophil-specific markers (2D7 and/or BB1). In some embodiments, the V cells are CD49b+ IgG+ IgE+ FcεR1+ cells that are negative for B-cell specific markers and basophil-specific markers (2D7 and/or BB1). In some embodiments, the V cell is IgG+ IgE+ CD16/32+ 2D7− BB1− and CD19−. In some embodiments, the V cell is IgG+ IgE+ CD16/32+ 2D7− and CD19−. In some embodiments, the V cell is IgG+ IgE+ CD16/32+ BB1− and CD19−. In some embodiments, the V cell is IgG+ IgE+ CD16/32+ 2D7− BB1− and CD20−. In some embodiments, the V cell is IgG+ IgE+ CD16/32+ 2D7− and CD20−. In some embodiments, the V cell is IgG+ IgE+ CD16/32+ BB1− and CD20−. In some embodiments, the V cell is IgG+ IgE+ CD16/32+ 2D7− BB1− and B220. In some embodiments, the V cell is IgG+ IgE+ CD16/32+ 2D7− and B220. In some embodiments, the V cell is IgG+ IgE+ CD24+ 2D7− BB1− and CD19−. In some embodiments, the V cell is IgG+ IgE+ CD24+ 2D7− and CD19−. In some embodiments, the V cell is IgG+ IgE+ CD24+ BB1− and CD19−. In some embodiments, the V cell is IgG+ IgE+ CD24+ 2D7− BB1− and CD20−. In some embodiments, the V cell is IgG+ IgE+ CD24+ 2D7− and CD20−. In some embodiments, the V cell is IgG+ IgE+ CD24+ BB1− and CD20−. In some embodiments, the V cell is IgG+ IgE+ CD24+ 2D7− BB1− and B220−. In some embodiments, the V cell is IgG+ IgE+ CD24+ 2D7− and B220−. In some embodiments, the V cell is IgG+ IgE+ CD43+ 2D7− BB1− and CD19−. In some embodiments, the V cell is IgG+ IgE+ CD43+ 2D7− and CD19−. In some embodiments, the V cell is IgG+ IgE+ CD43+ BB1− and CD19−. In some embodiments, the V cell is IgG+ IgE+ CD43+ 2D7− BB1− and CD20−. In some embodiments, the V cell is IgG+ IgE+ CD43+ 2D7− and CD20−. In some embodiments, the V cell is IgG+ IgE+ CD43+ BB1− and CD20−. In some embodiments, the V cell is IgG+ IgE+ CD43+ 2D7− BB1− and B220−. In some embodiments, the V cell is IgG+ IgE+ CD43+ 2D7− and B220−. In some embodiments, the V cell is IgG+ IgE+ CD43+ 2D7− BB1− and CD19−. In some embodiments, the V cell is IgG+ IgE+ CD43+ 2D7− and CD19−. In some embodiments, the V cell is IgG+ IgE+ CD43+ BB1− and CD19−. In some embodiments, the V cell is IgG+ IgE+ CD43+ 2D7− BB1− and CD20−. In some embodiments, the V cell is IgG+ IgE+ CD43+ 2D7− and CD20−. In some embodiments, the V cell is IgG+ IgE+ CD43+ BB1− and CD20−. In some embodiments, the V cell is IgG+ IgE+ CD43+ 2D7− and B220−. In some embodiments, the V cell is IgG+ IgE+ CD43+ BB1− and B220−. In some embodiments, the V cell is IgG+ IgE+ CD45+ 2D7− BB1− and CD19−. In some embodiments, the V cell is IgG+ IgE+ CD45+ 2D7− and CD19−. In some embodiments, the V cell is IgG+ IgE+ CD45+ BB1− and CD19−. In some embodiments, the V cell is IgG+ IgE+ CD45+ 2D7− BB1− and CD20−. In some embodiments, the V cell is IgG+ IgE+ CD45+ 2D7− and CD20−. In some embodiments, the V cell is IgG+ IgE+ CD45+ BB1− and CD20−. In some embodiments, the V cell is IgG+ IgE+ CD45+ 2D7− BB1− and B220−. In some embodiments, the V cell is IgG+ IgE+ CD45+ 2D7− and B220−. In some embodiments, the V cell is IgG+ IgE+ CD48+ 2D7− BB1− and CD19−. In some embodiments, the V cell is IgG+ IgE+ CD48+ 2D7− and CD19−. In some embodiments, the V cell is IgG+ IgE+ CD48+ BB1− and CD19−. In some embodiments, the V cell is IgG+ IgE+ CD48+ 2D7− BB1− and CD20−. In some embodiments, the V cell is IgG+ IgE+ CD48+ 2D7− and CD20−. In some embodiments, the V cell is IgG+ IgE+ CD48+ BB1− and CD20−. In some embodiments, the V cell is IgG+ IgE+ CD48+ 2D7− and B220−. In some embodiments, the V cell is IgG+ IgE+ CD54+2D7− BB1− and CD19−. In some embodiments, the V cell is IgG+ IgE+ CD54+ 2D7− and CD19−. In some embodiments, the V cell is IgG+ IgE+ CD54+ BB1− and CD19−. In some embodiments, the V cell is IgG+ IgE+ CD54+2D7− BB1− and CD20−. In some embodiments, the V cell is IgG+ IgE+ CD54+2D7− and CD20−. In some embodiments, the V cell is IgG+ IgE+ CD54+ BB1− and CD20−. In some embodiments, the V cell is IgG+ IgE+ CD54+2D7− and B220−. In some embodiments, the V cell is IgG+ IgE+ CD79b+ 2D7− BB1− and CD19−. In some embodiments, the V cell is IgG+ IgE+ CD79b+ 2D7− and CD19−. In some embodiments, the V cell is IgG+ IgE+ CD79b+ BB1− and CD19−. In some embodiments, the V cell is IgG+ IgE+ CD79b+ 2D7− BB1− and CD20−. In some embodiments, the V cell is IgG+ IgE+ CD79b+ 2D7− and CD20−. In some embodiments, the V cell is IgG+ IgE+ CD79b+ BB1− and CD20−. In some embodiments, the V cell is IgG+ IgE+ CD79b+ 2D7− BB1− and B220−. In some embodiments, the V cell is IgG+ IgE+ CD79b+ 2D7− and B220−. In some embodiments, the V cell is IgG+ IgE+ CD200R 2D7− BB1− and CD19−. In some embodiments, the V cell is IgG+ IgE+ CD200R 2D7− and CD19−. In some embodiments, the V cell is IgG+ IgE+ CD200R BB1− and CD19−. In some embodiments, the V cell is IgG+ IgE+ CD200R+2D7− BB1− and CD20−. In some embodiments, the V cell is IgG+ IgE+ CD200R+2D7− and CD20−. In some embodiments, the V cell is IgG+ IgE+ CD200R+2D7− and CD20−. In some embodiments, the V cell is IgG+ IgE+C200R+ 2D7− BB1− and B220−. In some embodiments, the V cell is IgG+ IgE+ C200R+ 2D7− and B220−. In some embodiments, the V cell is IgG+ IgE+ CD200R+ 2D7− BB1− and CD19−. In some embodiments, the V cell is IgG+ IgE+ CD200R+ 2D7− and CD19−. In some embodiments, the V cell is IgG+ IgE+ CD244.2+ BB1− and CD19−. In some embodiments, the V cell is IgG+ IgE+ CD200R+ 2D7− BB1− and CD20−. In some embodiments, the V cell is IgG+ IgE+ CD200R+ 2D7− and CD20. In some embodiments, the V cell is IgG+ IgE+ CD244.2+ BB1− and CD20−. In some embodiments, the V cell is IgG+ IgE+ CD200R+ 2D7− and B220−. In some embodiments, the V cell is IgG+ IgE+ FcεR1+ 2D7− BB1− and CD19−. In some embodiments, the V cell is IgG+ IgE+ FcεR1+2D7− and CD19−. In some embodiments, the V cell is IgG+ IgE+ FcεR1+ BB1− and CD19−. In some embodiments, the V cell is IgG+ IgE+ FcεR1+2D7− BB1− and CD20−. In some embodiments, the V cell is IgG+ IgE+ FcεR1+2D7− and CD20−. In some embodiments, the V cell is IgG+ IgE+ FcεR1+ BB1− and CD20−. In some embodiments, the V cell is IgG+ IgE+ FcεR1+ 2D7− and B220−. The V cells can also be CD49b+ 2D7− BB1−, CD49b+ 2D7−, or CD49b+ BB1−, though it is noted that some V cells, for example naïve human V cells, can be CD49b^{low} or CD49b

In some embodiments, the methods include removing at least one or more of the following cell types from a population: B cells, T cells, Monocytes (granulocytes and macrophages), dendritic cells, NK cells, erythrocytes, basophils, and hematopoietic stem cells (for example C-kit/Sca-1/CD150 positive cells). In some embodiments, the methods include removing cells that are positive for at least one marker listed in Table 1.2 from the population. In some embodiments, the method includes contacting a sample with an antibody or antibodies that bind specifically to the indicated cell type (or marker type), and separating at least one cell bound to the antibodies from other cells of the sample.

In some embodiments, cells are removed by contacting a sample with an antibody that removes at least one type of cell (such an antibody may be referred to herein as an “enrichment antibody”). In some embodiments, cells are removed by contacting a sample with an antibody that binds specifically to T cells. In some embodiments, cells are removed by contacting a sample with an antibody that binds specifically to monocytes. In some embodiments, cells are removed by contacting a sample with an antibody that binds specifically to dendritic cells. In some embodiments, cells are removed by contacting a sample with an antibody that binds specifically to NK cells. In some embodiments, cells are removed by contacting a sample with an antibody that binds specifically to erythrocytes cells. In some embodiments, cells are removed by contacting a sample with an antibody that binds specifically to hematopoietic stem cells. In some embodiments, the method includes contacting the sample with two or more enrichment antibodies, each of which bind specifically to two of the listed cell types (or a bispecific antibody that binds specifically to two of the listed cell types), for example two, three, four, five, or six of the listed cell types. Certain markers for the indicated cell types and antibodies thereto are shown in Table 2.

TABLE 2
Exemplary markers for indicated cell types

| Cell Type | Exemplary Markers/Antibodies |
|---|---|
| T Cells | CD1d, CD3, CD4, CD8, CD25, CD38 and CD134 |
| Monocytes | Ly-6G; |
| Dendritic Cells | CD11c and CD273 |
| NK Cells | NK1.1, CD49a, CD122 and CD226/NKp46 |
| | (in some embodiments, human CD57/mouseB3gat1 |
| | can also be used as an NK cell marker) |
| Hematopoietic | Sca-1, c-Kit and CD150 |
| Stem Cells | |
| Basophils | CD123, 2D7 (humans or mice), BB1 (humans) |

In some embodiments, contacting the sample with antibody includes adding antibody to the sample. In some embodiments, contacting the sample with antibody includes adding the sample to an antibody. In some embodiments, contacting the sample with antibody includes reconstituting an antibody, for example a lyophilized antibody in the sample. In some embodiments, contacting the sample with antibody includes adding to the sample a cell that secretes the indicated antibody. In some embodiments, the antibody is an antibody listed in Table 2.

In some embodiments, separating cells bound to the antibody from the sample includes using one or more cell separation system as described herein. In some embodiments, the method includes adding to the sample antibody attached to a separable phase, and removing the separable phase from the sample. Optionally, the antibody and separable phase can be incubated in the sample for a period of time to facilitate binding of antibody to cells. In some embodiments, the method includes antibody configured to attach to a separable phase as described herein. For example, the antibody can be biotinylated, GST-tagged, or marked with a detectable marker such as a fluorochrome. The method can include adding a separable phase to the sample as described herein, so that the antibody attaches to the separable phase. The method can include removing the separable phase from the sample. In some embodiments, the separable phase includes magnetic beads, and the magnetic beads are separated from the sample by applying a magnetic field.

In some embodiments, at least about 60% of the targeted cell type is removed from the sample, for example at least about 60%, 70%, 75%, 80%, 85%, 90%, 95%, 96%. 97%, 98%, 99%, or 99.95%. In some embodiments removing the targeted cell type provides a population that includes at least about 20% V cells, for example about 20%, 30%, 40%, 50%, 60%, 70%, 75%, 80%, 85%, 90%, 95%, 96%, 97%, 98%, 99%, or 99.95%.

In some embodiments, the method includes enriching a population of cells by flow cytometry. In some embodiments, the method includes enriching a population of cells via non-flow cytometry methods, for example if flow cytometry equipment is unavailable, or considerations of economy and/or scale favor other methods. Accordingly, in some embodiments, the methods include enriching a population of cells via a column. In some embodiments, the methods include enriching a population of cells in a solution by precipitating one or more undesired cell types from solution.

Kits for the Detection and/or Isolation of Antibody-Producing Cells or Cells Capable of Producing Antibody

Some aspects of the invention include kits for the detection and/or isolation of antibody-producing cells or cells capable of producing antibody. The antibody-producing cells can be V cells. In some embodiments, the kits include reagents and the like for detecting V cells via flow cytometry. If V cells are identified via flow cytometry, standard flow cytometry methods can be used to isolate V cells by isolating a population of cells that includes the profile of markers used to identify V cells. Accordingly, in some embodiments, a kit can be used for identification of V cells, and can also be used for the isolation of V cells.

In some embodiments, the kits include one or more reagents (for example antibodies, dyes, stains and the like) each of which targets a different cellular marker. In some embodiments, for example, embodiments in which kits include two or more types of molecules, each unique reagent is attached a different detectable marker. In some embodiments, each detectable marker is a fluorochrome.

Different hosts can have different cellular markers, for example hematopoietic lineage commitment markers. For example, a host can have a mutation that causes the host to be deficient in a certain lineage and/or marker, or to ectopically express one or more markers. For example, laboratory mice stains can be deficient for one or more hematopoietic cell lineages, allowing antibody-producing cells to be detected with a smaller subset of markers than in a comparable wild-type mouse. Accordingly, in some aspects of the invention, kits are provided for certain types or classes of hosts. In some embodiments, kits are provided for C57BL/6, FVB/N, and/or NZB mice strains, which represent commonly used mouse strains for human disease models, including immunological diseases. In some embodiments, kits for C57BL/6, FVB/N, and/or NZB mice strains include CD49b (clones DX5 and HMa2), NK1.1, IgG and a B-cell specific marker. In some embodiments, the B-cell specific marker is B220. In some embodiments, for example embodiments encompassing human V cells, the B-cell specific marker is CD19 or CD20, or the combination of CD19 and CD20. In some embodiments, the B-cell specific marker is one of B220, CD5, CD19, CD20, CD21/CD35, CD22.2, CD72, GL-7, IgD, IgM, Ly6-k, Ly6-D, Ly-51 CD127, CD138, CD154, AA4.1 and/or Pax-5. In some embodiments, the kits contain instructions for the identification of V cells. In some embodiments, identification of V cells can be determined by the absence of basophil-specific markers (2D7, and/or BB1). It is noted that 2D7 and BB1 can comprise intracellular markers. Accordingly, in some embodiments, cells are fixed prior to being contacted with an antibody that binds specifically to 2D7 and/or BB1. In some embodiments, V cells of C57BL/6, FVB/N, and/or NZB mice strains can be identified as: CD49b+, IgG+, NK1.1− and negative for a B-cell specific marker. In some embodiments, V cells of C57BL/6, FVB/N, and/or NZB mice strains can be identified as: CD49b+, IgG+, NK1.1− and B220−. In some embodiments, V cells of C57BL/6, FVB/N, and/or NZB mice strains can be identified as: CD49b+, IgG+, NK1.1− B220− and 2D7−. In some embodiments, the kit includes isolated CD49b+, IgG+, NK1.1− B220− 2D7− cells, which can be used as a positive control. In some embodiments, the kit includes isolated CD49b+, IgG+, NK1.1− B220− BB1− cells, which can be used as a positive control.

In some aspects, kits are provided for detecting antibody-producing cells, for example V cells, and are suitable for use in any (or substantially any) host organism. In some embodiments, kits are provided and are suitable for identifying V cells in a human. In some embodiments, kits are provided for detecting V cells in all (or substantially all strains of mice. In some embodiments, the kits contain instructions for the identification of V cells. In some embodiments, the kit includes identified V cells, which can be used as positive controls. Exemplary kits include, but are not limited to those shown in Table 3.

TABLE 3
Exemplary kits for detection of V cells

| Markers in kit (i.e. reagents that target . . .) | V cell can be identified as . . . |
|---|---|
| CD49b (clones DX5 or HMa2), IgG, IgE, at | CD49b+, IgG+, IgE+, negative for the |
| least one Basophil cell specific marker (e.g., | Basophil cell specific marker (e.g., 2D7 |
| 2D7 and/or BB1) and at least one B-cell | and/or BB1) and negative for the B-cell |
| specific marker (e.g., B220, CD19, CD20, or | specific marker (e.g., B220−, CD19−, CD20−, |
| CD19 and CD20)*^{,}** | or CD19− CD20−)*^{,}** |
| CD49b (clones DX5 or HMa2), IgG, IgE, | CD49b+, IgG+, IgE+ 2D7−, BB1− and B220− |
| 2D7, BB1 and B220 | |
| CD49b (clones DX5 or HMa2), IgG, IgE, | CD49b+, IgG+, IgE+ 2D7− and B220− |
| and B220, 2D7 | |
| CD49b (clones DX5 or HMa2), IgG, IgE, | CD49b+, IgG+, IgE+ BB1− and B220− |
| and B220, BB1 | |
| CD49b (clones DX5 or HMa2), IgG, IgE, | CD49b+, IgG+, IgE+ 2D7−, BB1− and CD19− |
| 2D7, BB1 and CD19 | |
| CD49b (clones DX5 or HMa2), IgG, IgE, | CD49b+, IgG+, IgE+ 2D7− and CD19− |
| and CD19, 2D7 | |
| CD49b (clones DX5 or HMa2), IgG, IgE, | CD49b+, IgG+, IgE+ BB1− and CD19− |
| and CD19, BB1 | |
| CD49b (clones DX5 or HMa2), IgG, IgE, | CD49b+, IgG+, IgE+ 2D7−, BB1− and CD20− |
| 2D7, BB1 and CD20 | |
| CD49b (clones DX5 or HMa2), IgG, IgE, | CD49b+, IgG+, IgE+ 2D7− and CD20− |
| 2D7 and CD20 | |
| CD49b (clones DX5 or HMa2), IgG, IgE, | CD49b+, IgG+, IgE+ BB1− and CD20− |
| BB1 and CD20 | |
| CD49b (clones DX5 or HMa2), IgG, IgE, | CD49b+, IgG+, IgE+, CD200R+, negative |
| CD200R, basophil specific markers (e.g., | for basophil specific markers (2D7 and/or |
| 2D7 and/or BB1) and at least one B-cell | BB1) and negative for the B-cell specific |
| specific marker (e.g., B220, CD19, CD20, or | marker (e.g., B220−, CD19−, CD20−, or |
| CD19 and CD20)* | CD19− CD20−) |
| CD49b (clones DX5 or HMa2), IgG, IgE, | CD49b+, IgG+, IgE+, CD200R+, negative |
| CD200R, at least one basophil specific | for the B-cell specific marker (e.g., B220−, |
| marker (e.g., 2D7 and BB1) and at least one | CD19−, CD20−, or CD19− CD20−), and |
| B-cell specific marker (e.g., B220, CD19, | negative for basophil specific markers (e.g., |
| CD20, or CD19 and CD20)*^{,}** | 2D7 or BB1)*^{,}** |
| CD49b (clones DX5 or HMa2), IgG, IgE, | CD49b+, IgG+, IgE+, CD200R+, 2D7− and |
| CD200R, 2D7 and B220 | B220− |
| CD49b (clones DX5 or HMa2), IgG, IgE, | CD49b+, IgG+, IgE+, CD200R+, 2D7−, |
| CD200R, 2D7, BB1, and B220 | BB1−, and B220− |
| CD49b (clones DX5 or HMa2), IgG, IgE, | CD49b+, IgG+, IgE+, CD200R+, BB1− and |
| CD200R, BB1 and B220 | B220− |
| CD49b (clones DX5 or HMa2), IgG, IgE, | CD49b+, IgG+, IgE+, CD200R+ 2D7−, BB1− |
| CD200R, 2D7, BB1 and CD19 | and CD19− |
| CD49b (clones DX5 or HMa2), IgG, IgE, | CD49b+, IgG+, IgE+, CD200R+ 2D7− and |
| CD200R, 2D7 and CD19 | CD19− |
| CD49b (clones DX5 or HMa2), IgG, IgE, | CD49b+, IgG+, IgE+, CD200R+ BB1− and |
| CD200R, BB1 and CD19 | CD19− |
| CD49b (clones DX5 or HMa2), IgG, IgE, | CD49b+, IgG+, IgE+, CD200R+, 2D7−, |
| CD200R, 2D7, BB1 and CD20 | BB1− and CD20− |
| CD49b (clones DX5 or HMa2), IgG, IgE, | CD49b+, IgG+, IgE+, CD200R+ 2D7− and |
| CD200R, 2D7 and CD20 | CD20− |
| CD49b (clones DX5 or HMa2), IgG, IgE, | CD49b+, IgG+, IgE+, CD200R+ BB1− and |
| CD200R, BB1 and CD20 | CD20− |
| CD49b (clones DX5 or HMa2), IgG, IgE, | CD49b+, IgG+, IgE+, CD244.2+ and |
| CD244.2, and at least one B-cell specific | negative for the B-cell specific marker (e.g., |
| marker (e.g., B220, CD19, CD20, or CD19 | B220−, CD19−, CD20−, or CD19− CD20−) |
| and CD20)* | |
| CD49b (clones DX5 or HMa2), IgG, IgE, | CD49b+, IgG+, IgE+, CD244.2+, negative |
| CD244.2, at least one basophil specific | for basophil specific markers (e.g., 2D7 or |
| marker (e.g., 2D7, and BB1) and at least one | BB1). and negative for the B-cell specific |
| B-cell specific marker (e.g., B220, CD19, | marker (e.g., B220−, CD19−, CD20−, or |
| CD20, or CD19 and CD20)*^{,}** | CD19− CD20−)*^{,}** |
| CD49b (clones DX5 or HMa2), IgG, IgE, | CD49b+, IgG+, IgE+, CD244.2+, 2D7− and |
| CD244.2, 2D7 and B220 | B220− |
| CD49b (clones DX5 or HMa2), IgG, IgE, | CD49b+, IgG+, IgE+, CD244.2+, 2D7−, |
| CD244.2, 2D7, BB1, and B220 | BB1−, and B220− |
| CD49b (clones DX5 or HMa2), IgG, IgE, | CD49b+, IgG+, IgE+, CD244.2+, BB1− and |
| CD244.2, BB1 and B220 | B220− |
| CD49b (clones DX5 or HMa2), IgG, IgE, | CD49b+, IgG+, IgE+, CD244.2+, 2D7−, |
| CD244.2, 2D7, BB1, and CD19 | BB1− and CD19− |
| CD49b (clones DX5 or HMa2), IgG, IgE, | CD49b+, IgG+, IgE+, CD244.2+ 2D7− and |
| CD244.2, 2D7 and CD19 | CD19− |
| CD49b (clones DX5 or HMa2), IgG, IgE, | CD49b+, IgG+, IgE+, CD244.2+ BB1− and |
| CD244.2, BB1 and CD19 | CD19− |
| CD49b (clones DX5 or HMa2), IgG, IgE, | CD49b+, IgG+, IgE+, CD244.2+ 2D7−, |
| CD244.2, 2D7, BB1 and CD20 | BB1− and CD20− |
| CD49b (clones DX5 or HMa2), IgG, IgE, | CD49b+, IgG+, IgE+, CD244.2+ 2D7− and |
| CD244.2, 2D7, and CD20 | CD20− |
| CD49b (clones DX5 or HMa2), IgG, IgE, | CD49b+, IgG+, IgE+, CD244.2+ BB1− and |
| CD244.2, BB1− and. CD20 | CD20− |
| CD49b (clones DX5 or HMa2), IgG, IgE, | CD49b+, IgG+, IgE+, FcεR1+ and negative |
| FcεR1, and at least one B-cell specific marker | for the B-cell specific marker (e.g., B220−, |
| (e.g., B220, CD19, CD20, or CD19 and | CD19−, CD20−, or CD19− CD20−) |
| CD20)* | |
| CD49b (clones DX5 or HMa2), IgG, IgE, | CD49b+, IgG+, IgE+, FcεR1+, negative for |
| FcεR1, at least one Basophil specific marker | at least one Basophil specific marker (e.g., |
| (e.g., 2D7, or BB1) and at least one B-cell | 2D7, or BB1) and negative for the B-cell |
| specific marker (e.g., B220, CD19, CD20, or | specific marker (e.g., B220−, CD19−, CD20−, |
| CD19 and CD20)*^{,}** | or CD19− CD20−)*^{,}** |
| CD49b (clones DX5 or HMa2), IgG, IgE, | CD49b+, IgG+, IgE+, FcεR1+, 2D7− and |
| FcεR1, 2D7 and B220 | B220− |
| CD49b (clones DX5 or HMa2), IgG, IgE, | CD49b+, IgG+, IgE+, FcεR1+, 2D7−, BB1− |
| FcεR1, 2D7, BB1 and B220 | and B220− |
| CD49b (clones DX5 or HMa2), IgG, IgE, | CD49b+, IgG+, IgE+, FcεR1+, BB1− and |
| FcεR1, BB1 and B220 | B220− |
| CD49b (clones DX5 or HMa2), IgG, IgE, | CD49b+, IgG+, IgE+, FcεR1+, 2D7−, BB1− |
| FcεR1, 2D7, BB1 and CD19 | and CD19− |
| CD49b (clones DX5 or HMa2), IgG, IgE, | CD49b+, IgG+, IgE+, FcεR1+ 2D7−and |
| FcεR1, 2D7 and CD19 | CD19− |
| CD49b (clones DX5 or HMa2), IgG, IgE, | CD49b+, IgG+, IgE+, FcεR1+ BB1− and |
| FcεR1, BB1 and CD19 | CD19− |
| CD49b (clones DX5 or HMa2), IgG, IgE, | CD49b+, IgG+, IgE+, FcεR1+, 2D7−, BB1− |
| FcεR1, 2D7, BB1, and CD20 | and CD20− |
| CD49b (clones DX5 or HMa2), IgG, IgE, | CD49b+, IgG+, IgE+, FcεR1+2D7− and |
| FcεR1, 2D7 and CD20 | CD20− |
| CD49b (clones DX5 or HMa2), IgG, IgE, | CD49b+, IgG+, IgE+, FcεR1+ and BB1− |
| FcεR1, BB1 and CD20 | CD20− |
| CD49b (clones DX5 or HMa2), IgG, and at | CD49b+, IgG+ and negative for the B-cell |
| least one B-cell specific marker (e.g., B220, | specific marker (e.g., B220−, CD19−, CD20−, |
| CD19, CD20, or CD19 and CD20)* | or CD19− CD20−) |
| CD49b (clones DX5 or HMa2), IgG, and at | CD49b+, IgG+ and negative for the Basophil |
| least one Basophil cell specific marker (e.g., | cell specific marker (e.g., 2D7 and/or BB1) |
| 2D7 and/or BB1)*^{,}** | *^{,}** |
| CD49b (clones DX5 or HMa2), NKp46, | CD49b+, IgG+, NKp46− 2D7−, BB1− and |
| 2D7, BB1 and at least one B-cell specific | negative for the B-cell specific marker (e.g., |
| marker (e.g., B220)* | B220−) |
| CD49b (clones DX5 or HMa2), NKp46, and | CD49b+, IgG+, NKp46− and negative for the |
| at least one Basophil cell specific marker | Basophil cell specific marker (e.g., 2D7 |
| (e.g., 2D7 and/or BB1)*^{,}** | and/or BB1)*^{,}** |
| CD49b (clones DX5 or HMa2), CD122, IgG, | CD49b+, IgG+, CD122− 2D7−, BB1− and |
| 2D7, BB1, and at least one B-cell specific | negative for the B-cell specific marker (e.g., |
| marker (e.g., B220, CD19, CD20, or CD19 | B220−) |
| and CD20)* | |
| CD49b (clones DX5 or HMa2), CD122, IgG, | CD49b+, IgG+, CD122− and negative for the |
| and at least one Basophil cell specific marker | Basophil cell specific marker (e.g., 2D7, |
| (e.g., 2D7 BB1)*^{,}** | and/or BB1)*^{,}** |
| IgE, IgG, 2D7, BB1 and a B-cell specific | IgE+ IgG+ 2D7−, BB1−, and negative for the |
| marker (e.g., B220, CD19, CD20, or CD19 | B-cell specific marker (e.g., B220−, CD19−, |
| and CD20) | CD20−, or CD19− CD20−) |
| IgE, IgG, and a Basophil specific marker | IgE+ IgG+ and negative for the Basophil cell |
| (e.g., 2D7 BB1) | specific marker (e. g., 2D7 and/or BB1) |
| CD200R, IgG, and at least one Basophil | CD200R+ IgG+ and negative for the |
| specific marker (e.g., 2D7 or BB1) | Basophil cell specific marker (e. g., 2D7 |
| | and/or BB1) |
| Basophil-specific markers (2D7 and/or BB1) | IgE+ and IgG+ and negative for the basophil |
| | specific marker (2D7 and/or BB1) |
| CD200R, IgG, at least one basophil specific | CD200R+ IgG+, negative for a basophil |
| marker (e.g., 2D7 or BB1)** and at least | specific marker (e.g., 2D7 or BB1)** and |
| one B-cell specific marker (e.g., CD19, | negative for the B-cell specific marker (e.g., |
| CD20, or CD19 and CD20)* | CD19−, CD20−, or CD129−CD20−)*; |

*While B220, CD19, and CD20 are shown as exemplary B-cell specific marker, other B-cell specific markers include, but are not limited to: CD5, CD21/CD35, CD22.2, CD72, GL-7, IgD, IgM, Ly6-k, Ly6-D, Ly-51, CD127, CD138, CD154, AA4.1 and/or Pax-5. In some embodiments, for human V cells, CD19 and CD20 are representative B-cell specific markers. In some embodiments, for murine V cells, B220 is a representative B-cell specific marker.
**2D7 and BB1 are shown as exemplary basophil-specific markers. In some embodiments, for human V cells, 2D7, BB1, or 2D7 and BB1 are representative basophil-specific markers. In some embodiments, for murine V cells, 2D7 is a representative basophil-specific marker. In some embodiments, for both murine and human V cells 2D7 is a representative basophil-specific marker. In some embodiments, for human V cells, BB1 is a representative basophil-specific marker.

In some embodiments, the kit further includes an antibody that binds specifically to CD200R.

In some embodiments, the kit further includes an antibody that binds specifically to CD244.2

In some embodiments, the kit further includes an antibody that binds specifically to FcεR1.

In some embodiments, the kit further includes at least one antibody that binds specifically to CD24, CD43, CD45, CD48, CD79b, CD123, CD16/CD32.

In some embodiments, the kit further includes an antibody that binds specifically to IgE.

In some embodiments, the kit further includes an antibody that binds specifically to one of CD1d, CD3, CD4, CD8, CD25, CD38 CD134, CD11c, CD273, CD49a, CD122, CD123, CD220R, CD226/NKp46, CD34, Sca-1, c-Kit, CD150, CD11b, Ly-6G, or NKP46.

In some embodiments, the kit further includes an antibody that binds specifically to CD123.

In some embodiments, the kit further includes an antibody that binds specifically to NKP46.

In some embodiments, the kit further includes an antibody that binds specifically to 2D7.

In some embodiments, the kit further includes an antibody that binds specifically to BB1.

In some embodiments, the kit further includes at least one antibody that binds specifically to a marker identified in Table 1.1. In some embodiments, the kit further includes at least one antibody that binds specifically to a marker identified in Table 1.2. In some embodiments, the kit further includes at least one antibody that binds specifically to a marker identified in Table 1.3.

### Cell Enrichment Kits

Some aspects of the invention include kits for enriching a population of cells for antibody-producing cells and/or cells capable of producing antibody. The antibody-producing cells can be V cells. Removing at least one type of non-V-cell from a population can enrich the population for V cells. Accordingly, in some embodiments, kits are provided for removing one or more non-V-cells from a population of cells, thus enriching the population for V cells. In some embodiments, the non-V-cells to be removed include one or more of B cells, T cells, Monocytes (granulocytes and macrophages), dendritic cells, NK cells, erythrocytes C-kit/Sca-1/CD150 positive cells, or basophils.

In some embodiments, the kits can be used for enriching a population of cells via flow cytometry. In some embodiments, the kits can be used for enriching a population of cells via non-flow cytometry methods, for example if flow cytometry equipment is unavailable, or considerations of economy and/or scale favor other methods. Accordingly, in some embodiments, the kits can be used for enriching a population of cells via a column. In some embodiments, the kits can be used for enriching a population of cells in a solution by precipitating one or more undesired cell types from solution.

In some embodiments, the kit includes antibodies (or other binding molecules) that target B cells, T cells, Monocytes (granulocytes and macrophages), dendritic cells, NK cells, erythrocytes and/or C-kit/Sca-1/CD150 positive cells. In some embodiments, the kit includes antibodies (or other binding molecules) that target B cells, and antibodies that target at least one of T cells, Monocytes (granulocytes and macrophages), dendritic cells, NK cells, erythrocytes and C-kit/Sca-1/CD150 positive cells. In some embodiments, the antibody or antibodies against these cell types are selected from the antibodies of Table 2. In some embodiments the kit includes antibodies that target B cells and antibodies that target at least two of the listed cell types, for example at least two, three, four, five, or six. In some embodiments, the kit includes antibodies (or other binding molecules) that target B cells, T cells, Monocytes (granulocytes and macrophages), dendritic cells, NK cells, erythrocytes and C-kit/Sca-1/CD150 positive cells. In some embodiments, the kit includes an antibody that specifically binds to an antigen selected from the group consisting of B220, CD5, CD19, CD20, CD21/CD35, CD22.2, CD72 GL-7, IgD, IgM, Ly6-k, Ly6-D, Ly-51 CD127, CD138, CD154, AA4.1 and Pax-5, and at least one of, for example at least one, two, three, four, five, or six of: an antibody that specifically binds to an antigen selected from the group consisting of CD1d, CD3, CD4, CD8, CD25, CD38 and CD134; an antibody that specifically binds to an antigen selected from the group consisting of Ly-6G; an antibody that specifically binds to an antigen selected from the group consisting of CD11c and CD273; an antibody that specifically binds to an antigen selected from the group consisting of NK1.1, NK1.2, CD49a, CD122 and CD226/NKp46 or CD57(human)/B3GAT1(mouse); an antibody that specifically binds to an antigen selected from the group consisting of Sca-1, c-Kit and CD150; an antibody that binds specifically to CD123, 2D7 or BB1.

In some embodiments, each antibody includes a marker. In some embodiments, the kit includes a cell separation system or collection of cell separation systems bound to or capable of specifically complexing with the antibodies of the kit.

In some embodiments, the kit includes a cell separation system as described herein for removing one or more non-V-cell from a population of cells. In some embodiments, the cell separation system is a magnetic bead system. For example, magnetic beads can be coated in streptavidin or glutathione or the like, and an antibody or antibodies binding to non-V-cells can be biotinlylated or GST-tagged or the like, so that magnetic beads can be used to remove non-V-cells from the population. In some embodiment, the antibodies are attached to fluorochromes, and anti-fluorochrome magnetic beads are used to remove non-V-cells recognized by the fluorochrome-tagged antibodies.

### Methods of Producing an Antibody

Some aspects of the invention include methods for generating antibodies, such as monoclonal antibodies. In some embodiments, an antibody is generated through the recovery of a mRNA and creating cDNA encoding an immunoglobulin binding region (or portion thereof) from isolated V cells. In some embodiments, a V cell that produces antigen-specific antibody is isolated. In some embodiments, a naïve V cell is isolated, exposed to antigen, and the V cell (or a cell derived therefrom) that produced antigen-specific antibody is isolated. In some embodiments, a V cell that produces an antigen-specific antibody is immortalized by fusing the V cell via hybridoma/fusion technology. In some embodiments, the variable regions of the antigen-specific antibody produced by a V cell are formatted into a desired protein format.

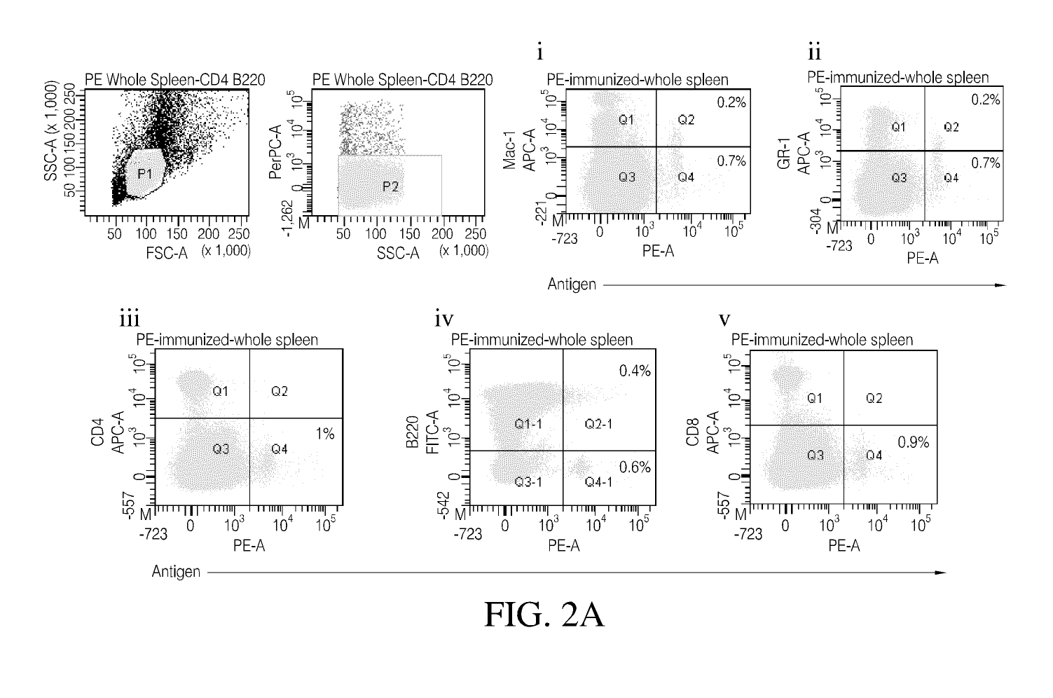

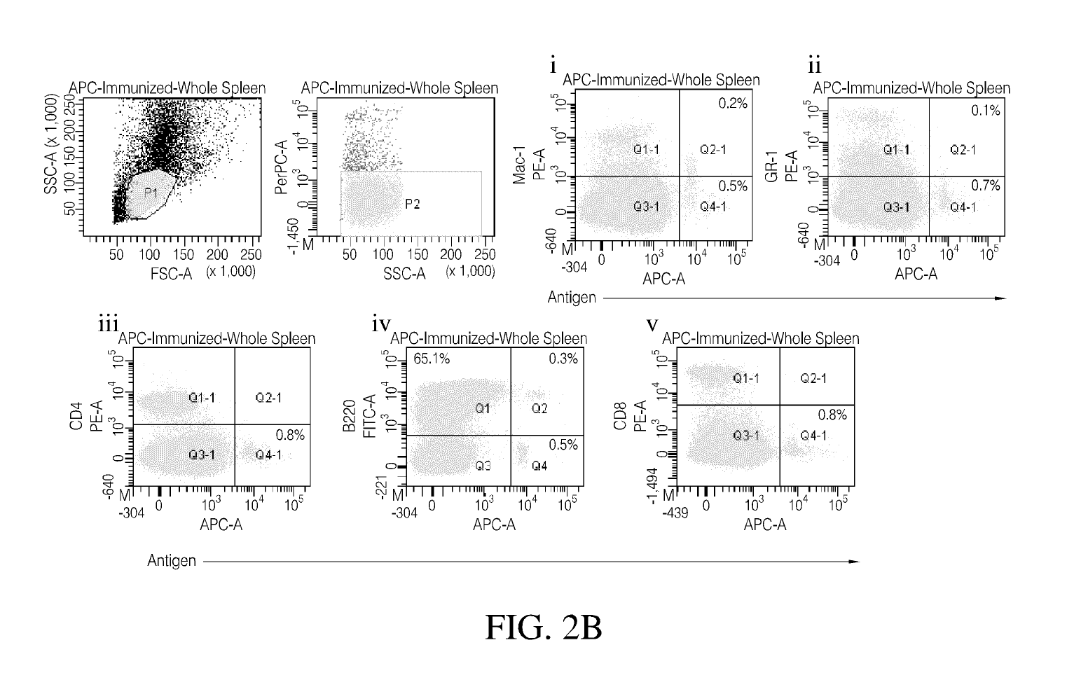

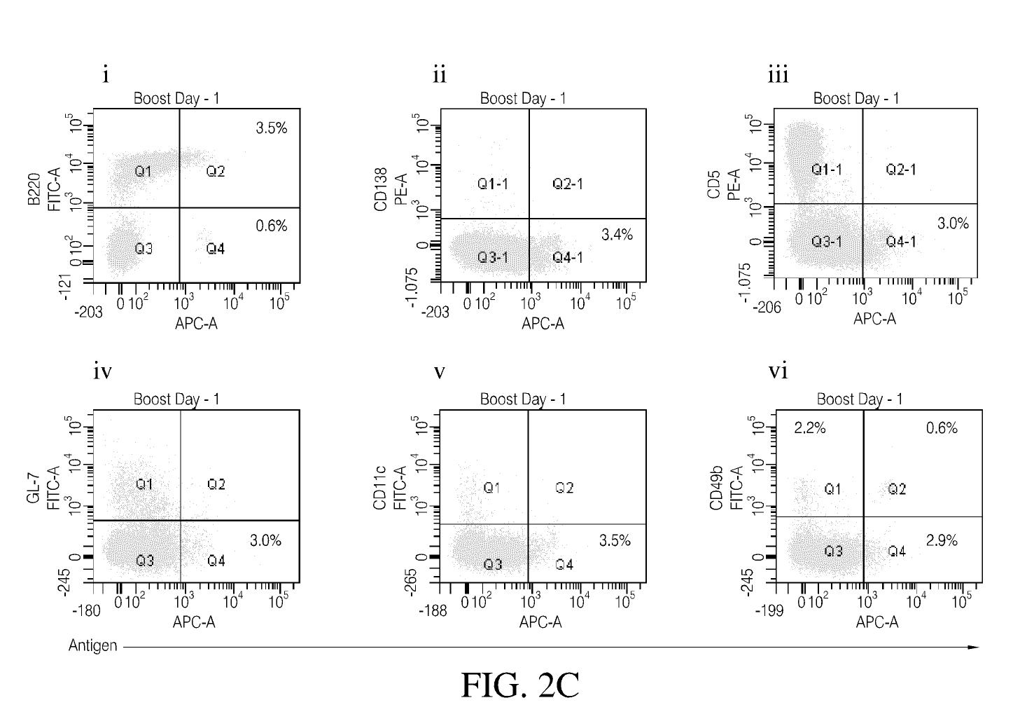

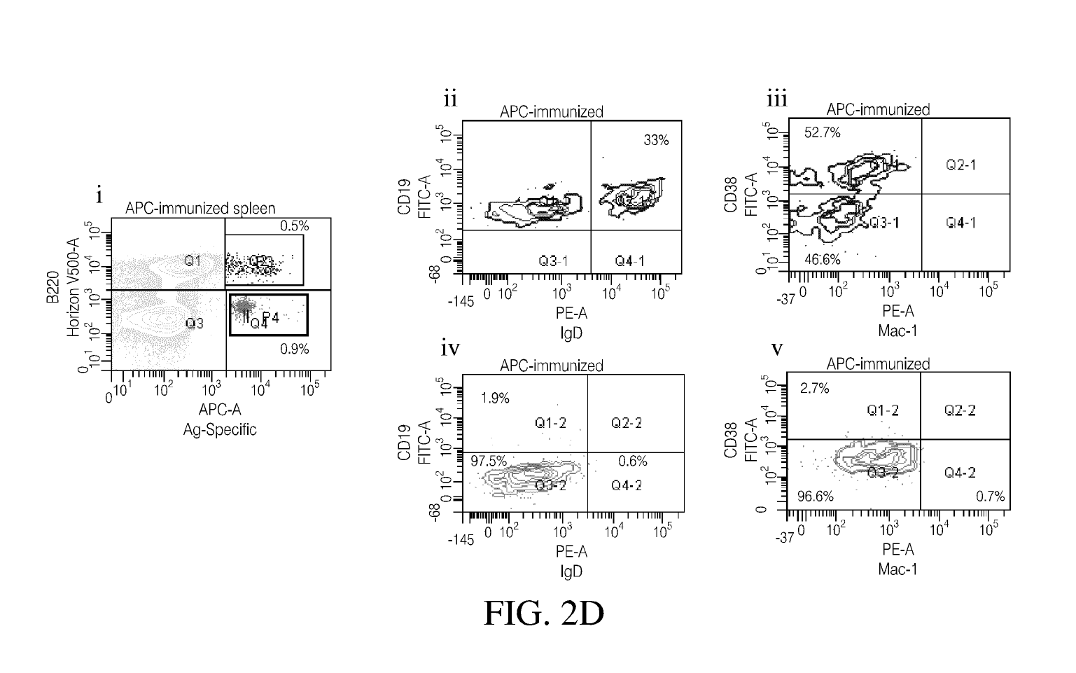

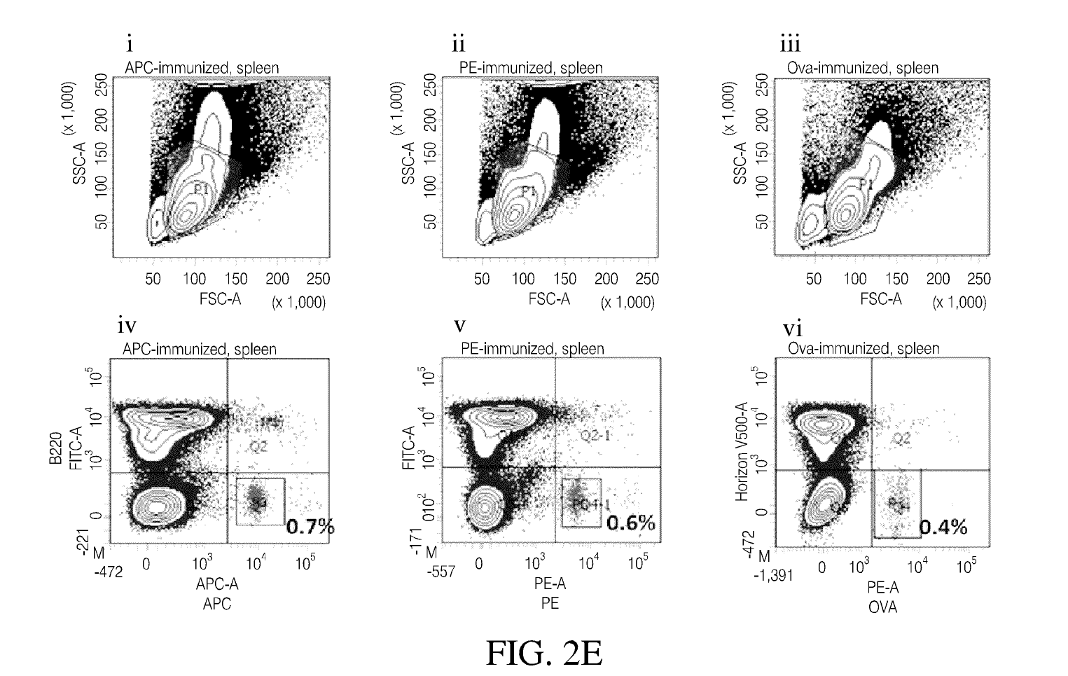

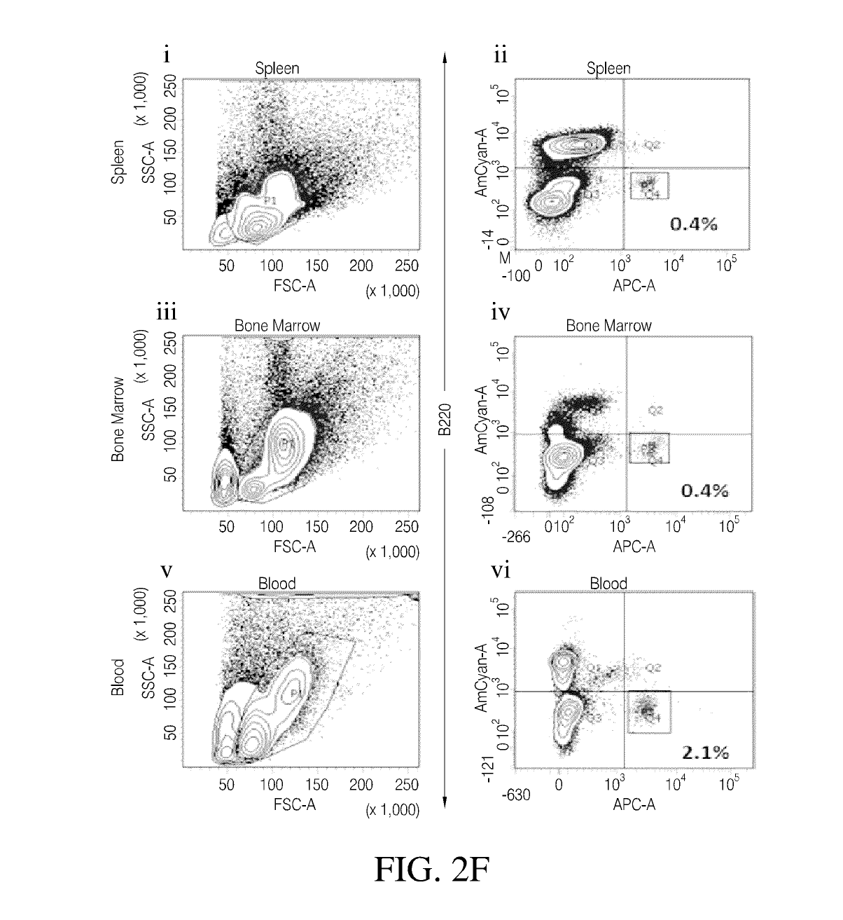

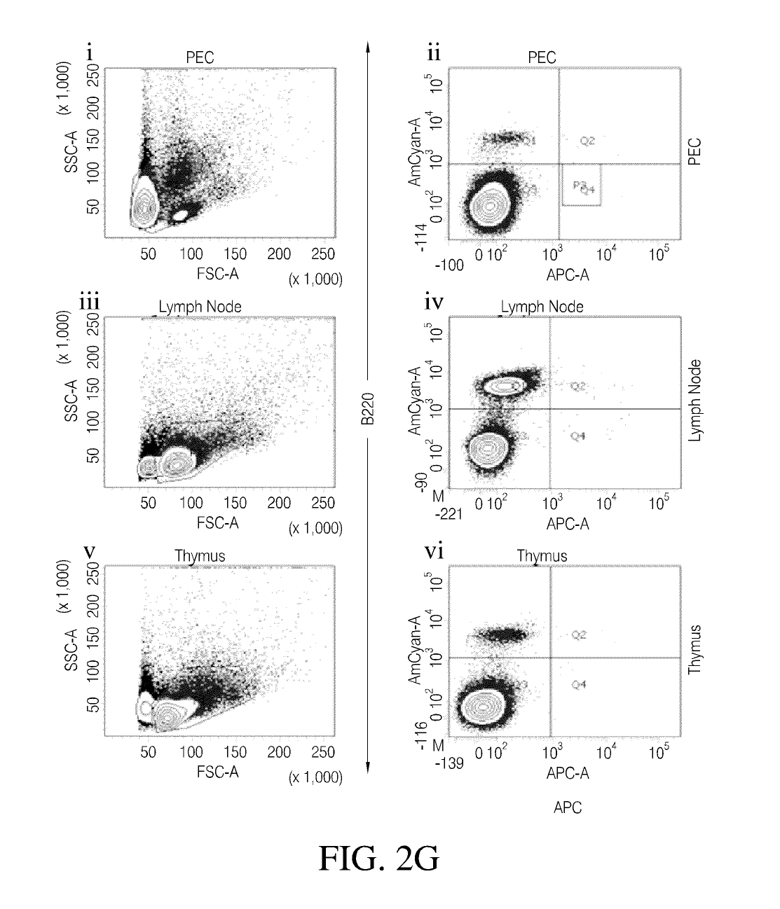

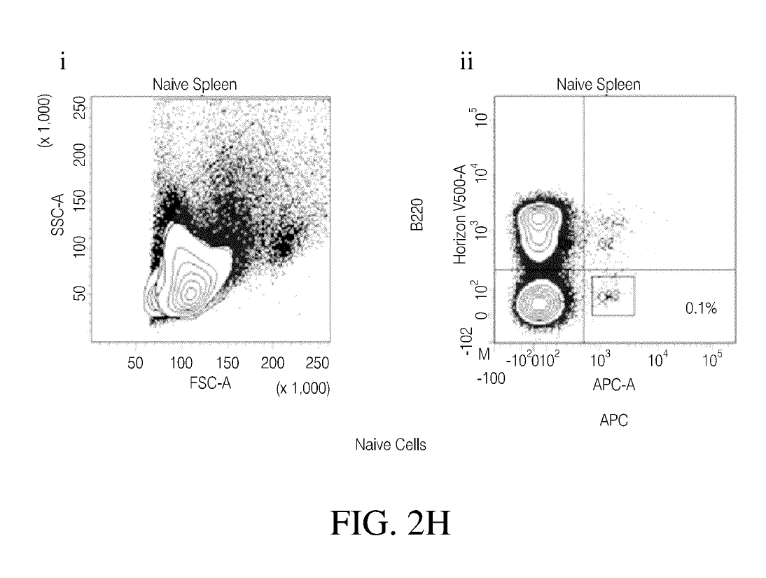

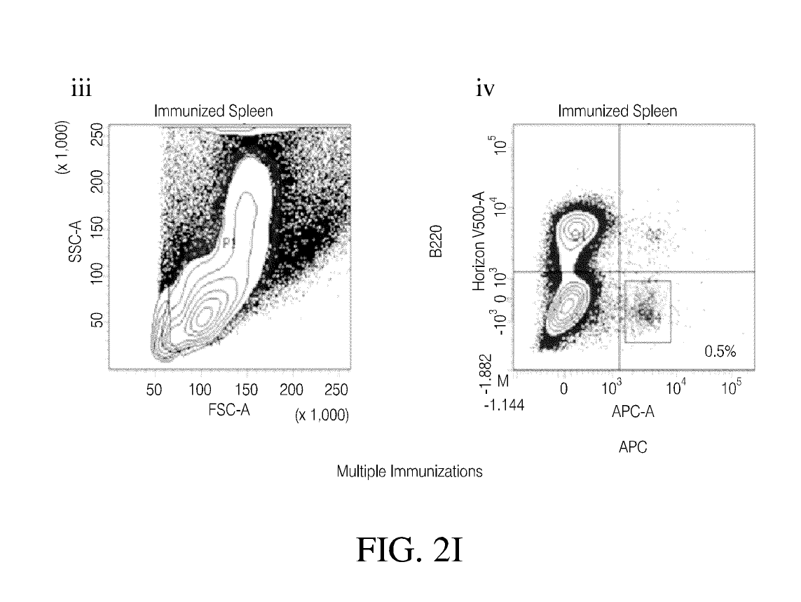

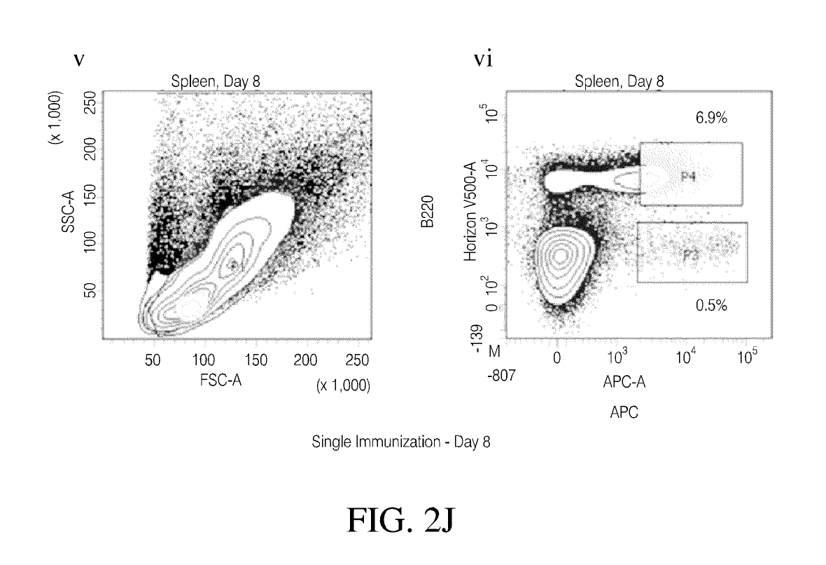

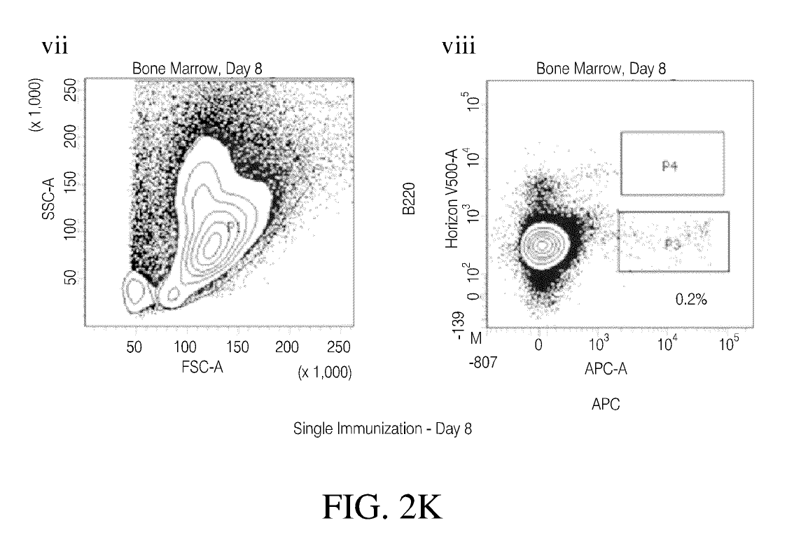

Upon immunization of a host with an antigen, V cells can be rapidly induced and can rapidly produce surface-bound antibody molecules with high-affinity for the antigen. Typically, antibodies derived from B cells require a first administration of antigen to induce an initial, low affinity IgM immune response after about 10 days, and a subsequent boost to induce somatic hypermutation, class switch to other isotypes than IgM and the production of antigen-specific antibody. As such, the production of antigen-specific IgG or other non IgM isotypes of antibody from B cells can typically take 20-30 days after first administering antigen to the host. It has been observed herein that V cells can produce antigen-specific antibody after just 8 days (see FIGS. 2H-2K and Example 2H). As such, it is appreciated herein that in some embodiments, V cells can be used to produce antigen-specific IgG antibody more rapidly that B cells, for example, in as few as 8 days after first administering antigen to the host, and thus, for example, at least within about 8, 9, 10, 11, 12, 13, 14, 15, 16, 17, 18, 19, or 20 days after first administering antigen to the host.

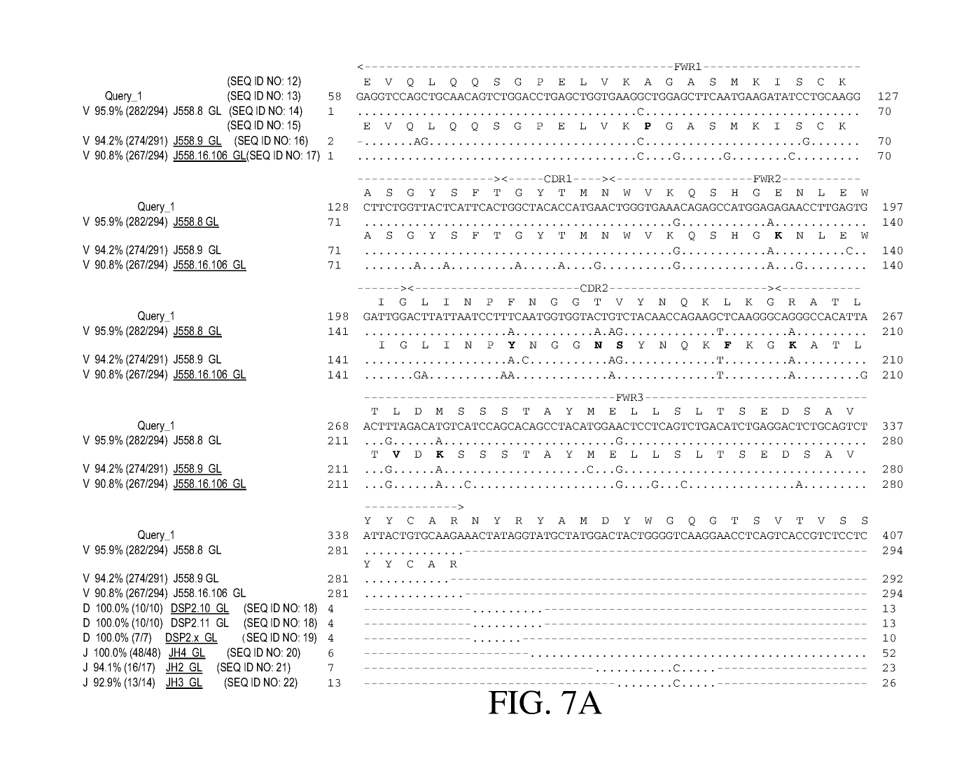

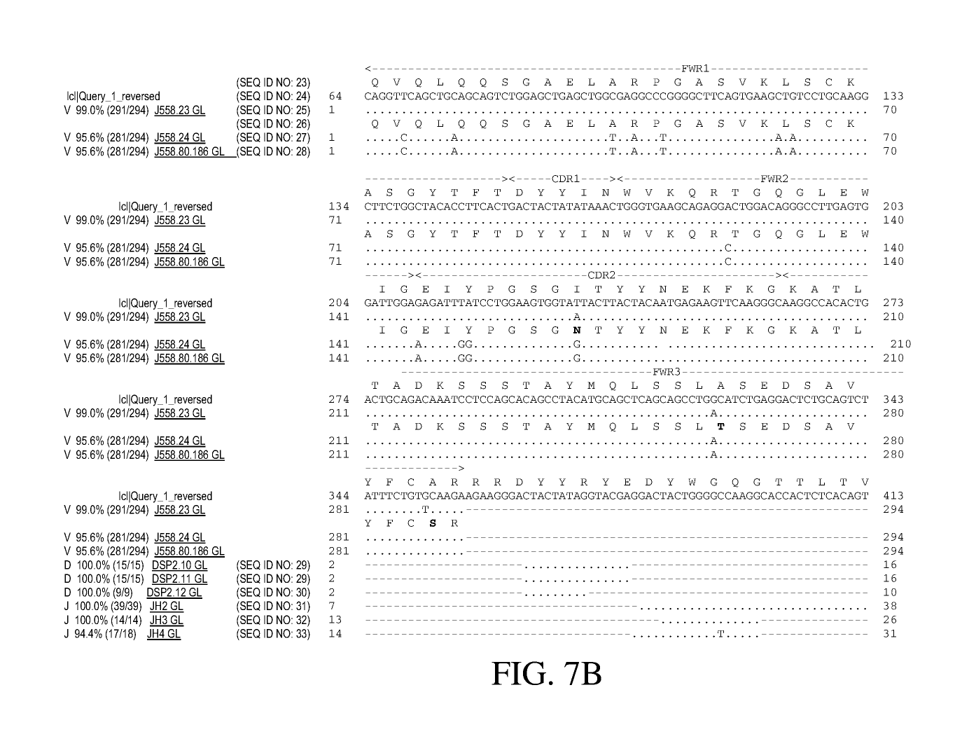

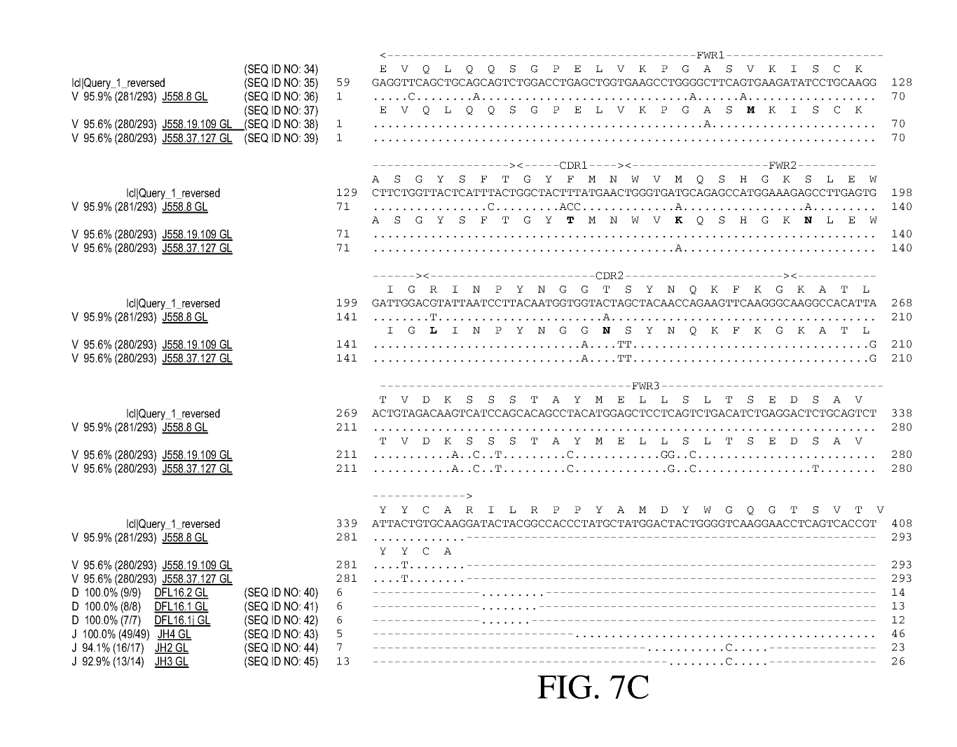

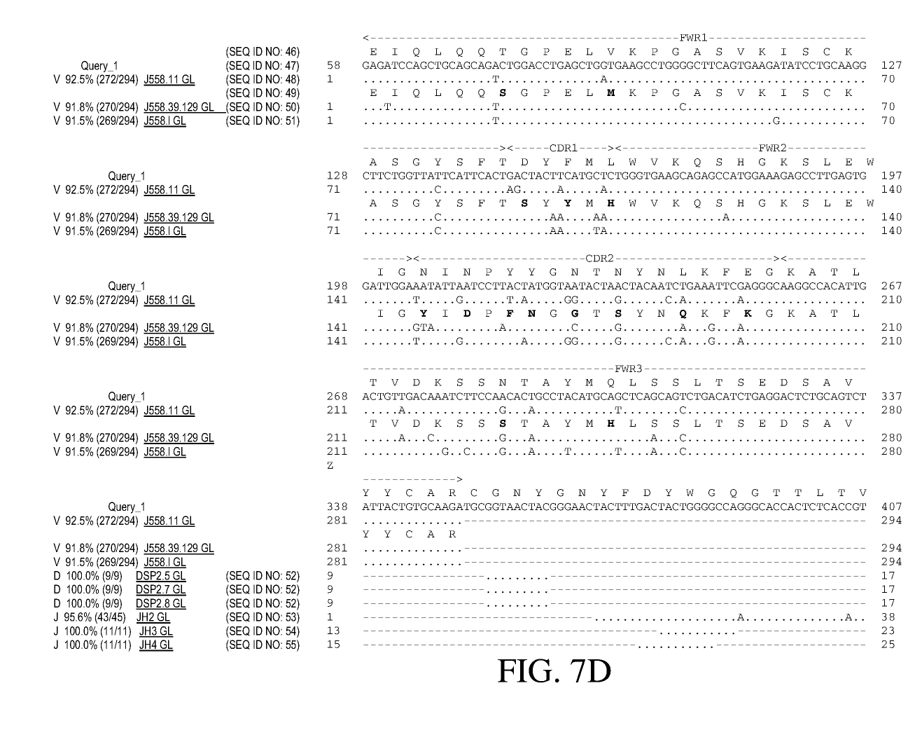

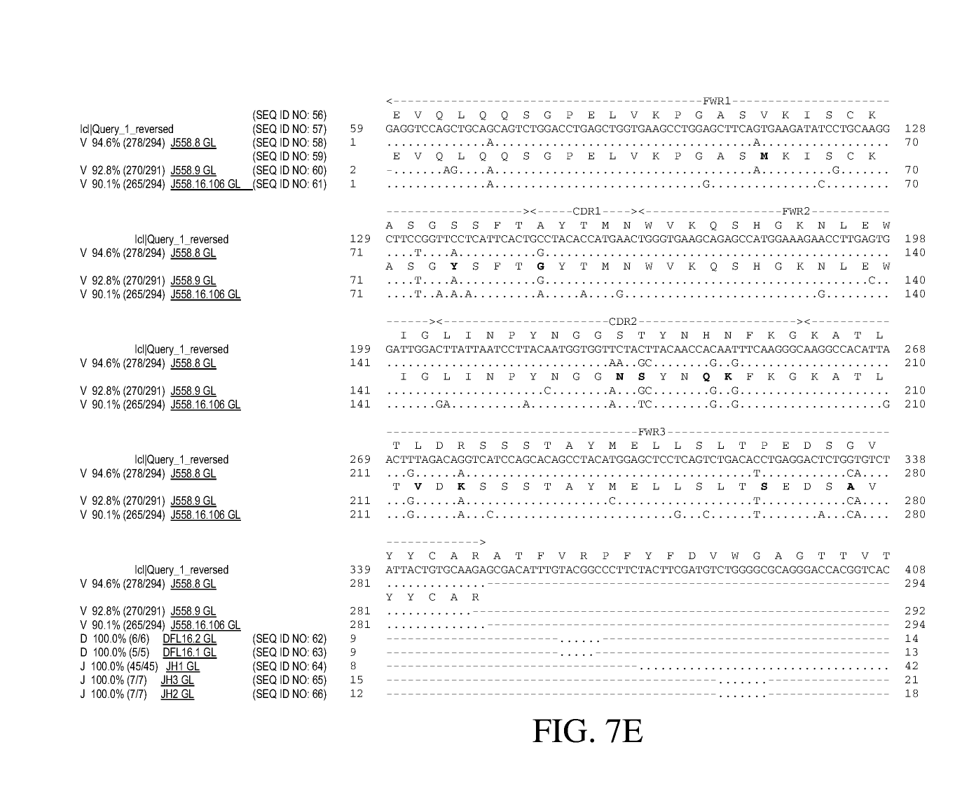

In some embodiments, only a single administration of antigen is given to the host organism, with no boosts. Without being limited to any particular theory, in comparison to germline genes, the immunoglobulin variable genes utilized by V cells typically contain numerous point mutations consistent with somatic hypermutation (see FIGS. 7A-7F), and which can allow for antibodies with focused variable regions with high specificity for antigen. In contrast, the B-1B cell population is characterized by largely germline V gene sequences, and production of affinity matured antibodies from normal B cells in T-dependent responses can involve additional rounds of boosting, affinity maturation, and selection. As such, it is contemplated herein that V cells can be used as for rapidly producing high-affinity antibody. In some embodiments, V cells can be used to produce high-affinity antibodies, while minimizing or eliminating steps such as antigen boosts or affinity maturation. It has been shown herein that V cells can produce antigen-specific antibody in as few as 8 days after administering antigen to the host. Accordingly, in some embodiments, a V cell can produce antigen-specific antibody within 20 days of the first administration of antigen, for example 8, 9, 10, 11, 12, 13, 14, 15, 16, 17, 18, 19, or 20 days, including ranges between any of the listed values. In some embodiments, the V cells produce antigen-specific antibody within 10 days of the first administration of antigen. At the time the antibody is first produced (e.g. within 8, 9, 10, 11, 12, 13, 14, 15, 16, 17, 18, 19, or 20 days after first administration), the variable region gene segments utilized by the V cell can be rearranged and can contain a plurality of point mutations (in comparison to the germline genes) consistent with somatic hypermutation.

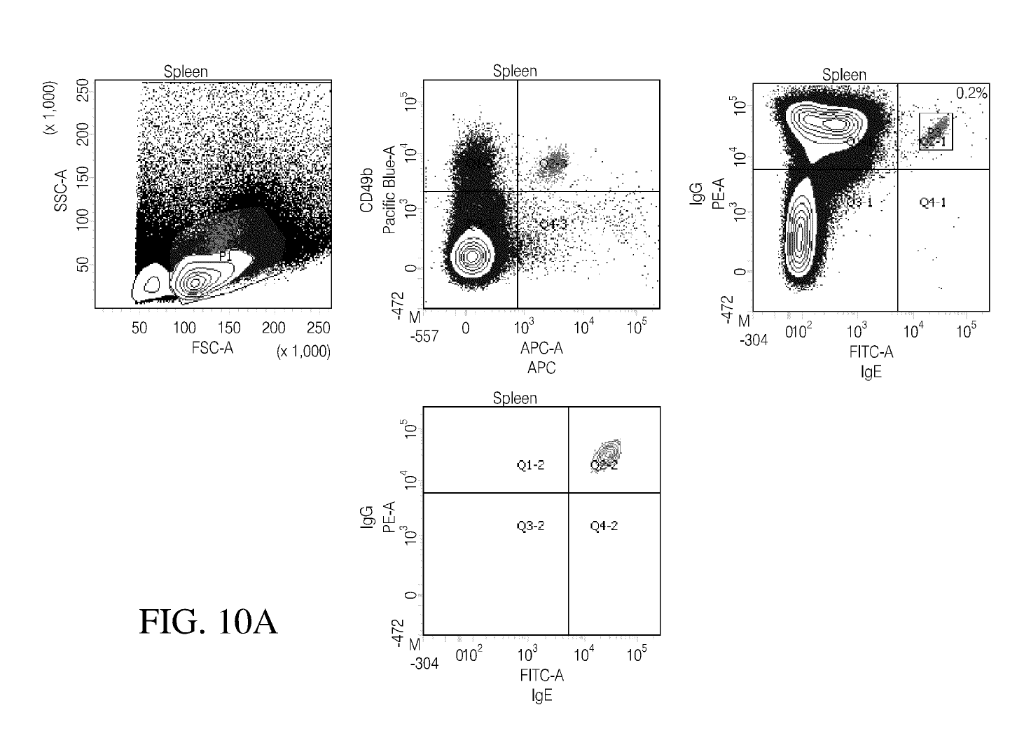

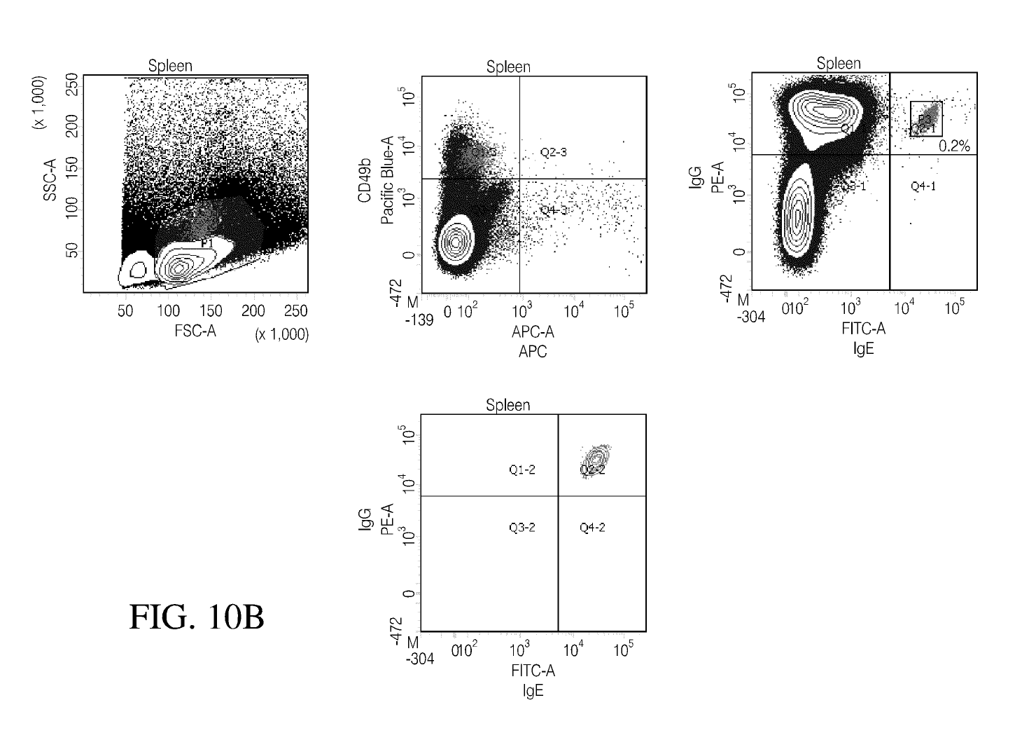

As V cells producing antigen-specific antibody can be identified based on phenotype, for example IgG+ IgE+ CD49b+, negative for B-cell-specific markers and negative for basophil-specific markers (see, e.g., Example 9 and FIGS. 10A-B), in some embodiments, V-cells that produce antigen-specific antibody can be identified without the use of labeled antigen. Typically, antigen-specific antibody can be identified through detection of binding to labeled antigen, for example biotinylated antigen or antigen labeled with a fluorophore. It is appreciated herein that the identification of V cells that produce antigen-specific antibody based on cell phenotype can optionally bypass the use of labeled antigen. It is noted that the labeling of antigen may introduce conformation changes in the antigen, or affect antigen solubility or restrict access to certain epitopes on the antigen, and as such, may bias the selection of antibody in favor of labeled antigen rather than the native antigen. As such, in some embodiments, V cells that produce antigen-specific antibody are identified using phenotypic markers only, and thus the antibody can be derived from V cells selected for affinity to native antigen rather than labeled antigen in particular. Without being limited to any particular theory, it is noted that for the V cells identified, for example in Example 9 and FIGS. 10A-10B, all or nearly all of the identified V cells produced antigen-specific immunoglobulin. It is contemplated that for populations of V cells identified using other combinations of markers, and/or cocktails of detection reagents, all or nearly all of the V cells will produce antigen-specific immunoglobulin. Accordingly, in some embodiments, at least about 90% of a population of V cells produces antigen-specific immunoglobulin, for example at least about 90%, 91%, 92%, 93%, 94%, 95%, 96%, 97%, 98%, 99%, 99.5%, 99.9%, or 99.99%. In some embodiments, the population of V cells in which all or nearly all of the V cells produce antigen-specific immunoglobulin, is at least CD49b+IgE+ IgG+, negative for B cell-specific markers, for example CD19, CD20, and B22.

According to some embodiments, a method of producing an antibody is provided. The method can include administering an antigen to a host organism, isolating at least one Ig-producing cell (for example a V cell) of the host organism, and at least one of (a) determining variable gene utilization of the cell or (b) culturing a lineage of antibody-producing cells derived from the cell.

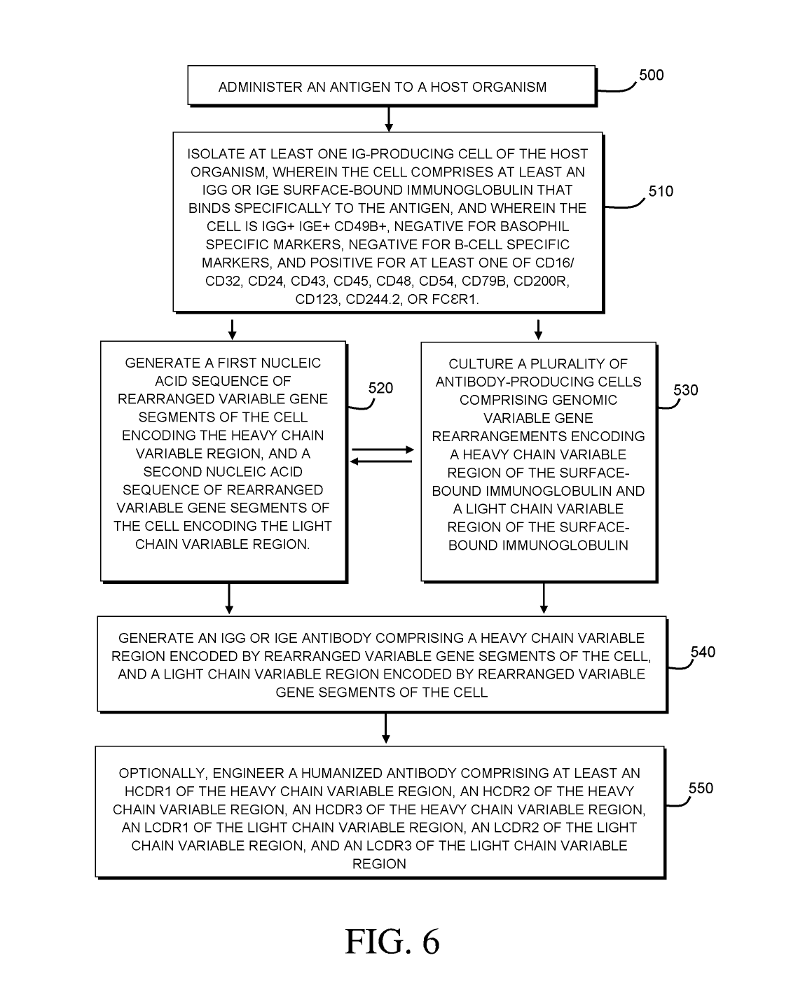

FIG. 6 is a flow diagram illustrating a method of producing an antibody according to some embodiments herein. The method can comprise administering an antigen to a host organism 500. In some embodiments, antigen is administered to the host only once. The method can comprise isolating at least one Ig-producing cell of the host organism, wherein the cell comprises at least an IgG or IgE immunoglobulin (e.g. an antibody) that binds specifically to the antigen, and wherein the cell is IgG+ IgE+ CD49b+, negative for B-cell-specific markers, negative for basophil-specific markers, and positive for at least one of CD16/CD32, CD24, CD43, CD45, CD48, CD54, CD79b, CD123, CD200R, CD244.2, or FcεR1 510. The immunoglobulin can be surface-bound. In some embodiments, the immunoglobulin that binds specifically to the antigen is produced within about 20 days after the host organism is first inoculated, for example about 8, 9, 10, 11, 12, 13, 14, 15, 16, 17, 18, 19, or 20 days, including ranges between the listed values. In some embodiments, the immunoglobulin that binds specifically to the antigen is produced within about 10 days after the host organism is first inoculated. In some embodiments, the antigen-specific antibody producing cell is identified without the use of labeled antigen, but instead is identified based on the cell's phenotype. The method can comprise at least one of (a) generating a first nucleic acid of variable gene segments from the V cell encoding a heavy chain variable region of the immunoglobulin and a second genomic sequence from the V cell encoding a light chain variable region of the surface-bound antigen specific immunoglobulin 520; or (b) culturing a plurality of antibody-producing cells comprising genomic variable gene rearrangements encoding a heavy chain variable region of the surface-bound immunoglobulin and a light chain variable region of the surface-bound immunoglobulin 530. In some embodiments, the method comprises both (a) and (b). In some embodiments (a) is performed followed by (b). In some embodiments, (b) is performed, followed by (a). In some embodiments, the method comprises generating an antibody comprising a heavy chain variable region encoded by rearranged IgG or IgE-variable gene segments from the cell, and a light chain variable region encoded by rearranged variable gene segments of the cell 540. Optionally, the method comprises engineering a humanized antibody comprising at least an HCDR1 of the heavy chain variable region, an HCDR2 of the heavy chain variable region, an HCDR3 of the heavy chain variable region, an LCDR1 of the light chain variable region, an LCDR2 of the light chain variable region, and an LCDR3 of the light chain variable region 550. One skilled in the art will appreciate that, for this and other processes and methods disclosed herein, the functions performed in the processes and methods may be implemented in differing order. Furthermore, the outlined steps are only provided as examples, and some of the steps and operations may be optional, combined into fewer steps and operations, or expanded into additional steps and operations without detracting from the essence of the disclosed embodiments.

In some embodiments, monoclonal antibodies are created through the recovery (cloning or identification) of the binding domain of an individual immunoglobulin from a larger polyclonal response. For example recovering the predominant heavy chain variable region genes and predominant light chain variable region genes from purified bulk V cells. These binding domains can be cloned by either immortalizing the cell (e.g., Hybridoma fusion to a myeloma, or EBV immortalization), selection for binding from an in vitro display library (f-phage, phagemid, yeast, ribosome display, whole bacterial display, mammalian cell display), or direct cloning from individually sorted cells via RT-PCR amplification (limiting dilution, FACS sorting to wells).

Hybridoma techniques are well known in the art. A host animal is typically injected with the antigen, and, after a period of time, antibody-making cell can be isolated, usually from the spleen. The antibody-making cell can be fused with myeloma (or other immortalized cell) cells to provide fused cells, referred to as hybridomas. The hybridomas can be separated from unfused antibody-making cells and myeloma cells. Specific hybridomas can be isolated and tested to confirm that the isolated hybridoma produces antibody specific for the antigen used in the immunization step. The hybridoma so produced combines the ability of the parent antibody-making cell to produce a specific single antibody with the ability of its parent myeloma (or other immortalized) cell to continually grow and divide, either in vitro as a cell culture or in vivo as a tumor after injection into the peritoneal cavity of an animal. Hybridoma lines can be used, for example to produce monoclonal antibodies.

Accordingly, some embodiments include a method of making a hybridoma. The method can include providing a V cell immunized with an antigen (Ag). The method can include fusing the immunized cell with an immortalized cell. The method can include generating an isolated culture derived from a single fusion. In some embodiments, the V cell is isolated. V cells can be isolated using methods described herein. In some embodiments, the V cell is identified as a IgG+ IgE+ CD49b+ CD200R+ cell that is negative for B-cell-specific markers and negative for basophil-specific markers, for example a B220− IgG+ IgE+ CD49b+ CD200R+2D7− BB1− cell, CD49b+ CD200R+ 2D7− cell, CD49b+ CD200R+ BB1− cell, CD19− IgG+ IgE+ CD49b+ CD200R+ 2D7− BB1− cell, CD19− IgG+ IgE+ CD49b+ CD200R+ 2D7− cell, CD19− IgG+ IgE+ CD49b+ CD200R+ BB1− cell, CD20−IgG+ IgE+ CD49b+ CD200R+ 2D7− BB1− cell, or CD20−IgG+ IgE+ CD49b+ CD200R+ 2D7− cell, CD20−IgG+ IgE+ CD49b+ CD200R+ BB1− cell, or otherwise as described herein.

Some embodiments include a hybridoma. The hybridoma can include the fusion product of a V cell (as described herein) and an immortalized cell. The hybridoma can be an isolated, immortalized antibody-producing cell population. In some embodiments, the hybridoma includes a fusion product of a IgG+ IgE+ CD49b+ CD200R+ cell that is negative for B-cell-specific markers and basophil specific markers, for example a B220− IgG+ IgE+CD49b+ CD200R+ 2D7− BB1− cell, B220− IgG+ IgE+ CD49b+ CD200R+ 2D7− cell, B220− IgG+ IgE+ CD49b+ CD200R+ BB1− cell, CD19− IgG+ IgE+ CD49b+ CD200R+ 2D7− BB1− cell, CD19− IgG+ IgE+ CD49b+ CD200R+ 2D7− cell, CD19− IgG+ IgE+ CD49b+ CD200R+ BB1− cell, CD20−IgG+ IgE+ CD49b+ CD200R+ 2D7− BB1− cell, CD20−IgG+ IgE+ CD49b+ CD200R+ 2D7− cell or a CD20−IgG+ IgE+ CD49b+ CD200R+ BB1− cell. In some embodiments, the V cell is Ag+.

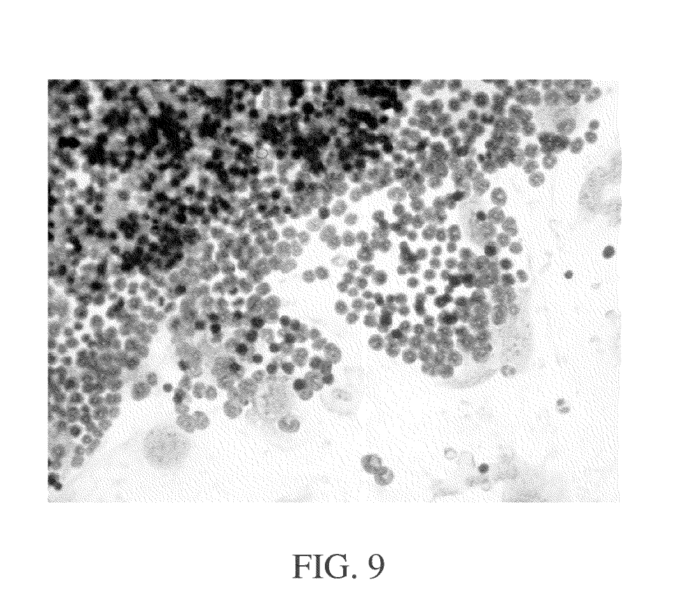

V cells can be cultured in vitro from a progenitor V cell (see, e.g., Examples 3S-3T and FIGS. 3S-3T). Moreover, colonies can be formed from isolated V cells (see, e.g., FIG. 9). As such, in some embodiments, one or more isolated V cells are cultured. In some embodiments, antibody-producing V cells are cultured. A culture can be derived from a single antibody-producing V cell. Antibody of interest can be obtained from the V cell in accordance with methods herein. In some embodiments, naïve V cells are cultured. The naïve V cells from culture can be used to produce antibody, for example by administering the V cells to a host organism and administering an antigen to the host in accordance with methods herein.

Some embodiments include a method of generating an immunoglobulin-encoding cDNA from a V cell, for example an IgG- or IgE-encoding cDNA. The method can include isolating at least one of an IgG heavy chain or light chain-encoding mRNA or an IgE heavy chain or light chain-encoding mRNA from a V cell as described herein. The method can include generating a cDNA complementary to the mRNA. In some embodiments, two or more of the following are isolated from a single V cell: mRNA encoding an IgG heavy chain; mRNA encoding an IgG light chain, mRNA encoding an IgE heavy chain; mRNA encoding an IgE light chain. In some embodiments, the V cell is Ag+. In some embodiments, the mRNA or cDNA encodes the products of V genes utilized by the V cell.

Isolation of cDNA encoding an immunoglobulin-binding region from an isolated V cell is described in Examples 3K-3L. In some embodiments, fresh V cells are sorted into a suitable physiological neutral buffer and then resuspended in RNA lysis buffer, the composition of which known to those skilled in the art. Total RNA can be isolated using a commercial total RNA isolation kit, for example a QIAGEN RNEasy™ mini spin column or the like. Optionally, PCR can be directly performed using a one-step Reverse transcriptase PCR kit. In some embodiments, first strand cDNA is synthesized using oligo dT primers or random hexamers or gene specific primers and a suitable RT reaction. The reverse transcription system can include Thermoscript, Superscript or other suitable commercial system. In some embodiments single cells are sorted into cells and cDNAs cloned individually using isotype specific back primers followed by total transcriptome sequencing or massively parallel sequencing.

In some embodiments, first strand cDNAs are amplified with oligonucleotide primers that anneal to relatively conserved regions of immunoglobulin gene cDNA, for example leader and Framework 1 regions. In some embodiments, corresponding isotype-specific back primers are designed for gamma class immunoglobulin heavy chains, which can be expressed by V cells. In some embodiments, oligonucleotide primers span introns present in the DNA copies of immunoglobulin chains, which cover a distance of approximately 1500 base pairs, in which the introns are spliced out of heterogeneous nuclear mRNA as message is generated. Since DNA rearrangement, immunoglobulin gene mRNA expression and IgG expression only occur in antibody producing cells, such intron-spanning primers are expected to only amplify nucleic acids of antibody-producing cells.

In some embodiments, the heavy chain forward primers are selected from MHcL1 ATGGACTT(GCT)G (GAT)A(CT)TGAGCT (SEQ ID NO: 1); MHcL2 ATGGAATGGA(GC)CTGG(GA)TCTTTCTCT (SEQ ID NO: 2); MHcL3 ATGAAAGTGTTGAGTCTGTTGTACCTG (SEQ ID NO: 3); and MHcL4 ATG(GA)A (GC)TT(GC)(TG)GG(TC)T(AC)A(AG)CT(TG)G(GA)TT (SEQ ID NO: 4). In some embodiments two or more of the listed primers are provided as a pool. In some embodiments, the heavy chain reverse primer includes MG1-3Seq AGA TGG GGG TGT CGT TTT GGC (SEQ ID NO 5), MG2ab-3Seq GAC YGA TGG GGS TGT TGT TTT GGC (SEQ ID NO 6), or a pool including both of the listed primers.

In some embodiments, the light chain forward primers are selected from MKcL-1 ATGAAGTTGCCTGTTAGGCTGT b(SEQ ID NO: 7); MKcL-2 ATGGACTTTCAGGTGCAGATCT (SEQ ID NO: 8); MKcL-3 TTGCTGTTCTGGGTATCTGGTA (SEQ ID NO: 9); MKcL-4 ATGGAGACAGAC ACACTCCTGCTAT (SEQ ID NO: 10). In some embodiments two or more of the listed primers are provided in a pool. In some embodiments, the heavy chain reverse primer includes MKC1 GGATACAGTTGGTGCAGC (SEQ ID NO: 11).

In some embodiments, immunoglobulin-encoding genomic DNA of the V cell is sequenced. In some embodiments, IgG heavy chain or light chain-encoding DNA and/or IgE heavy chain or light chain-encoding DNA is sequenced. In some embodiments, the whole genome of the V cell is sequenced. In some embodiments a nucleic acid encoding the IgE or IgG is generated based on the sequencing results. The nucleic acid can comprise an amplicon or clone from the sequencing, or can be synthesized.

Isolated oligonucleotides encoding a desired antibody of interest can be expressed in an expression system, for example a cellular expression system or a cell-free system. Exemplary cellular expression systems include yeast (e.g., mammalian cells, E. coli, insect cells, Saccharomyces, Pichia) transformed with recombinant yeast expression vectors containing the nucleotide sequences encoding antibodies; insect cell systems infected with recombinant virus expression vectors (e.g., baculovirus) containing sequences encoding antibodies; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing nucleotide sequences encoding antibodies; mammalian cell systems (e.g., COS, CHO, BHK, 293, 3T3) harboring recombinant expression constructs containing promoters derived from the genome of mammalian cells (e.g., metallothionein promoter) or from mammalian viruses. Exemplary cell free systems include E. coli extracts and yeast extracts. The extracts can be lysates. The extracts can be purified, for example, to enrich for ribosomes and/or to remove undesired materials such as debris or host genomic DNA. Nucleic acids encoding antibodies in cell-free systems can include plasmid DNA, linear DNA, or RNA.

In some embodiments, chimeric or humanized antibodies are produced. Chimeric antibodies include portions from two or more host organisms, for example murine CDRs and human framework and constant regions. Humanized antibodies comprise at least some human portions. One approach for producing chimeric or humanized antibodies includes CDR grafting. For example, an antigen can be delivered to a non-human host (for example a mouse), so that the host produces antibody against the antigen. In some embodiments, monoclonal antibody is generated using hybridoma technology. In some embodiments, V gene utilization in a single antibody producing cell of the host is determined. The CDR's of the host antibody can be grafted onto a human framework. The V genes utilized in the non-human antibody can be compared to a database of human V genes, and the human V genes with the highest homology can be selected, and incorporated into a human variable region framework. See, e.g., Queen, U.S. Pat. No. 5,585,089, hereby incorporated by reference in its entirety. Such humanized monoclonal antibodies can be produced by recombinant DNA techniques known in the art, for example using methods described in PCT Publication No. WO 87/02671; European Patent Application 184,187; European Patent Application. 171,496; European Patent Application 173,494; PCT Publication No. WO 86/01533; U.S. Pat. No. 4,816,567; European Patent Application 125,023; Better et al., 1988, Science 240:1041-1043; Liu et al., 1987, Proc Natl Acad. Sci. 84:3439-3443; Liu et al., 1987, J. Immunol. 139:3521-3526; Sun et al., 1987, Proc Natl Acad. Sci. 84:214-218; Nishimura et al., 1987, Cancer Res. 47:999-1005; Wood et al., 1985, Nature 314:446-449; and Shaw et al., 1988, J. Natl. Cancer Inst. 80:1553-1559; Morrison, 1985, Science 229:1202-1207: Oi et al., 1986, BioTechniques 4:214; U.S. Pat. No. 5,225,539; Jones et al., 1986, Nature 321:552-525; Verhoeyan et al., 1988, Science 239:1534; and Beidler et al., 1988, J. Immunol. 141:4053-4060.

Another approach is to produce human antibodies in engineered mouse strains deficient in mouse antibody production with large fragments of the human Ig loci so that such mice can produce a repertoire of human antibodies in the absence of mouse antibodies. By exploiting the mouse machinery for antibody diversification and selection and the lack of immunological tolerance to human proteins, the reproduced human antibody repertoire in these mouse strains can yield high affinity antibodies against any antigen of interest, including human antigens. Using the hybridoma technology, antigen-specific human mAbs with the desired specificity can then be produced and selected. This general strategy was demonstrated in Green et al. Nature Genetics 7:13-21 (1994), which is hereby incorporated by reference in its entirety. The XenoMouse™ strains were engineered with yeast artificial chromosomes (YACs) containing 245 kb and 190 kb-sized germline configuration fragments of the human heavy chain locus and kappa light chain locus, respectively, which contained core variable and constant region sequences. Id. The production of the XenoMouse™ mice is further discussed and delineated, for example, in U.S. Pat. Nos. 6,162,963, 6,150,584, 6,114,598, 6,075,181, and 5,939,598, each of which is hereby incorporated by reference in its entirety. As such, in some embodiments, an engineered host organism, for example mouse, as described herein is immunized with an antigen, and V cells that produce antigen-specific antibody are isolated.

Another approach is the use of a genetically engineered mouse comprising human V genes with mouse constant region genes. After administration of antigen, such a mouse can produce antibody comprising a human variable regions and mouse constant regions. The human heavy chain and light chain variable regions can then be reformatted onto a human constant region to provide a fully human antibody. For example, polynucleotides encoding the heavy and light chain variable regions can be operably linked to polynucleotide encoding human heavy and light chain constant regions. Such an approach has been used, for example by Regeneron Pharmaceuticals, Inc., and is described for example, in U.S. Pat. No. 6,787,637, which is hereby incorporated by reference in its entirety. As such, in some embodiments, an engineered host organism, for example mouse, comprising human variable genes and host organisms constant genes is immunized with an antigen, and V cells that produce antigen-specific antibody are isolated therefrom. The human variable regions can then be formatted onto human constant regions to produce a fully human antibody.

Another approach is a “minilocus” approach, used for example by GenPharm International, Inc. In the minilocus approach, an exogenous Ig locus is mimicked through the inclusion of pieces (individual genes) from the Ig locus. Thus, one or more VH genes, one or more DH genes, one or more HI genes, a mu constant region, and a second constant region (preferably a gamma constant region) are formed into a construct for insertion into an animal. This approach is described in U.S. Pat. No. 5,545,807 to Surani et al. and U.S. Pat. Nos. 5,545,806, 5,625,825, 5,625,126, 5,633,425, 5,661,016, 5,770,429, 5,789,650, 5,814,318, 5,877,397, 5,874,299, and 6,255,458 each to Lonberg and Kay, U.S. Pat. Nos. 5,591,669 and 6,023,010 to Krimpenfort and Berns, U.S. Pat. Nos. 5,612,205, 5,721,367, and 5,789,215 to Berns et al., and U.S. Pat. No. 5,643,763 to Choi and Dunn, each of which is hereby incorporated by reference in its entirety. As such, in some embodiments, a host strain comprising cells with a “minilocus” as described herein is immunized with an antigen, and V cells that produce antigen-specific antibody are isolated. In some embodiments, V cells or V cell precursors comprising a minilocus in their nuclei are delivered to a host organism, the host is immunized, and V cells producing antigen-specific antibody are isolated from the host.

Another approach, used for example by Xenerex Biosciences includes reconstituting SCID mice with human lymphatic cells, e.g., B and/or T cells. The mice are then immunized with an antigen and can generate an immune response against the antigen. See U.S. Pat. Nos. 5,476,996, 5,698,767, 5,958,765, and 6,537,809. As such in some embodiments, human V cells are delivered to a host immunocompromised mouse (e.g., SCID, nude, or the like), the host is immunized with an antigen, and V cells producing antigen-specific antibody are isolated from the host. The human V cells delivered to the host can be naïve V cells.

In some embodiments, a non-human host, for example a mouse, rat, guinea pig, rabbit, goat, sheep, donkey, horse, or camel is immunized with antigen as described herein. In some embodiments, the host organism comprises its endogenous immunoglobulin genes. In some embodiments, the host organism is genetically modified so as to comprise one or more human immunoglobulin genes. In some embodiments, the host organisms is genetically modified so as to comprise one or more human immunoglobulin genes, and further does not have any substantial host immunoglobulin gene activity (for example, if the host has had its immunoglobulin genes deleted, transcriptionally silenced, mutated, or otherwise inactivated). In some embodiments, the antigen is delivered at least one of intravenously, subcutaneously or intramuscularly as described herein. In some embodiments, the antigen is delivered on a dosing schedule.

In some embodiments, antibody-producing cells are isolated from the host. In some embodiments, V cells are isolated from the host. V cells can be isolated according to methods herein. In some embodiments, the V cells are isolated via flow cytometry or FACS. In some embodiments, the V cells are isolated via one or more antibodies attached to a solid phase. In some embodiments, the antibodies for isolating the V cells are selected for isolation of at least one of the following combinations of markers: CD49+ IgG+ IgE+ CD200+ and the absence of at least one B-cell-specific marker and the absence of at least one basophil-specific marker; CD49+ IgG+ IgE+ CD244.2+ and the absence of at least one B-cell-specific marker and the absence of at least one basophil-specific marker; or CD49+ IgG+ IgE+ FcεR1+ and the absence of at least one B-cell-specific marker and the absence of at least one basophil-specific marker, or any other combination of markers that identify a V cell as described herein.

In some embodiments, the isolated antibody-producing cells are further positive for one or more marker as identified in Table 1.1 or Table 1.3, or are further negative for one or more marker as identified in Table 1.2.

Cells such as V cells expressing surface-bound immunoglobulin with high affinity for an antigen can be identified in several ways. In some embodiments, a population of cells is contacted with labeled antigen, and cells with a high level of labeling are isolated, for example by flow cytometry or FACS. In some embodiments, a population of cells is contacted with antigen attached to a solid phase or substrate, and cells with affinity for the solid phase are isolated.

In some embodiments, V cells are isolated from the host, and then V cells expressing surface-bound antibody with high affinity for the antigen are identified. In some embodiments, V cells are isolated, and the V cells with a high affinity for the antigen are then isolated from the population of V cells. In some embodiments, cells with a high affinity for the antigen are isolated, and the V cells are then isolated from the population with high affinity for antigen are identified. In some embodiments, V cells with high affinity for antigen are isolated from a host population in a single step.

As isolated V cell expressing an immunoglobulin with high affinity for an antigen can be used to construct an antibody with high affinity for the antigen. In some embodiments, the V cell is fused with an immortalized cell to generate a hybridoma. As it has been shown that V cells characteristically cycle, in some embodiments, a culture of cells derived from a single isolated V cell is generated. In some embodiments V genes, encoding immunoglobulin variable regions with high affinity for the antigen, are identified in the V cell, and a polynucleotide encoding such a variable region is constructed, or a polypeptide comprising the variable region is constructed.

### EXAMPLES

The following methods were performed with reference to the following Examples, unless stated otherwise.

Immunization of hosts was performed as follows: Animals were immunized with 50 μg of recombinant protein in Complete Freund's Adjuvant (CFA) injected both via the subcutaneous route (base of the tail) and intraperitoneal route. Animals received three intraperitoneal boosts with 50 μg of antigen (Ag) in Incomplete Freund's Adjuvant (IFA) at two week intervals. In a small number of experiments, the animals were boosted with the antigen one day prior to collecting tissues. It is noted that Mycobacteria within CFA is optional for generating V cells. Similar results are obtained following the above protocol by inoculating animals with antigen in IFA.

Murine tissues were collected as follows:

Bone marrow: Femurs and tibias are removed from the mouse and all tissues scraped away from bones using scissors. The epiphyses (bone ends) are then cut to expose the medullary cavity. Bone marrow is flushed out of each bone using a 3 ml syringe with a 25-gauge needle filled with tissue culture media and collected in a 50 ml conical tube. The process is performed aseptically in cases where the cells will be used downstream for tissue culture.

Splenocytes were obtained by performing a standard mouse splenectomy and then processing the spleen. The spleen is place into a 70 um cell strainer. Using the plunger end of a syringe, the spleen is mashed through the cell strainer into a 50 ml conical tube and rinsed with 40 ml of media. The strainer is discarded and the cells are spun at 800×g for 3 minutes. The splenocyte suspension is then treated with an ammonium chloride lysing solution for 5 minutes to eliminate red blood cells, spun down and re-suspended in 10 ml of media. The process is performed aseptically in cases where the cells will be used downstream for tissue culture.

Blood: Blood was collected by heart puncture in EDTA treated blood collection tubes. Blood was then treated with an ammonium chloride lysing solution for 5 minutes, spun down at 800×g for 3 minutes and re-suspended in 1 ml of tissue culture media.

Immunofluorescent staining was performed as follows: Cells from various tissues (spleen, bone marrow, blood) were stained in a 96 well-plate (10⁶ cells per/well) with optimally titrated antibodies diluted in staining buffer (1×PBS, fetal bovine serum, sodium azide) for minimum 20 min at 4 C. Samples were acquired using either BD FACS Canto II or BD LSR II. Data analysis was performed using the DIVA software.

Immunofluorescent staining and cell sorting: Cells were stained in bulk at 50 million/ml using optimally titrated antibodies in IMAG buffer (1×PBS, EDTA, FBS, sodium azide) for minimum of 20 min at 4 C. Cells were sorted using a BD FACSAria™ III (100 micron nozzle, drop drive frequency 31.0 kHz, Sheath pressure 20.5 psi). In those cases where cells post sorting were used for tissue culture the tissue harvesting, processing, immunofluorescent staining and sorting were performed aseptically.

The cell culture of V cells was performed as follows. 25,000-30,000 sorted V cells were plated in a 24-well plate on a feeder layer of M2-10B4 cells (ATCC CRL-1972) treated for 3 hrs with 1 μg of Mitomycin C (SIGMA M4287). Treated M2-10B4 cells were washed twice with complete RPMI media prior to adding sorted cells. The V cells were grown in 50% MyeloCult media (Stemcell Technologies M5300) and 50% complete RPMI Media (RPMI-1640+7.5% FBS (low IgG Hyclone)+1% Penn/Strep/Glutamine+5×10⁻⁵ M 2ME). Colony formation was observed 3 days post sort.

### Example 1A

### Spleen Control

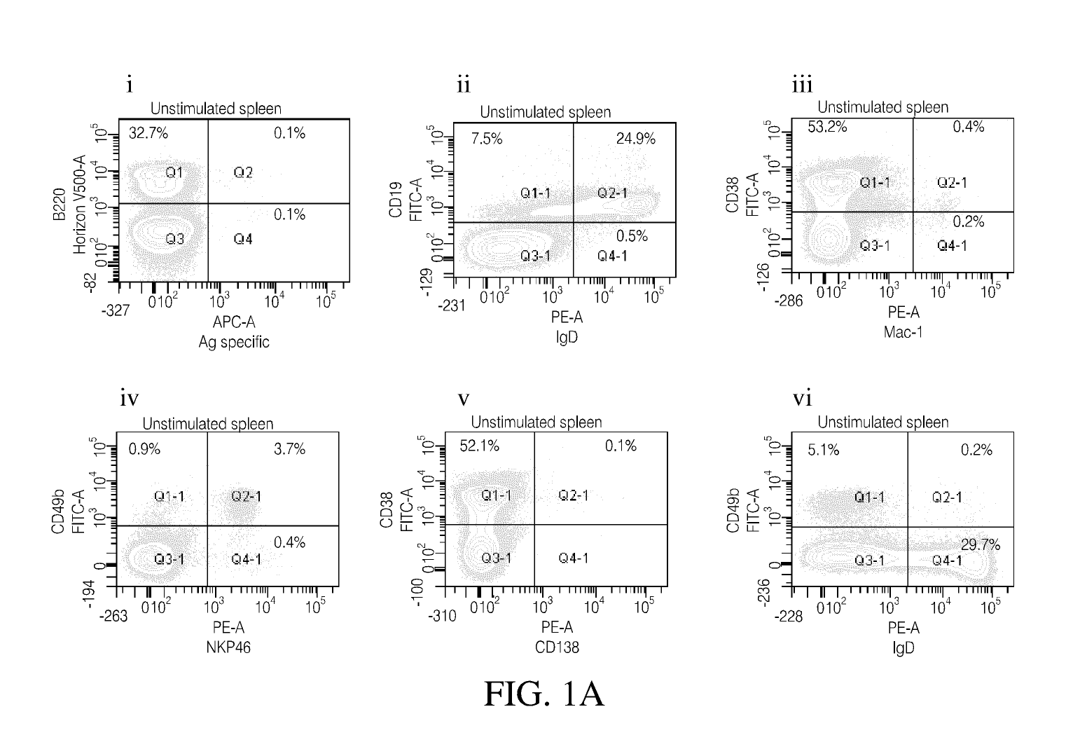

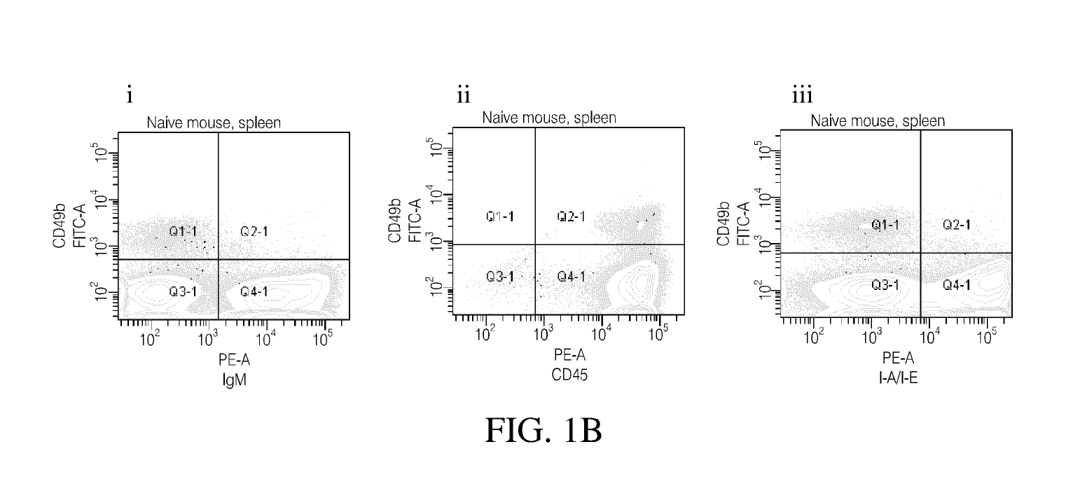

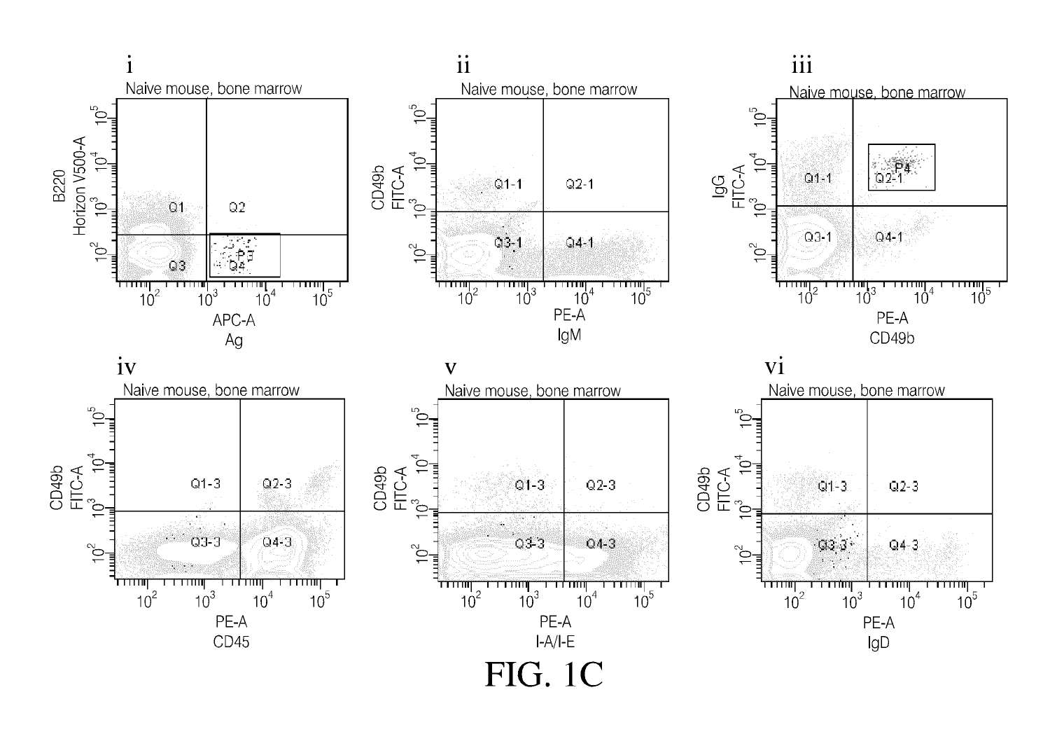

The spleen of a non-immunized BALB/C mouse, which served as a control for 9 different surface markers (Spleen Control). As shown in FIG. 1A, These markers identify B cells (B220+ and CD19+) (panels i and ii); cyclic ADP ribose hydroxylase (CD38) (panel iii) which is found on many immune cells including T cells (CD4+ and CD8+), B cells and Natural Killer cells; Syndecan-1 (CD138) (panel v) expressed on plasma cells; Natural Killer cells (Panel iv) (NKp46 and CD49b); Macrophages (CD11b) (panel iii) and Immunoglobulins G (IgG) and D (IgD) (panel vi) which are antibody isotypes expressed on the surface of B cells at different stages of differentiation.

### Example 1B

### Spleen Control

The spleen of a non-immunized BALB/C mouse served as a control for 3 additional markers (Spleen Control). These markers identify CD45+ cells (present on all differentiated hematopoietic cells with the exception of erythrocytes and plasma cells); Major-histocompatibility Complex class II+ cells (I-A^{d}/I-E^{d}) and Immunoglobulin M which is an antibody isotype expressed on the surface of B cells at different stages of differentiation (FIG. 1B panels i, ii and iii).

### Example 1C

### Bone Marrow Control

The profile of the bone marrow of a non-immunized BALB/C mouse served as a control for 7 different markers (Bone Marrow Control). As shown in FIG. 1C, these markers identify B cells (B220+) (panel i); Immunoglobulins G (IgG) and D (IgD) (panels iii and vi) which are antibody isotypes expressed on the surface of B cells at different stages of differentiation; CD45+ cells (present on all differentiated hematopoietic cells with the exception of erythrocytes and plasma cells) (panel iv); Major-histocompatibility Complex class II cells (I-A^{d}/I-E^{d}) (panel v) and Natural Killer cells (CD49b+) (panel iv).

### Example 2A

### Spleen from a Mouse Immunized with PE (4×)

The profile of the spleen of a Phycoerythrin (PE) immunized BALB/C mouse (Spleen from a mouse immunized with PE (4×)) was examined. As shown in FIG. 2A, five different markers identify B cells (B220+); T cells (CD4+ and CD8+); Macrophages (CD11b/Mac-1+) and Granulocytes (Ly-6G/Gr-1+). B cells producing PE specific antibodies can be observed in quadrant Q2-1 of panel iv stain making 0.4% of the total lymphocyte population. It is also observed that macrophages (panel i) and granulocytes (panel ii) can stain positive (0.2%) for the antigen. However; an antigen specific population is also observed on the lower right hand side quadrant of panels i through v (Q4 and Q4-1), in which the cells are negative for the five aforementioned markers. The population varies from 0.6% to 1% depending on the stain.

### Example 2B

### Spleen of an Allophycocyanin (APC) Immunized BALB/C Mouse

The profile of spleen of an Allophycocyanin (APC)-immunized BALB/C mouse (Spleen from a mouse immunized with APC (4×)) was examined. As shown in FIG. 2B, five different markers identify B cells (B220+) (panel iv); T cells (CD4+ and CD8+) (panels iii and v); Macrophages (CD11b/Mac-1+) (panel i) and Granulocytes (Ly-6G/Gr-1+) (panel ii). B cells producing APC specific antibodies can be observed in quadrant Q2 of panel iv making 0.3% of the total lymphocyte population. It is also observed that macrophages (panel i) and granulocytes (panel ii) can stain positive (0.1-0.2%) for the antigen. However; an antigen specific population is observed on the lower right hand side quadrant (Q4 and Q4-1) in each of the panels i through v, in which the cells are negative for the five aforementioned markers. The population varies from 0.5% to 0.8% depending on the stain.

### Example 2C

### Spleen of an Allophycocyanin (APC) Immunized BALB/C Mouse

The profile of the spleen of an Allophycocyanin (APC) immunized BALB/C mouse (Spleen from a mouse immunized with APC (4×)). Six different markers identify B cells (B220+) (panel i); B-1 cells (CD5+) (panel iii); Syndecan-1 (CD138) expressed by plasma cells (panel ii); T-cell and B-cell activation antigen (GL-7) (panel iv); CD11c+ cells (dendritic cells, CD4− CD8+ intestinal intraepithelial lymphocytes and some NK cells) (panel v) and CD49b (found on NK-T, NK cells and fibroblasts cells) (panel vi). APC staining occurs on the X axis for all six markers. B cells producing APC specific antibodies can be observed in quadrant Q2 of panel i making 3.5% of the total lymphocyte population analyzed. The same antigen specific population described on FIG. 2B is observed on the lower right hand side quadrant (Q4 and Q4-1) in each of panels i through vi, in which the cells are negative for five of the aforementioned markers but is positive for CD49b with 0.6% of the total lymphocyte population analyzed.

### Example 2D

### Profile of a Spleen of an Allophycocyanin (APC) Immunized BALB/C Mouse Focusing on Antigen-Specific Antibody-Producing Cells

Further analysis was performed on the profile of a spleen of an Allophycocyanin (APC)-immunized BALB/C mouse focusing on antigen specific antibody producing cells (Spleen from a mouse immunized with APC (4×)). As shown in FIG. 2D, a gate was placed on the B220+ Antigen specific+ cells (quadrant Q2 of panel i). A second gate was placed on B220− Antigen specific+ cells (quadrant Q4 of panel i). Both of these subpopulations were then analyzed against CD19 (panel ii), CD38 (panels iii and v), CD11b (panel v) and IgD (panel iv). Antigen specific B cells (top panels ii and iii) were positive for CD19 and partially positive for IgD (33%), CD38 (52.7%) and negative for CD11b. The B220−Ag+ cells (bottom panels iv and v) were negative to CD19, CD38, IgD and CD11b. B220−CD19−CD38−IgD−CD11b−Ag+ are labeled as “V cells” in subsequent Examples and FIGS. 2E-5B.

### Example 2E

### Immunization with Various Protein Antigens Induces Antigen Specific V Cells

It was shown that immunization with various protein antigens induces antigen specific V cells. Splenocytes derived from BALB/C mice immunized 4× with either APC (FIG. 2E, dot plots i, iv), PE (FIG. 2E, dot plots ii, v) or OVA (FIG. 2E, dot plots iii, vi) were stained with anti-B220−FITC (FIG. 2E, plots iv, v) or anti-B220−V500 (FIG. 2E, plot vi), antigen (APC, PE, OVA-PE depending on the immunogen used to induce the antigen specific cells) and 7AA-D. Gates were drawn to include events with forward and side scatter characteristics of viable cells (7-AAD−)(see FIG. 2E). The total number of events collected was 100,000 per sample. All three protein antigens indicated above could successfully induce V cells, identified as B220−Ag+7AAD−, in the spleen of immunized mice.

### Examples 2F-2G

### V Cell Distribution in Various Mouse Tissues

V cell distribution in various mouse tissues was examined. Cells derived from the spleen (FIG. 2F, plots i and ii), bone marrow (FIG. 2F, plots iii and iv), peripheral blood (FIG. 2F, plots v and vi), peritoneal exudate cells (PEC) (FIG. 2G, plots i and ii), lymph nodes (FIG. 2G, plots iii and iv), and thymus (FIG. 2G, plots v and vi) of 4× immunized mice with APC were stained with B220 V500 (clone RA3-6B2), APC, and 7AA-D. Gates were drawn to include events with forward and side scatter characteristics of viable cells (7-AAD−). The total number of events collected was 100,000 per sample. V cells (B220−Ag+7AAD−) were observed in the spleen (FIG. 2F, plot ii), bone marrow (FIG. 2F, plot iv), and peripheral blood (FIG. 2G, plot vi) of APC-inoculated mice but not in the PECs, lymph nodes and thymus.

### Example 2G

### V Cell Distribution in Various Mouse Tissues

V cell distribution in various mouse tissues was examined. As shown in FIG. 2G, cells derived from the peritoneal exudate cells (PEC) (plots vii and viii), lymph nodes (plots ix and x), and thymus (plots xi and xii) of 4× immunized mice with APC were stained with B220 V500 (clone RA3-6B2), APC, and 7AA-D. Gates were drawn to include events with forward and side scatter characteristics of viable cells (7-AAD−). The total number of events collected was 100,000 per sample. V cells (B220−Ag+7AAD−) were not observed in the PECs, lymph nodes and thymus.

### Example 211

### Antigen-Specific V Cells can be Detected 8 Days after a Single Immunization

As shown in FIG. 2H, splenocytes derived from either naïve (FIG. 2H, plots i, ii) or immunized (4×APC) 12 week old BALB/C mice (FIG. 2I, plots iii, iv) were stained with anti-B220−V500 (clone RA3-6B2), APC and 7-AAD. Gates were drawn to include events with forward and side scatter characteristics of viable cells (7-AAD−). The total number of events collected was 100,000 per sample. Following immunization, an APC-specific cell population was observed that is B220− (FIG. 2H, plots ii and FIG. 2I, plot iv). A single injection with APC in C57BL/6 mice was sufficient to induce V cells (B220− Ag+7AAD−) in both the spleen (FIG. 2J, plots v and vi) and the bone marrow (FIG. 2K, plots vii and viii) of inoculated mice, as early as day 8 following immunization.

### Examples 3A-3G

### Phenotypic Characterization of Cell Surface Markers Expressed on Antigen-Specific V Cells

Phenotypic characterization of cell surface markers expressed on antigen-specific V cells was performed. Cells derived from the spleen and bone marrow of C57BL/6 mice immunized 4× with APC were stained with anti-mouse B220, IgG, IgE, CD49b, APC, 7-AAD, and antibodies to cell surface markers. As shown in Table 1.2, V cells were negative for a variety of HSC (CD34, c-Kit, Sca-1, and CD150), T- and NKT-cell (CD1d, CD3, CD4, CD8, CD25, and CD134), NK-cell (CD49a, CD122, and CD226/NKp46), dendritic-cell (CD11c and CD273), monocyte (Ly-6G), and a variety of B-cell lineage (CD5, CD19, B220, CD22.2, CD23, CD62P, CD72, GL-7, IgD, IgM, Ly-6K, Ly-6D, Ly-51, CD127, CD138, CD154, AA4.1) markers. V cells were positive for CD24 shown in FIG. 3B (Column ii), CD43 (Column iii). As shown in FIGS. 3C-3D, V cells were positive for CD45 (Column iv), CD48 (Column v), CD79b (Column vi), CD200R (Column vii), FceR1 and IgE (Column viii) surface IgG and CD49b (shown in Panel i). As shown in FIG. 3E, V cells were positive for surface IgG and CD49b (shown in Panel xii). As shown in FIG. 3F, V cells were positive for CD54 (Column x), CD16/CD32 (Column xi), CD244.2 (Column xiii), IgE (present in columns x through xi). As shown in FIG. 3G, cells derived from the spleen and bone marrow of BALB/c mice immunized 4× with APC were stained with anti-mouse B220, IgE, CD49b, APC, 7-AAD, and antibodies to cell surface markers. V cells were positive for CD123 (Column L) in both spleen and bone marrow and negative for basophil-specific antibody 2D7. Antibodies that specifically bind to 2D7 were as described in Ma, et al., “Expression of the basophil-specific antibodies 2D7 and BB1 in patients with cutaneous Mastocytosis,” J Investig Allergol Clin Immunol. 2013; 23(6):392-7, which is incorporated herein by reference in its entirety. Accordingly, 2D7 can be used to differentiate V cells from basophils.

### Examples 3H-3K

### V Cells Cycle in the Bone Marrow and Spleen

The profile of the bone marrow of an immunized BALB/C mouse with B lymphoma Mo-MLV insertion region 1 homolog (BMI-1) recombinant protein pulsed for 24 hours with BrdU to detect V cell cycling was determined. BALB/C mice were injected with 1 mg BrdU in vivo (IP) for 24 hrs. Mice were sacrificed and single cell suspensions made from both spleen and bone marrow. Cells were surface stained for IgG and CD49b and then fixed/stained for BrdU using the BrdU flow kit staining procedure. It was observed that V cells cycle in the bone marrow of the immunized mouse. With reference to FIG. 3H, V cells (CD49b+ IgG+) are identified by gate P2 in plot i. Upon further analysis of the BrdU+ population in plot i, gate P4 in panel iv shows that approximately 53% of the V cell population has incorporated BrdU in 18 hrs. In comparison, CD49b+ IgG− cells (identified in plot i by gate P3), incorporate BrdU in 18 hrs at a lower level of 30% (panel v gate p5).

It was observed that V cells cycle in the spleen of an immunized mouse (24 hr BrdU pulsing). The profile of the spleen of an immunized BALB/C mouse with B lymphoma Mo-MLV insertion region 1 homolog (BMI-1) recombinant protein pulsed for 24 hours with BrdU to detect V cell cycling. BALB/C mice were injected with 1 mg BrdU in vivo (IP) for 24 hrs. Mice were sacrificed and single cell suspensions made from both spleen and bone marrow. With reference to FIG. 3I, cells were surface stained for IgG and CD49b and then fixed/stained for BrdU using the BrdU flow kit staining procedure. V cells (CD49b+ IgG+) are identified by gate P2 in plot i. Upon further analysis of the BrdU+ population in plot i, gate P4 in panel iv shows that approximately 22% of the V cell population has incorporated BrdU in 18 hrs. In comparison, CD49b+ IgG− cells (identified in plot i by gate P3), incorporate BrdU in 18 hrs at a lower level of 18% (panel v gate p5). Splenic V cells incorporate BrdU at a lower level then V cells found in the Bone Marrow.

It was observed that V cells cycle in the bone marrow of an immunized mouse (48 hr BrdU pulsing). This demonstrates that V cells are not terminally differentiated cells. The profile of the bone marrow of an immunized BALB/C mouse with B lymphoma Mo-MLV insertion region 1 homolog (BMI-1) pulsed for 48 hours with BrdU to detect V cell cycling. BALB/C mice were injected with 1 mg BrdU in vivo (IP) for 48 hrs. Mice were sacrificed and single cell suspensions made from both spleen and bone marrow. With reference to FIG. 3J, cells were surface stained for IgG and CD49b and then fixed/stained for BrdU using the BrdU flow kit staining procedure. V cells (CD49b+ IgG+) are identified by gate P2 in plot i. Upon further analysis of the BrdU+ population in plot i, gate P4 in panel iv shows that approximately 58% of the V cell population has incorporated BrdU in 42 hrs. In comparison, CD49b+ IgG− cells (identified in plot i by gate P3), incorporate BrdU in 18 hrs at a lower level of 37% (panel v gate p5). Each of the cell populations increased BrdU incorporation by approximately 5%.

It was observed that V cells cycle in the spleen of an immunized mouse (48 hr BrdU pulsing). The profile of the spleen of an immunized BALB/C mouse with B lymphoma Mo-MLV insertion region 1 homolog (BMI-1) pulsed for 48 hours with BrdU to detect V cell cycling. BALB/C mice were injected with 1 mg BrdU in vivo (IP) for 48 hrs. Mice were sacrificed and single cell suspensions made from both spleen and bone marrow. With reference to FIG. 3K, cells were surface stained for IgG and CD49b and then fixed/stained for BrdU using the BrdU flow kit staining procedure. V cells (CD49b+ IgG+) are identified by gate P2 in plot i. Upon further analysis of the BrdU+ population in plot i, gate P4 in panel iv shows that approximately 43% of the V cell population has incorporated BrdU in 42 hrs. In comparison, CD49b+ IgG− cells (identified in plot i by gate P3), incorporate BrdU in 18 hrs at a lower level of 17% (panel v gate p5). The additional 24 hour BrdU load shows a dramatic increase in splenic V cell BrdU incorporation. On the other hand, the CD49b+ IgG− subset remained unchanged.

### Example 3L

### Enrichment and Sorting of Antigen-Specific V Cells from Spleen and Bone Marrow from Immunized Mice

Enrichment and sorting of antigen-specific V cells from Spleen and Bone Marrow from immunized mice were performed. With reference to FIG. 3L, cells derived from both the spleen (i) and bone marrow (ii) of immunized C57BL/6 mice (injected 4× with APC) were enriched for V cells using the BD IMag™ buffer, a custom biotinylated cocktail containing CD3e, CD11b, LY-6G and LY-6C, TER-119, and BD Imag™ Streptavidin Particles Plus. Enriched cells were then stained with B220 V500, CD49b V450, IgE FITC, IgG PE, APC (Ag), and 7-AAD. V cells were identified as B220− CD49b+IgG+IgE+Ag+7AAD− in both spleen and bone marrow and then sorted using a BD FACSAria™ III system (100-micron nozzle, drop drive frequency 31.0 kHz, sheath pressure 20.5 psi).

### Examples 3M-3N

### Determination of V Gene Utilization in V Cells

Rearranged V gene cDNA was detected in from V cells obtained via sorting in a FACS Aria III from an Allophycocyanin (APC) immunized BALB/C mouse. Isolation and identification of expressed immunoglobulin gene mRNA in V cells was performed as follows: Fresh V cells were sorted into a suitable physiological neutral buffer and then resuspended in RNA lysis buffer, the composition of which known to those skilled in the art. Total RNA was isolated using a commercial total RNA isolation kit. This includes Qiagen RNEasy mini spin column or equivalent. RNA was stored at −80° C.

Reverse transcriptase polymerase chain reaction (RT-PCR) was used to generate cDNA and amplify the immunoglobulin variable region genes (V-genes) from both heavy and light chains. First strand cDNA was synthesized using oligo dT primers. Oligo dT primed and reverse transcribed cDNA was used for PCR amplification in order to test for the expression of immunoglobulin variable region expression. A total of 5 μl of cDNA isolated from 13,000-52000 bulk sorted V cells was mixed with appropriate oligonucleotide primers to amplify immunoglobulin variable region gene cDNA or actin, taq polymerase and buffers and amplified using PCR.

Oligonucleotide primers were designed based upon publicly available sequences and correspond to regions of the cDNA where they anneal in the relatively conserved leader and Framework 1 regions of immunoglobulin gene cDNA. Corresponding isotype-specific back primers were designed based upon the flow analysis which shows that V cells bear antigen specific gamma class immunoglobulin heavy chains. The oligonucleotide primers spanned introns present in the DNA copies of immunoglobulin chains, which cover a distance of approximately 1500 base pairs. The introns are spliced out of heterogeneous nuclear mRNA as message is generated. Polymerase chain reaction amplification of immunoglobulin variable region gene (V-gene) cDNA was performed using oligonucleotide primer sets as follows. Oligonucleotide primers specific to the IgG or kappa constant domains (reverse) were paired with immunoglobulin heavy or kappa chain variable region (VH or Vk); the variable region primers correspond to regions in the upstream leader or to framework 1 region.

The heavy chain forward primers were provided as a pool of leader primers:

MHcL1

(SEQ ID NO: 1)

ATGGACTT(GCT)G (GAT)A(CT)TGAGCT;
MHcL2

(SEQ ID NO: 2)

ATGGAATGGA(GC)CTGG(GA)TCTTTCTCT;
MHcL3

(SEQ ID NO: 3)

ATGAAAGTGTTGAGTCTGTTGTACCTG;
MHcL4

(SEQ ID NO: 4)

ATG(GA)A
(GC)TT(GC)(TG)GG(TC)T(AC)A(AG)CT(TG)G(GA)TT;
and IgG back primers:
MG1-3Seq

(SEQ ID NO: 5)

AGA TGG GGG TGT CGT TTT GGC;
MG2ab-3Seq

(SEQ ID NO: 6)

GAC YGA TGG GGS TGT TGT TTT GGC;

For the kappa chain forward primers were provided as a pool of leader primers

KcL-1

(SEQ ID NO: 7)

ATGAAGTTGCCTGTTAGGCTGT b;
MKcL-2

(SEQ ID NO: 8)

ATGGACTTTCAGGTGCAGATCT;
MKcL-3

(SEQ ID NO: 9)

TTGCTGTTCTGGGTATCTGGTA;
MKcL-4

(SEQ ID NO: 10)

ATGGAGACAGAC ACACTCCTGCTAT;
with the reverse primer MKC1

(SEQ ID NO: 11)

GGATACAGTTGGTGCAGC.

Without being limited by any particular theory, it is generally believed that DNA rearrangement, immunoglobulin gene mRNA expression and IgG expression only occur in antibody producing cells. These genes are not rearranged nor activated in non-antibody producing cells and the genes remain quiescent and buried in heterochromatin. Thus PCR amplification of a variable region cDNA of about 400 base pairs from an RT-PCR reaction showed that a V cell is producing immunoglobulin gamma mRNA and expresses antibody. This surprising finding demonstrates that the surface IgG is endogenously expressed in the V cell population. The recovery of re-arranged, expressed IgG mRNA from V cells was unexpected as this has previously only been seen in B cells.

With reference to FIG. 3M, an ethidium bromide stained 1% agarose gel showing immunoglobulin variable region gene cDNAs after amplification using heavy chain leader region primers and IgG CH1 isotype specific reverse primers and i) RNA from V cells sorted from bone marrow, or ii) RNA from V cells sorted from spleen. It is noted that FIG. 3L illustrates rearranged V gene cDNA from V cells from two compartments.

As shown in FIG. 3N, V cells express re-arranged immunoglobulin V-region mRNA. Cells derived from the bone marrow and spleen of BALB/C or C57BL/6 mice immunized 4× with APC (Ag) were magnetically enriched for V cells, and subsequently stained with B220 V500, anti-mouse IgE FITC, IgG PE, CD49b V450, APC and 7-AAD. V cells (B220−IgG+IgE+CD49b+APC+7-AAD−) were bulk sorted and then used for mRNA isolation, PCR amplification, cloning, and sequencing of their VH and VL genes. Bulk sorted V cells expressed re-arranged VH and VL genes in both the bone marrow and spleen. Recovery of rearranged IgG cDNA and rearranged kappa Light chain cDNA from V cells sorted from the bone marrow is shown in Panel i, while Nested IgE PCR with a gradient for optimization is shown on Panel ii.

Immunoglobulin V gene utilization of five representative V cell variable region sequences is illustrated in FIGS. 7A-E. The amplification reactions clearly show the V cells express rearranged immunoglobulin V gene cDNA in the gamma and kappa formats Analysis of amplified V genes compared to germline immunoglobulin variable regions genes from mice in public databases demonstrated several findings including: multiple distinct germ line variable regions genes are used to target this particular antigen by V cells, the variable region genes are somatically mutated away from their closest germ line counterparts (not in germ line state) unlike the B-1B cell population in which the V genes are essentially pristine. This suggests either affinity maturation has taken place and or a recruitment of already matured V genes with a fit for this antigen. Multiple different DH and HI elements are recombined in these V genes showing a polyclonal recruitment of these cells by antigen and that these are distinct clones (examine CDR3 amino acid translation FIGS. 7A-E).

### Examples 3O-3R

### Morphological Characterization of V Cells

Cells derived from the bone marrow and spleen of BALB/C or C57BL/6 mice immunized 4× with APC were magnetically enriched for V cells, and subsequently stained with B220 V500, anti-mouse IgE FITC, IgG PE, CD49b V450, APC and 7-AAD. V cells (B220−IgG+IgE+CD49b+APC+7-AAD−) were bulk sorted and then used for cytospins followed by methanol fixation and DAPI staining. Confocal microscopy analysis was performed. FIG. 3O is a series of confocal microscope images illustrating that V cells are polymorphonuclear and express IgG and IgE simultaneously on their surface. Confocal microscopy analysis indicated that V cells are polymorphonuclear (Panels i and ii) and confirmed presence of both antigen specific IgG and IgE on the cell surface. Antibody capping was observed on 95% of the cells analyzed (Panel i), while 5% of the cells showed dispersed antigen, IgG and IgE on the cell surface (Panel ii). V cell nuclear morphology is distinct when compared to classical B cell subsets.

Light microscopy analysis of V cells was also performed. Cells derived from the bone marrow and spleen of BALB/C mice immunized 4× with APC (Ag) were magnetically enriched for V cells, and subsequently stained with B220 V500, anti-mouse IgE FITC, IgG PE, CD49b V450, APC and 7-AAD. V cells (B220−IgG+IgE+CD49b+APC+7-AAD−) were bulk sorted and were then used for either cytospins followed by methanol fixation and Giemsa staining or DAPI staining. FIG. 3P is a series of microscope images illustrating that V cells have two distinct nuclear shapes. With reference to FIG. 3P, two distinct nuclear shapes can be observed: the first is an annular or ring shaped nucleus with a circular void running down through its center (panels i and ii), and the second is a multi-lobed nucleus (panels iii and iv) that shows no distinguishable chromatin filaments between each lobe (a characteristic shown by neutrophils).

Electron microscopy (EM) analysis of V cells was also performed. Cells derived from the bone marrow and spleen of BALB/C or C57BL/6 mice immunized 4× with APC were magnetically enriched for V cells, and subsequently stained with B220 V500, anti-mouse IgE FITC, IgG PE, CD49b V450, APC and 7-AAD. V cells (B220− IgG+IgE+CD49b+APC+7-AAD−) were bulk sorted in a BD FACSAria™ III and then used for EM. EM analysis performed on a Tecnai spirit TEM by FEI at 80 KV equipped with Gatan 4 k×4 k digital camera. The analysis showed that V cells have a distinct ultrastructure. In particular, the V cells having a different ultrastructure when compared to normal lymphocytes and appear to be richer in organelles, have more cytoplasm and many granular structures (see FIG. 3Q, Panels i and ii). Without being limited by any particular theory, it appears that the granular structures could be peroxisomes, but could also be primary or secondary lysosomes, or secretory granules. Two distinct types of nucleus are discernible: a multi-lobed mono-nuclear version on both spleen and bone marrow cells (FIG. 3Q, Panels i and ii) and a second ring shaped (annular) version (FIG. 3Q, Panel iii) confirmed by Giemsa stain and confocal microscopy (FIG. 3P).

FIG. 3R is a series of tailed EM images of organelles of V cells in spleen and bone marrow. Photomicrograph of organelles and general cellular ultrastructure of V cells from mouse spleen (panels i and ii) and bone marrow (panels iii and iv) taken at 10,000 times amplification. V cells have a characteristically large amount of rough endoplasmic reticulum (panel i), a large amount of cytoplasm which is very rich in organelles (panel ii) and many granular structures (panels iii and iv). The granular structures could be peroxisomes, but could also be primary or secondary lysosomes, or secretory granules.

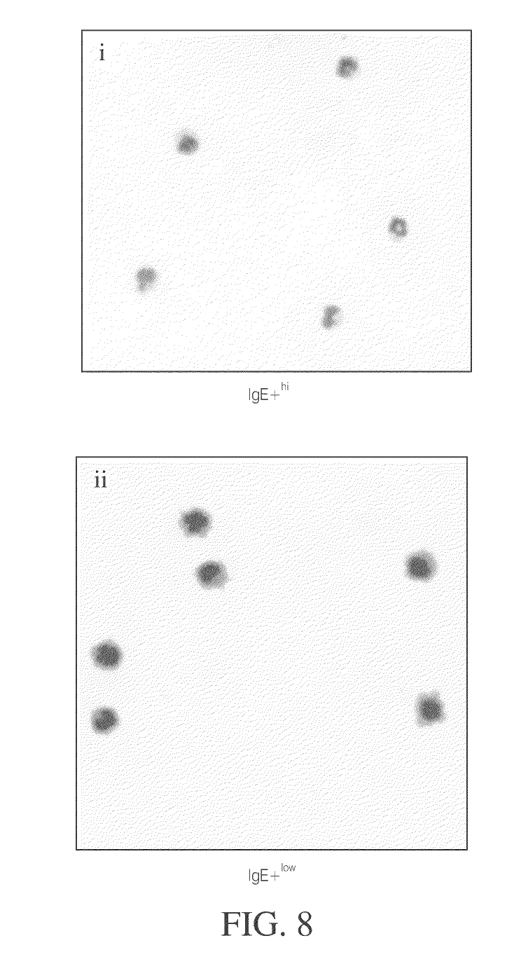

Human V cells were sorted based on the phenotype IgE+IgG+CD200R+CD49b+CD19−, and stained with May-Grüenwald Giemsa stains. Shown in FIG. 8 are cells with high levels of IgE (panel i) and low levels of IgE (panel ii). The nuclear morphology of human V cells was similar to that of mouse V cells. Accordingly, it is contemplated that polymorphonuclear morphology is characteristic of V cells across species.

### Examples 3S-3T

### Maintenance of V Cells in Culture

Cells derived from the bone marrow or spleen of BALB/C or C57BL/6 mice immunized 4× with APC were magnetically enriched for V cells, and subsequently stained with B220 V500, anti-mouse IgE FITC, IgG PE, CD49b V450, APC and 7-AAD. V cells (B220−IgG+IgE+CD49b+APC+7-AAD−) were bulk sorted aseptically in a BD FACSAria™ III and then used for tissue culture. 12,000-30,000 sorted V cells were plated in a 24-well plate on a feeder layer of M2-10B4 cells (ATCC CRL-1972) treated for 3 hrs. with 1 μg of Mitomycin C (SIGMA M4287). Treated M2-10B4 cells were washed twice with complete RPMI media prior to adding sorted cells. The V cells were grown in 50% MyeloCult media (Stemcell Technologies M5300) and 50% complete RPMI Media (RPMI-1640+7.5% FBS (low IgG Hyclone)+1% Penn/Strep/Glutamine+5×10⁻⁵ M 2ME). Colony formation was observed 3 days post sort. As shown in FIG. 3S, sorted V cells can be maintained in tissue culture.

FIG. 3T is a series of microscope images illustrating a comparison between V cells from bone marrow and spleen against hematopoietic stem cells (HSC) in tissue culture. Images of sorted V cell and HSC colonies grown on M2-10B4 feeder cells. Bulk sorted BALB/C V cells from bone marrow (panel i), spleen (panel ii) and bulk sorted C57BL/6 HSC (KLS) cells (panel iii) were plated on mitomycin C-treated M2-10B4 cells and cultured for 10 to 13 days using Myelocult medium (StemCell Technologies M5300). Equal numbers of bone marrow and spleen V cells were plated on M2-10B4 feeder layer. HSC were plated at ½ the cell concentration. Cell colonies grew in all 3 sorted cell populations. HSC colonies began to appear at day 3, V cell colonies began to appear between days 3 and 5.

Murine V cells formed colonies after 8 days in tissue culture. FIG. 9 illustrates light microscope images of the V cell colonies that formed.

### Example 4

### Isolation and Characterization of Naïve V Cells

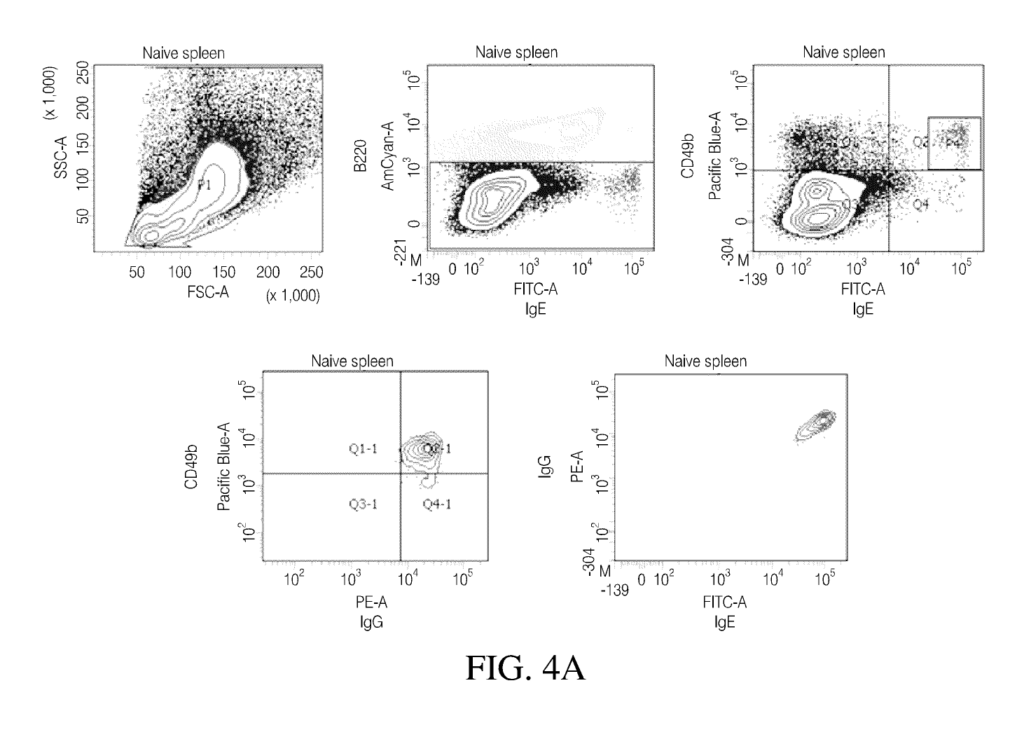

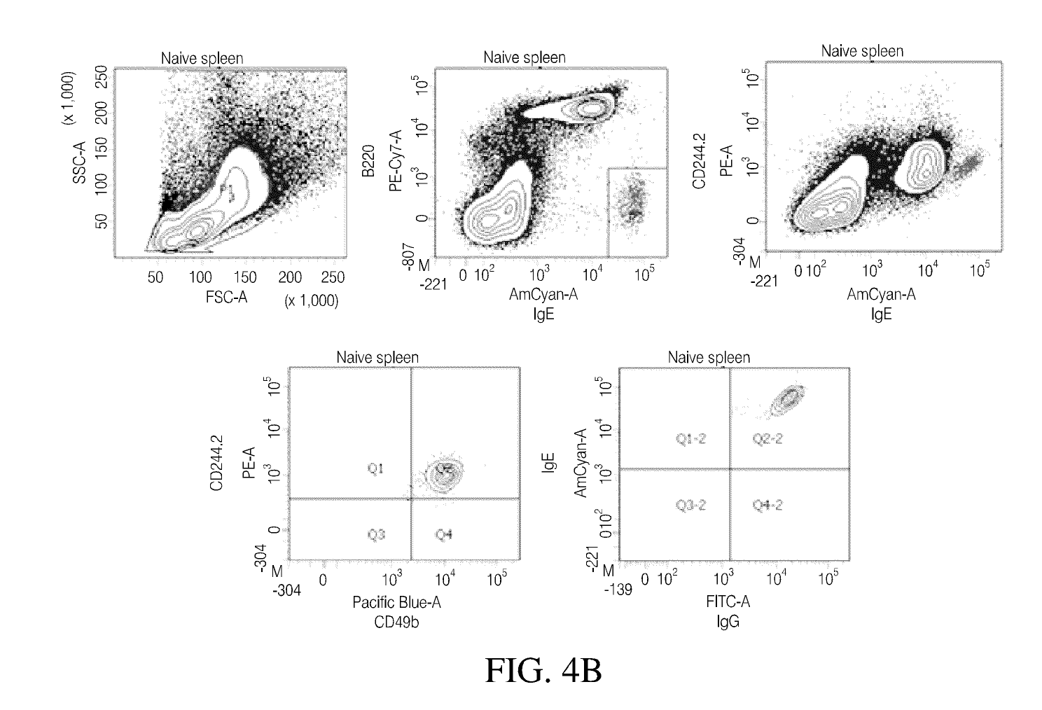

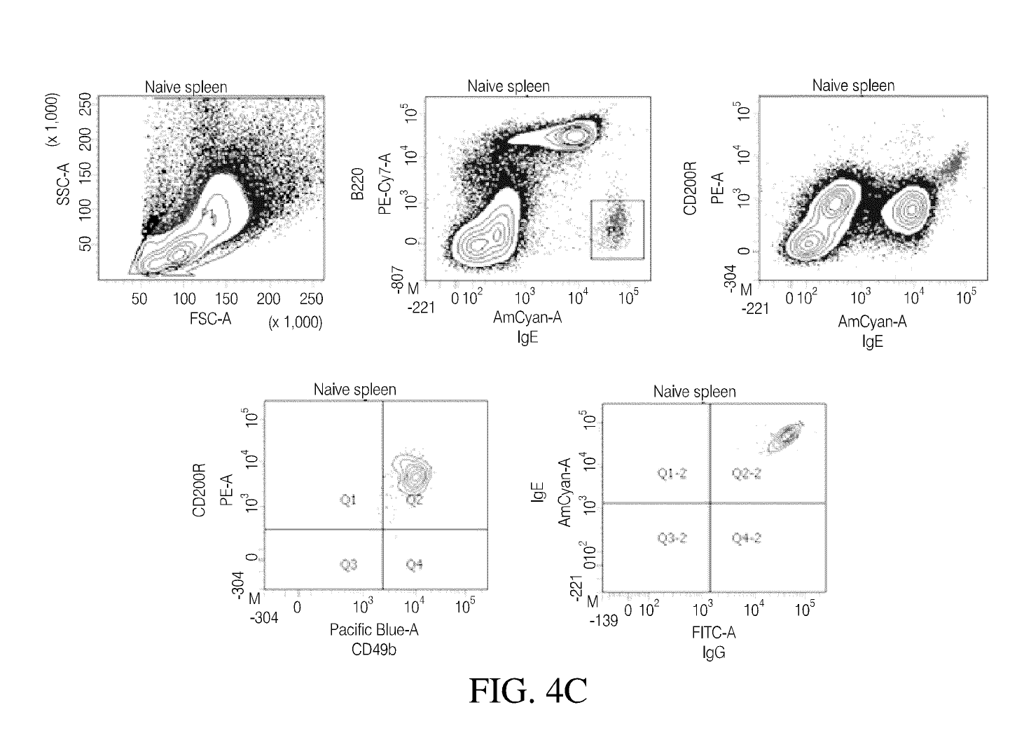

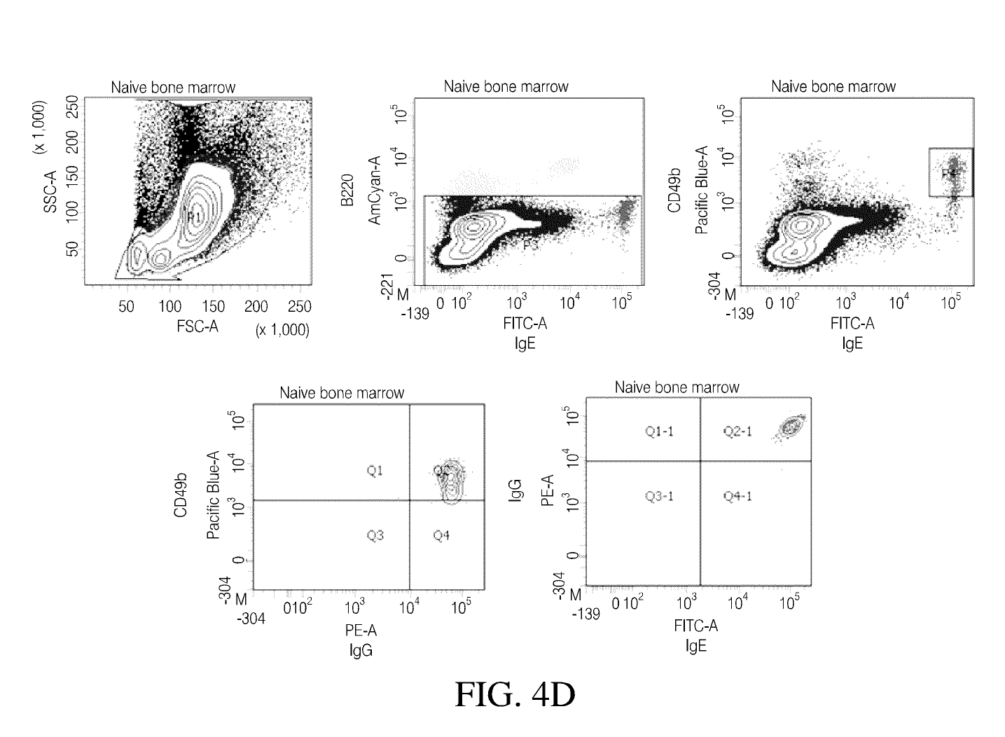

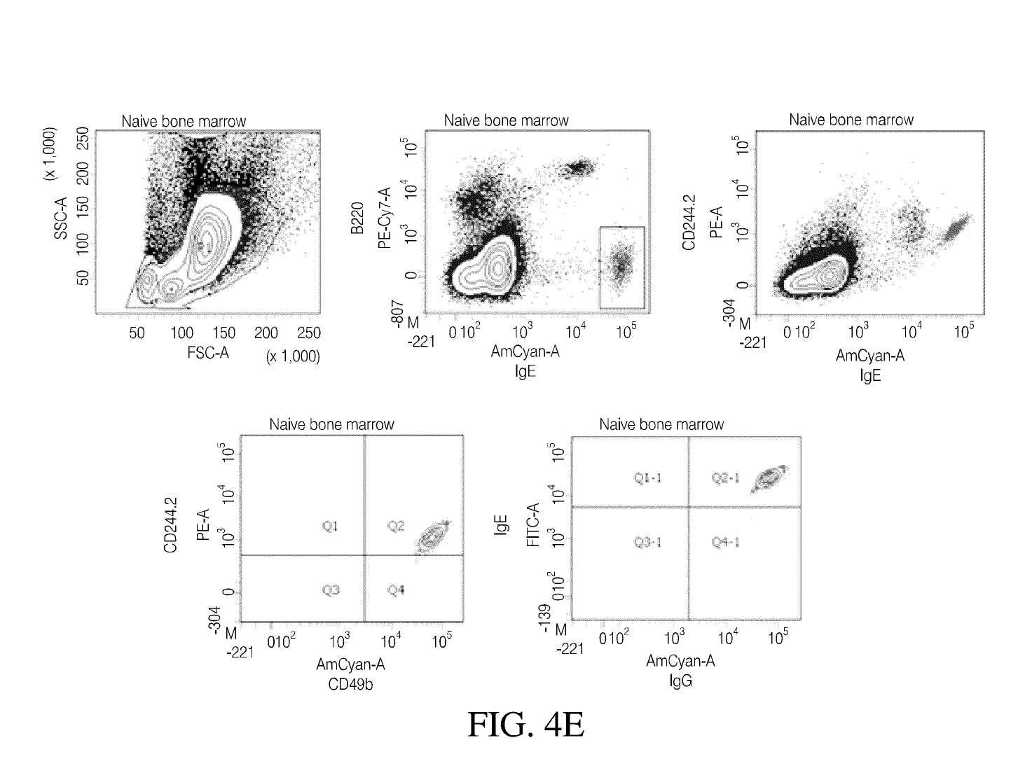

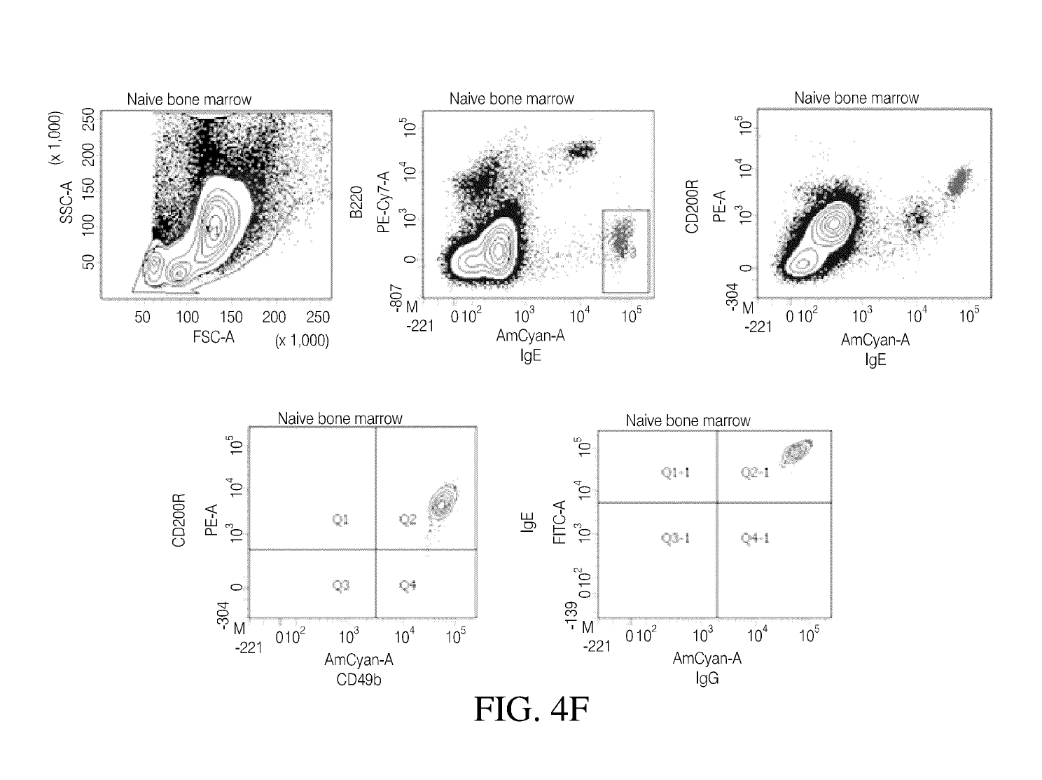

It was determined that naïve V cells are present in the spleen of nude mice. Due to a genetic mutation, nude mice (CD57BL/6 background) lack or have a severely deteriorated thymus and cannot generate mature T lymphocytes. This characteristic makes the mice unable to mount most types of immune responses, including: antibody formation that requires CD4+ helper T cells, cell-mediated immune responses (require CD4+ and/or CD8+ T cells) and delayed-type hypersensitivity responses (require CD4+ T cells) amongst others. Cells derived from the spleen of nude mice (C57BL/6 background) were stained with markers that characterized antigen specific V cells (anti-mouse B220, IgG, IgE, CD49b) and 7-AAD. With reference to FIG. 4A, an initial gate was drawn on all B220− cells, followed by a secondary gate that focused on CD49b+IgE+ cells. Naïve V cells from the spleen share the same phenotype as their antigen-specific counterpart and they are B220−IgG+IgE+CD49b+.

Further phenotypic characterization was performed on naïve V cells derived from the spleen. Cells derived from the spleen of nude mice (C57BL/6 background) were stained with markers that characterized antigen specific V cells (anti-mouse B220, IgG, IgE, CD244.2, CD200R) and 7-AAD. FIG. 4B and FIG. 4C are a series of graphs illustrating phenotypic characterization of naïve V cells in the spleen of nude mice. Gates were drawn on B220−IgE+, CD200R+IgE+^{hi} and CD244.2+IgE+^{hi} cells highlighting the V cell population. Naïve V cells from the spleen share the same phenotype markers as their antigen-specific counterpart and they are B220−IgG+IgE+CD49b+CD244.2+CD200R+.

It was also determined that naïve V cells are present in the bone marrow of nude mice. Following the same strategy to detect naïve V cells in spleen (see FIGS. 4C-4D), cells derived from the bone marrow of nude mice (C57BL/6 background) were stained with markers that characterized antigen specific V cells (anti-mouse B220, IgG, IgE, CD49b) and 7-AAD. FIG. 4D is a series of graphs illustrating that naïve V cells are present in the bone marrow of nude mice. An initial gate was drawn on all B220− cells, followed by a secondary gate that focused on CD49b+IgE+ cells. Naïve V cells from the bone marrow share the same phenotype as their antigen-specific counterpart and they are B220−IgG+IgE+CD49b+.

Further phenotypic characterization was performed on naïve V cells derived from the bone marrow. Cells derived from the bone marrow of nude mice (C57BL/6 background) were stained with markers that characterized antigen specific V cells (anti-mouse B220, IgG, IgE, CD244.2, CD200R) and 7-AAD. FIG. 4E and FIG. 4F are a series of graphs illustrating phenotypic characterization of naïve V cells in the bone marrow of nude mice. Gates were drawn on the subpopulation of IgE+, CD200R+IgE+hi and CD244.2+IgE+hi cells highlighting the V cell population. Naïve V cells from the bone marrow share the same phenotype markers as their antigen-specific counterpart and they are B220-IgG+IgE+CD49b+CD244.2+CD200R+.

### Example 5

### Identification and Characterization of Naïve V Cells in Humans

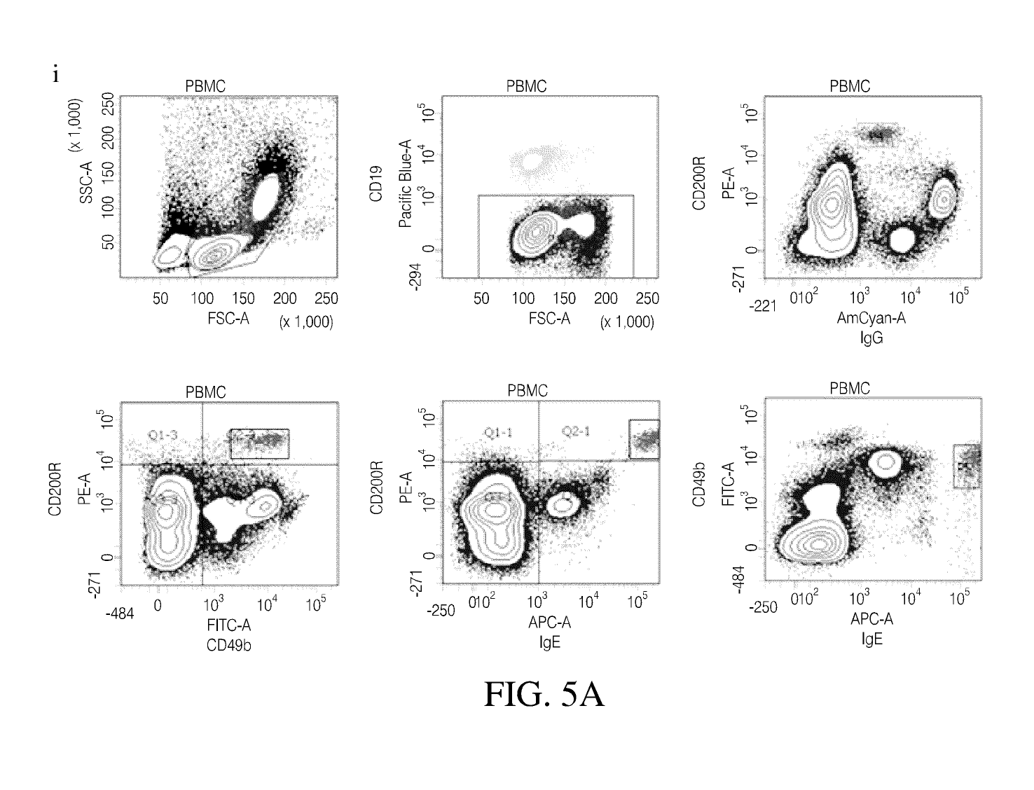

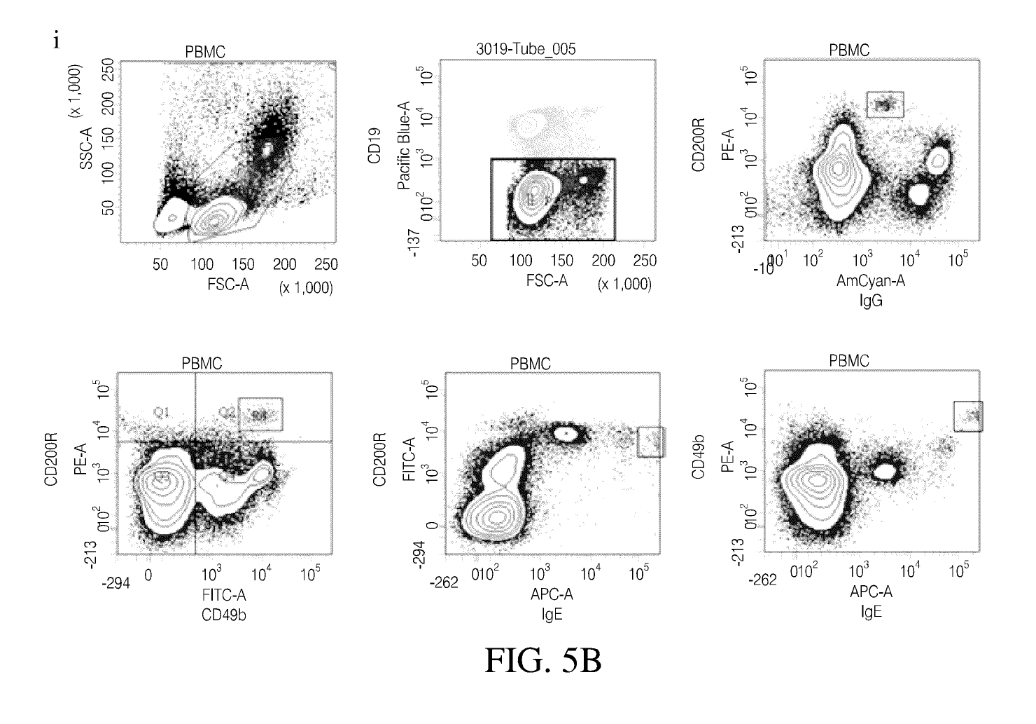

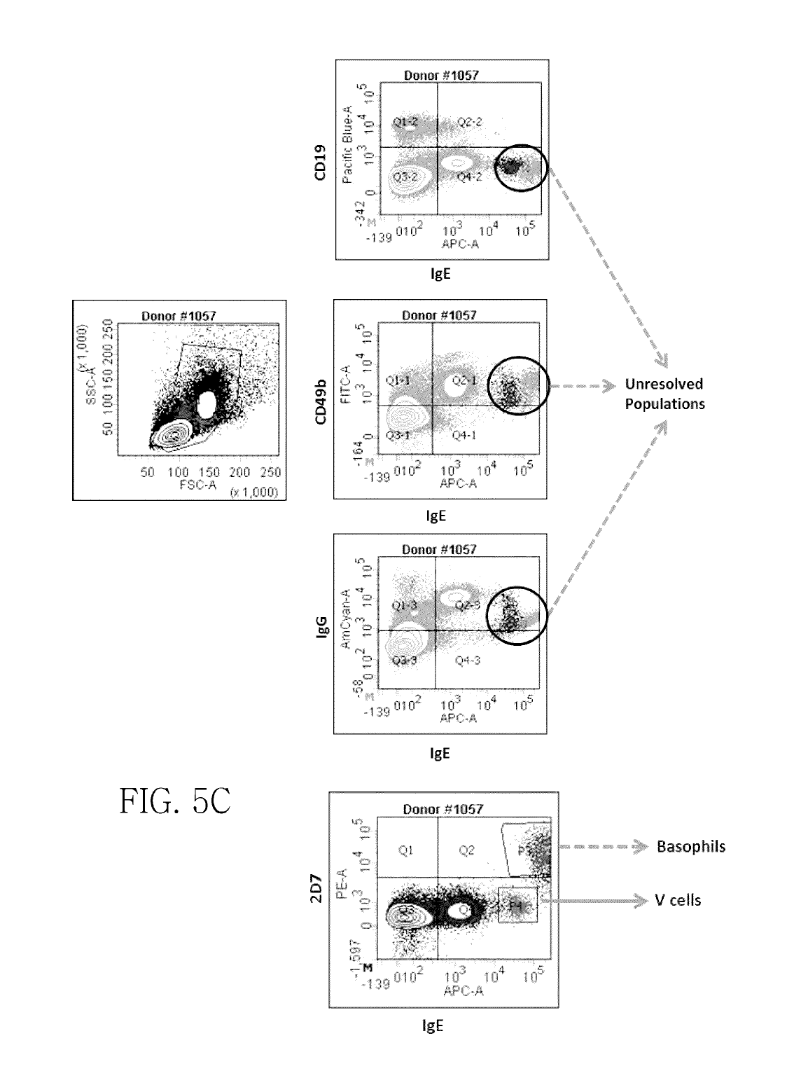

FIGS. 5A, 5B, and 5C are a series of graphs illustrating phenotypic characterization of naïve V cells in human peripheral blood. Human blood was collected from two different donors (FIGS. 5A and 5B, respectively) and PBMCs were isolated using the Ficoll-Paque protocol. PBMCs were then stained with CD19 and a cocktail of positive markers for V cells (CD49b, IgG, IgE and CD200R). Gates were drawn on CD19− cells and then on the V cell population to highlight their presence. V cells can be identified as CD19− CD49b+IgG+IgE+CD200R+. In FIG. 5C, human blood was collected from a donor and PBMCs were isolated using the Ficoll-Paque protocol. PBMCs were then stained with CD19 and a cocktail of positive markers for V cells (CD49b, IgG, IgE) and basophil-specific marker 2D7. Due to the fact that V-cells and basophils can share a large number of markers, 2D7 can serve as a useful negative marker to separate these two populations. V-cells can be identified as CD19−CD49b+IgG+IgE+2D7−. Antibodies that specifically bind to 2D7 and BB1 are described in Ma et al., “Expression of the basophil-specific antibodies 2D7 and BB1 in patients with cutaneous Mastocytosis,” J Investig Allergol Clin Immunol. 2013; 23(6):392-7, which is incorporated herein by reference in its entirety. Accordingly, 2D7 can distinguish naïve V cells from basophils.

### Example 6

### 24 and 48 Hour In Vivo BrdU Pulsing of BMI and APC Primed Mice

Previously immunized Balb/c {against either BMI or APC} mice (2 mice from each group) were boosted for either 24 or 48 hrs with both antigen or BrdU (1 mg IP/mouse). Mice were sacrificed and tissues harvested from Bone Marrow, Thymus, and spleen. Cells were stained for BrdU, IgG, CD49B, and APC. Results are summarized in Tables 4 and 5.

TABLE 4
BrdU pulsing of BMI primed mice

| | % BrdU+ | % NK+/IgG+ | % IgG + | % NK+/IgG− |
|---|---|---|---|---|
| Cell Type | (total cells) | BrdU+ | BrdU+ | BrdU+ |
| Bone | 43 | 47 | 17 | 26 |
| Marrow | | | | |
| 24 hrs | | | | |
| Spleen | 20 | 17 | 3 | 14 |
| 24 hrs | | | | |
| Thymus | 21 | — | 3 | 5 |
| 24 hrs | | | | |
| Bone | 67 | 60 | 39.5 | 37 |
| Marrow | | | | |
| 48 hrs | | | | |
| Spleen | 16 | 42 | 5 | 17 |
| 48 hrs | | | | |
| Thymus | 30 | — | 0.5% | 6.4 |
| 48 hrs | | | | |

TABLE 5
BrdU pulsing of APC primed mice

| | % BrdU+ (total | % NK+/IgG+ | % IgG + | % NK+/IgG− |
|---|---|---|---|---|
| Cell Type | cells) | BrdU+ | BrdU+ | BrdU+ |
| Bone | 38 | 48 | 13 | 25 |
| Marrow | | | | |
| 24 hrs | | | | |
| Spleen | 18 | 16 | 5 | 12 |
| 24 hrs | | | | |
| Thymus | 21 | — | 4 | 3 |
| 24 hrs | | | | |
| Bone | 44 | 37 | 41 | 21 |
| Marrow | | | | |
| 48 hrs | | | | |
| Spleen | 10 | 23 | 4 | 13 |
| 48 hrs | | | | |
| Thymus | 19 | — | 2 | 7 |
| 48 hrs | | | | |

### Example 7

### Cytokine Production by V Cells

Day 10-13 sorted V cells and HSCs were grown on an M2-10B4 feeder layer. Images of sorted V cell and HSC colonies grown on M2-10B4 feeder cells. Bulk sorted BALB/c V cells from bone marrow and spleen and bulk sorted C57BL/6 HSC (KLS) cells were plated on mitomycin C-treated M2-10B4 cells and cultured for 10 to 13 days using Myelocult medium (StemCell Technologies M5300). Equal numbers of bone marrow and spleen V cells were plated on an M2-10B4 feeder layer. HSCs were plated at ½ the cell concentration. Cell colonies grew in all three sorted cell populations. Although HSC colonies began to appear at day 3, V-cell colonies began to appear between days 5 and 7. Supernatants were harvested from the wells and assayed for cytokines using the enhanced sensitivity BD CBA (Table 6, below). V cells routinely produced IL-4, TNF, and occasionally IL-13. HSCs produced IL-10 and TNF but did not produce IL-13 or IL-4. Data is represented as femtogram per milliliter concentrations.

TABLE 6
Cytokine production of V cells and HSCs (KLS) grown on an M2-10B4
feeder layer (values are represented as fg/mL)

| Cell Type | IL-4 | TNF | IL-13 | IL-10 |
|---|---|---|---|---|
| M2-10B- | 0 | 0 | 0 | 0 |
| experiment 1 | | | | |
| BM V cells | 1,506 | 0 | 0 | 0 |
| Sp V cells 1 | 323 | 5,849 | 0 | 0 |
| MC-10B4- | 0 | 0 | 0 | 0 |
| experiment 2 | | | | |
| BM V cells 2 | 9,416 | 1,763 | 3,014 | 0 |
| SP V cells 2 | 13,901 | 6,193 | 347 | 0 |
| KLS C75BI/6 | 0 | 18,946 | 0 | 16,415 |
| HSCs | | | | |
| KLS BALB/c | 0 | 24,845 | 0 | 20,225 |
| HSCs | | | | |

### Example 8

### Analysis of Cell Surface Phenotypes of Antigen-Specific V Cells

Cells derived from the spleen and bone marrow of mice immunized 4× with APC were stained with B220 (clone RA3-6B2), anti-mouse IgG (polyclonal) or/and CD49b, APC, 7-AAD, and antibodies to cell surface markers listed in the Tables 1.1-1.3 above (as detailed in Materials and Methods). V cells were negative for a variety of HSC (CD34, c-Kit, Sca-1, and CD150), T and NKT cell (CD1d, CD3, CD4, CD8, CD25, and CD134), NK cell (CD49a, CD122, and CD226/NKp46), dendritic cell (CD11c and CD273), monocyte (Ly-6G), and a variety of B-cell lineage (CD5, CD19, B220, CD22.2, CD62P, CD72, GL-7, IgD, IgM, Ly-6K, Ly-6D, Ly-51, CD127, CD138, CD154, AA4.1) markers. V cells were positive for CD24, CD43, CD45, CD48, CD49b, CD79b, CD200R, surface IgG, and weakly positive for CD11b expression. Table 1.1 summarizes surface markers for which V cells are positive. Table 1.2 summarizes surface markers for which V cells are negative. Table 1.3 summarizes surface markers that appear to be expressed at low levels on V cells.

In some embodiments, a kit includes a combination of antibodies that target V-cell specific markers as identified in Table 1.1. In some embodiments, the kit further includes one or more antibodies that target markers for which V cells are negative, as identified in Table 1.2. In some embodiments, the kit further includes one or more antibodies that target markers that are expressed on V cells at low levels, as identified in Table 1.3.

### Example 9

### Detection of V Cells in the Presence or Absence of Labeled Antigen

Mice were immunized with APC (antigen) and their splenocytes were stained with antibodies against B220, IgE, IgG and CD49b. For one staining procedure, labeled antigen (Ag) was also added to the staining reaction. For another staining procedure, no antigen was provided. Show in FIG. 10A are results for a procedure in which labeled antigen (APC) was added to the tube during the staining procedure to detect the V cells. Shown in FIG. 10B are results for a procedure in which antigen was omitted during the staining procedure. V cells are identified as B220−IgE+IgG+CD49b+. V cells identified as Ag+ by staining with labeled APC represented about 0.2% of the population (FIG. 10A). V cells identified as IgG+ IgE+ in the absence of labeled antigen represented about 0.2% of a comparable population (FIG. 10B). Accordingly, it is contemplated herein that all or nearly all of the identified V cells produced antigen-specific immunoglobulin.

### Examples 10-12

The following methods were performed with reference to Examples 10-12, unless stated otherwise.

An EAE model was provided as follows: An inflammatory disease of the central nervous system was induced in rodents by injecting mice with central nervous system proteins (e.g. MBP, MOG) or peptides in complete Freud's adjuvant (CFA). EAE was used as a model of human CNS demyelinating diseases, including multiple sclerosis (MS) and acute disseminated encephalomyelitis (ADEM). In the EAE model used in this study humanized HLA-DR2 (DRB1*1501) mice were immunized s.c. at the lower back with 200 μg MBP-85-99 peptide emulsified in CFA followed by pertussis toxin on day 0 and 2 of immunization to break down the blood brain barrier and allow immune cells to access the CNS. The humanized transgenic mice bear chimeric MI-IC class II molecules encoded by human sequences HLA-DR2 (DRB1*1501) while the membrane proximal portion including the CD4-binding domain is encoded by mouse sequences on a C57BL/6 background. In addition, these mice also express a human TCR specific for the MBP-85-99 peptide so the majority of their T cell response following immunization is directed to this epitope. The MBP peptide is highly encephalitogenic in HLA-DR2 (DRB1*1501) mice. The HLA-DR2 allele, DRB1*1501 is present in ˜60% of the Caucasian MS patients, confers the highest risk in disease susceptibility.

Clinical EAE disease severity was accessed daily following the following scale: 0=normal, 1=limptail or mild hind limp weakness, 2=limp tail and moderate hind limp weakness, 3=limp tail and moderately severe hind limp weakness, 4=limp tail and severe hind limp or mild forelimp weakness or moderate ataxia, 5=limp tail and paraplegia with no more than moderate forelimp weakness, and 6=limp tail and paraplegia with severe forelimp weakness or severe ataxiaor moribund condition.

Splenocytes from the EAE mice were obtained by performing a standard mouse splenectomy and then processing the spleen. The spleen is place into a 70 um cell strainer. Using the plunger end of a syringe, the spleen is mashed through the cell strainer into a 50 ml conical tube and rinsed with 40 ml of media. The strainer is discarded and the cells are spun at 800×g for 3 minutes. The splenocyte suspension is then treated with an ammonium chloride lysing solution for 5 minutes to eliminate red blood cells, spun down and re-suspended in 10 ml of media. The process is performed aseptically in cases where the cells will be used downstream for tissue culture.

Immunofluorescent staining was performed as follows: Cells from spleen and spinal cord tissues were stained in a 96 well-plate (10⁶ cells per/well) with optimally titrated antibodies diluted in staining buffer (1×PBS, fetal bovine serum, sodium azide) for minimum 20 min at 4 C. Samples were acquired using either Accuri C6 (BD Biosciences). Data analysis was performed using the Accuri C6 software.

Immunofluorescent staining and cell sorting: Cells were stained in bulk at 50 million/ml using optimally titrated antibodies in IMAG buffer (1×PBS, EDTA, FBS, sodium azide) for minimum of 20 min at 4 C. Cells were sorted using a BD FACSAria™ III (100 micron nozzle, drop drive frequency 31.0 kHz, Sheath pressure 20.5 psi). In those cases where cells post sorting were used for tissue culture the tissue harvesting, processing, immunofluorescent staining and sorting were performed aseptically.

The cell culture of V cells was performed as follows. 25,000-30,000 sorted V cells were plated in a 24-well plate on a feeder layer of M2-10B4 cells (ATCC CRL-1972) treated for 3 hrs with 1 μg of Mitomycin C (SIGMA M4287). Treated M2-10B4 cells were washed twice with complete RPMI media prior to adding sorted cells. The V cells were grown in 50% MyeloCult media (Stemcell Technologies M5300) and 50% complete RPMI Media (RPMI-1640+7.5% FBS (low IgG Hyclone)+1% Penn/Strep/Glutamine+5×10⁻⁵ M 2ME). Colony formation was observed 3 days post sort.

Human blood was collected and assayed as follows. Human blood was collected from a donor and periferal blood mononuclear cells (PBMCs) were isolated using the Ficoll-Paque protocol. The cells were then stained with a cocktail of positive markers for V cells and basophil specific marker (2D7). Human blood was also collected from a donor with Type I diabetes and chronic inflammation. PBMCs were isolated with the Ficoll Paque protocol and PBMCs were stained with CD19 and a cocktail of positive markers for V cells and basophil specific markers.

### Example 10

### Spleen Control

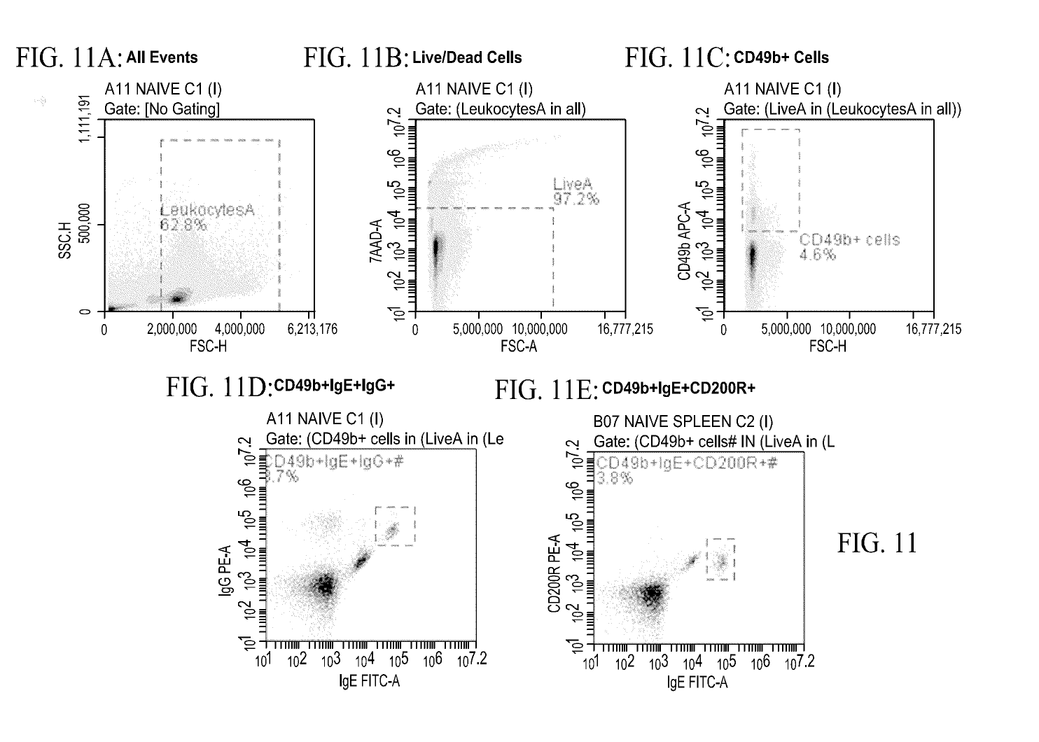

FIG. 11 shows a series of graphs of a population of V cells in the control HLA-DR2 mouse. Spleens from naïve HLA-DR2 1501 mice were isolated and single cell suspensions were prepared. Cells were then stained with an antibody cocktail that identifies V cells: IgG, IgE, CD49b and CD200R. Cells were also gated based on viability (7-AAD⁻). Vcells are IgG+IgE−CD49b+CD200R+. As shown in FIG. 11, the markers identify V cells, CD49b, IgG, and CD200R. Samples were acquired on Accuri C6 (BD Biosciences) and analyzed using the Accuri C6 software.

As such, a phenotype characteristic of the spleen of a healthy subject can be obtained.

### Example 11

### V Cell Population in the Spleen of Immunized HLA-DR2 1501 (EAE Model) Mouse

HLA-DR2 1501 male mice between age 8 and 12 wk were immunized subcutaneously at the lower back with MBP-85-99 peptide emulsified in CFA. On day 0 and 2 of immunization mice were injected with 200 ng of pertussis toxin. Tissues were isolated from EAE mice 14 days following immunization with clinical signs of disease severity of 2.5. Spleens from immunized (EAE mice) mice were isolated and single cell suspensions prepared. Cells were then stained with an antibody cocktail that identifies V cells: IgG, IgE, CD49b and CD200R. Cells were also gated based on viability (7-AAD⁻). Vcells are IgG+IgE− CD49b+CD200R+. Samples were acquired on Accuri C6 (BD Biosciences) and analyzed using the Accuri C6 software. The V-cell population in the spleen increased almost 2 fold in the disease state when compared to the control (3.7% vs 9%).

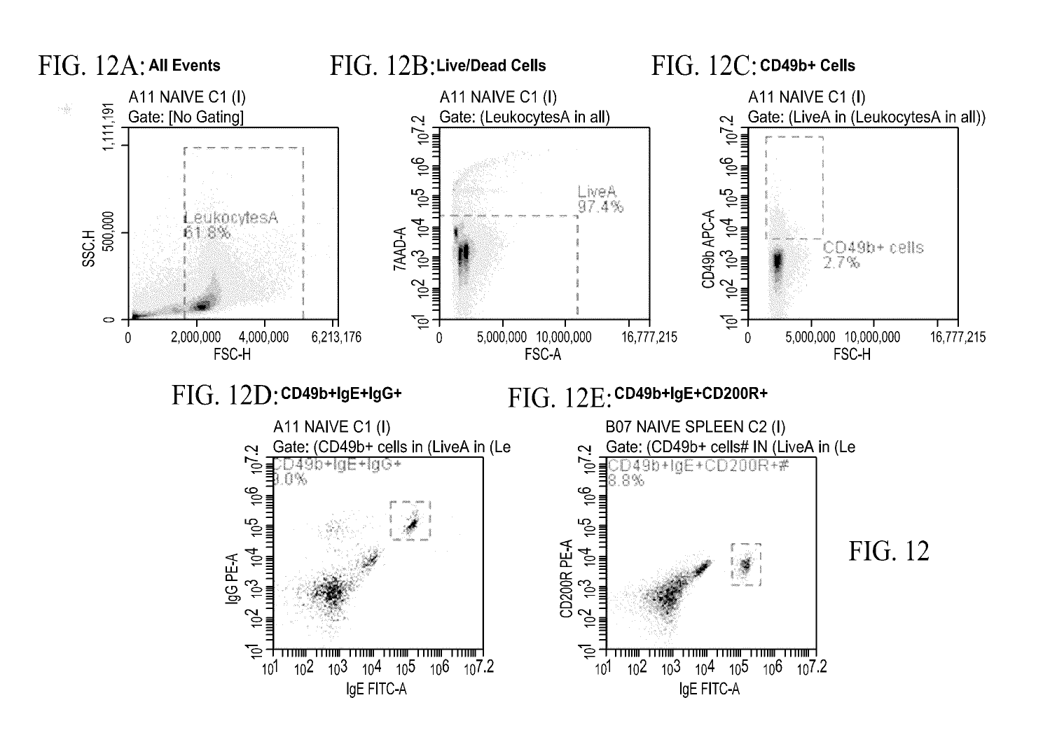

As shown in FIG. 12, Plot 4 (FIG. 12D) and Plot 5 (FIG. 12E), indicate an increase in V cell population (CD49b+ IgE+ IgG+, and CD49b+ IgE+ CD200R+, respectively) as compared to the control spleen sample of Example 10, FIG. 11, plot 4 (FIG. 11D) and Plot 5 (FIG. 11E)(CD49b+ IgE+ IgG+, and CD49b+ IgE+ CD200R+, respectively) for mice exhibiting disease symptoms of EAE.

As such, the V cell population was upregulated nearly two-fold (compared to healthy controls) in the spleen of a model of an inflammatory disease of the central nervous system.

### Example 12

### Presence of the V-Cell Population in the Spinal Cord of Immunized HLA-DR2 1501 (EAE Model) Mice

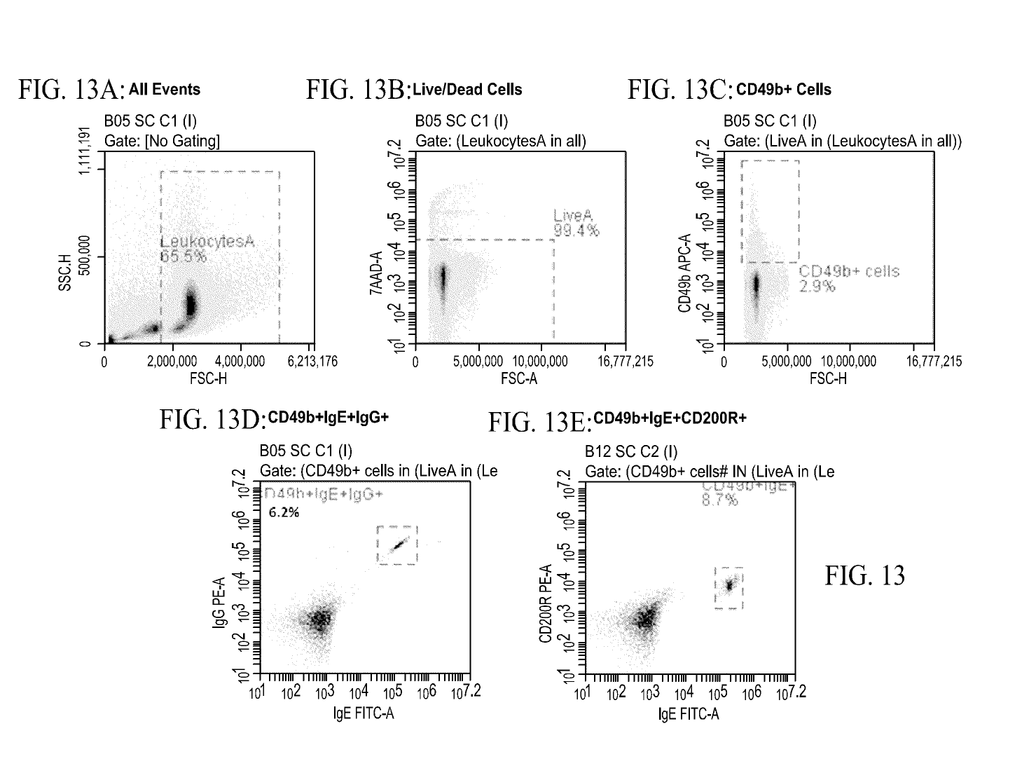

HLA-DR2 1501 male mice between age 8 and 12 wk were immunized subcutaneously at the lower back with MBP-85-99 peptide emulsified in CFA. On day 0 and 2 of immunization mice were injected with 200 ng of pertussis toxin. Tissues were isolated from EAE mice 14 days following immunization with clinical signs of disease severity of 2.5. Spinal cords from immunized animals (no cell infiltrate is observed in the spinal cord of a naïve animal) were isolated and single cell suspensions were prepared. Cells were then stained with an antibody cocktail that identifies V cells: IgG, IgE, CD49b and CD200R. Cells were also gated based on viability (7-AAD⁻). Vcells are IgG+IgE−CD49b+CD200R+. Samples were acquired on Accuri C6 (BD Biosciences) and analyzed using the Accuri C6 software. As shown in FIG. 13, markers were used to identify V cells as shown specifically in Plots 4 and 5 (CD49b+ IgE+ IgG+, and CD49b+ IgE+ CD200R+, respectively)(FIGS. 13D and 13E, respectively).

As such, V cells were present in the spinal cord in a model of an inflammatory disease of the central nervous system, while V cells were not present in the spinal cord of healthy controls.

### Example 13

### Phenotypic Characterization of Naïve V Cells in Human Peripheral Blood from a Donor not Suffering from any Major Illness or Disease

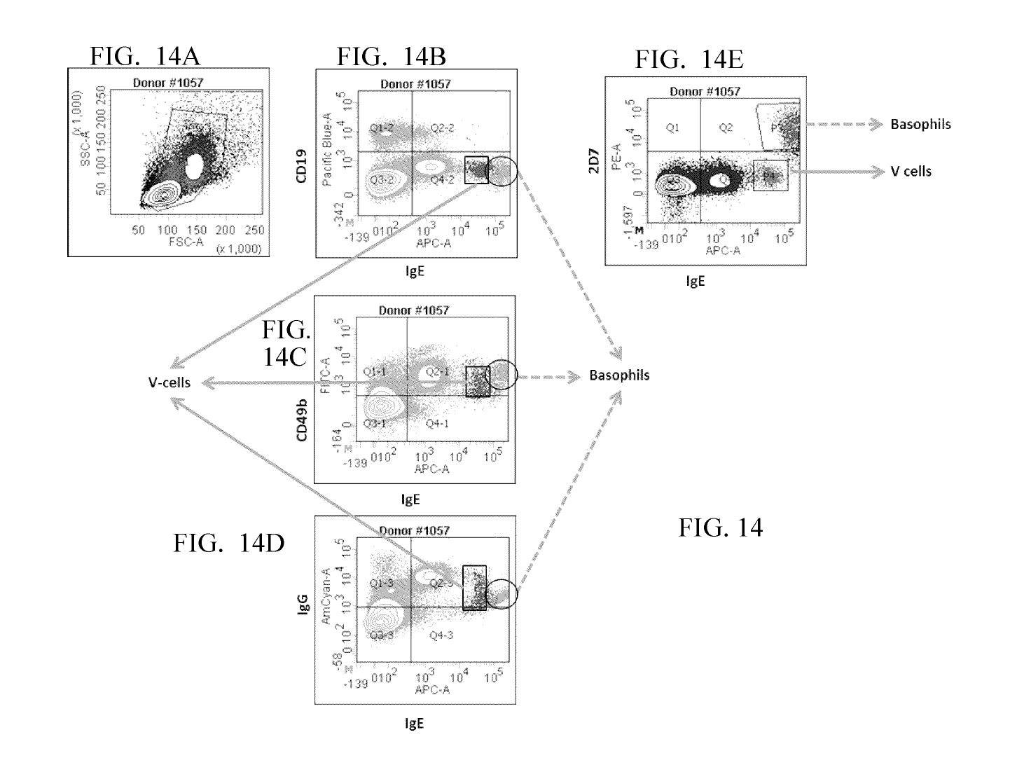

Human blood was collected from a donor and PBMCs were isolated using the Ficoll-Paque protocol. PBMCs were then stained with CD19 and a cocktail of positive markers for V cells (CD49b, IgG, IgE) and Basophil-specific marker 2D7. Due to the fact that V-cells and basophils can share a large number of cell surface markers, 2D7 can serve as a useful negative marker to separate these two populations. As such, V-cells can be identified as CD19−CD49b+IgG+IgE+2D7−. As shown in FIG. 14, the control has a presence of V cells as indicated by the presence of CD19, 2D7, CD49b, and IgG markers.

As such, V cells were present in PBMC samples of a healthy control.

### Example 14

### Phenotypic Characterization of a Patient Suffering Diabetes Mellitus Type I and Chronic Inflammation

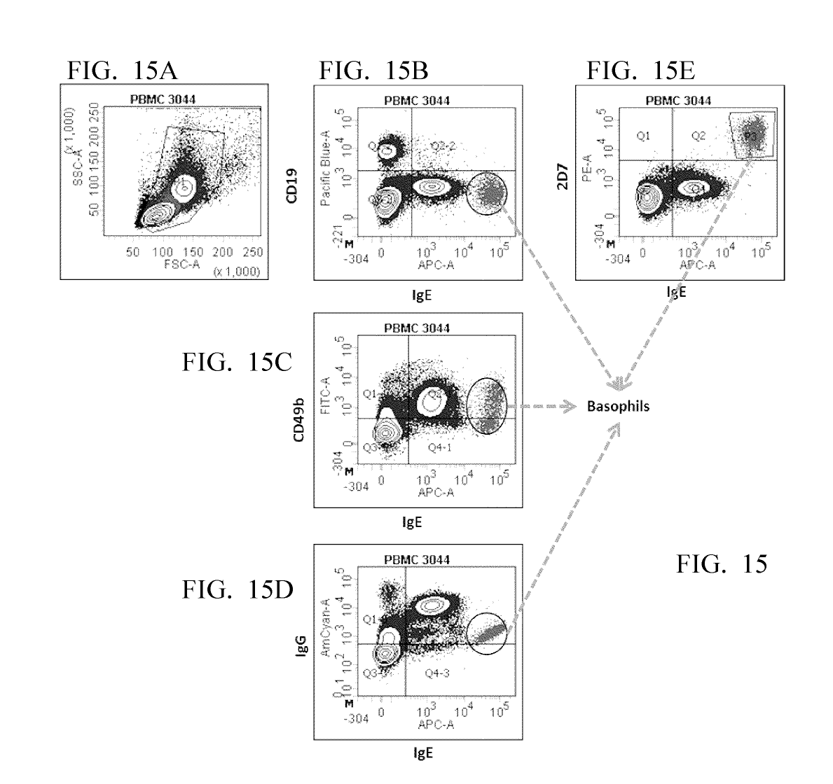

Human blood was collected from a donor that suffers Type I Diabetes and chronic inflamation from the extremities. PBMCs were isolated using the Ficoll-Paque protocol. PBMCs were then stained with CD19 and a cocktail of positive markers for V cells (CD49b, IgG, IgE) and Basophil-specific marker 2D7. When compared to a control sample (FIG. 4), it is easily established that this sample lacks the presence of V-cells and only basophils can be detected. As shown in FIG. 15, and in comparison to FIG. 14, there is a similar presence of basophils, but a lack of V cells in patient suffering from Diabetes mellitus Type I and chronic inflammation.

As such, V cells were absent from the PBMC samples of a subject suffering from Type I Diabetes and chronic inflammation from the extremities.

### ADDITIONAL EMBODIMENTS

In some aspects, a method of producing an antibody is provided. The method can comprise administering an antigen to a host organism. The method can comprise isolating at least one Ig-producing cell of the host organism, in which the cell comprises at least an IgG or IgE immunoglobulin that binds specifically to the antigen, and in which the cell is IgG+ IgE+ CD49b+, negative for B-cell specific markers and basophil-specific markers (2D7 and/or BB1), and positive for at least one of CD16/CD32, CD24, CD43, CD45, CD48, CD54, CD79b, CD123, CD200R, CD244.2, or FcεR1. The method can comprise generating an IgG or IgE immunoglobulin comprising a heavy chain variable region encoded by rearranged variable gene segments of the cell, and a light chain variable region encoded by rearranged variable gene segments of the cell. The method can comprise at least one of: (a) generating a first nucleic acid sequence of rearranged variable gene segments of the cell encoding the heavy chain variable region, and a second nucleic acid sequence of rearranged variable gene segments of the cell encoding the light chain variable region; or (b) culturing a plurality of antibody-producing cells comprising genomic variable gene rearrangements encoding a heavy chain variable region of immunoglobulin and a light chain variable region of the immunoglobulin. In some embodiments, the IgG or IgE immunoglobulin that binds specifically to the antigen is produced at least within about 10 days after first administering the antigen to the host organism. In some embodiments, the cell comprising at least an IgG or IgE immunoglobulin that binds specifically to the antigen is identified without the use of labeled antigen. In some embodiments, the immunoglobulin that binds specifically to the antigen is surface-bound. In some embodiments, the method further comprises engineering a humanized antibody comprising at least an HCDR1 of the heavy chain variable region, an HCDR2 of the heavy chain variable region, an HCDR3 of the heavy chain variable region, an LCDR1 of the light chain variable region, an LCDR2 of the light chain variable region, and an LCDR3 of the light chain variable region. In some embodiments, the host organism is immunocompromised, and wherein prior to administering an antigen to the host organism, a naïve IgG+ IgE+ cell negative for B-cell specific markers and basophil-specific markers (2D7 and BB1), and positive for at least one of CD16/CD32, CD24, CD43, CD45, CD48, CD54, CD79b, CD123, CD200R, CD244.2, or FcεR1 is delivered to the host organism. In some embodiments, the antigen is administered to the host organism only once.

According to some aspects, a complex is provided. The complex can comprise an isolated antibody-producing cell; at least one of an IgE-specific antibody, CD49b-specific antibody, or CD200R-specific antibody bound to the cell; and an IgG-specific antibody bound to the cell, in which the complex is not specifically bound by any of an antibody targeting B220, CD19, or CD20. In some embodiments, the complex is not specifically bound by an antibody targeting a B cell-specific marker. In some embodiments, each of the bound antibodies comprises a detectable marker, is attached to a separable phase, or comprises a detectable marker and is attached to a separable phase. In some embodiments, the separable phase comprises a magnetic bead. In some embodiments, the cell comprises a polymorphonuclear or annular-shaped nucleus.

According to some aspects, a method of detecting the presence of a cell capable of producing antigen-specific antibody. The method can comprise providing a population of mammalian cells. The method can comprise detecting from the population the presence or absence of one or more IgG+ IgE+ cells, in which the IgG+ IgE+ cells are negative for B-cell specific markers and basophil-specific markers (2D7 and BB1) and positive for at least one of CD49b, CD16/CD32, CD24, CD43, CD45, CD48, CD54, CD79b, CD123, CD200R, CD244.2, or FcεR1, and in which the IgG+ IgE+ cells are capable of producing an antibody. In some embodiments, the IgG+ IgE+ cells are positive for CD49b. In some embodiments, the method comprises at least one of: (a) contacting the population of mammalian cells with: an antibody that specifically binds to CD49b; an antibody that specifically binds IgE, an antibody that specifically binds to IgG; and an antibody that specifically binds to a B cell, and determining the presence or absence of one or more IgG+ IgE+ CD49b+ cells that are negative for B-cell specific markers and basophil-specific markers (2D7 and BB1); or (b) contacting the population of mammalian cells with: an antibody that specifically binds to CD49b, an antibody that specifically binds to IgG, an antibody that specifically binds IgE, and an antibody that specifically binds to B220, and determining the presence or absence of one or more B220− IgG+ IgE+ CD49b+ 2D7− BB1− cells; or (c) contacting the population of mammalian cells with: an antibody that specifically binds to CD49b, an antibody that specifically binds to IgG; an antibody that specifically binds IgE, and an antibody that specifically binds to CD19 or CD20, and determining the presence or absence of one or more CD19− IgG+ IgE+ CD49b+ cells or CD20− IgG+ IgE+ CD49b+ 2D7− BB1− cells; or (d) contacting the population of mammalian cells with: an antibody that specifically binds to IgE, an antibody that specifically binds to IgG, and an antibody that specifically binds to a CD19 or CD20, and determining the presence or absence of one or more IgE+ IgG+ CD19− cell or IgE+ IgG+CD20− 2D7− BB1− cells; or (e) contacting the population of mammalian cells with: an antibody that specifically binds to IgE, an antibody that specifically binds to IgG, and an antibody that specifically binds to CD20, and determining the presence or absence of a IgE+ IgG+ CD20− 2D7− BB1− cell; or (f) contacting the population of mammalian cells with an antibody that specifically binds to IgE, an antibody that specifically binds to IgG, and an antibody that specifically binds to B220, and determining the presence or absence of one or more IgE+ IgG+ B220− 2D7− BB1− cell; or (g) contacting the population of mammalian cells with: an antibody that specifically binds to IgE, an antibody that specifically binds to IgG, and an antibody that specifically binds to CD19 or CD20, and determining the presence or absence of a IgE+ IgG+ CD19− 2D7− BB1− cell or IgE+ IgG+ CD20− 2D7− BB1− cell; or (e) contacting the population of mammalian cells with: an antibody that specifically binds to IgG, an antibody that specifically binds to CD200R, and an antibody that specifically binds to B220, and determining the presence or absence of a IgG+ CD200R+ B220− 2D7− BB1− cell; or (f) contacting the population of mammalian cells with an antibody that specifically binds to IgG, an antibody that specifically binds to at least one of CD16/CD32, CD24, CD43, CD45, CD48, CD54, CD79b, CD123, CD200R, CD244.2, or FcεR1, and with an antibody that specifically binds to a CD19 or CD20; and determining the presence or absence of a IgG+CD19− or IgG+ CD20− cell that is positive for at least one of at least one of CD16/CD32, CD24, CD43, CD45, CD48, CD54, CD79b, CD123, CD200R, CD244.2, or FcεR1 and negative for B cell markers and basophil markers (e.g., 2D7 and/or BB1); or (g) contacting the population of mammalian cells with antigen (Ag) and detecting binding or an absence of binding of the IgG+ IgE+ cell to Ag; or (h) contacting the population of mammalian cells with an antibody that specifically binds to CD49b+, and determining the presence or absence of CD49b+ IgG+ IgE+ cells that are negative for B-cell specific markers and basophil-specific markers (2D7 and/or BB1). In some embodiments, at least one of: (a) the antibody that specifically binds to a B cell specifically binds to an antigen selected from the group consisting of B220, CD5, CD19, CD20, CD21/CD35, CD22.2, CD72, GL-7, IgD, IgM, Ly6-k, Ly6-D, Ly-51 CD127, CD138, CD154, AA4.1 and Pax-5; or (b) the antibody that specifically binds to a B cell specifically binds to B220; or (c) the antibody that specifically binds to a B cell specifically binds to CD19 or CD20; or (d) the presence or absence of IgE is detected at the same time as the presence or absence of IgG; or (e) the population of mammalian cells comprises human cells; or (f) the population of mammalian cells comprises murine cells.

According to some aspects, a method of enriching a cell-containing sample for IgG+ IgE+ cells capable of producing antibodies, in which the IgG+ IgE+ cells are negative for B-cell specific markers and negative for basophil-specific markers (2D7 and/or BB1) and positive for at least one of CD49b, CD16/CD32, CD24, CD43, CD45, CD48, CD54, CD79b, CD123, CD200R, CD244.2, or FcεR1 is provided. The method can comprise contacting the sample with an enrichment antibody that specifically binds to B cells. The method can comprise contacting the sample with at least one of: an enrichment antibody that specifically binds to T cells, an enrichment antibody that specifically binds to monocytes; an enrichment antibody that specifically binds to dendritic cells; an enrichment antibody that specifically binds to NK cells, an enrichment antibody that specifically binds to erythrocytes, an enrichment antibody that specifically binds to hematopoietic stem cells, and an enrichment antibody that specifically binds to basophils, in which none of the enrichment antibodies binds specifically to B220−IgG+ IgE+ CD49b+ CD200R+ cells, CD19− IgG+ IgE+ CD49b+ CD200R+ cells, or CD20−IgG+ IgE+ CD49b+ CD200R+ cells. In some embodiments, the sample is contacted with two of the listed enrichment antibodies. In some embodiments, the sample is contacted with three of the listed enrichment antibodies. In some embodiments, the sample is contacted with four of the listed enrichment antibodies. The method can and separating at least one of the IgG+ IgE+ cells capable of producing antibody with at least one enrichment antibody bound to said at least one IgG+ IgE+ cell from other cells of the sample. In some embodiments, the IgG+ IgE+ cells are also CD49b+ CD200R+. In some embodiments, the method further comprises detecting the presence of at least one of the IgG+ IgE+ CD49b+ 2D7− BB1− cells. In some embodiments, the enrichment antibody that specifically binds to B cells binds specifically to one of B220, CD19, CD20, CD5, CD21/CD35, CD22.2, CD72, GL-7, IgD, IgM, Ly6-k, Ly6-D, Ly-51, CD127, CD138, CD154, AA4.1 or Pax-5. In some embodiments, the enrichment antibody that specifically binds to T cells binds specifically to one of CD1d, CD3, CD4, CD8, CD25, CD38 or CD134. In some embodiments, the enrichment antibody that specifically binds to dendritic cells binds specifically to one of CD11c or CD273. In some embodiments, the enrichment antibody that specifically binds to NK cells binds specifically to one of NK1.1, NK1.2, CD49a, CD122 or CD226/NKp46. In some embodiments, the enrichment antibody that specifically binds to hematopoietic stem cells binds specifically to one of CD34, Sca-1, c-Kit or CD150. In some embodiments, the enrichment antibody that specifically binds to basophils specifically binds to CD123.

According to some aspects, a kit for the detection of antibody-producing cells is provided. The kit can comprise a first antibody that specifically binds to IgG, in which the first antibody comprises a first detectable marker. The kit can comprise a second antibody that specifically binds to IgE, in which the second antibody comprises a second detectable marker. The kit can comprise a third antibody that specifically binds to CD49b, in which the third antibody comprises a third detectable marker. The kit can comprise a fourth antibody that specifically binds to an antigen selected from the group consisting of CD19, CD20, or B220, in which the fourth antibody comprises a fourth detectable marker. In some embodiments, the first detectable marker, the second detectable marker, the third detectable marker, and the fourth detectable marker are each different from one another. In some embodiments, the kit further comprises at least one of: (a) a fifth antibody that binds specifically to CD200R, CD244.2, or FcεR1, wherein the fifth antibody comprises a fifth detectable marker that is different from the first, second, third, and fourth detectable markers; (b) at least one of an antibody that binds specifically to CD24, CD43, CD45, and CD48; or (c) at least one of an antibody that binds specifically to CD1d, CD3, CD4, CD8, CD25, CD38 CD134, CD11c, CD273, CD49a, CD122, CD123, CD200R, CD226/NKp46, CD34, Sca-1, c-Kit, CD150, CD11b, Ly-6G, or NKP46; or (d) at least one of an antibody that binds specifically to CD5, CD21/CD35, CD22.2, CD72, GL-7, IgD, IgM, Ly6-k, Ly6-D, Ly-51, CD127, CD138, CD154, AA4.1 and Pax-5 and (d) at least one of an antibody that binds specifically to 2D7 and/or BB1.

According to some aspects, a kit for enriching a sample for a population of IgG+ IgE+ CD49b+ CD200R+ antibody-producing cells that are negative for B cell-specific markers is provided. The kit can comprise an enrichment antibody that specifically binds to an antigen selected from the group consisting of B220, CD19, CD20, CD5, CD21/CD35, CD22.2, CD72, GL-7, IgD, IgM, Ly6-k, Ly6-D, Ly-51, CD127, CD138, CD154, AA4.1 and Pax-5. The kit can comprise at least one of: an enrichment antibody that specifically binds to T Cells; an enrichment antibody that specifically binds to Monocytes, an enrichment antibody that specifically binds to Dendritic Cells; an enrichment antibody that specifically binds to NK Cells, an enrichment antibody that specifically binds to hematopoietic stem cells; or an enrichment antibody that specifically binds to basophils, and a collection of separable phases bound to or capable of specifically complexing with the antibodies of the kit, in which wherein the enrichment antibodies of the kit do not bind to the IgG+ IgE+ CD49b+ antibody producing cells that are negative for B-cell specific markers. In some embodiments, at least one of (a) the collection of separable phases comprises magnetic beads; or (b) the enrichment antibodies are biotinylated and the separable phase comprises streptavidin; or (c) the enrichment antibodies comprise a detectable marker, and the separable phase comprises a collection of separable phase particles that bind specifically to the detectable marker; or (d) the enrichment antibodies comprise a detectable marker, and the separable phase comprises a collection of magnetic particles that bind specifically to the detectable marker.

### Methods of Determining Disease States

It is reported herein that V cells have been found in spinal cords in mice suffering from Experimental Autoimmune Encephalomyelitis (EAE), but not in healthy controls. EAE can model a variety of human CNS demyelinating diseases, including multiple sclerosis (MS) and acute disseminated encephalomyelitis (ADEM). Without being limited to any theory, it is contemplated herein that detecting the presence or absence of V cells in the spinal cord of a mammalian subject (for example a human or a mouse) can be useful for determining a disease state in a subject, for example a CNS demyelinating disease such as multiple sclerosis (MS) or acute disseminated encephalomyelitis (ADEM). Accordingly, in some embodiments an inflammatory disease state of the CNS is determined, for example a demyelinating disease such as MS or ADEM. The presence or absence of the disease state can be determined by determining the presence or absence of V cells in a sample from a subject, or by determining an increase or upregulation of V cells in a sample from the subject.

It is reported herein that peripheral blood mononuclear cell (PBMC) samples from subjects suffering from both type I diabetes and chronic inflammation lack a V-cell population when compared to control healthy subjects. Without being limited to any theory, it is contemplated herein that detecting the presence or absence of V cells in a sample of a mammalian subject can be useful for determining a disease state in the subject, for example type I diabetes and/or chronic inflammation. Accordingly, in some embodiments a type I diabetes or chronic inflammatory disease state is determined. The presence or absence of the disease state can be determined by determining the absence or presence of V cells in a sample from a subject.

When determining a disease state in a subject, it can be useful to determine the presence or absence of antibody-producing cells or cells capable of producing antibody, for example V cells or subpopulations thereof. Methods of determining disease states by identifying antigen-specific antibody producing cells or cells capable of producing antibody such as V cells can also be useful for various other applications, for example, researching antibody producing cells, and drug screening.

It is contemplated herein that V cells express IgE, which is implicated in allergy, and that V cells can be associated with a number of inflammatory diseases. Exemplary inflammatory diseases contemplated to be associated with V cells, and which can be determined in accordance with some embodiments herien include, but are not limited to type I diabetes, chronic inflammation, multiple sclerosis (MS) or acute disseminated encephalomyelitis (ADEM), Alzheimer's disease, ankylosing spondylitis, aneurysm, arthritis (osteoarthritis, rheumatoid arthritis (RA), psoriatic arthritis, asthma, atherosclerosis, Crohn's disease, immune response in cystic fibrosis, pelvic inflammatory disease, colitis, dermatitis, diverticulitis, fibromyalgia, hepatitis, irritable bowel syndrome (IBS), systemic lupus erythematous (SLE), stroke, nephritis, Parkinson's disease, ulcerative colitis, autoinflammatory diseases, familial Mediterranean fever (FMf), neonatal onset multisystem inflammatory disease (NOMID), tumor necrosis factor receptor-associated periodic syndrome (TRAPS), deficiency of the interleukin-1 receptor antagonist (DIRA), or Behçet's disease.

In some embodiments, ectopic V cells (i.e. the presence of V cells in a tissue that does not contain V cells in healthy individuals), and/or a fold increase of V cells in a tissue that normally comprises V cells can be determinative or predictive of an inflammatory disease state. The presence or absence of V cells can be determined in a variety of tissues in which inflammation or cell inflammation can be observed in accordance with some embodiments herein. Exemplary tissues include, but are not limited to blood, spinal cord, spleen, synovial fluid, bronchial lavage cells, and thyroid cell aspirate. In some embodiments, the tissue comprises a tissue in which inflammation and cell infiltration is observed and a cell sample can be taken. In some embodiments, the cell sample can come from a cervical cytobrush. In some embodiments, a fold increase of V cells is detected in a tissue that contains V cells in healthy individuals, for example, spleen, bone marrow, or blood (e.g. PBMC's). In some-embodiments a fold increase in V-cells of at least about 1.5× compared to a healthy control, for example 1.5×, 2×, 2.5×, 3×, 3.5×, 4×, 5×, 6×, 7×, 8×, 9×, or 10×, in bone marrow, spleen, or blood indicates the inflammatory disease state. In some embodiments, ectopic V cells in the central nervous system, spinal cord, synovial fluid, bronchial lavage cells, or thyroid cell aspirate indicates the inflammatory disease state. In some embodiments, ectopic V cells in the central nervous system, for example spinal cord tissues, indicates the inflammatory disease state. In some embodiments, a fold increase of V cells in any of the spleen, bone marrow, or blood (e.g. PBMC's) indicates an autoinflammatory disease state. In some embodiments, the disease state comprises multiple sclerosis (MS) or acute disseminated encephalomyelitis (ADEM). In some embodiments, the increase indicating the presence of a disease state comprises a statistically significant increase.

In some embodiments, decreased expression or an absence of V cells in a tissue that normally comprises V cells indicates a disease state. In some embodiments, absence of V cells from spleen, bone marrow, or blood (e.g. PBMC's) indicates a disease state. In some embodiments at least a 2-fold decrease in V cells (that is a decrease in which the fold number is 2 or larger) compared to a healthy control, for example, 2×, 2.5×, 3×, 3.5×, 4×, 5×, 6×, 7×, 8×, 9×, or 10×, in bone marrow, spleen, or blood indicates the inflammatory disease state. In some embodiments, the disease state comprises chronic inflammation or type I diabetes. In some embodiments, the decrease indicating the presence of a disease state comprises a statistically significant decrease.

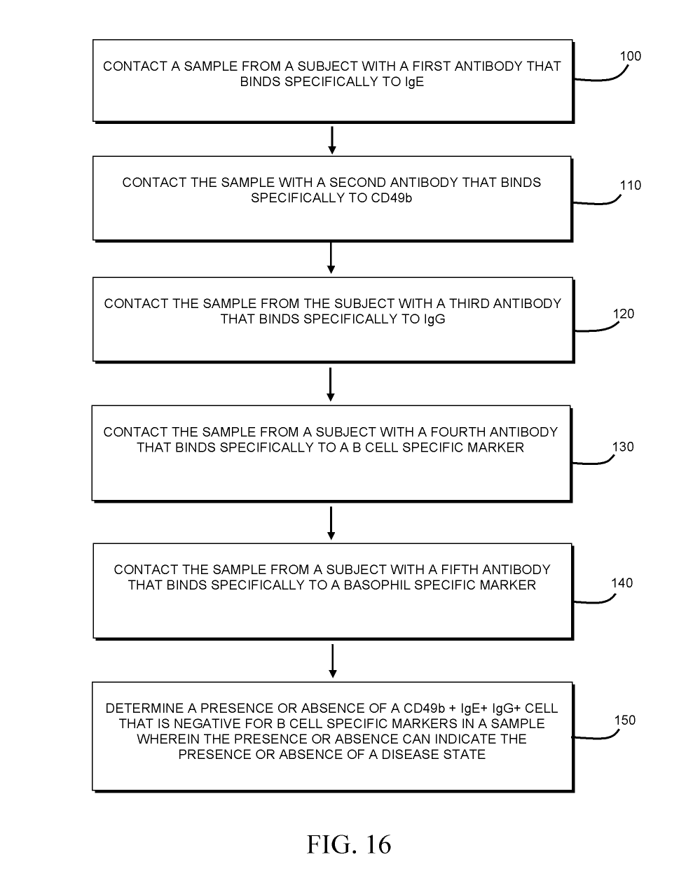

FIG. 16 is a flow diagram illustrating a method of determining a disease state according to some embodiments herein. The method can comprise contacting a sample from a subject with a first antibody that binds specifically to IgE 100. In some embodiments, the sample comprises a spinal cord sample, spleen sample, or PBMCs. The method can comprise contacting the sample with a second antibody that binds specifically to CD49b 110. The method can comprise contacting the sample with a third antibody that binds specifically to IgG 120. The method can comprise contacting the sample with a fourth antibody that binds specifically to a B cell-specific marker 130. The method can comprise contacting the sample with a fourth antibody that binds specifically to a basophil-specific marker 140. The method can comprise determining the presence or absence of a CD49b+ IgE+ IgG+ cell that is negative for B cell-specific markers in the sample, wherein the presence or absence indicates a disease state in a subject 150. One skilled in the art will appreciate that, for this and other processes and methods disclosed herein, the functions performed in the processes and methods may be implemented in differing order. Furthermore, the outlined steps are only provided as examples, and some of the steps and operations may be optional, combined into fewer steps and operations, or expanded into additional steps and operations without detracting from the essence of the disclosed embodiments.

Accordingly, some aspects include methods of determining a disease state by the presence or absence of cell types disclosed herein, for example V cells. The methods can include detecting the presence or absence of one or more V cells from in a sample from a subject. In some embodiments, for example methods of determining an inflammatory disease of the central nervous system, the sample comprises a spinal cord sample or spleen sample. In some embodiments, the sample comprises cells from a cervical cytobrush.

In some embodiments, the sample is freshly harvested from the subject. In some embodiments, the sample is harvested from the subject, and then preserved for subsequent determination of the presence or absence of V cells. In some embodiments, the sample is frozen to preserve it for subsequent determination of the presence or absence of V cells. The presence of V cells in the spinal cord sample, or upregulation of V cells in the spleen can indicate an inflammatory disease of the central nervous system, for example MS or ADEM as described herein. The presence of V cells in a sample from a cervical cytobrush can also indicate an inflammatory disease. In some embodiments, for example methods of determining type I diabetes or chronic inflammation, the sample comprises PBMCs. The absence of V cells from the sample comprising PBMCs can determine the presence of type I diabetes or chronic inflammation. In some embodiments, the method comprises determining the presence or absence of one or more V cells in the sample. The V cells can be identified as described herein, for example IgG+ IgE+ B220− 2D7− cells that are positive for at least one additional marker indicated in Table 1.1.

### Methods of Increasing Counts of V Cells in a Subject

It is reported herein that some disease states, for example type I diabetes and chronic inflammation are characterized by an absence of V cells from tissues or cell populations that normally comprise V cells, for example PBMCs. Without being limited by any theory, it is contemplated that V cells might be responsible for keeping in control the cell homeostasis of the subject by regulating other cell subsets. Lack of these cells could be associated with autoimmunity, inflammation or allergy. Accordingly, it is contemplated herein that restoration of a V cell population in the subject can be useful in ameliorating or treating the disease state.

As such, some embodiments include methods of increasing the V cells that produce antigen-specific antibody in a subject. A population of cells can be administered to host mammal. The population can comprise at least 5% non-B-cell lineage antibody producing cells, wherein the non-B-cell-lineage antibody-producing cells are IgG+, IgE+, CD49b+, negative for B cell-specific markers, and negative for basophil-specific markers. The non-B-cell-lineage antibody-producing cells can be IgG+, IgE+, CD49b+, negative for B cell-specific markers, and negative for basophil-specific markers. In some embodiments, the cells are negative for DD7 and/or BB1. In some embodiments, the cells are negative for at least one of CD19 and/or CD20. In some embodiments, the cells are allogeneic cells. In some embodiments, the cells are autologous cells. In some embodiments, the cells are positive for at least one of CD49b, CD16/CD32, CD24, CD45, CD48, CD54, CD79b, CD123, CD200R, CD244.2, or FcεR1. In some embodiments, the administering is performed by injection of a population of cells. In some embodiments, the injection is intravenous. In some embodiments, the injection is peritoneal. In some embodiments, the cells are infused. In some embodiments, the cells are delivered via a catheter. In some embodiments, the administered V cell population expands after administration to the subject.

In some embodiments, the subject is a mammal. In some embodiments, the subject is a human. In some embodiments, subject is a non-human mammal, for example a non-human primate, a cow, a horse, a dog, a cat, or a rodent. In some embodiments, the subject is a rodent. In some embodiments, the subject is determined to have a reduction or absence of V cells in at least one tissue or cell population that comprises V cells in healthy subjects, for example PBMCs.

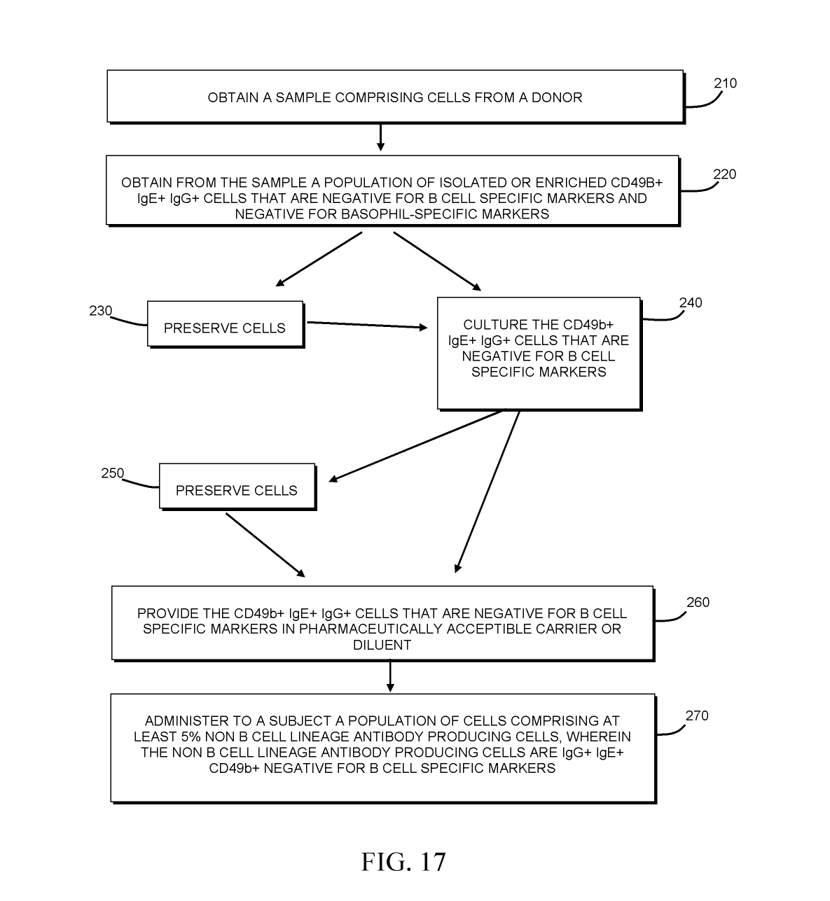

FIG. 17 is a flow diagram illustrating a method of increasing an antibody-producing cell count in a subject according to some embodiments herein. The method can comprise obtaining from a sample comprising cells from a donor 210. The method can comprise obtaining from the sample a population of isolated or enriched CD49b+ IgE+ IgG+ cells that are negative for B cell specific markers 220. In some embodiments the CD49b+ IgE+ IgG+ cells are negative for basophil-specific markers. The method can optionally comprise preserving the cells for future use 230. The method can comprise culturing the CD49b+ IgE+ IgG+ cells that are negative for B cell specific markers 240. In some embodiments, the CD49b+ IgE+ IgG+ cells are cultured for up to about two weeks, for example, 1, 2, 3, 4, 5, 6, 7, 8, 9, 10, 11, 12, 13, or 14 days. The method can optionally comprise preserving the cultured cells for future use 250. In some embodiments, preserving comprises cryogenically preserving. In some embodiments, the CD49b+ IgE+ IgG+ cells are provided in a pharmaceutically acceptable carrier or diluent 260. The method can comprise administering the cells comprising at least 5% non B cell lineage antibody producing cells wherein the non-B cell lineage antibody producing cells are IgG+ IgE+ CD49b+, negative for B cell-specific markers to a subject 270. In some embodiments, the non-B cell lineage antibody producing cells are negative for basophil-specific markers. One skilled in the art will appreciate that, for this and other processes and methods disclosed herein, the functions performed in the processes and methods may be implemented in differing order. Furthermore, the outlined steps are only provided as examples, and some of the steps and operations may be optional, combined into fewer steps and operations, or expanded into additional steps and operations without detracting from the essence of the disclosed embodiments.

In some embodiments, a sample comprising cells of the hematopoietic lineage is harvested from a donor. In some embodiments, the sample comprises a sample from at least one V-cell containing tissue, for example spleen, bone marrow, or blood. In some embodiment, the sample is enriched for V cells. In some embodiments, the sample is enriched for naïve V cells as described herein. For example, non-V cells can be removed from the population, for example based on markers for which V cells are negative (see Table 1.2). In some embodiments, one or more of B cells, T cells, basophils, dendritic cells, NK cells, and/or hematopoietic stem cells are removed. The non-V cells can be removed via a cell separation device. In some embodiments, V cells are isolated from the sample, for example via flow cytometry. In some embodiments, isolated or enriched V cells are cultured in vitro. In some embodiments, V cells are preserved for later administration, for example via cryopreservation. V cells are administered to a subject via a variety of means as described herein. In some embodiments, the subject receiving the V cells is also the donor. In some embodiments, the subject is different from the donor.

In some embodiments, the subject has a genetic disease. In some embodiments, the subject suffers from type I diabetes. In some embodiments, the subject suffers from chronic inflammation. In some embodiments, the subject suffers from an inflammatory disease of the nervous system. In some embodiments, the subject suffers from human CNS demyelinating disease, multiple sclerosis, or acute disseminated encephalomyelitis. In some embodiments, the subject suffers from an autoimmune disease.

### V Cells for Administration

Some embodiments include isolated or enriched populations cells capable of producing antibody, for example V cells. In some embodiments, a method of increasing an antibody-producing cell count in a subject comprises administering a population of cells comprising at least 5% non-B-cell lineage antibody producing cells, for example about 5%, 6%, 7%, 8%, 9%, 10%, 15%, 20%, 25%, 30%, 35%, 40%, 45%, 50%, 60%, 70%, 80%, 90%, 95%, 98%, 99%, or 99.9, including ranges between any two of the listed values. The non-B-cell-lineage antibody-producing cells can be IgG+, IgE+, CD49b+, negative for B cell-specific markers, and negative for basophil-specific markers. In some embodiments, a composition or medicament for administration comprises the isolated V cells and a pharmaceutically acceptable diluent or carrier. In some embodiments, the isolated cells capable of producing antibody comprise naïve V cells. The isolated or enriched populations of V cells can be administered to a subject as described herein. In some embodiments, the administering is performed by injection of a population of cells. In some embodiments, the injection is intravenous. In some embodiments, the injection is peritoneal. In some embodiments, the cells are infused. In some embodiments, the cells are delivered via a catheter. In some embodiments, the administered V cell population expands after administration to the subject.

The following references relate to identification and characterization of cells of the hematopoietic lineage:
Mc-Heyzer-Williams, L. J., M. Cool, M. G. Mc-Heyzer-Williams. 2000. Antigen-specific B cell memory: expression and replenishment of a novel B220(−) memory B cell compartment. J. Exp. Med. 191:1149-1166.
Cascalho, M., J. Wong, J. Brown, H. M. Jack, C. Steinberg, M. Wabl. 2000. A B220(−), CD19(−) population of B cells in the peripheral blood of quasimonoclonal mice. Int. Immunol. 12:29-35.
Driver, D. J., L. J. Mc-Heyzer-Williams, M. Cool, D. B. Stetson, M. G. Mc-Heyzer-Williams. 2001. Development and maintenance of a B220-memory B cell compartment. J. Immunol. 167:1393-1405.
Bell J., D. Gray. Antigen-capturing Cells Can Masquerade as Memory B cells. J. Exp. Med. 197:1233-1244
Mack M., Schneider M. A., Moll C. et al. 2005. Identification of Antigen-Capturing Cells as Basophils. J. Immunol. 174:735-741.
Lee J. J., P. McGarry. 2006. When is a mouse basophil not a basophil? Blood. 109:859-861.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

While the present invention has been described in some detail for purposes of clarity and understanding, one skilled in the art will appreciate that various changes in form and detail can be made without departing from the true scope of the invention.

The term “comprising” as used herein is synonymous with “including,” “containing,” or “characterized by,” and is inclusive or open-ended and does not exclude additional, unrecited elements or method steps.

All numbers expressing quantities of ingredients, reaction conditions, and so forth used in the specification are to be understood as being modified in all instances by the term “about.” Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention.

The foregoing description and Examples detail certain embodiments. It will be appreciated, however, that no matter how detailed the foregoing may appear in text, the invention may be practiced in many ways and the invention should be construed in accordance with the appended claims and any equivalents thereof.

What is claimed is:

1. A kit for the detection of IgG+ IgE+ CD49b+ cells negative for B-cell-specific markers, negative for basophil-specific markers, and capable of producing antibody, the kit comprising:
a first antibody that specifically binds to IgG, wherein the first antibody comprises a first detectable marker;
a second antibody that specifically binds to IgE, wherein the second antibody comprises a second detectable marker;
a third antibody that specifically binds to CD49b, wherein the third antibody comprises a third detectable marker;
a fourth antibody that specifically binds to a B-cell-specific marker, wherein the fourth antibody comprises a fourth detectable marker; and
a fifth antibody that binds specifically to a basophil-specific marker, wherein the fifth antibody comprises a fifth detectable marker,
wherein the first detectable marker, the second detectable marker, the third detectable marker, the fourth detectable marker, are each different from one another, and
wherein the fourth detectable marker and the fifth detectable marker are either the same as each other or different from each other.

2. The kit of claim 1, wherein the fourth antibody specifically binds to a B-cell-specific marker selected from the group consisting of: B220, CD19, and CD20.

3. The kit of claim 1, wherein the basophil-specific marker comprises 2D7, BB1, or both.

4. The kit of claim 1, further comprising a sixth antibody that binds specifically to CD16/CD32, CD24, CD43, CD45, CD48, CD54, CD79b, CD123, CD200R, CD244.2, or FcεR1, wherein the sixth antibody comprises a sixth detectable marker that is different from the first, second, third, and fourth detectable markers.

5. The kit of claim 1, further comprising a seventh antibody that binds specifically to CD24, CD43, CD45, or CD48.

6. The kit of claim 1, further comprising an antibody that specifically binds to a cytokine selected from the group consisting of IL-4, TNF, IL-13, and Il-10, and that comprises a seventh detectable marker different from the first, second, third, fourth, and fifth detectable markers.

7. A kit for enriching a sample for a population of IgG+ IgE+ cells capable of producing antibody wherein the IgG+ IgE+ cells are negative for B-cell specific markers, negative for basophil-specific markers, and positive for at least one of CD16/CD32, CD24, CD43, CD45, CD48, CD54, CD79b, CD123, CD200R, CD244.2, or FcεR1, the kit comprising:
an enrichment antibody that specifically binds to an antigen selected from the group consisting of B220, CD19, CD20, CD5, CD21/CD35, CD22.2, CD72, GL-7, IgD, IgM, Ly6-k, Ly6-D, Ly-51, CD127, CD138, CD154, AA4.1 and Pax-5;
at least one of:
an enrichment antibody that specifically binds to T Cells;
an enrichment antibody that specifically binds to Monocytes;
an enrichment antibody that specifically binds to Dendritic Cells;
an enrichment antibody that specifically binds to NK Cells;
an enrichment antibody that specifically binds to hematopoietic stem cells; or
an enrichment antibody that specifically binds to basophils; and
a collection of separable phases bound to or capable of specifically complexing with the antibodies of the kit,
wherein the enrichment antibodies of the kit do not bind to the IgG+ IgE+ cells.

8. The kit of claim 7, wherein the enrichment antibody that specifically binds to basophils specifically binds to a marker from the group consisting of: 2D7 or BB1.

9. The kit of claim 7, wherein the IgG+ IgE+ cells are further CD49b+, and wherein the enrichment antibodies of the kit do not bind to the IgG+ IgE+ CD49b+ cells.

10. The kit of claim 7, wherein the collection of separable phases comprises magnetic particles.

11. The kit of claim 7, wherein the enrichment antibodies are biotinylated and the separable phase comprises streptavidin.

12. The kit of claim 7, wherein the enrichment antibodies comprise a detectable marker, and the separable phase comprises a collection of separable phase particles that bind specifically to the detectable marker.

13. The kit of claim 7, wherein the enrichment antibodies comprise a detectable marker, and the separable phase comprises a collection of magnetic particles that bind specifically to the detectable marker.

14. The kit of claim 7, wherein the kit comprises at least three of:
an enrichment antibody that specifically binds to T Cells;
an enrichment antibody that specifically binds to Monocytes;
an enrichment antibody that specifically binds to Dendritic Cells;
an enrichment antibody that specifically binds to NK Cells;
an enrichment antibody that specifically binds to hematopoietic stem cells; or
an enrichment antibody that specifically binds to basophils.

15. A method of detecting the presence of a cell capable of producing antigen-specific antibody, the method comprising:
providing a population of mammalian cells; and
detecting from the population the presence or absence of one or more IgG+ IgE+ cells, wherein the IgG+ IgE+ cells are negative for B-cell specific markers, negative for basophil-specific markers, and positive for at least one of CD49b, CD16/CD32, CD24, CD43, CD45, CD48, CD54, CD79b, CD123, CD200R, CD244.2, or FcεR1, and wherein the IgG+ IgE+ cells are capable of producing an antibody.

16. The method of claim 15, wherein the IgG+ IgE+ cells are positive for at least one of CD49b or CD200R.

17. The method of claim 15, wherein the detecting comprises:
contacting the population of mammalian cells with:
an antibody that specifically binds to CD49b;
an antibody that specifically binds IgE;
an antibody that specifically binds to IgG;
an antibody that specifically binds to a B cell; and
an antibody that binds specifically to a basophil; and
determining the presence or absence of one or more IgG+ IgE+ CD49b+ cells that are negative for B-cell specific markers and basophil-specific markers.

18. The method of claim 15, wherein the detecting comprises:
contacting the population of mammalian cells with:
an antibody that specifically binds to IgG;
an antibody that specifically binds to at least one of CD16/CD32, CD24, CD43, CD45, CD48, CD54, CD79b, CD123, CD200R, CD244.2, or FcεR1; and
an antibody that specifically binds to a CD19 or CD20; and
determining the presence or absence of a IgG+ CD19− or IgG+ CD20− cell that is positive for at least one of at least one of CD16/CD32, CD24, CD43, CD45, CD48, CD54, CD79b, CD123, CD200R, CD244.2, or FcεR1.

19. The method of claim 15, wherein the basophil-specific marker comprises 2D7, BB1, or both.

20. The method of claim 15, wherein the population of mammalian cells comprises at least one of: human cells or murine cells.
